(19) United States
(12) Patent Application Publication
(10) Pub. No.: US 2005/0288347 A1
(43) Pub. Date: Dec. 29, 2005

(54) Certain triazole-based compounds, compositions, and uses thereof

(76) Inventors: Carl Nicholas Hodge, Los Gatos, CA (US); John K. Dickson JR., Apex, NC (US); Ioana G. Popa-Burke, Durham, NC (US); Jose Serafin Mendoza, Chapel Hill, NC (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/090,956

(22) Filed: Mar. 25, 2005

Related U.S. Application Data
(60) Provisional application No. 60/556,795, filed on Mar. 26, 2004.
(60) Provisional application No. 60/638,944, filed on Dec. 23, 2004.

(51) Int. Cl. A61K 31/4196; C07D 249/08; C07D 249/14

(52) U.S. Cl. 514/383; 548/263.2; 548/264.8

(57) ABSTRACT

Thiotriazole-based chemical entities exhibiting ATP-utilizing enzyme inhibitory activity, methods of using such chemical entities, and compositions comprising such chemical entities, are described.

This application claims the benefit of U.S. Provisional Patent Application No. 60/556,795, filed Mar. 26, 2004 and of U.S. Provisional Patent Application No. 60/638,944, filed Dec. 23, 2004, each of which is incorporated herein by reference for all purposes.

Protein kinases encompass a large family of functionally and structurally related enzymes that are responsible for the control of a wide variety of cellular processes including signal transduction, metabolism, transcription, cell cycle progression, cytoskeletal rearrangement and cell movement, apoptosis, and differentiation. In general, protein kinases control protein activity by catalyzing the addition of a negatively charged phosphate group from a phosphate-containing molecule such as cyclic adenosine monophosphate (cAMP), adenosine diphosphate (ADP), and ATP, to other proteins. Protein phosphorylation in turn can modulate or regulate the functioning of a target protein. Protein phosphorylation is known to play a role in intercellular communication during development, in physiological responses and in homeostasis, and in the functioning of the nervous and immune systems.

The unregulated phosphorylation of proteins is known to be a cause of, or associated with the etiology of major diseases, such as Alzheimer's disease, stroke, diabetes, obesity, inflammation, cancer, and rheumatoid arthritis. Deregulated protein kinase activity and over expression of protein kinases has been implicated in the pathophysiology of a number of important human disorders. Furthermore, genetic mutations in protein kinases are implicated in a number of disorders and many toxins and pathogens exert their effects by altering the phosphorylation of intracellular proteins.

ATP-utilizing enzymes, such as protein kinases, therefore, represent a broad class of pharmacological targets of interest for the treatment of human disease. Most human protein kinases can further be grouped into seven major groups based on the deoxyribonucleic acid (DNA) sequence homologies identified as CAMK (calcium/calmodulin-dependent protein kinases), AGC (including PKA (protein kinase A), PKG (protein kinase G), PKC (protein kinase C) kinases), CK1 (casein kinases), CMGC (containing CDK (cyclin-dependent)), MAPK (mitogen activated), GSK3 (glycogen synthase) and CLK (CDC2-like) kinases), STE (homologs of yeast Sterile 7, Sterile 11, and Sterile 20 kinases), TK (tyrosine kinases), and TKL (tyrosine-kinase like).

The AGC protein kinase family includes AKT1, AKT2, AKT3, AURORA-A, MSK1, MSK2, P70S6K, PAK1, PKA, ROCK2, SGK1, PDK1, and RSK2 protein kinases. The CMGC protein kinase family includes the CDK1, CDK2/cyclinA, CDK2/cyclinE, CDK5, DYRK2, GSK3-α, GSK3-β, p38-α, p38-β, p38-δ, and p38-γ, and MAPK1 protein kinases. The CAMK protein kinase family includes the DAPK1, MAPKAPK2, MAPKAPK3, CHEK1, CHEK2, PRAK, c-TAK1, and PIM-1-kinase protein kinases. The TK protein kinase family includes the ABL1, CSK, FLT3, FYN, HCK, INSR, KIT, LCK, PDGFRR-α, LYNA, SYK, and SRC protein kinases. The STE protein kinase family includes PAK2 protein kinase.

The identification and development of chemical entities that inhibit the functioning of ATP-utilizing enzymes is therefore of considerable interest.

Provided is at least one chemical entity chosen from compounds of Formula I, 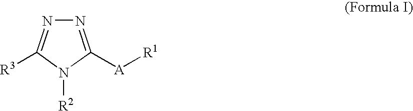
and pharmaceutically acceptable salts, solvates, crystal forms, chelates, non-covalent complexes, and prodrugs thereof, wherein:
A is chosen from S, O, and —NR¹⁷— wherein R¹⁷ is chosen from hydrogen, alkyl, substituted alkyl, cycloalkyl, and substituted cycloalkyl;
R¹ is chosen from —(CR⁴R⁵)ₙQ, wherein
   n is an integer chosen from 0 to 8;
   each R⁴ and R⁵ is independently chosen from hydrogen, hydroxy, alkyl, and substituted alkyl;
   Q is chosen from hydrogen, sulfanyl, sulfonyl, alkoxy, substituted alkyl, optionally substituted amino, —CN, —SCN, —C(O)Z, alkenyl, substituted alkenyl, cycloalkyl, substituted cycloalkyl, aryl, substituted aryl, heterocycloalkyl, substituted heterocycloalkyl, heteroaryl, and substituted heteroaryl, wherein Z is chosen from —OR¹⁰, —R¹¹, —NR¹²R¹³, and —NHNHY, wherein
      R¹⁰ is chosen from hydrogen, alkyl, substituted alkyl, cycloalkyl, substituted cycloalkyl, aryl, substituted aryl, heterocycloalkyl, substituted heterocycloalkyl, heteroaryl, and substituted heteroaryl;
      R¹¹ is chosen from alkyl, substituted alkyl, aryl, substituted aryl, heteroaryl, and substituted heteroaryl;
      R¹² is chosen from hydrogen, alkyl, substituted alkyl, cycloalkyl, substituted cycloalkyl, aryl, and substituted aryl;
      R¹³ is chosen from hydrogen, alkyl, substituted alkyl, alkenyl, substituted alkenyl, cycloalkyl, substituted cycloalkyl, aryl, substituted aryl, heteroaryl, and substituted heteroaryl;
      or optionally R¹² and R¹³ together with the nitrogen atom to which R¹² and R¹³ are attached form a 5 to 7 member unsubstituted heterocyclic ring, or a 5 to 7 member substituted heterocyclic ring; and
      Y is chosen from hydrogen and —C(O)R¹⁶, wherein
         R¹⁶ is chosen from alkyl, substituted alkyl, cycloalkyl, substituted cycloalkyl, aryl, and substituted aryl;
R² is chosen from hydrogen, alkyl, substituted alkyl, alkenyl, substituted alkenyl, cycloalkyl, substituted cycloalkyl, aryl, substituted aryl, heterocycloalkyl, substituted heterocycloalkyl, heteroaryl, substituted heteroaryl, and —NH₂; and
R³ is chosen from hydrogen, alkyl, substituted alkyl, alkenyl, substituted alkenyl, cycloalkyl, substituted cycloalkyl, heterocycloalkyl, substituted heterocycloalkyl, aryl, substituted aryl, heteroaryl, and substituted heteroaryl.

Also provided is at least one chemical entity that exhibits selective activity for a protein kinase chosen from ABL1, AKT1, AKT2, AKT3, AURORA-A, c-TAK1, CDK1, CDK2/cyclinA, CDK2/cyclinE, CDK5, CHEK1, CHEK2, CSK, DAPK1, DYRK2, FLT-3, FYN, GSK3-α, GSK3-β, HCK, INSR, KIT, LCK, LYNA, MAPKAPK2, MAPKAPK3, MSK1, MSK2, p38-α, p38-β, p38-δ, p38-γ, P70S6K, PAK2, PDGFR-α, PAK1, PKA, PRAK, ROCK2, SGK1, SRC, SYK, PIM-1-kinase, PDK1, and RSK2.

Also provided is a pharmaceutical composition comprising at least one chemical entity described herein, and at least one pharmaceutically acceptable vehicle chosen from carriers, adjuvants, and excipients.

Also provided is a method of treating a patient having at least one disease responsive to inhibition of at least one ATP-utilizing enzyme comprising administering to the patient a therapeutically effective amount of at least one chemical entity described herein.

Also provided is a method of inhibiting at least one ATP-utilizing enzyme in a subject comprising administering to the subject at least one chemical entity described herein.

Also provided is a method of inhibiting at least one ATP-utilizing enzyme comprising contacting the ATP-utilizing enzyme with at least one chemical entity described herein.

Also provided is a method of treating at least one disease regulated by at least one ATP-utilizing enzyme in a subject in need of such treatment comprising administering to the subject a therapeutically effective amount of at least one chemical entity described herein.

Also provided is the use of at least one chemical entity for the manufacture of a medicament for the treatment of a patient having a disease responsive to inhibition of at least one ATP-utilizing enzyme, wherein the at least one chemical entity is a chemical entity described herein.

Also provided is a method for the manufacture of a medicament for the treatment of a patient having a disease responsive to inhibition of at least ATP-utilizing enzyme, comprising including in said medicament at least one chemical entity described herein.

Additional embodiments of the invention are set forth in the description which follows, or may be learned by practice of the invention.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the standard deviation found in their respective testing measurements. At the very least, and not as an attempt to limit the application of the doctrine of equivalents to the scope of the claims, each numerical parameter as set forth in the claims should at least be construed in light of the number of reported significant digits and by applying ordinary rounding techniques.

As used herein, when any variable occurs more than one time in a chemical formula, its definition on each occurrence is independent of its definition at every other occurrence. In accordance with the usual meaning of “a” and “the” in patents, reference, for example, to “a” kinase or “the” kinase is inclusive of one or more kinases.

A dash (“-”) that is not between two letters or symbols is used to indicate a point of attachment for a substituent. For example, —CONH₂ is attached through the carbon atom.

“Acyl” refers to a radical —C(O)R, where R is hydrogen, alkyl, substituted alkyl, substituted cycloalkyl, substituted heterocycloalkyl, substituted aryl, or substituted heteroaryl group as defined herein. Representative examples include, but are not limited to, formyl, acetyl, cylcohexylcarbonyl, cyclohexylmethylcarbonyl, benzoyl, benzylcarbonyl, and the like.

“Alkenyl” refers to an unsaturated branched, straight-chain or cyclic alkyl group having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl; and the like. In certain embodiments, an alkenyl group has from 2 to 20 carbon atoms and in other embodiments, from 2 to 6 carbon atoms.

“Alkoxy” refers to a radical —OR, where R represents an alkyl, substituted alkyl, substituted cycloalkyl, substituted heterocycloalkyl, substituted aryl, or substituted heteroaryl group as defined herein. Representative examples include, but are not limited to, methoxy, ethoxy, propoxy, butoxy, cyclohexyloxy, and the like.

“Alkoxycarbonyl” refers to a radical —C(O)— alkoxy where alkoxy is as defined herein.

“Alkyl” refers to a saturated, branched or straight-chain monovalent hydrocarbon group derived by the removal of one hydrogen atom from a single carbon atom of a parent alkane. Typical alkyl groups include, but are not limited to, methyl, ethyl, propyls such as propan-1-yl, propan-2-yl, and cyclopropan-1-yl, butyls such as butan-1-yl, butan-2-yl, 2-methyl-propan-1-yl, 2-methyl-propan-2-yl, cyclobutan-1-yl, and the like. In certain embodiments, an alkyl group comprises from 1 to 20 carbon atoms. In other embodiments, an alkyl group comprises from 1 to 6 carbon atoms, and is referred to as a lower alkyl group.

“Sulfonyl” refers to a radical —S(O)₂R where R is an alkyl, substituted alkyl, substituted cycloalkyl, substituted heterocycloalkyl, substituted aryl, or substituted heteroaryl group as defined herein. Representative examples include, but are not limited to methylsulfonyl, ethylsulfonyl, propylsulfonyl, butylsulfonyl, and the like.

“Sulfanyl” refers to a radical —SR where R is an alkyl, substituted alkyl, substituted cycloalkyl, substituted heterocycloalkyl, substituted aryl, or substituted heteroaryl group as defined herein that may be optionally substituted as defined herein. Representative examples include, but are not limited to, methylthio, ethylthio, propylthio, butylthio, and the like.

“Amino” refers to the radical —NH₂.

The term “substituted amino” refers to the group —NHR^{d} or —NR^{d}R^{d} where each R^{d} is independently chosen from: optionally substituted alkyl, optionally substituted cycloalkyl, optionally substituted acyl, optionally substituted aryl, optionally substituted heteroaryl, optionally substituted heterocycloalkyl, alkoxycarbonyl, and sulfonyl.

“Aryl” refers to a monovalent aromatic hydrocarbon group derived by the removal of one hydrogen atom from a single carbon atom of a parent aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group can comprise from 6 to 20 carbon atoms.

“Arylalkyl” or “aralkyl” refers to an acyclic alkyl group in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkyl, arylalkenyl, and/or arylalkynyl is used. In certain embodiments, an arylalkyl group can be (C₆₋₃₀) arylalkyl, e.g., the alkyl group of the arylalkyl group can be (C₁₋₁₀) and the aryl moiety can be (C₅₋₂₀).

“Carbonyl” refers to a radical —C(O) group.

“Carboxy” refers to the radical —C(O)OH.

When the chemical structure and chemical name conflict, the chemical structure is determinative of the identity of the compound. The chemical entities of the present disclosure may contain one or more chiral centers and/or double bonds and therefore, may exist as stereoisomers, such as double-bond isomers (i.e., geometric isomers), enantiomers or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Further, when partial structures of the chemical entities of the present disclosure are illustrated, asterisks indicate the point of attachment of the partial structure to the rest of the molecule. Enantiomeric and stereoisomeric mixtures can be resolved into the component enantiomers or stereoisomers using separation techniques or chiral synthesis techniques well known to the skilled artisan.

Compounds of Formula I include, but are not limited to optical isomers of compounds of Formula I, racemates, and other mixtures thereof. In those situations, the single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, compounds of Formula I include Z- and E-forms (or cis- and trans-forms) of compounds with double bonds. Where compounds of Formula I exists in various tautomeric forms, chemical entities of the present invention include all tautomeric forms of the compound.

Chemical entities of the present disclosure include, but are not limited to compounds of Formula I and all pharmaceutically acceptable forms thereof. Pharmaceutically acceptable forms of the compounds recited herein include pharmaceutically acceptable salts, solvates, crystal forms (including polymorphs and clathrates), chelates, non-covalent complexes, prodrugs, and mixtures thereof. In certain embodiments, the compounds described herein are in the form of pharmaceutically acceptable salts. Hence, the terms “chemical entity” and “chemical entities” also encompass pharmaceutically acceptable salts, solvates, crystal forms, chelates, non-covalent complexes, prodrugs, and mixtures thereof.

As noted above, prodrugs also fall within the scope of chemical entities, for example ester or amide derivatives of the compounds of Formula I. The term “prodrugs” includes any compounds that become compounds of Formula I when administered to a patient, e.g., upon metabolic processing of the prodrug. Examples of prodrugs include, but are not limited to, acetate, formate, and benzoate and like derivatives of functional groups (such as alcohol or amine groups) in the compounds of Formula I.

The term “solvate” refers to the compound formed by the interaction of a solvent and a compound. Suitable solvates are pharmaceutically acceptable solvates, such as hydrates, including monohydrates and hemi-hydrates.

“Cyano” refers to the radical —CN.

“Cycloalkyl” refers to a saturated or unsaturated cyclic alkyl group. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane, and the like. In certain embodiments, the cycloalkyl group can be C₃₋₁₀ cycloalkyl, such as, for example, C₃₋₆ cycloalkyl.

“Heterocycloalkyl” refers to a saturated or unsaturated cyclic alkyl group in which one or more carbon atoms (and any associated hydrogen atoms) are independently replaced with the same or different heteroatom. Typical heteroatoms to replace the carbon atom(s) include, but are not limited to, N, P, O, S, and Si. Where a specific level of saturation is intended, the nomenclature “cycloheteroalkanyl” or “cycloheteroalkenyl” is used. Typical cycloheteroalkyl groups include, but are not limited to, groups derived from epoxides, imidazolidine, morpholine, piperazine, piperidine, pyrazolidine, pyrrolidine, quinuclidine, and the like.

“Disease” refers to any disease, disorder, condition, symptom, or indication.

“Enzyme” refers to any naturally occurring or synthetic macromolecular substance composed wholly or largely of protein, that catalyzes, more or less specifically, one or more biochemical reactions. The substances upon which the enzyme acts are referred to “substrates,” for which the enzyme possesses a specific binding or “active site,” or “catalytic domain.” Enzymes can also act on macromolecular structures such as muscle fibers.

“Extended release” refers to dosage forms that provide for the delayed, slowed, over a period of time, continuous, discontinuous, or sustained release of the compounds of the present disclosure.

“Halo” refers to a fluoro, chloro, bromo, or iodo group.

“Heteroaryl” refers to a monovalent heteroaromatic group derived by the removal of one hydrogen atom from a single atom of a parent heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, the heteroaryl group can be between 5 to 20 membered heteroaryl, such as, for example, a 5 to 10 membered heteroaryl. In certain embodiments, heteroaryl groups can be those derived from thiophene, pyrrole, benzothiophene, benzofuran, indole, pyridine, quinoline, imidazole, oxazole, and pyrazine.

“Heteroarylalkyl” or “heteroaralkyl” refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl, and/or heteroarylalkynyl is used. In certain embodiments, the heteroarylalkyl group can be a 6 to 30 membered heteroarylalkyl, e.g., the alkanyl, alkenyl or alkynyl moiety of the heteroarylalkyl can be 1 to 10 membered and the heteroaryl moiety can be a 5 to 20-membered heteroaryl.

“Leaving group” refers to an atom or a group capable of being displaced by a nucleophile and includes halo, such as chloro, bromo, fluoro, and iodo, alkoxycarbonyl (e.g., acetoxy), aryloxycarbonyl, mesyloxy, tosyloxy, trifluoromethanesulfonyloxy, aryloxy (e.g., 2,4-dinitrophenoxy), methoxy, N,O-dimethylhydroxylamino, and the like.

“Optional” or “optionally” means that the subsequently described event or circumstance may but need not occur, and that the description includes instances where the event or circumstance occurs and instances in which the event does not.

“Pharmaceutically acceptable” refers to approved or approvable by a regulatory agency of the Federal or a state government or listed in the U.S. Pharmacopeia or other generally recognized pharmacopeia for use in animals, and more particularly in humans.

“Pharmaceutically acceptable salt” refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, dicyclohexylamine, and the like.

“Pharmaceutically acceptable excipient, carrier or adjuvant” refers to an excipient, carrier or adjuvant that can be administered to a subject, together with a at least one chemical of the present disclosure, and which does not destroy the pharmacological activity thereof and is nontoxic when administered in doses sufficient to deliver a therapeutic amount of the at least one chemical entity.

“Pharmaceutically acceptable vehicle” refers to a diluent, adjuvant, excipient or carrier with which at least one chemical entity of the present disclosure is administered.

“Promoiety” refers to a form of protecting group that when used to mask a functional group within a drug molecule converts the drug into a prodrug. For example, the promoiety can be attached to the drug via bond(s) that are cleaved (or broken) by enzymatic or non-enzymatic means in vivo.

“Protecting group” refers to a grouping of atoms that when attached to a reactive group in a molecule masks, reduces or prevents that reactivity. Examples of protecting groups can be found in Green et al., “Protective Groups in Organic Chemistry,” (Wiley, 2^{nd} ed. 1991) and Harrison et al., “Compendium of Synthetic Organic Methods,” Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”), and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

“Protein kinase” and “kinase” refers to any enzyme that phosphorylates one or more hydroxyl or phenolic groups in proteins, ATP being the phosphoryl-group donor.

“Stereoisomer” refers to an isomer that differs in the arrangement of the constituent atoms in space. Stereoisomers that are mirror images of each other and optically active are termed “enantiomers,” and stereoisomers that are not mirror images of one another are termed “diastereoisomers.”

“Subject” includes mammals and humans. The terms “human” and “subject” are used interchangeably herein.

“Substituted” refers to a group in which one or more hydrogen atoms are each independently replaced with the same or different substituent(s). Typical substituents include, but are not limited to, —X, —R³³, —O⁻, ═O, —OR³³, —SR³³, —S⁻, S,

—NR³³R³⁴, ═NR³³, —CX₃, —CF₃, —CN, —OCN, —SCN, —NO, —NO₂, ═N₂, —N₃, —S(O)₂O⁻, —S(O)₂OH, —S(O)₂R³³, —OS(O₂)O⁻, —OS(O)₂R³³—P(O)(O⁻)₂, —P(O)(OR³³)(O⁻), —OP(O)(OR³³)(OR³⁴), —C(O)R³³, —C(S)R³³, —C(O)OR³³, —C(O)NR³³R³⁴, —C(O)O⁻, —C(S)OR³³, —NR³⁵C(O)NR³³R³⁴, —NR³⁵C(S)NR³³R³⁴, —NR³⁵C(NR³³)NR³³R³⁴, —C(NR³³)NR³³R³⁴, —S(O)₂NR³³R³⁴, —NR³⁵S(O)₂R³³, —NR³⁵C(O)R³³, and —S(O)R³³ where each X is independently a halo; each R³³ and R³⁴ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, substituted heteroarylalkyl, —NR³⁵R³⁶, —C(O)R³⁵ or —S(O)₂R³⁵ or optionally R³³ and R³⁴ together with the atom to which R³³ and R³⁴ are attached form one or more cycloheteroalkyl, substituted cycloheteroalkyl, heteroaryl, or substituted heteroaryl rings; and R³⁵ and R³⁶ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl or substituted heteroarylalkyl, or optionally R³⁵ and R³⁶ together with the nitrogen atom to which R³⁵ and R³⁶ are attached form one or more cycloheteroalkyl, substituted cycloheteroalkyl, heteroaryl, or substituted heteroaryl rings. In certain embodiments, a tertiary amine or aromatic nitrogen may be substituted with on or more oxygen atoms to form the corresponding nitrogen oxide.

In certain embodiments, substituted aryl and substituted heteroaryl include one or more of the following substitute groups: F, Cl, Br, C₁₋₃ alkyl, substituted alkyl, C₁₋₃ alkoxy, —S(O)₂NR³³R³⁴, —NR³³R³⁴, —CF₃, —OCF₃, —CN, —NR³⁵S(O)₂R³³, —NR³⁵C(O)R³³, C₅₋₁₀ aryl, substituted C₅₋₁₀ aryl, C₅₋₁₀ heteroaryl, substituted C₅₋₁₀ heteroaryl, —C(O)OR³³, —NO₂, —C(O)R³³, —C(O)NR³³R³⁴, —OCHF₂, C₁₋₃ acyl, —SR³³, —S(O)₂OH, —S(O)₂R³³, —S(O)R³³, —C(S)R³³, —C(O)O⁻, —C(S)OR³³, —NR³⁵C(O)NR³³R³⁴, —NR³⁵C(S)NR³³R³⁴, and —C(NR³⁵)NR³³R³⁴, C₃₋₈ cycloalkyl, and substituted C₃₋₈ cycloalkyl, as defined herein.

In certain embodiments, substituted arylalkyl, and substituted heteroarylalkyl include one or more of the following substitute groups: F, Cl, Br, C₁₋₃ alkyl, C₁₋₃ alkoxy, —S(O)₂NR³³R³⁴, —NR³³R³⁴, CF₃, —OCF₃, CN, —NR³⁵S(O)₂R³³, —NR³⁵C(O)R³³, C₅₋₁₀ aryl, substituted alkyl, substituted C₅₋₁₀ aryl, C₅₋₁₀ heteroaryl, substituted C₅₋₁₀ heteroaryl, —C(O)OR³³, —NO₂, —C(O)R³³, —C(O)NR³³R³⁴, —OCHF₂, C₁₋₃ acyl, —SR³³, —S(O)₂OH, —S(O)₂R³³, —S(O)R³³, —C(S)R³³, —C(O)O⁻, —C(S)OR³³, —NR³⁵C(O)NR³³R³⁴, —NR³⁵C(S)NR³³R³⁴, and —C(NR³⁵)NR³³R³⁴, C₃₋₈ cycloalkyl, and substituted C₃₋₈ cycloalkyl, as defined herein.

In certain embodiments, substituted alkyl, substituted cycloalkyl, and substituted heterocycloalkyl includes one or more of the following substitute groups: C₁₋₃ alkoxy, —NR³³R³⁴, substituted C₅₋₁₀ heteroaryl, —SR³³, C₁₋₃ alkoxy, —S(O)₂NR³³R³⁴, CN, F, Cl, —CF₃, —OCF₃, —NR³⁵S(O)₂R³³, —NR³⁵C(O)R³³, C₅₋₁₀ aryl, substituted C₅₋₁₀ aryl, C₅₋₁₀ heteroaryl, substituted C₅₋₁₀ heteroaryl, —C(O)OR³³, —NO₂, —C(O)R³³, —C(O)NR³³R³⁴, —OCHF₂, C₁₋₃ acyl, —S(O)₂OH, —S(O)₂R³³, —S(O)R³³, —C(S)R, —C(O)O⁻, C(S)OR³³, —NR³⁵C(O)NR³³R³⁴, —NR³⁵C(S)NR³³R³⁴ and —C(NR³⁵)NR³³R³⁴, C₃₋₈ cycloalkyl, and substituted C₃₋₈ cycloalkyl, as defined herein.

In certain embodiments, substituted alkenyl includes one or more of the following substitute groups: C₁₋₈ alkyl, substituted C₁₋₈ alkyl, C₅₋₁₀ aryl, substituted C₅₋₁₀ aryl, C₅₋₁₀ heteroaryl, substituted C₅₋₁₀ heteroaryl, C₃₋₈ cycloalkyl, substituted C₃₋₈ cycloalkyl, cycloheteroalkylalkyl, and substituted cycloheteroalkylalkyl, as defined herein.

“Therapeutically effective amount” refers to the amount of a compound that, when administered to a subject for treating a disease, or at least one of the clinical symptoms of a disease or disorder, is sufficient to affect such treatment for the disease, disorder, or symptom. The “therapeutically effective amount” can vary depending on the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be readily apparent to those skilled in the art or capable of determination by routine experimentation.

“Therapeutically effective dosage” refers to a dosage that provides effective treatment of a condition and/or disease in a subject. The therapeutically effective dosage can vary somewhat from compound to compound, and from subject to subject, and can depend upon factors such as the condition of the subject and the route of delivery. A therapeutically effective dosage can be determined in accordance with routine pharmacological procedures known to those skilled in the art.

“Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease or disorder or at least one of the clinical symptoms of a disease or disorder. “Treating” or “treatment” also refers to inhibiting the disease or disorder, either physically, (e.g., stabilization of a discernible symptom), physiologically, (e.g., stabilization of a physical parameter), or both, and inhibit at least one physical parameter which may not be discernible to the subject. Further, “treating” or “treatment” refers to delaying the onset of the disease or disorder or at least symptoms thereof in a subject which may be exposed to or predisposed to a disease or disorder even though that subject does not yet experience or display symptoms of the disease or disorder.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

The compounds of Formula I can be named and numbered in the manner (e.g., using ChemDraw Ultra 9.0 Struct=Name algorithm) described below. For example, the compound: 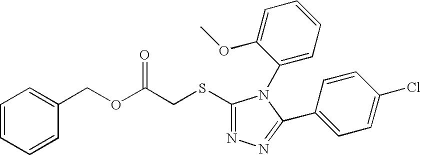
i.e., the compound according to Formula I where A is S, n is 1, R⁴ and R⁵ are hydrogen, Q is —C(O)Z, Z is —OR¹⁰, R¹⁰ is benzyl, R² is 2-methoxyphenyl, and R³ is 4-chlorophenyl is named benzyl 2-(5-(4-chlorophenyl)-4-(2-methoxyphenyl)-4H-1,2,4-triazol-3-ylthio)acetate.

Certain embodiments of the present disclosure provide at least one chemical entity chosen from compounds of Formula I, 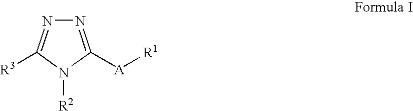
and pharmaceutically acceptable salts, solvates, crystal forms, chelates, non-covalent complexes, and prodrugs thereof, wherein:
A is chosen from S, O, and —NR¹⁷— wherein R¹⁷ is chosen from hydrogen, alkyl, substituted alkyl, cycloalkyl, and substituted cycloalkyl;
R¹ is chosen from —(CR⁴R⁵)ₙQ, wherein
   n is an integer chosen from 0 to 8;
   each R⁴ and R⁵ is independently chosen from hydrogen, hydroxy, alkyl, and substituted alkyl;
   Q is chosen from hydrogen, sulfanyl, sulfonyl, alkoxy, substituted alkyl, optionally substituted amino, —CN, —C(O)Z, alkenyl, substituted alkenyl, cycloalkyl, substituted cycloalkyl, aryl, substituted aryl, heterocycloalkyl, substituted heterocycloalkyl, heteroaryl, substituted heteroaryl, and —SCN, wherein Z is chosen from —OR¹⁰, —R¹¹, —NR¹²R¹³, and —NHNHY, wherein
      R¹⁰ is chosen from hydrogen, alkyl, substituted alkyl, cycloalkyl, substituted cycloalkyl, aryl, substituted aryl, heterocycloalkyl, substituted heterocycloalkyl, heteroaryl, and substituted heteroaryl;
      R¹¹ is chosen from alkyl, substituted alkyl, aryl, substituted aryl, heteroaryl, and substituted heteroaryl;
      R¹² is chosen from hydrogen, alkyl, substituted alkyl, cycloalkyl, substituted cycloalkyl, aryl, and substituted aryl;
      R¹³ is chosen from hydrogen, alkyl, substituted alkyl, alkenyl, substituted alkenyl, cycloalkyl, substituted cycloalkyl, aryl, substituted aryl, heteroaryl, and substituted heteroaryl;
      or optionally R¹² and R¹³ together with the nitrogen atom to which R¹² and R¹³ are attached form a 5 to 7 member unsubstituted heterocyclic ring, or a 5 to 7 member substituted heterocyclic ring; and
      Y is chosen from hydrogen and —C(O)R¹⁶, wherein
         R¹⁶ is chosen from alkyl, substituted alkyl, cycloalkyl, substituted cycloalkyl, aryl, and substituted aryl;
R² is chosen from hydrogen, alkyl, substituted alkyl, alkenyl, substituted alkenyl, cycloalkyl, substituted cycloalkyl, aryl, substituted aryl, heterocycloalkyl, substituted heterocycloalkyl, heteroaryl, substituted heteroaryl, and —NH₂; and
R³ is chosen from hydrogen, alkyl, substituted alkyl, alkenyl, substituted alkenyl, cycloalkyl, substituted cycloalkyl, heterocycloalkyl, substituted heterocycloalkyl, aryl, substituted aryl, heteroaryl, and substituted heteroaryl,
provided that
when A is S, R¹ is not chosen from SCN, an aminopyridopyrimidine derivative, dopamine derivative, a dopa derivative, quinazoline derivative, a quinazolinone derivative, a benzoquinoxaline derivative, a phthalazine derivative, a pyrimidinyl derivative, a fused pyrimidine derivative, substituted pyridinyl and substituted aryl wherein the substituent on the substituted aryl is chosen from ether-, thio-, or amino-substituted groups, wherein the substituent is a 3-cyanoquinoline or aromatic tricyclic derivative;
when A is S, R² is not chosen from substituted alkenyl, wherein the substituent is chosen from an indolinone derivative,
when A is S, R³ is not chosen from substituted diarylamine and 1,2,3-triazole derivatives;
when A is S, R¹ is —(CR⁴R⁵)ₙQ, R² is H and R³ is hydrogen, then Q is not chosen from substituted alkyl, wherein the substituent is chosen from an amidothioxanthene, an alkylthioxanthene ether, a carbazole derivative, and a quinazolinone derivative;
when A is S, R¹ is —(CR⁴R⁵)ₙQ, Q is not chosen from substituted arylalkyl wherein the substituent on the arylalkyl group is chosen from an aminopyridopyrimidine derivative; substituted alkyl wherein the substituent on the alkyl group is a quinazolinone derivative; substituted heteroarylalkyl and substituted arylalkyl, wherein the substituent on the substituted heteroarylalkyl and on the substituted arylalkyl is chosen from ether, thio, and amino; 3-cyanoquinoline, an aromatic tricyclic derivative; a 3-substituted phenyl group wherein the 3-substituent is chosen from —C(O)NH and —NHCO; an indolocarbazole derivative; substituted pyridinyl, pyrimidinyl, and phenyl wherein the substituent is chosen from ether, thio, and amino, wherein the substituent is chosen from a 3-cyanoquinoline derivative and an aromatic tricyclic derivative; a phthalazine derivative; and substituted cycloheteroalkyl and substituted cycloheteroalkylalkyl, wherein the substituent is chosen from a phenylaminopyridopyrimidine derivative and an indolocarbazole derivative;
when A is S, Q is —C(O)Z, Z is —R¹¹, and R² and R³ are phenyl, then R¹¹ is not α-benzeneacetonitrile;
when A is S, Q is —C(O)Z, Z is —NR¹²R¹³, R² and R³ are phenyl, and R¹² is H; then R¹³ is not 2-benzoic acid methyl ester;
when A is S, Q is —C(O)Z, Z is —NR¹²R¹³, R² is 3-trifluoromethylphenyl, R³ is 4-methoxyphenyl, and R¹² is hydrogen, then R¹³ is not chosen from 4-cyclohexylphenyl and 4-benzoylphenyl;
when A is S, Q is —C(O)Z, Z is —NR¹²R¹³, R² is phenyl, R³ is chosen from 4-[[(phenylamino) thioxomethyl]amino]phenyl and 4-chloro-2-methoxyphenyl, and R¹² is hydrogen, then R¹³ is not chosen from 4-benzoyl L-aspartic acid and 4-benzoyl L-glutamic acid;
when A is S, Q is —C(O)Z, Z is —NR¹²R¹³, R² is chosen from phenyl and 4-chlorophenyl, R³ is 4-[(1H-indol-3-ylmethylene)amino]phenyl, and R¹² is hydrogen, then R¹³ is not chosen from phenyl, 2-methylphenyl, 4-methoxyphenyl, 2-methoxyphenyl, 4-chlorophenyl, 3-chlorophenyl, and 3-nitrophenyl;
when A is S, Q is —C(O)Z, Z is —NR¹²R¹³ and R², R³, and R¹² are hydrogen, then R¹³ is not chosen from a thioxanthene derivative;
when A is O, R² is substituted alkyl, then R¹ is not chosen from alkyl-G¹, wherein G¹ is chosen from a phenyl-substituted oxadiazolyl and phenyl-substituted isoxazolyl;
when A is O, R³ is chosen from substituted imidazo[1,2-a]pyridyl, and R² is methyl; then R¹ is not methyl;
when A is O, R² is chosen from aryl, and R³ is biphenyl, then R¹ is not methyl;
when A is O, R³ is chosen from alkyl, alkenyl, and cycloalkyl, and R² is chosen from phenyl and pyridyl, then R¹ is not N-benzylpiperidin-4-yl-methyl;
when A is O, R³ is chosen from 4-heteroarylmethoxy-phenyl, and R² is methyl, then R¹ is not chosen from methyl and trifluoromethyl;
when A is O, R³ is chosen from aryl and heteroaryl, and R² is chosen from alkyl and cycloalkyl, then R¹ is not chosen from alkylene-B—Ar², wherein B is chosen from piperidinyl, piperazinyl, and tetrahydropyridinyl, and Ar² is chosen from phenyl, pyridyl, pyrimidinyl, and triazinyl;
when A is O, R³ is chosen from phenyl and pentafluoroethyl, and R² is methyl, then R¹ is not 4-(N-sulfonamido) phenyl;
when A is O, R³ is trifluoromethyl, and R² is 2-biphenyl, then R¹ is not methoxymethyl;
when A is O, R³ is N-sulfonamido-substituted phenyl, and R² is chosen from hydrogen, alkyl, and substituted alkyl, then R¹ is not chosen from alkyl, substituted alkyl, and phenyl;
when A is O, R³ is n-butyl, and R² is 2′-tetrazolyl-4-biphenylmethyl, then R¹ is not chosen from benzyl and phenethyl;
when A is O, R³ is phenyl, and R² is chosen from n-propyl, tert-butyl, and phenyl, then R¹ is not chosen from —CH₂CO₂CH₂CH₃, —CH₂CONH₂NH₂, and CH₂-G², wherein G² is chosen from 1,2,4-triazole-3-thione, 1,3,4-oxadiazole-2-thione, and 1,24-triazolo[3,4b][1,3,4]thiadiazole;
when A is O, R³ is cyclohexyl, and R² is cyclohexyl, then R¹ is not methyl;
when A is O, R³ is phenyl, and R² is phenyl, then R¹ is not chosen from phenyl, substituted, phenyl and methyl;
when A is O, R³ is 3-(4-biphenyloxycarbonyl)phenyl, and R² is n-butyl, then R¹ is not methyl;
when A is O, R³ is phenyl, and R² is methyl, then R¹ is not methyl;
when A is O, R³ is methyl, and R² is methyl, then R² is not methyl;
when A is O, R³ is 2-furyl, and R² is methyl, then R¹ is not methyl;
when A is NR¹⁷, R³ is phenyl; and R² is chosen from phenyl and substituted phenyl, then R¹ and R¹⁷ are not both methyl;
when A is NR¹⁷, R³ is chosen from 2-hydroxyphenyl and 2-furyl and R² is phenyl, then R¹ and R¹⁷ are not both ethyl;
when A is NR¹⁷, R¹⁷ is hydrogen, R³ is 2-hydroxyphenyl, and R² is phenyl, then R¹ is not chosen from isopropyl, 4-(4-pyridinyl)butyl, and 3,4-dimethoxyphenethyl;
when A is NR¹⁷, R¹⁷ is H; R³ is chosen from 4-pyridyl and 4-pyrimidinyl, and R² is hydrogen, then R¹ is not chosen from —CH₂CONHG³, wherein G³ is chosen from aryl and heteroaryl;
when A is NR¹⁷, R¹⁷ is hydrogen, R³ is hydrogen, and R² is methyl, then R¹ is not 3-[2-(dimethylamino)ethyl]-1H-indol-5-ylmethyl; and
when A is NR¹⁷, then the compound is not chosen from 1-(3-Amino-[1,2,4]triazol-4-yl)-2-(4-chloro-phenyl)-ethanone and 5-(2-Methoxy-phenyl)-4H[1,2,4]triazol-3-ylamine.

In certain embodiments of compounds of Formula I, A is S. In certain embodiments of compounds of Formula I, A is O. In certain embodiments of compounds of Formula I, A is —NR¹⁷. In certain embodiments of compounds of Formula I, A is —NR¹⁷ and R¹⁷ is hydrogen.

In certain embodiments, n is 0. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is chosen from 1 and 2. In certain embodiments of compounds, n is chosen from 3, 4, and 5.

In certain embodiments of compounds of Formula I, A is S and n is 0.

In certain embodiments of compounds of Formula I, A is S, n is 0 and Q is H.

In certain embodiments of compounds of Formula I, A is S, n is 0 and Q is substituted heteroaryl (for example, in certain embodiments, Q is chosen from 5-bromo-2-phenyl-2H-pyridazin-3-one-4-yl, 2-hydroxy-4-phenyl-quinolin-3-yl, and 8-nitro-quinolin-5-yl.

In certain embodiments of compounds of Formula I, A is S and n is 1.

In certain embodiments of compounds of Formula I, A is S, n is 1 and Q is —SCN.

In certain embodiments of compounds of Formula I, A is S, n is 1 and Q is —CN.

In certain embodiments of compounds of Formula I, A is S, n is chosen from 1 and 2, and Q is chosen from hydrogen, heterocycloalkyl and substituted heterocycloalkyl. In certain of such embodiments, Q is chosen from hydrogen, piperidin-1-yl, morpholin-4-yl, cyclohexyl, pyrrolidin-1-yl, cyclopropyl, and tetrahydrofuran-2-yl.

In certain embodiments of compounds of Formula I, A is S, n is 1 and Q is chosen from aryl, substituted aryl, heteroaryl, and substituted heteroaryl. In certain of such embodiments, Q is chosen from phenyl and phenyl substituted with one or two groups chosen from nitro, halo, lower alkyl, carboxy, cyano, alkoxycarbonyl, sulfonyl, lower alkoxy, trifluoromethyl, trifluoromethoxy, and difluoromethoxy.

In certain embodiments, R² is chosen from hydrogen, lower alkyl, substituted lower alkyl, alkenyl, cycloalkyl, substituted cycloalkyl, aryl, substituted aryl, heteroaryl, and substituted heteroaryl. In certain embodiments, R² is chosen from hydrogen, lower alkyl, substituted lower alkyl, alkenyl, cyclohexyl, phenyl, and substituted phenyl. In certain embodiments, R² is chosen from aryl and substituted aryl. In certain embodiments, R² is chosen from phenyl and phenyl substituted with one or two groups chosen from —OH, halo, —CN, carboxy, trifluoromethyl, trifluoromethoxy, C₁₋₈ alkyl, and C₁₋₈ alkoxy. In certain embodiments, R² is chosen from phenyl and phenyl substituted with one or two groups chosen from lower alkyl, lower alkoxy, halo, trifluoromethyl, and trifluoromethoxy. In certain embodiments, the substituent is chosen from C₁₋₄ alkoxy.

In certain embodiments, R² is chosen from hydrogen, methyl, ethyl, propyl, propen-3-yl, propen-2-yl, isobutyl, isobutene-3-yl, phenyl, 4-chlorophenyl-acetyl, benzyl, cyclohexyl, phenethyl, 1-propen-3-yl, 1-isobuten-3-yl, 2-methoxyethyl, 2-methoxypropyl, propyloxymethyl, pyridin-2-yl, pyridin-3-yl, tetrahydrofuran-2-yl-methyl, furan-2-ylmethyl, N-propen-3-yl-morpholine, amino, N,N-dimethylaminopropyl, phenyl, and substituted phenyl wherein the substituents are independently chosen from halo, methyl, trifluoromethyl, ethyl, cyclohexyl, —NH₂, carboxy, cyano, methoxy, ethoxy, methoxypropyl, benzyl, phenethyl, methoxyethyl, furan-2-ylmethyl, tetrahydrofuran-2-yl-methyl, furan-2-yl-ethyl, 3-cyclohexylmethyl-furan-2-yl, 1H-benzimidazol-2-yl-methyl, 3,4-methylenedioxyphenyl, and morpholin-4-yl-propyl.

In certain embodiments, R³ is chosen from hydrogen, substituted lower alkyl, cycloalkyl, substituted cycloalkyl, aryl, and substituted aryl.

In certain embodiments, R³ is —CH₂X wherein X is chosen from aryl, heteroaryl, —OR⁶, —SR⁷, and —NR⁸R⁹, wherein
R⁶ is chosen from aryl, and substituted aryl;
R⁷ is chosen from heteroaryl, and substituted heteroaryl;
R⁸ is H; and
R⁹ is substituted aryl.

In certain embodiments, R³ is chosen from cycloalkyl, substituted cycloalkyl, aryl, and substituted aryl. In certain embodiments, R³ is chosen from aryl and aryl substituted with a group chosen from —OH, halo, —CN, —CF₃, C₁₋₈ alkyl, and C₁₋₈ alkoxy. In certain embodiments, R³ is chosen from phenyl and phenyl substituted with a group chosen from —OH, halo, —CN, —CF₃, C₁₋₈ alkyl, and C₁₋₈ alkoxy. In certain embodiments, R³ is chosen from phenyl and phenyl substituted with a group chosen from halo, —OH and C₁₋₈ alkoxy.

In certain embodiments, R³ is hydrogen.

In certain embodiments, R⁴ and R⁵ are independently chosen from hydrogen and lower alkyl. In certain embodiments, R⁴ and R⁵ are independently chosen from hydrogen and methyl. In certain embodiments, R⁴ and R⁵ are hydrogen.

In certain embodiments, the compound of Formula I is chosen from any one of the compounds set forth in Tables 1, 2, and 3.

Certain embodiments of the present disclosure provide at least one chemical entity chosen from compounds of Formula II, 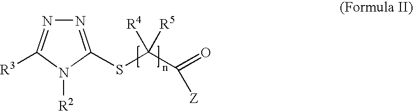
and pharmaceutically acceptable salts, solvates, crystal forms, chelates, non-covalent complexes, and prodrugs thereof, wherein n, Z, R⁴, R⁵, R², and R³ are as described for compounds of Formula I.

In certain embodiments of compounds of Formula II, n is 1 and Z is —NHNHY. In certain of such embodiments, Y is —C(O)R¹⁶ wherein R¹⁶ is chosen from cyclohexyl, aryl, substituted aryl, arylalkyl, and substituted arylalkyl. In certain of such embodiments, R¹⁶ is chosen from benzyl and substituted phenyl wherein the phenyl is substituted with one, two, or three groups chosen from hydroxy, lower alkoxy, halo, and lower alkyl.

In certain embodiments of compounds of Formula II, n is 2. In certain of such embodiments, n is 2 and Z is —OR¹⁰ wherein R¹⁰ is chosen from hydrogen and lower alkyl.

Certain embodiments of the present disclosure provide at least one chemical entity chosen from compounds of Formula III, 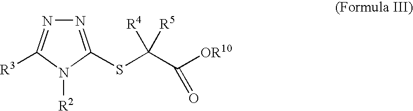
and pharmaceutically acceptable salts, solvates, crystal forms, chelates, non-covalent complexes, and prodrugs thereof, wherein: wherein R¹⁰, R⁴, R⁵, R², and R³ are as described for compounds of Formula I.

In certain embodiments of compounds of Formula III, R¹⁰ is chosen from hydrogen, lower alkyl, benzyl, phenethyl, substituted benzyl, and substituted phenethyl, wherein the phenyl group of the substituted benzyl and substituted phenethyl is independently substituted with one or two groups chosen from halo, lower alkyl, lower alkoxy, and hydroxy.

Certain embodiments of the present disclosure provide at least one chemical entity chosen from compounds of Formula IV, 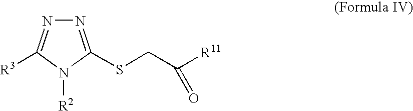
and pharmaceutically acceptable salts, solvates II, crystal forms, chelates, non-covalent complexes, and prodrugs thereof, wherein R¹¹, R², and R³ are as described for compounds of Formula I.

In certain embodiments of compounds of Formula IV, R¹¹ is chosen from heteroaryl, substituted heteroaryl, phenyl, and substituted phenyl. In certain of such embodiments, R¹¹ is is chosen from phenyl, 2,3-dihydrobenzo[b][1,4]dioxine-6-yl, benzo[d][1,3]dioxole-5-yl, and phenyl substituted with one or two groups chosen from lower alkoxy, lower alkyl, halo, and hydroxy.

Certain embodiments of the present disclosure provide at least one chemical entity chosen from compounds of Formula V, 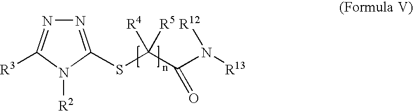
and pharmaceutically acceptable salts of compounds, solvates, crystal forms, chelates, non-covalent complexes, and prodrugs thereof, wherein n, R¹², R¹³, R⁴, R⁵, R², and R³ are as described for compounds of Formula I.

In certain embodiments of Formula V, n is 1 and R¹² is chosen from hydrogen and alkyl; and R¹³ is chosen from aryl, substituted aryl, arylalkyl, heteroarylalkyl, and substituted heteroarylalkyl. In certain of such embodiments, R¹² is hydrogen, and R¹³ is chosen from aryl, substituted aryl, heteroarylalkyl, and substituted heteroarylalkyl. In certain of such embodiments, R¹³ is chosen from hydrogen, methyl, ethyl, propyl, isopropyl, tert-butyl, butyl, methoxyethyl, 2-hydroxyethyl, 3-hydroxypropyl, propene-3-yl, phenyl, substituted phenyl, benzyl, substituted benzyl, substituted cyclohexyl, cyclopentyl, phenethyl, substituted phenethyl, cyclohexylmethyl, thiophen-2-ylmethyl, substituted [1,3,4]-thiadiazol-2-yl, 10,11-dihydro-5H-dibenzo[b,f]azepine-N-yl, morpholin-4-ylpropyl, morpholin-4-yl-ethyl, substituted benzothiazol-2-yl, substituted benzothiazol-5-yl, substituted propyl, furan-2-ylmethyl, tetrahydrofuran-2-yl-methyl, naphthalen-1-yl, thiazol-2-yl, substituted [1,3,4]thiadiazol-2-yl, 10H-phenothiazine-N-yl, 1,2,3,4-tetrahydroquinolin-1-yl, isoxazol-3-yl, substituted isoxazol-3-yl, 4,5,6,7-tetrahydrobenzothiazol-2-yl, substituted piperazin-1-yl, substituted piperidin-1-yl, substituted 5,6,-dihydro-4H-cyclopenta[b]thiophen-2-yl, 2-thiophen-2-ylmethyl, 3,4-methylenedioxyphenyl, substituted thiophen-2-yl, (3,4-methylenedioxyphenyl)methyl, substituted dibenzofuran-3-yl, 4,5,6,7-tetrahydro-benzo[b]thiophen-2-yl, —NHCOCH₂CH₃, 3-(furan-2-yl-carbonylamino)phenyl, and 3-(furan-2-yl-carbonylamino)-6-methylphenyl.

In certain embodiments of Formula V, n is 1 and R¹² and R¹³ together with the nitrogen atom to which R¹² and R¹³ are attached form a heterocyclic ring or substituted heterocyclic ring, wherein the heterocyclic ring is chosen from morpholine, quinoline; pyrrolidone, pyrrolidine, substituted piperazine, 2,3-dihydro-1H-indole, piperidine, substituted pyridine, pyridine, substituted pyrazine, 10H-phenthiazine, azepane, 1,2,3,4,-tetrahydroisoquinoline, and 1,2,3,4-tetrahydroquinoline. In certain of such embodiments, the substituents on the substituted heterocyclic ring are independently chosen from halo, —NH₂, —OH, —CF₃, —CN, —NO₂, —COOH, methyl, ethyl, methoxy, ethoxy, propoxy, phenyl, —COCH₃, —COOCH₃, —COOCH₂CH₃, —CONH₂, —CH₂COOCH₂CH₃, —NHCO-tetrahydrofuran-2-yl, 2-hydroxyethyl, —NHCO-furan-2-yl, —NHCO-thiophen-2-yl, —NHCO-furan-2-yl, and 4-methoxyphenyl.

Certain embodiments of the present disclosure provide at least one chemical entity chosen from compounds of Formula VI, 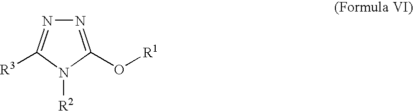
and pharmaceutically acceptable salts, solvates, crystal forms, chelates, non-covalent complexes, and prodrugs thereof, wherein R¹, R², and R³ are as described for compounds of Formula I.

In certain embodiments of compounds of Formula VI, n is 1.

In certain embodiments of compounds of Formula VI, n is 1 and Q is —C(O)Z wherein Z is —OR¹⁰. In certain of such embodiments, R¹⁰ is chosen from C₁₋₄ alkyl-phenyl, for example, in certain embodiments, R¹⁰ is chosen from benzyl and phenethyl.

In certain embodiments of compounds of Formula VI, n is 1 and Q is —C(O)Z wherein Z is —NR¹²R¹³. In certain of such embodiments, R¹² is hydrogen and R¹³ is chosen from furan-2-ylmethyl and substituted phenyl. In certain of such embodiments, the substituents on the substituted phenyl are chosen from hydroxy, halo, lower alkyl, and lower alkoxy.

In certain embodiments of compounds of Formula VI, n is chosen from 3, 4, and 5.

In certain embodiments of compounds of Formula VI, n is chosen from 3, 4, and 5, and Q is chosen from phenyl and substituted phenyl. In certain of such embodiments, Q is phenyl.

Certain embodiments of the present disclosure provide at least one chemical entity chosen from compounds of Formula VII, 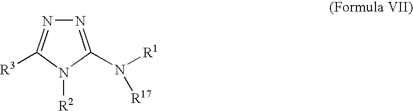
and pharmaceutically acceptable salts, solvates, crystal forms, chelates of compounds of Formula XI, non-covalent complexes, and prodrugs thereof, wherein R¹, R², R³, and R¹⁷ are as described for compounds of Formula I.

In certain embodiments of compounds of Formula VII, R¹⁷ is hydrogen.

In certain embodiments of compounds of Formula VII, n is 0.

In certain embodiments of compounds of Formula VII, n is 0 and Q is hydrogen.

In certain embodiments of compounds of Formula VII, n is 1.

In certain embodiments of compounds of Formula VII, n is 1 and Q is —C(O)Z wherein Z is chosen from —OR¹⁰ and —NR¹²R¹³.

As used herein, the compounds of the present disclosure, including the compounds of Formula I can include pharmaceutically acceptable derivatives or prodrugs thereof. A “pharmaceutically acceptable derivative or prodrug” refers to any appropriate pharmaceutically acceptable salt, ester, salt of an ester, hydrate, solvate, or other derivative of a compound of this present disclosure that, upon administration to a subject, is capable of providing, directly or indirectly, a compound of the present disclosure. Particularly favored derivatives and prodrugs include those that increase the bioavailability of the chemical entities of the present disclosure when such compounds are administered to a subject, for example by allowing an orally administered compound to be more readily absorbed into the blood, or which enhance delivery of the parent compound to a biological compartment, such as the brain or lymphatic system, relative to the parent species. Prodrugs can include derivatives where a group which enhances aqueous solubility or active transport through the gut membrane is appended to the compound of Formula I. Other prodrugs can include a promoiety that modifies the ADME (absorption, distribution, metabolism and excretion) of the parent compound and thereby enhances the therapeutic effectiveness of the parent compound.

In certain embodiments, chemical entities of the present disclosure can be modified by appending appropriate functionalities to enhance selective biological properties. Such modifications are known in the art and include those which can increase biological penetration into a given biological compartment, such as blood, lymphatic system, central nervous system, to increase oral availability, increase solubility to allow administration by injection, alter metabolism, and alter the rate of excretion.

In some embodiments, chemical entities of the present disclosure can be modified to facilitate use in biological assay, screening, and analysis protocols. Such modifications can include, for example, derivatizing to effect or enhance binding to physical surfaces such as beads or arrays, or modifying to facilitate detection such as by radiolabeling, affinity labeling, or fluorescence labeling.

Chemical entities of the present disclosure possess inhibitory activity with at least one ATP-utilizing enzyme. An ATP-utilizing enzyme refers to an enzyme that catalyzes the transfer of a phosphate group from an ATP molecule to a biomolecule such as a protein or carbohydrate. Examples of ATP-utilizing enzymes include, but are not limited to, synthetases, ligases, and kinases. The kinases can be animal kinases, including mammalian protein kinases, and human protein kinases.

In certain embodiments, chemical entities of the present disclosure exhibited human protein kinase inhibitory activity. Certain chemical entities of the present disclosure exhibited selectivity for one or more protein kinases, where selectivity is as defined herein. Certain chemical entities of the present disclosure exhibited selective activity for at least one of the following protein kinases, or pair of protein kinases: ABL1, AKT1, AKT2, AKT3, AURORA-A, c-TAK1, CDK1, CDK2/cyclinA, CDK2/cyclinE, CDK5, CHEK1, CHEK2, CSK, DAPK1, DYRK2, FLT-3, FYN, GSK3-α, GSK3-β, HCK, INSR, KIT, LCK, LYNA, MAPKAPK2, MAPKAPK3, MSK1, MSK2, p38-α, p38-β, p38-δ, p38-γ, P70S6K, PAK2, PDGFR-α, PAK1, PKA, PRAK, ROCK2, SGK1, SRC, SYK, PIM-1-kinase, PDK1, and RSK2.

Chemical entities of the present disclosure can be prepared by methods well known in the art. Chemical entities of the present disclosure can be prepared from readily available starting materials using the flowing general methods and procedures. It will be appreciated that where typical or preferred process conditions, such as, reaction temperatures, times, mole ratios of reactants, solvents, pressures, are given, other process conditions can also be used unless otherwise stated. Reaction conditions may vary with the reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, 3^{rd} Edition, John Wiley & Sons, 1999, and references cited therein.

Furthermore, chemical entities of the present disclosure can contain one or more chiral centers. Accordingly, if desired, such compounds can be prepared or isolated as pure stereoisomers, i.e., as individual enantiomers or diastereomers, or as stereoisomer-enriched mixtures. All such stereoisomers, and enriched mixtures thereof, are included within the scope of the present disclosure, unless otherwise indicated. Pure stereoisomers, and enriched mixtures thereof, can be prepared using, for example, optically active starting materials or stereoselective reagents well-known in the art. Alternatively, racemic mixtures of such compounds can be separated using, for example, chiral column chromatography, chiral resolving agents and the like.

General synthetic schemes and specific reaction protocols used to prepare chemical entities of the present disclosure are presented in the reaction schemes and Examples provided herein. In addition, general references for the preparation of substituted 1,2,4-triazoles, such as Science of Synthesis 2004, 13, 603-639, are available to those skilled in the art.

A compound of Formula I (where X is S) can be prepared as illustrated in Schemes 1 and 2 below. Reaction of hydrazides 1 with isothiocyanates 2 can provide compounds of structure 3, which may be cyclized under basic conditions to provide triazoles 4. Compounds of Formula Ia are tautomeric with 4. Reaction of 4 with the appropriate alkylating agent TCR⁴R⁵Q, where T is a leaving group such as Br, Cl, I, mesylate, or tosylate, can provide compounds of Formula Ib.

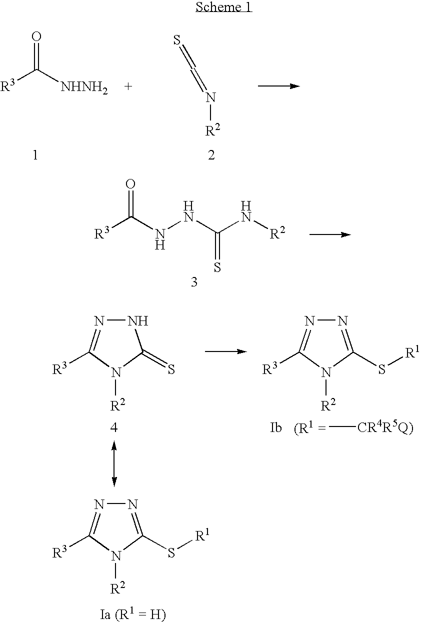

Hydrazides 1, when not commercially available, can be prepared via known procedures, e.g. from the corresponding esters by treatment with hydrazine. Isothiocyanates 2, when not commercially available, can be prepared via known procedures, e.g. from the corresponding amine by treatment with thiophosgene and a base. Alkylating agents TCR⁴R⁵Q, when not commercially available, can be prepared via known procedures by those skilled in the art.

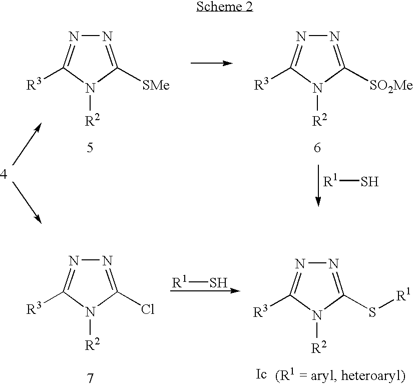

Reaction of 4 with a methylation agent, such as methyl iodide, can afford the thioether 5, which upon reaction with an oxidation agent such as hydrogen peroxide or peracetic acid can provide the sulfone 6. Treatment with an appropriate aryl or heteroaryl thiol can afford compounds of Formula Ic. Alternatively, reaction of 4 with chlorine can provide a chloride 7, which can react with the appropriate thiol to give compounds of Formula Ic.

A compound of Formula I (where X is O) can be prepared as illustrated in Schemes 3 and 4 below. Reaction of sulfones 6 or chlorides 7 with the appropriate alcohols under basic conditions can afford compounds of Formula Id. Preferably, conditions whereby the alkoxide of the corresponding alcohol is generated in situ are utilized.

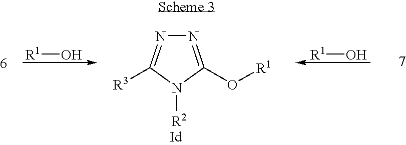

Alternatively, hydrazides 1 can be reacted with isocyanates 8, which under strongly basic conditions, cyclize to provide the triazoles 9, which are tautomeric with the hydroxytriazoles 10. Reaction of 9/10 with the appropriate alkylating agent TCR⁴R⁵Q, as described for the thiol derivatives in Scheme 1, can provide compounds of Formula Ie. It can be anticipated that under certain alkylation conditions a mixture of Ie and 11 would result. In those instances, separation of Ie from the reaction mixtures may be accomplished by those skilled in the art utilizing one or more of a variety of purification procedures (e.g. HPLC, silica gel chromatography, crystallization).

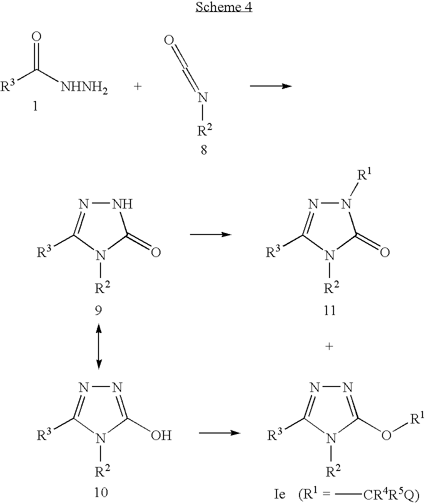

A compound of Formula I (where X is NR¹⁷) can be prepared as illustrated in Schemes 5 to 8 below. Reaction of sulfones 6 or chlorides 7 with the appropriate amines, preferably at temperatures above room temperature, can afford compounds of Formula If.

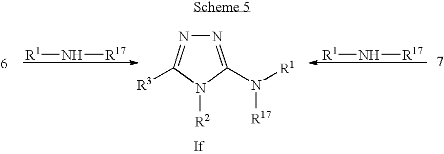

The core aminotriazole heterocycle 14, containing the appropriate R² and R³ groups, can be prepared directly from the simple amide 12, via dehydration/chlorination with phosphorus pentachloride and reaction with hydrazine to give amidrazone 13, followed by alkylation/cyclization with cyanogen bromide. Treatment of 14 with an acylating agent can provide intermediates 15 (e.g. J=OtBu, OBn, CF₃), which may be alkylated with the appropriate alkylating agent TCR⁴R⁵Q (see Scheme 1) using a strong, non-nucleophilic base such as lithium bis(trimethylsilyl)amide or lithium diisopropylamide to give 16. Removal of the acyl protecting group can provide compound of Formula Ig.

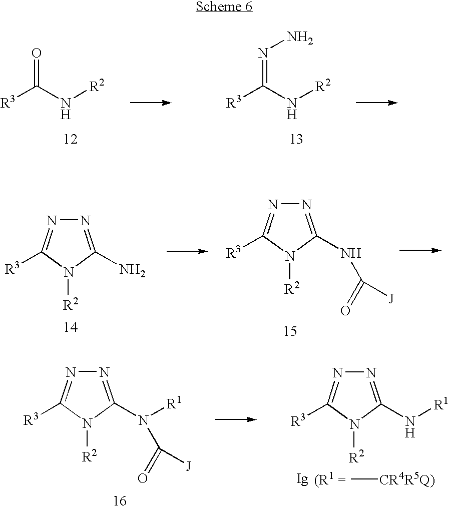

Compounds where R¹⁷ is not hydrogen can be prepared by in one step by reaction of the appropriate hydrazide 1 with the appropriate isothioureas 17 (see Eur. J. Med. Chem. 1978, 13, 469-74) to provide compounds of Formula Ih.

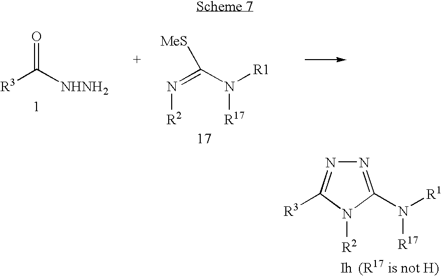

Alternatively, compounds of Formula Ih can be prepared directly from compounds of Formula Ig via alkylation with the appropriate R¹⁷-T reagent, where T is a leaving group such as Br, Cl, I, mesylate, or tosylate,

It can be anticipated that under certain alkylation conditions a mixture of Ih and 18 would result. In those instances, separation of Ih from the reaction mixtures may be accomplished by those skilled in the art utilizing one or more of a variety of purification procedures (e.g. HPLC, silica gel chromatography, crystallization).

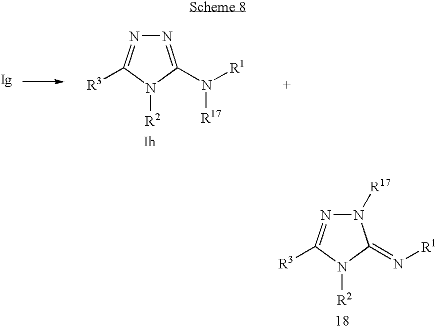

In accordance with certain embodiments, chemical entities of the present disclosure exhibit ATP-utilizing enzyme inhibitory activity. Thus, one use of the chemical entities of the present present disclosure includes the administration of at least one chemical entity of the present disclosure to a subject, such as a human. This administration serves to arrest, ameliorate, reduce the risk of acquiring, reduce the development of or at least one of the clinical symptoms of, or reduce the risk of developing or at least one of the clinical symptoms of diseases or conditions regulated by ATP-utilizing enzymes, such as, protein kinases.

For example, unregulated or inappropriately high protein kinase activity has been implicated in many diseases resulting from abnormal cellular function. Unregulated or inappropriately high protein kinase activity can arise either directly or indirectly, for example, by failure of the proper control mechanisms of a protein kinase, related, for example, to mutation, over-expression or inappropriate activation of the enzyme; or by over- or under-production of cytokines or growth factors also participating in the transduction of signal upstream or downstream of the protein kinase. In all of these instances, selective inhibition of the action of a protein kinase can be expected to have a beneficial effect.

According to certain embodiments, the present disclosure relates to methods of treating a disease regulated by at least one ATP-utilizing enzyme in a subject. ATP-utilizing enzyme regulated diseases include, for example, those where the ATP-utilizing enzyme participates in the signaling, mediation, modulation, control or otherwise involved in the biochemical processes affecting the manifestation of a disease. In certain embodiments, the methods are useful in treating diseases regulated by protein kinase enzymes. Protein kinase regulated diseases include, for example, the following general disease classes: cancer, autoimmunological, metabolic, inflammatory, infection, diseases of the central nervous system, degenerative neural disease, allergy/asthma, angiogenesis, neovascularization, vasculogenesis, cardiovascular, and the like. Without being limited by theory, specific examples of diseases that are known or believed to be regulated by protein kinase enzymes, include, transplant rejection, osteoarthritis, rheumatoid arthritis, multiple sclerosis, diabetes, diabetic retinopathy, asthma, inflammatory bowel disease such as Crohn's disease, and ulcerative colitis, renal disease cachexia, septic shock, lupus, diabetes mellitus, myasthenia gravis, psoriasis, dermatitis, eczema, seborrhea, Alzheimer's disease, Parkinson's disease, amyotrophic lateral sclerosis, depression, anxiety, obsessive compulsive disorder, stem cell protection during chemotherapy, ex vivo selection or ex vivo purging for autologous or allogeneic bone marrow transplantation, leukemia including, but not limited to, acute myeloid leukemia, chronic myeloid leukemia, and acute lymphoblastic leukemia, cancer including but not limited to, breast cancer, lung cancer, colorectal cancer, ovary cancer, prostate cancer, renal cancer, squamous cell cancer, glioblastoma, melanoma, pancreatic cancer, and Kaposi's sarcoma, enhancement of anti-cancer treatment, ocular disease, corneal disease, glaucoma, bacterial infections, viral infections, fungal infections, pain (including dental pain and neuropathic pain), heart disease, stroke, neuronal damage, spinal cord injury, and obesity.

Chemical entities of the present disclosure can be used in the treatment of diseases in which inappropriate protein kinase activity plays a role, including, for example, Alzheimer's disease, stroke, diabetes, obesity, inflammation, and cancer.

Certain embodiments of the present disclosure are directed to methods of treating disease in a subject comprising the step of administering to a subject, in need of such treatment, a therapeutically effective dosage of at least one compound of the present disclosure. In some embodiments, a disease can be regulated by at least one ATP-utilizing enzyme such as a protein kinase. Certain diseases can be regulated by one or more ATP-utilizing enzymes. In such cases, treatment of the disease or disorder can include administering a therapeutically effective amount of at least one compound of the present disclosure that inhibits the activity of one or more ATP-utilizing enzymes, or more than one compound of the present disclosure, wherein each compound inhibits at least one different ATP-utilizing enzyme.

Other embodiments of the present disclosure are related to methods of inhibiting at least one ATP-utilizing enzyme, including for example, a protein kinase. In certain embodiments, the ATP-utilizing enzyme can be inhibited by the method of administering to a subject, at least one chemical entity described herein, or a composition comprising at least chemical entity describe herein.

In certain embodiments, the present disclosure relates to methods of inhibiting ATP-utilizing enzyme activity by contacting at least one ATP-utilizing enzyme with at least one chemical entity of the present disclosure. ATP-utilizing enzymes include phosphotransferase enzymes that catalyze the phosphorylation of a biological molecule by transferring a phosphate group from an ATP substrate. ATP-utilizing enzymes include for example, synthetases, ligases, and kinases. Certain methods of the present disclosure are useful in inhibiting protein kinase enzymes, including, for example, the following protein kinase enzymes ABL1, AKT1, AKT2, AKT3, AURORA-A, c-TAK1, CDK1, CDK2/cyclinA, CDK2/cyclinE, CDK5, CHEK1, CHEK2, CSK, DAPK1, DYRK2, FLT-3, FYN, GSK3-α, GSK3-β, HCK, INSR, KIT, LCK, LYNA, MAPKAPK2, MAPKAPK3, MSK1, MSK2, p38-α, p38-β, p38-δ, p38-γ, P70S6K, PAK2, PDGFR-α, PAK1, PKA, PRAK, ROCK2, SGK1, SRC, SYK, PIM-1-kinase, PDK1, and RSK2.

Some methods of the present disclosure can be used to inhibit ATP-utilizing enzymes that are present in a living organism, such as a mammal; contained in a biological sample such as a cell, cell culture, or extract thereof, biopsied material obtained from a mammal or extracts thereof, and blood, saliva, feces, semen, tears or other body fluids or extracts thereof; contained within a reagent, or bound to a physical support. In certain embodiments, an ATP-utilizing enzyme can regulate a disease or disorder and in other embodiments, the ATP-utilizing enzyme may not regulate a disease or disorder.

According to the methods of the present disclosure, at least one ATP-utilizing enzyme can be inhibited by contact with at least one chemical entity of the present disclosure. In vivo ATP-utilizing enzymes can be inhibited by administration through routes and using compositions comprising at least one chemical entity of the present disclosure previously described. For in vitro systems, contacting an ATP-utilizing enzyme with at least one chemical entity of the present disclosure can include, for example, combining liquid reagents or combining a reagent and an ATP-utilizing enzyme and/or chemical entity of the present disclosure attached to a solid support. The ATP-utilizing enzyme and chemical entity of the present disclosure can be contacted in any appropriate device such as an affinity chromatography column, a microarray, a microfluidic device, assay plate, or other appropriate chemical or biotechnology apparatus used to perform biochemical analysis, assay, screening, and the like.

In certain embodiments, pharmaceutical compositions of the present disclosure may be administered orally, parenterally, by inhalation spray, topically, rectally, nasally, buccally, vaginally, via an implanted reservoir, or by any other appropriate route. Pharmaceutical compositions of the present disclosure can contain any conventional non-toxic pharmaceutically acceptable, excipients carriers, adjuvants and/or vehicles. In some embodiments, the pH of the formulation can be adjusted with pharmaceutically acceptable acids, bases or buffers to enhance the stability of the formulated compound or the delivery form. The term parenteral as used herein includes subcutaneous, intracutaneous, intravenous, intramuscular, intra-articular, intra-arterial, interasynovial, intrastemal, interathecal, intralesional, and intracranial injection or infusion techniques.

In certain embodiments, chemical entities disclosed herein can be delivered orally. Suitable dosage ranges for oral administration can depend on the potency of the chemical entity, but generally can range from 0.1 mg to 20 mg of a chemical entity per kilogram of body weight. Appropriate dosages can be in the range of 25 to 500 mg/day and the dose of chemical entity administered can be adjusted to provide an equivalent molar quantity of chemical entity in the plasma of a subject. Dosage ranges can be readily determined by methods known to those skilled in the art.

A dosage can be delivered in a composition by a single administration, by multiple applications, by sustained release or by controlled sustained release, or any other appropriate intervals and/or rates of release.

Chemical entities of the present disclosure can be assayed in vitro and in vivo, for the desired therapeutic or prophylactic activity prior to therapeutic use in mammals. For example, in vitro assays can be used to determine whether administration of one chemical entity of the present disclosure or a combination of such chemical entities is effective for inhibiting the activity of certain ATP-utilizing enzymes or treating at least one disease. Chemical entities of the present disclosure can also be demonstrated to be effective and safe using animal model systems. A therapeutically effective dose of a chemical entity of the present disclosure can, in certain embodiments, provide therapeutic benefit without causing substantial toxicity. Toxicity of chemical entities of the present disclosure can be determined using standard pharmaceutical procedures and can be readily ascertained by the skilled artisan. The dose ratio between toxic and therapeutic effect is the therapeutic index. Chemical entities of the present disclosure can exhibit high therapeutic indices in treating diseases and disorders. The dosage of a chemical entity of the present present disclosure can be within a range of circulating concentrations that include an effective dose with little or no toxicity.

When employed as pharmaceuticals, chemical entities of the present disclosure can be administered in the form of pharmaceutical compositions. Such compositions can be prepared in a manner well known in the pharmaceutical art and can comprise at least one chemical entity of the present disclosure.

Pharmaceutical compositions of the present disclosure can comprise a therapeutically effective amount of at least one chemical entity of the present disclosure, and at least one pharmaceutically acceptable excipient, such as, for example, diluents, carriers, or adjuvants. Pharmaceutical compositions of the present disclosure can additionally comprise at least one chemical entity that enhances the therapeutic efficacy of one or more chemical entities of the present disclosure. For example, such chemical entities can enhance the therapeutic efficacy of chemical entities of the present disclosure by effectively increasing the plasma concentration of the chemical entities. Without being limited by theory, certain chemical entities can decrease the degradation of the chemical entities of the present disclosure prior to administration or during transport to the plasma, or within the plasma. Certain chemical entites can increase the plasma concentration by increasing the absorption of chemical entities in the gastrointestinal tract. Pharmaceutical compositions of the present disclosure can also include additional therapeutic agents that are normally administered to treat a disease or disorder.

In some embodiments, chemical entities and compositions of the present disclosure can be administered by oral routes. The compositions can be prepared in a manner well known in the pharmaceutical art and can comprise at least one chemical entity of the present disclosure. In some embodiments, compositions of the present disclosure contain a therapeutically effective amount of one or more thiatriazole-based chemical entities of the present disclosure, which can be in purified form, together with a therapeutically effective amount of at least one additional therapeutic agent, and a suitable amount of at least one pharmaceutically acceptable excipient, so as to provide the form for proper administration to a subject

Some embodiments of the present disclosure are directed to compositions that contain, as the active ingredient, of at least one chemical entity of the present disclosure associated with pharmaceutically acceptable excipients. In making certain compositions of the present disclosure, the active ingredient can be mixed with an excipient, diluted by an excipient, or enclosed within such a carrier that can be in the form of a capsule, sachet, paper or other container. When the excipient serves as a diluent, the excipient can be a solid, semi-solid, or liquid material, which acts as a vehicle, carrier or medium for the active ingredient. Thus, for example, the compositions can be in the form of tablets, pills, powders, lozenges, sachets, cachets, elixirs, suspensions, emulsions, solutions, and syrups containing, for example, from 1% to 90% by weight of at least one chemical entity of the present disclosure using, for example, soft and hard gelatin capsules.

In preparing a composition, it can be necessary to mill the active chemical entity to provide the appropriate particle size prior to combining with other ingredients. If the active chemical entity is insoluble, the active component ordinarily can be milled to a particle size of less than 200 mesh. If the active chemical entity is water soluble, the particle size can be adjusted by milling to provide a uniform distribution in the formulation, e.g. 40 mesh.

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. Some compositions can additionally include, lubricating agents such as talc, magnesium stearate, and mineral oil, wetting agents, emulsifying and suspending agents, preserving agents such as methyl- and propylhydroxy-benzoates, sweetening agents, and flavoring agents. Compositions of the present disclosure can be formulated so as to provide quick, sustained or delayed release of the active ingredient after administration to the subject by employing procedures known in the art.

Some compositions of the present disclosure can be formulated in unit dosage form, each dosage containing, for example, 0.1 mg to 2 g of the active ingredient. As used herein, “unit dosage forms” refers to physically discrete units suitable as unitary dosages for human subjects and other mammals, each unit containing a predetermined quantity of active material calculated to produce the desired therapeutic effect, in association with a suitable pharmaceutical excipient, diluent, carrier and/or adjuvant. In certain embodiments, compositions of the present disclosure can be formulated in multiple dosage forms. The amount of the chemical entities of the present disclosure that can be combined with other materials and therapeutic agents to produce compositions of the present disclosure in a single dosage form will vary depending upon the subject and the particular mode of administration.

In the treatment of disease, chemical entities of the present disclosure can be administered in a therapeutically effective amount. It will be understood, however, that the amount of the chemical entity administered will be determined by a physician, in the light of the relevant circumstances, including the condition to be treated, the chosen route of administration, the actual chemical entity administered, the age, weight, and response of the individual subject, the severity of the subject's symptoms, and the like.

For preparing solid compositions such as tablets, the principal active ingredient can be mixed with a pharmaceutical excipient to form a solid preformulation composition containing a homogeneous mixture of a chemical entity of the present present disclosure. When referring to these preformulation compositions as homogeneous, it is meant that the active ingredient is dispersed evenly throughout the composition so that the composition may be readily subdivided into equally effective unit dosage forms such as tablets, pills and capsules. The solid preformulation can then subdivided into unit dosage forms of the type described above containing from, for example, 0.1 mg to 2 g of the therapeutically effective chemical entity of the present present disclosure.

The tablets or pills comprising certain compositions of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials include a number of polymeric acids and mixtures of polymeric acids with such materials as shellac, cetyl alcohol, and cellulose acetate.

The liquid forms in which the compositions of the present disclosure may be incorporated for administration orally or by injection include aqueous solutions suitably flavored syrups, aqueous or oil suspensions, and flavored emulsions with edible oils such as cottonseed oil, sesame oil, coconut oil, or peanut oil, as well as elixirs and similar pharmaceutical vehicles.

As used herein, a “pharmaceutically acceptable derivative or prodrug” refers to any pharmaceutically acceptable salt, ester, salt of an ester or other derivative of a compound of Formula 1 that, upon administration to a recipient, is capable of providing, either directly or indirectly, a compound of the present disclosure or an inhibitory active metabolite or residue thereof. Examples of such derivatives or prodrugs include those that increase the bioavailability of the chemical entities of the present disclosure when such compounds are administered to a mammal, e.g., by allowing an orally administered compound to be more readily absorbed into the blood, or which enhance delivery of the parent compound to a biological compartment, e.g., the brain or lymphatic system, relative to the parent species.

In certain embodiments, acceptable formulation materials can be nontoxic to recipients at the dosages and concentrations employed.

In certain embodiments, a pharmaceutical composition of the present disclosure can contain formulation materials for modifying, maintaining, or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids such as glycine, glutamine, asparagine, arginine or lysine; antimicrobials; antioxidants such as ascorbic acid, sodium sulfite, or sodium hydrogen-sulfite; buffers such as borate, bicarbonate, Tris-HCl, citrates, phosphates or other organic acids; bulking agents such as mannitol or glycine; chelating agents such as ethylenediamine tetraacetic acid (EDTA); complexing agents such as caffeine, polyvinylpyrrolidone, beta-cyclodextrin or hydroxypropyl-beta-cyclodextrin; fillers; monosaccharides; disaccharides; and other carbohydrates such as glucose, mannose, or dextrins; proteins such as serum albumin, gelatin or immunoglobulins; coloring, flavoring and diluting agents; emulsifying agents; hydrophilic polymers such as polyvinylpyrrolidone; low molecular weight polypeptides; salt-forming counterions such as sodium; preservatives such as benzalkonium chloride, benzoic acid, salicylic acid, thimerosal, phenethyl alcohol, methylparaben, propylparaben, chlorhexidine, sorbic acid or hydrogen peroxide; solvents such as glycerin, propylene glycol or polyethylene glycol; sugar alcohols such as mannitol or sorbitol; suspending agents; surfactants or wetting agents such as pluronics, PEG, sorbitan esters, polysorbates such as polysorbate 20, polysorbate 80, triton, tromethamine, lecithin, cholesterol, tyloxapal; stability enhancing agents such as sucrose or sorbitol; tonicity enhancing agents such as alkali metal halides, such as sodium or potassium chloride, mannitol, sorbitol; delivery vehicles; diluents; excipients and/or pharmaceutical adjuvants. (Remington's Pharmaceutical Sciences, 18^{th} Edition, A. R. Gennaro, ed., Mack Publishing Company (1990)).

In certain embodiments, the optimal pharmaceutical composition can be determined by one skilled in the art depending upon, for example the intended route of administration, delivery format, and desired dosage. See, for example, Remington's Pharmaceutical Sciences, supra. In certain embodiments, such compositions may influence the physical state, stability, rate of in vivo release, and rate of in vivo clearance of the antibodies of the present disclosure.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In certain embodiments, neutral buffered saline or saline mixed with serum albumin are further exemplary vehicles. In certain embodiments, pharmaceutical compositions comprise Tris buffer of pH 7 to 8.5, or acetate buffer of pH 4 to 5.5, which can further comprise sorbitol or a suitable substitute thereof. In certain embodiments, buffers are used to maintain the composition at physiological pH or at a slightly lower pH, typically within a pH range of from 5 to 8.

In certain embodiments, the pharmaceutical compositions of the present disclosure can be selected for parenteral delivery. In other embodiments, the compositions can be selected for inhalation or for delivery through the digestive tract, such as orally. The preparation of such pharmaceutically acceptable compositions is within the skill of the art.

In certain embodiments, the composition components cam be present in concentrations that are acceptable to the site of administration. In certain embodiments, when parenteral administration is contemplated, a therapeutic composition can be in the form of a pyrogen-free, parenterally acceptable aqueous solution comprising at least one chemical entity of the present disclosure, with or without additional therapeutic agents, in a pharmaceutically acceptable vehicle. In other embodiments, a vehicle for parenteral injection can be sterile distilled water in which at least one chemical entity of the present disclosure, with or without at least one additional therapeutic agent, is formulated as a sterile, isotonic solution, properly preserved. In still other embodiments, the pharmaceutical composition can include encapsulation of a at least one chemical entity of the present disclosure with an agent, such as injectable microspheres, bio-erodible particles, polymeric compounds such as polyacetic acid or polyglycolic acid, beads or liposomes, that can provide the controlled or sustained release of the chemical entity of the present disclosure which can then be delivered via a depot injection. In certain embodiments, implantable drug delivery devices can be used to introduce a chemical entity of the present disclosure to the plasma of a subject, within a target organ, or to a specific site within the subject's body.

In certain embodiments, a pharmaceutical composition can be formulated for inhalation. In certain embodiments, a chemical entity of the present disclosure, with or without at least one additional therapeutic agent, can be formulated as a dry powder for inhalation. In certain embodiments, an inhalation solution comprising a at least one chemical entity of the present disclosure with or without at least one additional therapeutic agent can be formulated with a propellant for aerosol delivery. In other embodiments, solutions can be nebulized. In still other embodiments, solutions, powders or dry films of chemical entities of the present disclosure can be aerosolized or vaporized for pulmonary deliver.

In certain embodiments, it is contemplated that formulations can be administered orally. In certain embodiments, at least one chemical entity of the present disclosure, with or without at least one additional therapeutic agent that can be administered orally, can be formulated with or without carriers customarily used in the compounding of solid dosage forms such as tablets and capsules. In other embodiments, a capsule may be designed to release the active portion of the formulation in the region of the gastrointestinal tract where bioavailability can be maximized and pre-systemic degradation minimized. In still other embodiments, at least one additional agent can be included in the formulation to facilitate absorption of at least one chemical entity of the present disclosure and/or any additional therapeutic agents into the systemic circulation. In certain embodiments, diluents, flavorings, low melting pint waxes, vegetable oils, lubricants, suspending agents, tablet disintegrating agents, and binders can be employed.

In certain embodiments, a pharmaceutical composition of the present disclosure can include an effective quantity of at least one chemical entity of the present disclosure, with or without at least one additional therapeutic agent, in a mixture with non-toxic excipients which are suitable for the manufacture of tablets. In certain embodiments, by dissolving the tablets in sterile water, or other appropriate vehicle, solutions can be prepared in unit-dose form. In certain embodiments, suitable excipients include inert diluents, such as calcium carbonate, sodium carbonate or bicarbonate, lactose, or calcium phosphate; or binding agents, such as starch, gelatin, or acacia; and lubricating agents such as magnesium stearate, stearic acid or talc.

In certain embodiments, the frequency of dosing will take into account the pharmacokinetic parameters of the chemical entity and/or any additional therapeutic agents in the pharmaceutical composition used. In certain embodiments, a clinician can administer the composition until a dosage is reached that achieves the desired effect. The composition can be administered as a single dose, or as two or more doses, which may or may not contain the same amount of the therapeutically active compound time, or as a continuous infusion via an implantation device or catheter. Further refinement of an appropriate dosage can be routinely made by those of ordinary skill in the art. For example, therapeutically effective dosages and dosage regiments can be determined through use of appropriate dose-response data.

In certain embodiments, the route of administration of the pharmaceutical composition can be in accord with known methods, e.g. orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. In certain embodiments, the compositions can be administered by bolus injection or continuously by infusion, or by an implantation device.

In certain embodiments, the composition can be administered locally via implantation of a membrane, sponge or another appropriate material onto which the desired chemical entity of the present disclosure has been absorbed or encapsulated. In certain embodiments, where an implantation device is used, the device can be implanted into any suitable tissue or organ, and delivery of the desired molecule via diffusion, timed-release bolus, or continuous administration.

In certain embodiments, it can be desirable to use a pharmaceutical composition comprising at least one chemical entity of the present disclosure, with or without at least one additional therapeutic agent, in an ex vivo manner. For example, cells, tissues and/or organs that have been removed from a subject are exposed to a pharmaceutical composition comprising at least one chemical entity of the present disclosure, with or without at least one additional therapeutic agent, after which the cells, tissues and/or organs are subsequently implanted back into the subject.

In certain embodiments, at least one chemical entity of the present disclosure and/or any additional therapeutic agents can be delivered by implanting certain cells that have been genetically engineered, using methods known in the art, to express and secrete at least one chemical entity of the present disclosure. In certain embodiments, such cells can be animal or human cells, and can be autologous, heterologous, or xenogeneic. In certain embodiments, the cells can be immortalized. In certain embodiments, in order to decrease the chance of an immunological response, the cells can be encapsulated to avoid infiltration of surrounding tissues. In certain embodiments, the encapsulation materials can be biocompatible, semi-permeable polymeric enclosures or membranes that enable the release of the protein product(s) while preventing the destruction of the cells by the subject's immune system or by other detrimental factors originating from the surrounding tissues.

Pharmaceutical compositions according to the present disclosure can take a form suitable for oral, buccal, parenteral, nasal, topical or rectal administration, or a form suitable for administration by inhalation or insufflation.

The compositions of the present disclosure can, if desired, be presented in a pack or dispenser device that can contain one or more unit dosage forms containing the active ingredient. The pack or dispensing device can be accompanied by instructions for administration.

The quantity of at least one chemical entity of the present disclosure required for the treatment of a particular condition can vary depending on the chemical entity, and the condition of the subject to be treated. In general, daily dosages can range from 100 ng/kg to 100 mg/kg, e.g., 0.01 mg/kg to 40 mg/kg body weight, for oral or buccal administration; from 10 ng/kg to 50 mg/kg body weight, e.g., 0.001 mg/kg to 20 mg/kg body weight, for parenteral administration; and from 0.05 mg to 1,000 mg for nasal administration or administration by inhalation or insufflation.

Certain chemical entities of the present disclosure and/or compositions of the present disclosure can be administered as sustained release systems. In certain embodiments, the chemical entities of the present disclosure can be delivered by oral sustained release administration. In this embodiment, at least one chemical entity of the present disclosure can be administered, for example, twice per day and, once per day.

The methods of the present disclosure can be practiced with a number of different dosage forms, which can be adapted to provide sustained release of at least one chemical entity upon oral administration.

In one embodiment of the present disclosure, the dosage form comprises beads that on dissolution or diffusion release at least one chemical entity of the present disclosure over an extended period of hours, for example, over a period of at least 6 hours, over a period of at least 8 hours or over a period of at least 12 hours. The compound-releasing beads can include a central composition or core comprising at least one chemical entity of the present disclosure and pharmaceutically acceptable vehicles, including an optional lubricant, antioxidant and buffer. The beads can be medical preparations with a diameter of 1 to 2 mm. Individual beads can comprise doses of a compound of the present disclosure, for example, doses of up to 40 mg of the compound. In certain embodiments, the beads can be formed of non-cross-linked materials to enhance discharge of the beads from the gastrointestinal tract. The beads can be coated with a release rate-controlling polymer that gives a timed-release profile.

The timed-release beads can be manufactured into a tablet for therapeutically effective administration of a compound of the present disclosure. The beads can be formed into matrix tablets by the direct compression of a plurality of beads coated with, for example, an acrylic resin, and blended with excipients such as hydroxypropylmethyl cellulose.

In other embodiments, an oral sustained release pump can be used.

In other embodiments, polymeric materials can be used. In other embodiments, polymeric materials appropriate for oral sustained release delivery can be used. Examples of useful polymers include sodium carboxymethylcellulose, hydroxypropylcellulose, hydroxypropylmethylcellulose, hydroxyethylcellulose, and hydroxypropylmethylcellulose. Factors affecting controlled drug release are well known to the skilled artisan.

In other embodiments, enteric-coated preparations can be used for oral sustained release administration. Enteric coating materials include polymers exhibiting a pH-dependent solubility (i.e., pH-controlled release), polymers exhibiting a slow or pH-dependent rate of swelling, dissolution or erosion (i.e., time-controlled release), polymers that can be degraded by enzymes (i.e., enzyme-controlled release), and polymers capable of forming firm layers that can be destroyed by an increase in pressure (i.e., pressure-controlled release).

In still other embodiments, drug-releasing lipid matrices can be used for oral sustained release administration. In one example, chemical entities of the present disclosure can be coated with a thin controlled release layer of a lipid to form solid microparticles, such as glyceryl behenate and/or glyceryl palmitostearate. The lipid-coated particles can optionally be compressed to form a tablet. Another controlled release lipid-based matrix material which can be suitable for sustained-release oral administration comprises polyglycolized glycerides.

In still other embodiments, compound-releasing waxes can be used for oral sustained release administration. Examples of suitable sustained drug-releasing waxes include carnauba wax, candedilla wax, esparto wax, ouricury wax, hydrogenated vegetable oil, bees wax, paraffin, castor wax, ozokerite, and mixtures thereof.

In still other embodiments, osmotic delivery systems can be used for oral sustained release administration.

In other embodiments, a controlled-release system can be placed in proximity to the target of the compound of the present disclosure, thus requiring only a fraction of the systemic dose.

In other embodiments, the dosage form can comprise a compound of the present disclosure coated on a polymer substrate. The polymer can be an erodible, or a nonerodible polymer. The coated substrate can be folded to provide a bilayer polymer drug dosage form. For example, a compound of the present disclosure can be coated onto a polymer such as a polypeptide, collagen, gelatin, polyvinyl alcohol, polyorthoester, polyacetyl, or a polyorthocarbonate, and the coated polymer folded to provide a bilaminated dosage form. In practice, the bioerodible dosage form can erode at a controlled rate to dispense the compound over a sustained release period. Representative biodegradable polymers include a polymer chosen from biodegradable poly(amides), poly(amino acids), poly(esters), poly(lactic acid), poly(glycolic acid), poly(carbohydrate), poly(orthoester), poly (orthocarbonate), poly(acetyl), poly(anhydrides), biodegradable poly(dehydropyrans), and poly(dioxinones).

In other embodiments, the dosage form can comprise a compound of the present disclosure loaded into a polymer that can release the compound by diffusion through a polymer, by flux through pores, or by rupture of a polymer matrix. The drug delivery polymeric dosage form can comprise a concentration of from 10 mg to 2,500 mg of the compound, homogenously contained in or on a polymer. The dosage form can comprise at least one exposed surface at the beginning of dose delivery. The non-exposed surface, when present, can be coated with a pharmaceutically acceptable material impermeable to the passage of the compound of the present disclosure. The dosage form can be manufactured by procedures known in the art. An example of providing a dosage form includes blending a pharmaceutically acceptable carrier such as polyethylene glycol, with a known dose of a compound of the present disclosure at an elevated temperature, such as 37° C., and adding the blend to a Silastic® medical grade elastomer with a cross-linking agent, for example, octanoate, followed by casting in a mold. The step can be repeated for each optional successive layer. The system can be allowed to set for 1 hour, to provide the dosage form. Representative polymers for manufacturing the dosage form include olefin, and vinyl polymers, addition polymers, condensation polymers, carbohydrate polymers, and silicon polymers as represented by polyethylene, polypropylene, polyvinyl acetate, polymethylacrylate, polyisobutylmethacrylate, polyalginate, polyamide, and polysilicon.

In other embodiments, the dosage form can comprise a plurality of tiny pills. Tiny time-released pills can provide a number of individual doses characterized by different temporal release profiles for achieving a sustained-release profile over an extended period of time, such as up to 24 hours. The matrix can comprise a hydrophilic polymer, such as a polysaccharide, agar, agarose, natural gum, alkali alginate including sodium alginate, carrageenan, fucoidan, furcellaran, laminaran, hypnea, gum arabic, gum ghatti, gum karaya, gum tragacanth, locust bean gum, pectin, amylopectin, gelatin, or a hydrophilic colloid. A hydrophilic matrix can comprise a plurality of 4 to 50 tiny pills, each tiny pill containing a dose of from 10 ng, 0.5 mg, 1 mg, 1.2 mg, 1.4 mg, 1.6 mg, 5.0 mg, or greater. The tiny pills can comprise a release rate-controlling wall ranging from 0.001 mm to 10 mm thickness to enable the timed release of a compound of the present disclosure. Representative wall-forming materials include a triglyceryl ester such as glyceryl tristearate, glyceryl monostearate, glyceryl dipalmitate, glyceryl laureate, glyceryl didecenoate, and glyceryl tridenoate. Other wall-forming materials include polyvinyl acetate, phthalate, methylcellulose phthalate, and microporous olefins.

In other embodiments, the dosage form can comprise an osmotic dosage form, which can include a semipermeable wall surrounding a therapeutic composition comprising at least one compound of the present disclosure. An osmotic dosage form comprising a homogenous composition can imbibe fluid through the semipermeable wall into the dosage form in response to concentration gradients across the semipermeable wall. The therapeutic composition in the dosage form can develop osmotic energy that can cause the therapeutic composition to be administered through an exit from the dosage form over a prolonged period of time, such as up to 24 hours, to provide controlled and sustained release of a compound of the present disclosure.

In other embodiments, the dosage form can comprise an osmotic dosage form comprising a wall surrounding a compartment, the wall having a semipermeable polymeric composition permeable to the passage of fluid and impermeable to the passage of a compound of the present disclosure contained within the compartment, a compound-containing layer composition in the compartment, a hydrogel layer composition in the compartment comprising an osmotic formulation for imbibing and absorbing fluid for expanding in size for pushing the prodrug or derivative composition layer from the dosage form, and at least one passageway in the wall for releasing the composition containing a compound of the present disclosure. This method can deliver a compound of the present disclosure by imbibing fluid through the semipermeable wall at a fluid imbibing rate determined by the permeability of the semipermeable wall and the osmotic pressure across the semipermeable wall causing the push layer to expand, thereby delivering the compound from the dosage form through the exit passageway to a subject over a prolonged period of time, such as up to 24 hours.

The hydrogel layer composition can comprise 10 mg to 1,000 mg of a hydrogel such as a polyalkylene oxide of 1,000,000 to 8,000,000 weight-average molecular weight, for example, a polyethylene oxide of 1,000,000 weight-average molecular weight, a polyethylene oxide of 2,000,000 molecular weight, a polyethylene oxide of 4,000,000 molecular weight, a polyethylene oxide of 5,000,000 molecular weight, a polyethylene oxide of 7,000,000 molecular weight and a polypropylene oxide of the 1,000,000 to 8,000,000 weight-average molecular weight; or 10 mg to 1000 mg of an alkali carboxymethylcellulose of 10,000 to 6,000,000 weight average molecular weight, such as sodium carboxymethylcellulose or potassium carboxymethylcellulose. The hydrogel expansion layer can comprise 0.1 mg to 350 mg of a polymer, for example, 0.1 mg to 250 mg of a hydroxyalkylcellulose of 7,500 to 4,500,00 weight-average molecular weight such as hydroxymethylcellulose, hydroxyethylcellulose, hydroxypropylcellulose, hydroxybutylcellulose or hydroxypentylcellulose; 0.1 mg to 50 mg of an osmagent chosen from sodium chloride, potassium chloride, potassium acid phosphate, tartaric acid, citric acid, raffinose, magnesium sulfate, magnesium chloride, urea, inositol, sucrose, glucose and sorbitol; 0.1 to 5 mg of a colorant, such as ferric oxide; 0.1 to 1.5 mg of an antioxidant including ascorbic acid, butylated hydroxyanisole, butylatedhydroxyquinone, butylhydroxyanisol, hydroxycoumarin, butylated hydroxytoluene, cephalm, ethyl gallate, propyl gallate, octyl gallate, lauryl gallate, propyl-hydroxybenzoate, trihydroxybutylrophenone, dimethylphenol, dibutylphenol, vitamin E, lecithin and ethanolamine; and 0.1 mg to 7 mg of a lubricant inluding calcium stearate, magnesium stearate, zinc stearate, magnesium oleate, calcium palmitate, sodium suberate, potassium laureate, salts of fatty acids, salts of alicyclic acids, salts of aromatic acids, stearic acid, oleic acid, palmitic acid, a mixture of a salt of a fatty, alicyclic or aromatic acid, and a fatty, alicyclic, or aromatic acid.

In the osmotic dosage forms, the semipermeable wall can comprise a composition that is permeable to the passage of fluid and impermeable to the passage of the compound of the present disclosure. The wall is nontoxic and comprises a polymer such as a cellulose acylate, cellulose diacylate, cellulose triacylate, cellulose acetate, cellulose diacetate or cellulose triacetate. The wall can comprise 75 wt % (weight percent) to 100 wt % of the cellulosic wall-forming polymer; or, the wall can additionally comprise 0.01 wt % to 80 wt % of polyethylene glycol, or 1 wt % to 25 wt % of a cellulose ether including, for example, hydroxypropylcellulose or a hydroxypropylalkycellulose such as hydroxypropylmethylcellulose. The total weight percent of all components comprising the wall is equal to 100 wt %. The internal compartment can comprise the compound or composition of the present disclosure alone or in layered position with an expandable hydrogel composition. The expandable hydrogel composition in the compartment can increase in dimension upon imbibing the fluid through the semipermeable wall, causing the hydrogel to expand and occupy space in the compartment, whereby a pharmaceutical composition is pushed from the dosage form. The therapeutic layer and the expandable layer can act together to release of a compound of the present disclosure to a subject over time. The dosage form comprises a passageway in the wall that connects the exterior of the dosage form with the internal compartment.

As used herein, “passageway” refers to means and methods suitable for the metered release of the chemical entities of the present disclosure from the compartment of the dosage form. The exit means can comprise at least one passageway, including orifice, bore, aperture, pore, porous element, hollow fiber, capillary tube, channel, porous overlay, or porous element that can provide for the osmotic controlled release of a compound of the present disclosure. The passageway can include a material that erodes or is leached from the wall in a fluid environment of use to produce at least one controlled-release dimensioned passageway. Representative materials suitable for forming a passageway, or a multiplicity of passageways include a leachable poly(glycolic) acid or poly(lactic) acid polymer in the wall, a gelatinous filament, poly(vinyl alcohol), leach-able polysaccharides, salts, and oxides. A pore passageway, or more than one pore passageway, can be formed by leaching a leachable compound, such as sorbitol, from the wall. The passageway can possess controlled-release dimensions, such as round, triangular, square and elliptical, for the metered release of a compound of the present disclosure from the dosage form. The dosage form can be constructed with one or more passageways in spaced apart relationship on a single surface or on more than one surface of the wall. As used herein, “fluid environment” refers to an aqueous or biological fluid as in a subject, including the gastrointestinal tract.

Regardless of the specific form of sustained release oral dosage form used, the compounds and composition of the present disclosure can be released from the dosage form over an extended period of time. In certain embodiments, sustained release oral dosage forms can provide a therapeutically effective amount of a compound of the present disclosure over a period of at least several hours. In certain embodiments the extended release dosage form can provide a constant therapeutically effective concentration of a compound of the present disclosure in the plasma of a subject for a prolonged period of time, such as at least several hours. In other embodiments, the sustained release oral dosage form can provide a controlled and constant concentration of a therapeutically effective amount of a compound of the present disclosure in the plasma of a subject.

Dosage forms comprising compositions and chemical entities of the present disclosure can be administered at certain intervals such as, for example, twice per day or once per day.

Exemplary dosage ranges for oral administration are dependent on the potency of the compound of the present disclosure, but can range from 0.1 mg to 20 mg of the compound per kilogram of body weight. Dosage ranges may be readily determined by methods known to those skilled in the art.

Chemical entities of the present disclosure can be assayed in vitro and in vivo, to determine and optimize therapeutic or prophylactic activity prior to use in subjects. For example, in vitro assays can be used to determine whether administration of a specific compound of the present disclosure or a combination of such compounds exhibits therapeutic efficacy. Chemical entities of the present disclosure can also be demonstrated to be effective and safe using animal model systems.

It is desirable that a therapeutically effective dose of a compound of the present disclosure provide therapeutic benefit without causing substantial toxicity. Toxicity of chemical entities of the present disclosure can be determined using standard pharmaceutical procedures and can be readily ascertained by the skilled artisan. The dose ratio between toxic and therapeutic effect is the therapeutic index. In certain embodiments, chemical entities of the present disclosure can exhibit particularly high therapeutic indices in treating diseases and disorders. In certain embodiments, the dosage of a compound of the present disclosure can be within a range of circulating concentrations that exhibit therapeutic efficacy with limited or no toxicity.

### EXAMPLES

Embodiments of the present disclosure can be further defined by reference to the following examples, which describe in detail preparation of compounds and compositions of the present disclosure and assays for using compounds and compositions of the present disclosure. It will be apparent to those skilled in the art that may modifications, both to materials and methods, may be practiced without departing from the scope of the present disclosure.

In the examples below, the following abbreviations have the following meanings. If an abbreviation is not defined, it has its generally accepted meaning.
AcOH=acetic acid
Atm=atmosphere
ATP=adenosine triphosphate
Boc=tert-butyloxycarbonyl
BSA=bovine serum albumin
Da=Dalton
DMF=N,N-dimethylformamide
DMSO=dimethylsulfoxide
DTT=(R,R)-dithiothrietol
EDTA=ethylenediaminetetraacetic acid
g=gram
hr=hour
HEPES=4-(2-hydroxyethyl)-1-piperazin-ethanesulfonic acid
HPLC=high performance liquid chromatography
HTS=high throughput screen
kDa=kilo Dalton
L=liter
LC/MS=liquid chromatography/mass spectroscopy
M=molar
MS=mass spectroscopy
mg=milligram
min=minute
mL=milliliter
mm=millimeter
mmol=millimoles
mM=millimolar
nM=nanomolar
NaOH=sodium hydroxide
μL=microliter
μM=micromolar
psi=pounds per square inch
RT=room temperature
TCB=trough circulating buffer
THF=tetrahydrofuran
TFA=trifluoroacetic acid
TLC=thin layer chromatography
TMS=trimethylsilyl
UV=ultraviolet
v/v=volume to volume

### General Procedure for Hydrazide/Isothiocyanate Condensation

A mixture of the appropriate hydrazide (1 mmol) and isothiocyanate (1 mmol) in dioxane (1 mL) was stirred at either room temperature or 50° C. until the reaction was complete, as monitored by conventional methods (e.g. TLC, LC/MS). The cooled reaction mixture was then concentrated to provide the desired crude product.

### General Procedure for Cyclization to a 1,2,4-Triazole-3-thione

To the hydrazide/isothiocyanate condensation product was added an excess of 1M aqueous NaOH, and the mixture was heated at 60° C. until complete, cooled to room temperature, then quenched with an excess of AcOH. Concentration in vacuo provided the desired crude product.

### Alternative Procedure for Cyclization to a 1,2,4-Triazole-3-thione

To the hydrazide/isothiocyanate condensation product was added an excess of 2.5M aqueous NaOH, and the mixture was heated in a microwave apparatus at 80° C. for 10 min, cooled to room temperature, then quenched with an excess of concentrated HCl. In many cases, the product precipitated and was isolated by filtration; otherwise, concentration in vacuo provided the desired crude product.

### General Procedure for Alkylation of the 1,2,4-Triazole-3-thione

To a mixture of the appropriate 1,2,4-triazole-3-thione (1 mmol) in DMSO (1 mL) was added a mixture of the appropriate alkylating agent (1 mmol) in DMSO (1 mL), followed by addition of N,N-diisopropylethylamine (1 mmol). The reaction mixture was maintained at room temperature until complete, then purified directly by reverse phase HPLC to give the desired product.

### Example 1

### Synthesis of

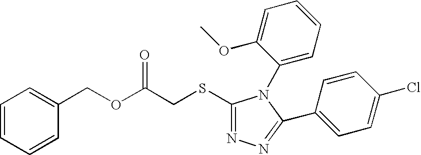

To a suspension of 4-chlorobenzhydrazide (8.5 mg, 0.05 mmol) in dioxane (0.05 mL) in one well of a 96-well polypropylene plate was added 2-methoxyphenyl isothiocyanate (8.3 mg, 0.05 mmol). The plate was covered and the reaction mixture was heated at 50° C. for 30 min, cooled to room temperature, and concentrated in vacuo. To the residue was added 1N aqueous NaOH (0.1 mL), and the reaction mixture was covered and heated at 60° C. for 24 hr, then cooled to room temperature. The reaction was quenched with an excess of acetic acid, then concentrated in vacuo. To the resulting residue was added a suspension of benzyl 2-bromoacetate (22.9 mg, 0.1 mmol) in DMSO (0.1 mL), followed by N,N-diisopropylethylamine (0.07 mL, 0.054 mmol). The reaction mixture was covered and incubated at room temperature for 24 hr, then directly purified by reverse phase HPLC to give the title compound (2.0 mg, 10%) as a colorless glass. LC/MS (ESI) m/z 466.3 [M+H]. HPLC retention time=3.66 min.

The following compounds listed in the following table were prepared by the general procedures as set forth in the general procedures and as exemplified in Example 1, utilizing the appropriate starting materials.

TABLE 1

| | | HPLC | |
|---|---|---|---|
| | LC/MS m/z | retention time | Compound |
| Compound Structure | [M + H] | (min) | Number |
| Benzyl 2-(5-(3-hydroxyphenyl)-4-phenyl- | 418.3 | 3.1 | 14.1 |
| 4H-1,2,4-triazol-3-ylthio)acetate | | | |
| Benzyl 2-(5-(2-methoxyphenyl)-4-phenyl- | 432.3 | 3.35 | 14.2 |
| 4H-1,2,4-triazol-3-ylthio)acetate | | | |
| Benzyl 2-(5-(4-chlorophenyl)-4-(2- | 466.3 | 3.66 | 14.3 |
| methoxyphenyl)-4H-1,2,4-triazol-3- | | | |
| ylthio)acetate | | | |
| Benzyl 2-(4-(2-methoxyphenyl)-5-o-tolyl- | 446.3 | 3.45 | 14.4 |
| 4H-1,2,4-triazol-3-ylthio)acetate | | | |
| Benzyl 2-(4-(2,4-difluorophenyl)-5-(4- | 468.3 | 3.56 | 14.5 |
| methoxyphenyl)-4H-1,2,4-triazol-3- | | | |
| ylthio)acetate | | | |
| Benzyl 2-(4-(2,4-difluorophenyl)-5-(2- | 453.9 | 3.52 | 14.6 |
| hydroxyphenyl)-4H-1,2,4-triazol-3- | | | |
| ylthio)acetate | | | |
| Benzyl 2-(4-(2-chlorophenyl)-5-(4- | 466.3 | 3.49 | 14.7 |
| methoxyphenyl)-4H-1,2,4-triazol-3- | | | |
| ylthio)acetate | | | |
| Benzyl 2-(4-(2-chlorophenyl)-5-(2- | 451.9 | 3.67 | 14.8 |
| hydroxyphenyl)-4H-1,2,4-triazol-3- | | | |
| ylthio)acetate | | | |
| Benzyl 2-(4-(4-chlorophenyl)-5-(4- | 466.3 | 3.63 | 14.9 |
| methoxyphenyl)-4H-1,2,4-triazol-3- | | | |
| ylthio)acetate | | | |
| Benzyl 2-(4-(4-chlorophenyl)-5-(2- | 451.9 | 3.69 | 14.10 |
| hydroxyphenyl)-4H-1,2,4-triazol-3- | | | |
| ylthio)acetate | | | |
| Benzyl 2-(4-(2-cyanophenyl)-5-(4- | 457.1 | 4.36 | 14.11 |
| methoxyphenyl)-4H-1,2,4-triazol-3- | | | |
| ylthio)acetate | | | |
| Benzyl 2-(4,5-diphenyl-4H-1,2,4-triazol-3- | 402.3 | 3.4 | 14.12 |
| ylthio)acetate | | | |
| Benzyl 2-(5-(4-fluorophenyl)-4-phenyl-4H- | 419.9 | 3.49 | 14.13 |
| 1,2,4-triazol-3-ylthio)acetate | | | |
| Benzyl 2-(5-(2-chlorophenyl)-4-phenyl-4H- | 436.6 | 3.5 | 14.14 |
| 1,2,4-triazol-3-ylthio)acetate | | | |
| Benyl 2-(4-phenyl-5-m-tolyl-4H-1,2,4- | 416.3 | 3.55 | 14.15 |
| triazol-3-ylthio)acetate | | | |
| Benzyl 2-(5-(3-chlorophenyl)-4-phenyl-4H- | 436.6 | 3.67 | 14.16 |
| 1,2,4-triazol-3-ylthio)acetate | | | |
| Benzyl 2-(4-phenyl-5-p-tolyl-4H-1,2,4- | 416.3 | 3.53 | 14.17 |
| triazol-3-ylthio)acetate | | | |
| Benzyl 2-(4-phenyl-5-o-tolyl-4H-1,2,4- | 416.3 | 3.48 | 14.18 |
| triazol-3-ylthio)acetate | | | |
| Benzyl 2-(5-(3-methoxyphenyl)-4-phenyl- | 432.3 | 3.44 | 14.19 |
| 4H-1,2,4-triazol-3-ylthio)acetate | | | |
| Benzyl 2-(5-(4-hydroxyphenyl)-4-phenyl- | 418.3 | 3 | 14.20 |
| 4H-1,2,4-triazol-3-ylthio)acetate | | | |
| Benzyl 2-(5-(2,4-difluorophenyl)-4-phenyl- | 437.9 | 3.52 | 14.21 |
| 4H-1,2,4-triazol-3-ylthio)acetate | | | |
| Benzyl 2-(4-(2-methoxyphenyl)-5-phenyl- | 432.3 | 3.39 | 14.22 |
| 4H-1,2,4-triazol-3-ylthio)acetate | | | |
| Benzyl 2-(4-(2-methoxyphenyl)-5-(4- | 462.3 | 3.34 | 14.23 |
| methoxyphenyl)-4H-1,2,4-triazol-3- | | | |
| ylthio)acetate | | | |
| Benzyl 2-(5-(4-fluorophenyl)-4-(2- | 449.9 | 3.47 | 14.24 |
| methoxyphenyl)-4H-1,2,4-triazol-3- | | | |
| ylthio)acetate | | | |
| Benzyl 2-(5-(2-chlorophenyl)-4-(2- | 466.3 | 3.48 | 14.25 |
| methoxyphenyl)-4H-1,2,4-triazol-3- | | | |
| ylthio)acetate | | | |
| Benzyl 2-(4-(2-methoxyphenyl)-5-m-tolyl- | 446.3 | 3.52 | 14.26 |
| 4H-1,2,4-triazol-3-ylthio)acetate | | | |
| Benzyl 2-(5-(3-chlorophenyl)-4-(2- | 466.3 | 3.65 | 14.27 |
| methoxyphenyl)-4H-1,2,4-triazol-3- | | | |
| ylthio)acetate | | | |
| Benzyl 2-(4-(2-methoxyphenyl)-5-p-tolyl- | 446.3 | 3.51 | 14.26 |
| 4H-1,2,4-triazol-3-ylthio)acetate | | | |
| Benzyl 2-(5-(3-hydroxyphenyl)-4-(2- | 448.3 | 3.1 | 14.29 |
| methoxyphenyl)-4H-1,2,4-triazol-3- | | | |
| ylthio)acetate | | | |
| Benzyl 2-(4,5-bis(2-methoxyphenyl)-4H- | 436.3 | 3.3 | 14.30 |
| 1,2,4-triazol-3-ylthio)acetate | | | |
| Benzyl 2-(4-(2-methoxyphenyl)-5-(3- | 436.3 | 3.42 | 14.31 |
| methoxyphenyl)-4H-1,2,4-triazol-3- | | | |
| ylthio)acetate | | | |
| Benzyl 2-(5-(4-hydroxyphenyl)-4-(2- | 447.9 | 3 | 14.32 |
| methoxyphenyl)-4H-1,2,4-triazol-3- | | | |
| ylthio)acetate | | | |
| Benzyl 2-(5-(2,4-difluorophenyl)-4-(2- | 468.3 | 3.5 | 14.33 |
| methoxyphenyl)-4H-1,2,4-triazol-3- | | | |
| ylthio)acetate | | | |
| Benzyl 2-(4-(3-chlorophenyl)-5-(4- | 466.3 | 3.58 | 14.34 |
| methoxyphenyl)-4H-1,2,4-triazol-3- | | | |
| ylthio)acetate | | | |
| Benzyl 2-(4-(3-chlorophenyl)-5-(2- | 451.9 | 3.66 | 14.35 |
| hydroxyphenyl)-4H-1,2,4-triazol-3- | | | |
| ylthio)acetate | | | |
| Benzyl 2-(4-(3-methoxyphenyl)-5-(4- | 436.3 | 3.42 | 14.36 |
| methoxyphenyl)-4H-1,2,4-triazol-3- | | | |
| ylthio)acetate | | | |
| Benzyl 2-(5-(2-hydroxyphenyl)-4-(3- | 448.3 | 3.55 | 14.37 |
| methoxyphenyl)-4H-1,2,4-triazol-3- | | | |
| ylthio)acetate | | | |
| Benzyl 2-(4-(4-fluorophenyl)-5-(2- | 436.3 | 3.52 | 14.38 |
| hydroxyphenyl)-4H-1,2,4-triazol-3- | | | |
| ylthio)acetate | | | |
| Benzyl 2-(5-(4-methoxyphenyl)-4-o-tolyl- | 446.3 | 3.51 | 14.39 |
| 4H-1,2,4-triazol-3-ylthio)acetate | | | |
| Benzyl 2-(5-(2-hydroxyphenyl)-4-o-tolyl- | 432.3 | 3.75 | 14.40 |
| 4H-1,2,4-triazol-3-ylthio)acetate | | | |
| Benzyl 2-(5-(4-methoxyphenyl)-4-p-tolyl- | 446.3 | 3.51 | 14.41 |
| 4H-1,2,4-triazol-3-ylthio)acetate | | | |
| Benzyl 2-(5-(4-methoxyphenyl)-4-m-tolyl- | 446.3 | 3.49 | 14.42 |
| 4H-1,2,4-triazol-3-ylthio)acetate | | | |
| Benzyl 2-(5-(2-hydroxyphenyl)-4-m-tolyl- | 432.3 | 3.64 | 14.43 |
| 4H-1,2,4-triazol-3-ylthio)acetate | | | |
| Benzyl 2-(5-(4-methoxyphenyl)-4-(3- | 500.3 | 3.65 | 14.44 |
| (trifluoromethyl)phenyl)-4H-1,2,4-triazol-3- | | | |
| ylthio)acetate | | | |
| Benzyl 2-(5-(2-hydroxyphenyl)-4-(3- | 486.3 | 3.66 | 14.45 |
| (trifluoromethyl)phenyl)-4H-1,2,4-triazol-3- | | | |
| ylthio)acetate | | | |
| Benzyl 2-(4,5-bis(4-methoxyphenyl)-4H- | 436.3 | 3.35 | 14.46 |
| 1,2,4-triazol-3-ylthio)acetate | | | |
| Benzyl 2-(5-(2-hydroxyphenyl)-4-(4- | 448.3 | 3.52 | 14.47 |
| methoxyphenyl)-4H-1,2,4-triazol-3- | | | |
| ylthio)acetate | | | |
| N-(3-(2-(5-(4-methoxyphenyl)-4-phenyl- | 530.3 | 3.05 | 14.48 |
| 4H-1,2,4-triazol-3- | | | |
| ylthio)acetamido)phenyl)-tetrahydrofuran-2- | | | |
| carboxamide | | | |
| N-(3-(2-(5-(4-methoxyphenyl)-4-phenyl- | 526.3 | 3.1 | 14.49 |
| 4H-1,2,4-triazol-3- | | | |
| ylthio)acetamido)phenyl)furan-2- | | | |
| carboxamide | | | |
| N-benzyl-2-(5-(4-methoxyphenyl)-4- | 445.1 | 3.24 | 14.50 |
| phenyl-4H-1,2,4-triazol-3-ylthio)-N- | | | |
| methylacetamide | | | |
| N-(3-(2-(5-(2-hydroxyphenyl)-4-(2- | 546.3 | 3.05 | 14.51 |
| methoxyphenyl)-4H-1,2,4-triazol-3- | | | |
| ylthio)acetamido)phenyl)-tetrahydrofuran-2- | | | |
| carboxamide | | | |
| N-(3-(2-(5-(2-hydroxyphenyl)-4-(2- | 542.3 | 3.15 | 14.52 |
| methoxyphenyl)-4H-1,2,4-triazol-3- | | | |
| ylthio)acetamido)phenyl)furan-2- | | | |
| carboxamide | | | |
| N-(3-(2-(5-(2-hydroxyphenyl)-4-(2- | 556 | 3.18 | 14.53 |
| methoxyphenyl)-4H-1,2,4-triazol-3- | | | |
| ylthio)acetamido)-4-methylphenyl)furan-2- | | | |
| carboxamide | | | |
| 2-(5-(2-hydroxyphenyl)-4-(2- | 447.1 | 3.21 | 14.54 |
| methoxyphenyl)-4H-1,2,4-triazol-3-ylthio)- | | | |
| N-methyl-N-phenylacetamide | | | |
| N-benzyl-2-(5-(2-hydroxyphenyl)-4-(2- | 461.1 | 3.29 | 14.55 |
| methoxyphenyl)-4H-1,2,4-triazol-3-ylthio)- | | | |
| N-methylacetamide | | | |
| 1-(4-(2-methoxyphenyl)-5-phenyl-4H-1,2,4- | 416.3 | 3.24 | 14.56 |
| triazol-3-ylthio)-3-phenylpropan-2-one | | | |
| 1-(4-(2-methoxyphenyl)-5-phenyl-4H-1,2,4- | 430.3 | 3.38 | 14.57 |
| triazol-3-ylthio)-4-phenylbutan-2-one | | | |

### Example 2

### Synthesis of

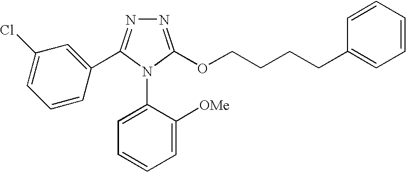

A mixture of 3-chlorobenzhydrazide (500 mg, 2.92 mmol) and 2-methoxyphenyl isothiocyanate (0.4 mL, 2.92 mmol) in 1,4-dioxane (5 mL) was irradiated in a microwave oven (max. power 300 W, 80° C.) for 30 min, then cooled to room temperature. A solution of 1N aqueous NaOH (5 mL) was added, and the mixture was irradiated in a microwave oven (max. power 300 W, 170° C.) for 10 min, then cooled to 0° C. in an ice bath. The mixture was made acidic with aqueous 1N HCl and extracted into EtOAc (3×). The combined organic layers were dried over MgSO₄, then concentrated to provide the thione (650 mg) as a white solid.

To a mixture of the thione prepared above and N,N-diisopropylethylamine (0.71 mL, 4.10 mmol) in CHCl₃ (5 mL) was added methyl iodide (0.14 mL, 2.25 mmol). The mixture was stirred at room temperature for 30 min, concentrated in vacuo, and the residue was purified by flash chromatography on silica gel, eluting with 1:1 ethyl acetate/hexane to provide the thiomethyl intermediate (540 mg) as a solid.

To a solution of the thiomethyl intermediate prepared above in acetone (20 mL) was added sodium molybdate dihydrate (10 g, 41.0 mmol). The mixture was brought to reflux, and 30% H₂O₂ (500 mL) was added slowly while refluxing over the course of 5 h. After an additional 12 h at reflux, the reaction mixture was cooled to room temperature, and the acetone solvent was removed in vacuo. The remaining aqueous mixture was extracted with EtOAc (3×), and the combined organic layers were dried over MgSO₄, then concentrated in vacuo. The crude product was purified by flash chromatography on silica gel, eluting with 1:1 ethyl acetate/hexane containing 1% tri ethylamine to provide the sulfone intermediate (445 mg) as a white solid.

To a solution of 4-phenyl-1-butanol (0.18 mL, 1.16 mmol) in THF (5 mL) was added sodium hydride (45 mg, 60 wt % in mineral oil, 1.16 mmol). The mixture was stirred at room temperature for 20 min, at which time the reaction solution was clear. The sulfone intermediate prepared above (210 mg, 0.58 mmol) was added, and the reaction mixture was stirred at room temperature for 12 h, then quenched by the addition of water. The reaction mixture was extracted with EtOAc (3×), and the combined organic layers were dried over MgSO₄, then concentrated in vacuo. The crude product was purified by flash chromatography on silica gel, eluting with 2:1 ethyl acetate/hexane containing 1% triethylamine to provide the title compound (175 mg) as a viscous oil. LC/MS (ESI) m/z 434.3 [M+H]. HPLC retention time=3.78 min. ¹H NMR (DMSO-d₆) δ 1.55-1.70 (m, 4H), 2.55 (t, J=7.5 Hz, 2H), 3.58 (s, 3H), 4.38-4.45 (m, 2H), 7.07-7.53 (m, 13H).

### Example 3

### Synthesis of

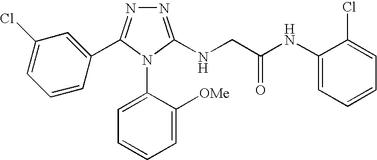

To a solution of 3-chlorobenzoyl chloride (0.37 mL, 2.9 mmol) in THF (10 mL) at 0° C. was added N,N-diisopropylethylamine (0.53 mL, 3.0 mmol) followed by 2-methoxyaniline (0.33 mL, 2.9 mmol). The reaction mixture was stirred at room temperature for 4 h, filtered, and diluted with EtOAc (50 mL). The solution was washed with water (2×), dried over MgSO₄, then concentrated in vacuo to provide the amide (720 mg) as a yellow solid.

Phosphorus pentachloride (572 mg, 2.75 mmol) was added to a solution of the amide prepared above (650 mg, 2.5 mmol) in benzene (10 mL), and the mixture was maintained at reflux for 3 h. The reaction mixture was cooled to room temperature and concentrated in vacuo to remove POCl₃. The residue was then dissolved in THF (15 mL) and cooled to 0° C. Anhydrous hydrazine (0.78 mL, 25 mmol) was added and the reaction was stirred at room temperature for 1 h, then poured into water (50 mL). The mixture was extracted with ethyl acetate (3×) and the combined organic phases were washed with brine, then dried over MgSO₄. Evaporation of the solvent furnished the benzanilide hydrazone (644 mg) as a colorless oil.

To a solution of the benzanilide hydrazone prepared above (400 mg, 1.45 mmol) in 1,4-dioxane (5 mL) was added cyanogen bromide (154 mg, 1.45 mmol). A solution of NaHCO₃ (128 mg, 1.52 mmol) in water (5 mL) was added dropwise, and the reaction mixture was stirred at room temperature for 3 h. Additional water (5 mL) was added and the heterogenous mixture was filtered and the filtrate was concentrated in vacuo. The resulting solids were then rinsed with CH₃CN and filtered. The filtrate was concentrated in vacuo to furnish the crude aminotriazole (260 mg) as a yellow solid.

A portion of the aminotriazole prepared above (60 mg, 0.2 mmol) was dissolved in a solution of lithium bis(trimethylsilyl)amide (0.25 mL, 1.0M in THF) and stirred at room temperature for 30 min. To the resulting red solution was added di-tert-butyl dicarbonate (65 mg, 0.3 mmol). The reaction mixture was stirred at room temperature for 2 h, diluted with water (2 mL), and extracted with EtOAc (2×). The combined organic layers were dried over MgSO₄ and concentrated in vacuo. The residue was purified by flash chromatography on silica gel, eluting with 1:1 ethyl acetate/hexane to provide the carbamate (51 mg) as a viscous oil.

A portion of the carbamate prepared above (30 mg, 0.075 mmol) in THF (1 mL) was treated with sodium hydride (6 mg, 60 wt % in mineral oil, 0.15 mmol), stirred at room temperature for 10 min, then treated with methyl bromoacetate (0.015 mL, 0.15 mmol). The reaction mixture was stirred at room temperature for 12 h, diluted with water, and extracted with EtOAc (2×). The combined organic layers were dried over MgSO₄ and concentrated in vacuo to provide the ester (26 mg) as a colorless oil.

The crude ester prepared above was dissolved in 1,4-dioxane (3 mL) and treated with aqueous 2N NaOH for 30 min, then the reaction mixture was adjusted to pH 4 with dilute aqueous HCl and extracted with EtOAc (3×). The combined organic layers were dried over MgSO₄ and concentrated in vacuo to provide the ester (25 mg) as an oil.

To the crude acid prepared above in CHCl₃ (0.5 mL) was added N,N-diisopropylethylamine (0.01 mL, 0.055 mmol), followed by thionyl chloride (0.004 mL, 0.055 mmol). The reaction mixture was stirred at room temperature for 10 min, followed by addition of 2-chloroaniline (0.012 mL, 0.11 mmol). The reaction mixture was stirred at room temperature for 3 h, then concentrated in vacuo. The resulting residue (22 mg) was dissolved in 1:1 TFA/CH₂Cl₂ (5 mL), stirred at room temperature for 20 min, then concentrated in vacuo. The crude product was purified by preparative HPLC (Phenomenex Synergi 4 μm Max-RP column (10 mm×50 mm); flow rate=6 mL/min; injection volume=100 μL; mobile phase A: 100% water, 0.1% trifluoroacetic acid (TFA); mobile phase B: 100% acetonitrile, 0.1% trifluoroacetic acid (TFA); gradient elution from 5% B to 100% B over 8 min) to provide the title compound (1 mg) as a colorless gum. LC/MS (ESI) m/z 468.3 [M+H]. HPLC retention time=2.90 min.

### Example 4

The following compounds listed in the following table were prepared by the general procedure for solid phase parallel synthesis or by the general procedures as exemplified in Examples 1-3, utilizing the appropriate starting materials.

TABLE 2

| | LC/MS | HPLC | |
|---|---|---|---|
| | m/z | retention | HPLC |
| Compound Structure | [M + H] | time (min) | Method |
| 4-(2-methoxyphenyl)-3-phenyl-5-(5- | 414.3 | 3.64 | B |
| phenylpentyloxy)-4H-1,2,4-triazole | | | |
| 3-cyclohexyl-4-(2-methoxyphenyl)-5-(5- | 420.3 | 3.54 | B |
| phenylpentyloxy)-4H-1,2,4-triazole | | | |
| 4-(2-methoxyphenyl)-3-phenyl-5-(4- | 400.3 | 3.49 | B |
| phenylbutoxy)-4H-1,2,4-triazole | | | |
| 4-(2-methoxyphenyl)-3-phenyl-5-(3- | 386.3 | 3.36 | B |
| phenylpropoxy)-4H-1,2,4-triazole | | | |
| 3-cyclohexyl-4-(2-methoxyphenyl)-5-(3- | 392.3 | 3.31 | B |
| phenylpropoxy)-4H-1,2,4-triazole | | | |
| 3-(3-chlorophenyl)-4-(2-methoxyphenyl)-5-(5- | 448.3 | 3.94 | B |
| phenylpentyloxy)-4H-1,2,4-triazole | | | |
| 3-(3-chlorophenyl)-4-(2-methoxyphenyl)-5-(4- | 434.3 | 3.78 | B |
| phenylbutoxy)-4H-1,2,4-triazole | | | |
| 3-(3-chlorophenyl)-4-(2-methoxyphenyl)-5-(3- | 420.3 | 3.68 | B |
| phenylpropoxy)-4H-1,2,4-triazole | | | |
| 3-cyclohexyl-4-(2-methoxyphenyl)-5-(4- | 406.3 | 3.41 | B |
| phenylbutoxy)-4H-1,2,4-triazole | | | |
| N-(furan-2-ylmethyl)-2-(4-(2-methoxyphenyl)-5- | 405.5 | 4.60 | C |
| phenyl-4H-1,2,4-triazol-3-yloxy)acetamide | | | |
| 2-(5-(3-chlorophenyl)-4-(2-methoxyphenyl)-4H- | 439.1 | 5.17 | C |
| 1,2,4-triazol-3-yloxy)-N-(furan-2-ylmethyl)acetamide | | | |
| 2-(4-(2-methoxyphenyl)-5-phenyl-4H-1,2,4- | 415.5 | 5.18 | C |
| triazol-3-yloxy)-N-o-tolylacetamide | | | |
| 2-(5-(3-chlorophenyl)-4-(2-methoxyphenyl)-4H- | 449.1 | 5.74 | C |
| 1,2,4-triazol-3-yloxy)-N-o-tolylacetamide | | | |
| phenethyl 2-(5-(3-chlorophenyl)-4-(2- | 463.9 | 6.41 | C |
| methoxyphenyl)-4H-1,2,4-triazol-3-yloxy)acetate | | | |
| benzyl 2-(4-(2-methoxyphenyl)-5-phenyl-4H- | 416.3 | 5.64 | C |
| 1,2,4-triazol-3-yloxy)acetate | | | |
| benzyl 2-(5-(3-chlorophenyl)-4-(2- | 450.3 | 6.20 | C |
| methoxyphenyl)-4H-1,2,4-triazol-3-yloxy)acetate | | | |
| phenethyl 2-(4-(2-methoxyphenyl)-5-phenyl-4H- | 430.3 | 5.84 | C |
| 1,2,4-triazol-3-yloxy)acetate | | | |
| 5-(2-methoxyphenyl)-4H-1,2,4-triazol-3-amine | 191.0 | 1.1 | E |
| 1-(3-amino-4H-1,2,4-triazol-4-yl)-2-(4- | 237.1 | 1.4 | E |
| chlorophenyl)ethanone | | | |
| N-(2-chlorophenyl)-2-(5-(3-chlorophenyl)-4-(2- | 468.3 | 2.9 | B |
| methoxyphenyl)-4H-1,2,4-triazol-3- | | | |
| ylamino)acetamide | | | |
| N-(3-(2-(5-(3-chlorophenyl)-4-(2- | 557.2 | 2.9 | B |
| methoxyphenyl)-4H-1,2,4-triazol-3- | | | |
| ylamino)acetamido)-4-methylphenyl)furan-2- | | | |
| carboxamide | | | |
| benzyl 2-(5-(3-chlorophenyl)-4-(2- | 449.1 | 3.0 | B |
| methoxyphenyl)-4H-1,2,4-triazol-3-ylamino)acetate | | | |
| 3-(3-chlorophenyl)-4-(2-methoxyphenyl)-5-(3- | 436.3 | 3.8 | B |
| phenylpropylthio)-4H-1,2,4-triazole | | | |
| 4-(2-methoxyphenyl)-3-phenyl-5-(4- | 416.3 | 3.7 | B |
| phenylbutylthio)-4H-1,2,4-triazole | | | |
| 3-cyclohexyl-4-(2-methoxyphenyl)-5-(4- | 421.9 | 3.7 | B |
| phenylbutylthio)-4H-1,2,4-triazole | | | |
| 3-(3-chlorophenyl)-4-(2-methoxyphenyl)-5-(4- | 450.3 | 3.9 | B |
| phenylbutylthio)-4H-1,2,4-triazole | | | |
| 3-cyclohexyl-4-(2-methoxyphenyl)-5-(3- | 408.3 | 3.5 | B |
| phenylpropylthio)-4H-1,2,4-triazole | | | |
| 4-(2-methoxyphenyl)-3-phenyl-5-(3- | 402.3 | 3.6 | B |
| phenylpropylthio)-4H-1,2,4-triazole | | | |
| 3-(3-chlorophenyl)-4-(2-methoxyphenyl)-5-(5- | 464.3 | 4.1 | B |
| phenylpentylthio)-4H-1,2,4-triazole | | | |
| N-(furan-2-ylmethyl)-3-(4-(2-methoxyphenyl)-5- | 435.1 | 2.8 | B |
| phenyl-4H-1,2,4-triazol-3-ylthio)propanamide | | | |
| 3-(5-(3-chlorophenyl)-4-(2-methoxyphenyl)-4H- | 469.1 | 3.1 | B |
| 1,2,4-triazol-3-ylthio)-N-(furan-2- | | | |
| ylmethyl)propanamide | | | |
| N-benzyl-3-(4-(2-methoxyphenyl)-5-phenyl-4H- | 445.1 | 3.0 | B |
| 1,2,4-triazol-3-ylthio)propanamide | | | |
| N-benzyl-3-(5-(3-chlorophenyl)-4-(2- | 479.1 | 3.2 | B |
| methoxyphenyl)-4H-1,2,4-triazol-3- | | | |
| ylthio)propanamide | | | |
| 3-(4-(2-methoxyphenyl)-5-phenyl-4H-1,2,4- | 451.1 | 3.0 | B |
| triazol-3-ylthio)-N-(thiophen-2- | | | |
| ylmethyl)propanamide | | | |
| 3-(5-(3-chlorophenyl)-4-(2-methoxyphenyl)-4H- | 485.1 | 3.2 | B |
| 1,2,4-triazol-3-ylthio)-N-(thiophen-2-ylmethyl)propanamide | | | |
| N-(3-(3-(4-(2-methoxyphenyl)-5-phenyl-4H- | 554.4 | 3.0 | B |
| 1,2,4-triazol-3-ylthio)propanamido)-4- | | | |
| methylphenyl)furan-2-carboxamide | | | |
| N-(3-(3-(5-(3-chlorophenyl)-4-(2- | 588.0 | 3.2 | B |
| methoxyphenyl)-4H-1,2,4-triazol-3- | | | |
| ylthio)propanamido)-4-methylphenyl)furan-2- | | | |
| carboxamide | | | |
| N-(3-(3-(4-(2-methoxyphenyl)-5-phenyl-4H- | 540.3 | 3.0 | B |
| 1,2,4-triazol-3-ylthio)propanamido)phenyl)furan-2-carboxamide | | | |
| N-(furan-2-ylmethyl)-2-(4-(2-methoxyphenyl)-5- | 435.1 | 3.0 | B |
| phenyl-4H-1,2,4-triazol-3-ylthio)propanamide | | | |
| 2-(5-cyclohexyl-4-(2-methoxyphenyl)-4H-1,2,4- | 437.1 | 3.3 | B |
| triazol-3-ylthio)-N-phenylpropanamide | | | |
| N-(4-chlorophenyl)-2-(4-(2-methoxyphenyl)-5- | 465.1 | 3.5 | B |
| phenyl-4H-1,2,4-triazol-3-ylthio)propanamide | | | |
| N-(2-chlorophenyl)-2-(5-cyclohexyl-4-(2- | 471.5 | 3.5 | B |
| methoxyphenyl)-4H-1,2,4-triazol-3-ylthio)propanamide | | | |
| 2-(5-cyclohexyl-4-(2-methoxyphenyl)-4H-1,2,4- | 451.1 | 3.4 | B |
| triazol-3-ylthio)-N-o-tolylpropanamide | | | |
| 2-(5-cyclohexyl-4-(2-methoxyphenyl)-4H-1,2,4- | 451.1 | 3.2 | B |
| triazol-3-ylthio)-N-methyl-N-phenylpropanamide | | | |
| N-(3-(2-(4-(2-methoxyphenyl)-5-phenyl-4H- | 554.4 | 3.2 | B |
| 1,2,4-triazol-3-ylthio)propanamido)-4- | | | |
| methylphenyl)furan-2-carboxamide | | | |
| N-(3-(2-(5-cyclohexyl-4-(2-methoxyphenyl)-4H- | 560.4 | 3.2 | B |
| 1,2,4-triazol-3-ylthio)propanamido)-4- | | | |
| methylphenyl)furan-2-carboxamide | | | |
| N-(3-(2-(5-(3-chlorophenyl)-4-(2- | 588.4 | 3.4 | B |
| methoxyphenyl)-4H-1,2,4-triazol-3- | | | |
| ylthio)propanamido)-4-methylphenyl)furan-2- | | | |
| carboxamide | | | |
| N-(3-(2-(4-(2-methoxyphenyl)-5-phenyl-4H- | 540.3 | 3.1 | B |
| 1,2,4-triazol-3- | | | |
| ylthio)propanamido)phenyl)furan-2-carboxamide | | | |
| N-(3-(2-(5-(3-chlorophenyl)-4-(2- | 574.4 | 3.4 | B |
| methoxyphenyl)-4H-1,2,4-triazol-3- | | | |
| ylthio)propanamido)phenyl)furan-2-carboxamide | | | |
| benzyl 2-(5-(3-chlorophenyl)-4-(3- | 503.9 | 3.8 | B |
| (trifluoromethyl)phenyl)-4H-1,2,4-triazol-3-ylthio)acetate | | | |
| 1-phenylethyl 2-(5-(3-chlorophenyl)4-(3- | 517.9 | 3.8 | D |
| (trifluoromethyl)phenyl)-4H-1,2,4-triazol-3-ylthio)acetate | | | |
| 2-(pyridin-2-yl)ethyl 2-(5-(4-methoxyphenyl)-4- | 515.1 | 2.7 | D |
| (3-(trifluoromethyl)phenyl)-4H-1,2,4-triazol-3-ylthio)acetate | | | |
| thiophen-2-ylmethyl 2-(5-(4-methoxyphenyl)-4- | 505.9 | 3.4 | D |
| (3-(trifluoromethyl)phenyl)-4H-1,2,4-triazol-3-ylthio)acetate | | | |
| 3-fluorobenzyl 2-(5-(4-methoxyphenyl)-4-(3- | 517.9 | 3.5 | D |
| (trifluoromethyl)phenyl)-4H-1,2,4-triazol-3-ylthio)acetate | | | |
| 2-chloro-4-fluorobenzyl 2-(5-(4- | 552.3 | 3.7 | D |
| methoxyphenyl)-4-(3-(trifluoromethyl)phenyl)- | | | |
| 4H-1,2,4-triazol-3-ylthio)acetate | | | |
| furan-2-ylmethyl 2-(5-(4-methoxyphenyl)-4-(3- | 490.3 | 3.3 | D |
| (trifluoromethyl)phenyl)-4H-1,2,4-triazol-3-ylthio)acetate | | | |
| furan-3-ylmethyl 2-(5-(4-methoxyphenyl)-4-(3- | 490.3 | 3.3 | D |
| (trifluoromethyl)phenyl)-4H-1,2,4-triazol-3-ylthio)acetate | | | |
| chroman-4-yl 2-(5-(4-methoxyphenyl)-4-(3- | 542.3 | 3.5 | D |
| (trifluoromethyl)phenyl)-4H-1,2,4-triazol-3-ylthio)acetate | | | |
| 3-methylphenethyl 2-(5-(4-methoxyphenyl)-4- | 528.3 | 3.7 | D |
| (3-(trifluoromethyl)phenyl)-4H-1,2,4-triazol-3-ylthio)acetate | | | |
| 4-fluorobenzyl 2-(5-(4-methoxyphenyl)-4-(3- | 517.9 | 3.5 | D |
| (trifluoromethyl)phenyl)-4H-1,2,4-triazol-3-ylthio)acetate | | | |
| 2-(thiophen-3-yl)ethyl 2-(5-(4-methoxyphenyl)- | 520.3 | 3.5 | D |
| 4-(3-(trifluoromethyl)phenyl)-4H-1,2,4-triazol-3-ylthio)acetate | | | |
| 4-chlorophenethyl 2-(5-(4-methoxyphenyl)-4-(3- | 548.3 | 3.7 | D |
| (trifluoromethyl)phenyl)-4H-1,2,4-triazol-3-ylthio)acetate | | | |
| 2-methoxyphenethyl 2-(5-(4-methoxyphenyl)-4- | 544.3 | 3.6 | D |
| (3-(trifluoromethyl)phenyl)-4H-1,2,4-triazol-3-ylthio)acetate | | | |
| 3-chlorobenzyl 2-(5-(4-methoxyphenyl)-4-(3- | 533.9 | 3.6 | D |
| (trifluoromethyl)phenyl)-4H-1,2,4-triazol-3-ylthio)acetate | | | |
| 2-(2-chlorophenoxy)ethyl 2-(5-(4- | 564.4 | 3.6 | D |
| methoxyphenyl)-4-(3-(trifluoromethyl)phenyl)- | | | |
| 4H-1,2,4-triazol-3-ylthio)acetate | | | |
| 3-methylbenzyl 2-(5-(4-methoxyphenyl)-4-(3- | 514.3 | 3.6 | D |
| (trifluoromethyl)phenyl)-4H-1,2,4-triazol-3-ylthio)acetate | | | |
| (2,3-dihydrobenzo[b][1,4]dioxin-2-yl)methyl 2- | 558.0 | 3.5 | D |
| (5-(4-methoxyphenyl)-4-(3- | | | |
| (trifluoromethyl)phenyl)-4H-1,2,4-triazol-3-ylthio)acetate | | | |
| cycloheptyl 2-(5-(4-methoxyphenyl)-4-(3- | 505.9 | 3.8 | D |
| (trifluoromethyl)phenyl)-4H-1,2,4-triazol-3-ylthio)acetate | | | |
| (4H-benzo[d][1,3]dioxin-2-yl)methyl 2-(5-(4- | 558.0 | 3.5 | D |
| methoxyphenyl)-4-(3-(trifluoromethyl)phenyl)- | | | |
| 4H-1,2,4-triazol-3-ylthio)acetate | | | |
| 2-methylphenethyl 2-(5-(4-methoxyphenyl)-4- | 528.3 | 6.5 | C |
| (3-(trifluoromethyl)phenyl)-4H-1,2,4-triazol-3-ylthio)acetate | | | |
| 2-chlorophenethyl 2-(5-(4-methoxyphenyl)-4-(3- | 548.3 | 6.6 | C |
| (trifluoromethyl)phenyl)-4H-1,2,4-triazol-3-ylthio)acetate | | | |
| 3-chlorophenethyl 2-(5-(4-methoxyphenyl)-4-(3- | 548.3 | 6.6 | C |
| (trifluoromethyl)phenyl)-4H-1,2,4-triazol-3-ylthio)acetate | | | |
| 2-chlorobenzyl 2-(5-(4-methoxyphenyl)-4-(3- | 534.3 | 6.3 | C |
| (trifluoromethyl)phenyl)-4H-1,2,4-triazol-3-ylthio)acetate | | | |
| 2-methylbenzyl 2-(5-(4-methoxyphenyl)-4-(3- | 514.3 | 6.3 | C |
| (trifluoromethyl)phenyl)-4H-1,2,4-triazol-3-ylthio)acetate | | | |
| phenethyl 2-(5-(4-methoxyphenyl)-4-(3- | 514.3 | 6.3 | C |
| (trifluoromethyl)phenyl)-4H-1,2,4-triazol-3- | | | |
| ylthio)acetate | | | |
| phenyl 2-(5-(3-chlorophenyl)-4-(2- | 466.3 | 3.7 | B |
| methoxyphenyl)-4H-1,2,4-triazol-3-ylthio)propanoate | | | |
| benzyl 2-(4-(2-methoxyphenyl)-5-phenyl-4H- | 446.3 | 3.4 | B |
| 1,2,4-triazol-3-ylthio)propanoate | | | |
| phenethyl 2-(4-(2-methoxyphenyl)-5-phenyl-4H- | 460.3 | 3.5 | B |
| 1,2,4-triazol-3-ylthio)propanoate | | | |
| phenethyl 2-(5-cyclohexyl-4-(2-methoxyphenyl)- | 466.3 | 3.6 | B |
| 4H-1,2,4-triazol-3-ylthio)propanoate | | | |
| phenethyl 2-(5-(3-chlorophenyl)-4-(2- | 494.3 | 3.8 | B |
| methoxyphenyl)-4H-1,2,4-triazol-3-ylthio)propanoate | | | |
| 5-benzyl-2-((5-(3-chlorophenyl)-4-(2- | 489.1 | 3.5 | B |
| methoxyphenyl)-4H-1,2,4-triazol-3-ylthio)methyl)oxazole | | | |
| 5-benzyl-2-((5-(4-methoxyphenyl)-4-(3- | 523.1 | 3.5 | B |
| (trifluoromethyl)phenyl)-4H-1,2,4-triazol-3- | | | |
| ylthio)methyl)oxazole | | | |
| 2-((5-(3-chlorophenyl)-4-(2-methoxyphenyl)- | 503.1 | 3.6 | B |
| 4H-1,2,4-triazol-3-ylthio)methyl)-5- | | | |
| phenethyloxazole | | | |

### Example 5

### Characterization of Compounds

The following HPLC conditions were used for characterizing compounds of listed in Table 1 above: Phenomenex Chromolith SpeedRod RP-18e C18 analytical column (4.6 mm×50 mm); flow rate=1.5 mL/min; injection volume=10 μL; mobile phase A: 100% water, 0.1% trifluoroacetic acid (TFA); mobile phase B: 100% acetonitrile, 0.12% TFA); gradient elution from 5% B to 100% B over 4.4 min, with a stay at 100% B for 1 min, then equilibration to 5% B over 0.6 min. MS ions were detected using a Sciex API-100 electrospray single quadrupole mass spectrometer interfaced to the HPLC system.

The following HPLC conditions were used for characterizing compounds of the present disclosure, including the compounds listed in Table 2: Phenomenex Chromolith SpeedRod RP-18e C18 analytical column (4.6 mm×50 mm); injection volume=10 μL; mobile phase A: 100% water, 0.1% trifluoroacetic acid (TFA); mobile phase B: 100% acetonitrile, 0.12% TFA). MS ions were detected using a Sciex API-100 electrospray single quadrupole mass spectrometer interfaced to the HPLC system.

Method A: gradient elution from 5% B to 100% B over 4.4 min, with a stay at 100% B for 1 min, then equilibration to 5% B over 0.6 min; flow rate=1.5 mL/imin.

Method B: gradient elution from 5% B to 100% B over 4.3 min, with a stay at 100% B for 1 min, then equilibration to 5% B over 0.7 min; flow rate=1.5 mL/min.

Method C: gradient elution from 5% B to 100% B over 10.3 min, with a stay at 100% B for 1 min, then equilibration to 5% B over 0.7 min; flow rate=1.5 mL/min.

Method D: gradient elution from 10% B to 100% B over 4 min, with a stay at 100% B for 1.1 min, then equilibration to 10% B over 0.9 min; flow rate=1.5 mL/min.

Method E: gradient elution from 0% B to 100% B over 2.6 min, with a stay at 100% B for 0.9 min, then equilibration to 0% B over 0.5 min; flow rate=4 mL/min.

### Example 6

### HTS ATP-Utilizing Enzyme Assays

The following procedures describe the reagent and plate preparation for a HTS of an ATP-utilizing enzyme, such as a protein kinase, run in an off-chip mobility-shift assay format. The following provides an HTS protocol for running a protein kinase HTS screen on a Caliper HTS 250 microfluidics system. The following parameters are dependent on the protein kinase used and can be determined by one skilled in the art as part of a typical assay development process. For example, the peptide substrate used can be identified from the current literature, by screening a peptide library of potential protein kinase substrates, or by other applicable means accepted in the field.

The following table provides typical screen assay parameters appropriate for a Caliper HTS 250 microfluidics system.

| Reaction | | |
|---|---|---|
| Concentration | | |
| Inhibitor concentration | 10 | μM |
| Enzyme concentration | 18 | nM |
| Substrate/Peptide Conc. | 1 | μM |
| Reaction Properties | | |
| Inhibitor Volume | 5 | μL |
| Enzyme Volume | 10 | μL |
| Substrate Volume | 10 | μL |
| Termination Volume | 45 | μL |
| Reaction Time | 1-24 | hrs |
| Reaction Temperature | 20-25 | ° C. |
| Sipper Properties | | |
| Initial Delay | 18 | sec |
| Buffer | 18 | sec |
| Sample | 0.2 | sec |
| Final Delay | 120 | sec |
| Dye Well | | |
| Dye | 0.2 | sec |
| Script Properties | | |
| Electrode 1 | −250 | Volts |
| Electrode 2 | −2250 | Volts |
| Electrode 3 | −2250 | Volts |
| Electrode 4 | −250 | Volts |
| Laser Properties | yes/no | |
| UV | no | |
| Blue | yes | |
| Red | no | |
| Data Collection | yes/no | |
| CCD1 | no | |
| CCD2 | yes | |
| CCD3 | no | |
| Inhibitor | | |
| Concentrations | | |
| Inhibitor: EDTA | | |
| 100% | 20 | mM |
| Inhibitor Staurosporine | | |
| 70% | 138 | nM |
| Pressure Driven Flow | | |
| Pressure | −2 | psi |
| Base Pressure | −2 | psi |

The reagents and buffers listed in the following table are generally applicable for developing and running an HTS screen on a human protein kinase using the Caliper HTS 250 system.

| Reagent | Reagent Name | Manufacturer | Catalog # | MW | Storage |
|---|---|---|---|---|---|
| 4 sipper | FS266 | Caliper Tech. Inc. | 760077- | — | 2-8° C. |
| LABCHIP | | | 0266 | | |
| Enzyme | Specific protein | — | | kDa | −20° C. |
| | kinase | | | | |
| Substrate | Specific peptide | — | | Da | −20° C. |
| Control | Specific | LKT | S7600 | 466.5 | 2-8° C. |
| Inhibitor | compound | | | | |
| Buffer | HEPES (free | Calbiochem | 391338 | 238.3 | RT |
| Components | acid) | | | | |
| | HEPES (Na Salt) | Calbiochem | 391333 | 260.3 | RT |
| | DMSO | Sigma | D8418 | — | RT |
| | Triton X-100 | Sigma | T8787 | — | RT |
| | BSA | Sigma | A8806 | — | 2-8° C. |
| | DTT (Cleland's | Calbiochem | 233153 | 154.2 | 2-8° C. |
| | Reagent) | | | | |
| | EDTA (0.5M) | Sigma | E7889 | n/a | RT |
| | Coating Reagent 3 | Caliper Tech. Inc. | 760050 | n/a | 2-8° C. |
| | 6 N HCl | VWR | JT5619-2 | n/a | RT |
| | ATP disodium | Sigma | A7699 | 551.1 | −20° C. |
| | salt | | | | |
| | Na₃VO₄ | Calbiochem | 567540 | 183.9 | −20° C. |
| | β- | Calbiochem | 35675 | 306.1 | −20° C. |
| | Glycerophosphate | | | | |
| | MgCl₂.6H₂O | Sigma | M2670 | 203.3 | RT |

The following reagents were prepared using the previously described buffers.

A 2× Master Buffer solution was prepared by combining 200 mL of 1 M HEPES, pH 7.5, 2 mL of 10% Triton X-100, 20 mL of 10% BSA, and 778 mL of H₂O.

A 2.5× Enzyme Buffer solution was prepared by combining 177.408 mL of 2× Master Buffer, 0.887 mL of 1 M DTT, 0.089 mL of 100 mM ATP, 8.870 mL of 1 M MgCl₂, 0.089 mL of 100 mM β-glycerophosphate, 0.089 mL of 100 mM Na₃VO₄, 0.254 mL of 62.8 μM enzyme, and 167.13 mL H₂O.

A 2.5× Substrate Buffer solution was prepared by combining 177.408 mL of 2× Master Buffer, 0.887 mL of 1 mM peptide-X, and 176.521 mL of H₂O.

A 1.55× Termination Buffer solution was prepared by combining 762.05 mL of 2× Master Buffer, 95.1 mL of 0.5 M EDTA, and 666.94 mL of H₂O.

A TCB Buffer solution was prepared by combining 125 mL of 2× Master Buffer, 10 mL of 0.5 M EDTA, 6.25 mL of 4% coating reagent, 1.01 mL of 100% DMSO, and 107.74 mL H₂O.

A Dye Trough solution was prepared by combining 0.5 μL of peptide-X, and 2,999.5 μL of 1× Master Buffer.

A 1.06× Assay Buffer solution was prepared by combining 205.15 mL of 2× Master Buffer, and 181.92 mL of H₂O.

Assays to determine the kinase inhibitory activity of chemical entities of the present disclosure were performed using a Caliper HTS 250 microfluidics device, Greiner U-bottom assay plates, a Multidrop for transfer of reagents, and Biomek FX (AMNCBM03) software. Initially, 2.4 μL of a 1 mM solution of a test compound in 100% DMSO was added to a well of the Greiner U-bottom plate. A single Greiner U-bottom plate having 24×16 wells could include multiple test compounds. Next, 40 μL of 1.06× Assay Buffer was added to each well of the assay plate. Using the Biomek FX, 10 μL of 0.5 M EDTA was added by the span-8 to wells, indicated as 100% Control and 2.4 μL of 100% DMSO was added by the span-8 to wells, indicated as 0% Control. Using the Multidrop, 10 μL of 2.5× Enzyme Buffer, followed by 10 μL of 2.5× Substrate Buffer was added to each well of the assay plate. The total reaction volume in each well was 25 μL, and the concentration of the test compound was 10 μM. The assay plate was incubated for 2.5 hrs at 20° C. to 22° C. After the incubation period, using the Multidrop, 45 μL of 1.55× Termination Buffer was added to each well of the assay plate to stop the reaction. The inhibition of the ATP-utilizing enzyme, such as a particular protein kinase, was determined by measuring the ratio of the peptide substrate to phosphorylated product for each well of the assay plate using the Caliper HTS 250 system.

Compounds exhibiting an activity for a particular target ATP-utilizing enzyme greater than three-sigma from the mean activity for the population of predominately inactive compounds for the same target ATP-utilizing enzyme were considered to be active. The use of three-sigma statistical limits represents a conservative method for declaring potential hits among targets. The three-sigma activity, as well as the mean population activity, can be different for each target enzyme. This method has an expected false positive rate, from an in-control measurement process, of 1 in one million. Compounds were considered to show selectivity between a primary target and one or more other targets if the activity (e.g. % inhibition, IC₅₀, Kᵢ, EC₅₀, etc.) for that compound against the primary target was significantly different than that for the other target(s) within the error of the activity measurement.

Certain compounds of Formula I which exhibit protein kinase inhibitory activity are provided in Table 3.

TABLE 3

| Compound | Kinase |
|---|---|
| phenethyl 2-(5-(3-chlorophenyl)-4-(2-methoxyphenyl)-4H-1,2,4-triazol-3- | p38-α |
| yloxy)acetate | |
| benzyl 2-(4-(2-methoxyphenyl)-5-phenyl-4H-1,2,4-triazol-3-yloxy)acetate | p38-α |
| benzyl 2-(5-(3-chlorophenyl)-4-(2-methoxyphenyl)-4H-1,2,4-triazol-3- | p38-α |
| yloxy)acetate | MAPKAPK-3 |
| phenethyl 2-(4-(2-methoxyphenyl)-5-phenyl-4H-1,2,4-triazol-3-yloxy)acetate | p38-α |
| N-(furan-2-ylmethyl)-2-(4-(2-methoxyphenyl)-5-phenyl-4H-1,2,4-triazol-3- | p38-α |
| yloxy)acetamide | |
| 2-(5-(3-chlorophenyl)-4-(2-methoxyphenyl)-4H-1,2,4-triazol-3-yloxy)-N- | p38-α |
| (furan-2-ylmethyl)acetamide | |
| 2-(4-(2-methoxyphenyl)-5-phenyl-4H-1,2,4-triazol-3-yloxy)-N-o- | p38-α |
| tolylacetamide | |
| 2-(5-(3-chlorophenyl)-4-(2-methoxyphenyl)-4H-1,2,4-triazol-3-yloxy)-N-o- | p38-α |
| tolylacetamide | |
| 4-(2-methoxyphenyl)-3-phenyl-5-(5-phenylpentyloxy)-4H-1,2,4-triazole | p38-α |
| | MAPKAPK-3 |
| | p38-β |
| 3-cyclohexyl-4-(2-methoxyphenyl)-5-(5-phenylpentyloxy)-4H-1,2,4-triazole | p38-α |
| 4-(2-methoxyphenyl)-3-phenyl-5-(4-phenylbutoxy)-4H-1,2,4-triazole | p38-α |
| | p38-β |
| 4-(2-methoxyphenyl)-3-phenyl-5-(3-phenylpropoxy)-4H-1,2,4-triazole | p38-α |
| | p38-β |
| 3-cyclohexyl-4-(2-methoxyphenyl)-5-(3-phenylpropoxy)-4H-1,2,4-triazole | p38-α |
| 3-(3-chlorophenyl)-4-(2-methoxyphenyl)-5-(5-phenylpentyloxy)-4H-1,2,4- | p38-α |
| triazole | |
| 3-(3-chlorophenyl)-4-(2-methoxyphenyl)-5-(4-phenylbutoxy)-4H-1,2,4- | p38-α |
| triazole | p38-β |
| | GSK-3-β |
| 3-(3-chlorophenyl)-4-(2-methoxyphenyl)-5-(3-phenylpropoxy)-4H-1,2,4- | p38-α |
| triazole | ROCK2 |
| | p38-β |
| | GSK-3-β |
| 3-cyclohexyl-4-(2-methoxyphenyl)-5-(4-phenylbutoxy)-4H-1,2,4-triazole | p38-α |
| 5-(2-methoxyphenyl)-4H-1,2,4-triazol-3-amine | AURORA-A |
| | CDK2/CyclinE |
| | CHEK2 |
| 1-(3-amino-4H-1,2,4-triazol-4-yl)-2-(4-chlorophenyl)ethanone | GSK-3-α |
| | GSK-3-β |
| benzyl 2-(5-(3-chlorophenyl)-4-(2-methoxyphenyl)-4H-1,2,4-triazol-3- | p38-α |
| ylamino)acetate | ROCK2 |
| N-(2-chlorophenyl)-2-(5-(3-chlorophenyl)-4-(2-methoxyphenyl)-4H-1,2,4- | p38-α |
| triazol-3-ylamino)acetamide | |
| N-(3-(2-(5-(3-chlorophenyl)-4-(2-methoxyphenyl)-4H-1,2,4-triazol-3- | p38-α |
| ylamino)acetamido)-4-methylphenyl)furan-2-carboxamide | p38-β |
| | MAPKAPK3 |
| 4-(4-(4-methyl-5-(thiocyanatomethylthio)-4H-1,2,4-triazol-3- | GSK3-β |
| yl)phenylsulfonyl)morpholine | |
| 5-bromo-4-(4-cyclohexyl-5-phenyl-4H-1,2,4-triazol-3-ylthio)-2- | GSK3-α |
| phenylpyridazin-3(2H)-one | |
| 3-(4-allyl-5-(3,4-dichlorophenyl)-4H-1,2,4-triazol-3-ylthio)-4- | FLT-3 |
| phenylquinolin-2-o | |
| 8-nitro-5-(5-(phenoxymethyl)-4-phenyl-4H-1,2,4-triazol-3-ylthio)quinoline | FLT-3 |
| | PDGFRR-α |
| 4-allyl-5-((4-chlorophenoxy)methyl)-4H-1,2,4-triazole-3-thiol | GSK3-α |
| 5-(3,4-dimethoxyphenyl)-4-(furan-2-ylmethyl)-4H-1,2,4-triazole-3-thiol | MSK2 |
| 4-allyl-5-(3,4,5-trimethoxyphenyl)-4H-1,2,4-triazole-3-thiol | GSK3-β |
| | GSK3-α |
| 4-(4-bromophenyl)-5-(pyridin-4-yl)-4H-1,2,4-triazole-3-thiol | p38-α |
| 5-((3-chlorophenylamino)methyl)-4-phenyl-4H-1,2,4-triazole-3-thiol | GSK3-β |
| | GSK3-α |
| 4-ethyl-5-(4-(4-methylpiperazin-1-ylsulfonyl)phenyl)-4H-1,2,4-triazole-3- | GSK3-α |
| thiol | GSK3-β |
| 4-(4-methoxyphenyl)-5-(pyridin-3-yl)-4H-1,2,4-triazole-3-thiol | ABL1 |
| 5-((4,6-dimethylpyrimidin-2-ylthio)methyl)-4-ethyl-4H-1,2,4-triazole-3-thiol | GSK3-α |
| 3-(4-(furan-2-ylmethyl)-5-mercapto-4H-1,2,4-triazol-3-yl)naphthalen-2-ol | AURORA-A |
| 5-(2-(1H-benzo[d]imidazol-2-yl)ethyl)-4-p-tolyl-4H-1,2,4-triazole-3-thiol | GSK3-α |
| | GSK3-β |
| 4-methyl-5-((naphthalen-2-yloxy)methyl)-4H-1,2,4-triazole-3-thiol | GSK3-α |
| 5-cyclohexyl-4-methyl-4H-1,2,4-triazole-3-thiol | AKT1 |
| 5-((4-bromophenoxy)methyl)-4-methyl-4H-1,2,4-triazole-3-thiol | CDK2-cyclinE |
| 4-(4-methoxyphenyl)-5-((4-methoxyphenylamino)methyl)-4H-1,2,4-triazole- | PDGFRR-α |
| 3-thiol | AURORA-A |
| | GSK3-α |
| 4-(5-mercapto-4-phenyl-4H-1,2,4-triazol-3-yl)phenol | CDK5 |
| | GSK3-α |
| | CDK2-cyclinA |
| 5-((m-toluidino)methyl)-4-phenyl-4H-1,2,4-triazole-3-thiol | GSK3-α |
| 5-((3-chlorophenylamino)methyl)-4-ethyl-4H-1,2,4-triazole-3-thiol | GSK3-β |
| | GSK3-α |
| 5-((4-methoxyphenoxy)methyl)-4-p-tolyl-4H-1,2,4-triazole-3-thiol | AURORA-A |
| | GSK3-α |
| 4-(2-methoxyphenyl)-5-(pyridin-3-yl)-4H-1,2,4-triazole-3-thiol | AURORA-A |
| 5-((4-fluorophenylamino)methyl)-4-(4-methoxyphenyl)-4H-1,2,4-triazole-3- | GSK3-α |
| thiol | p38-β |
| | PDGFRR-α |
| 4-benzyl-5-m-tolyl-4H-1,2,4-triazole-3-thiol | GSK3-α |
| 5-(2-methoxyphenyl)-4-p-tolyl-4H-1,2,4-triazole-3-thiol | CDK2-cyclinA |
| | CDK2-cyclinE |
| | CDK5 |
| 5-((3-chloro-4-methylphenylamino)methyl)-4-p-tolyl-4H-1,2,4-triazole-3- | GSK3-α |
| thiol | AURORA-A |
| 4-benzyl-5-((4-ethylphenoxy)methyl)-4H-1,2,4-triazole-3-thiol | AKT1 |
| 2-(5-mercapto-4-p-tolyl-4H-1,2,4-triazol-3-yl)phenol | AURORA-A |
| 5-((3-chloro-4-methylphenylamino)methyl)-4-((tetrahydrofuran-2- | GSK3-β |
| yl)methyl)-4H-1,2,4-triazole-3-thiol | GSK3-α |
| 5-((4-nitrophenoxy)methyl)-4-phenyl-4H-1,2,4-triazole-3-thiol | PDGFRR-α |
| | FLT-3 |
| 5-((4-chlorophenylamino)methyl)-4-(4-methoxyphenyl)-4H-1,2,4-triazole-3- | AURORA-A |
| thiol | GSK3-α |
| 3-(4-ethyl-5-mercapto-4H-1,2,4-triazol-3-yl)naphthalen-2-ol | AURORA-A |
| 4-(3-chlorophenyl)-5-((4-ethoxyphenylamino)methyl)-4H-1,2,4-triazole-3- | PDGFRR-α |
| thiol | FLT-3 |
| 5-(2-methoxyphenyl)-4-(4-methoxyphenyl)-4H-1,2,4-triazole-3-thiol | CDK2-cyclinA |
| | CDK2-cyclinE |
| | CDK5 |
| 5-((4-ethoxyphenylamino)methyl)-4-(4-methoxyphenyl)-4H-1,2,4-triazole-3- | FLT-3 |
| thiol | PDGFRR-α |
| | AURORA-A |
| 5-((4-fluorophenylamino)methyl)-4-p-tolyl-4H-1,2,4-triazole-3-thiol | AURORA-A |
| | GSK3-α |
| | GSK3-β |
| 5-((p-toluidino)methyl)-4-(4-methoxyphenyl)-4H-1,2,4-triazole-3-thiol | AURORA-A |
| | PDGFRR-α |
| | GSK3-β |
| 4-benzyl-5-((4-fluorophenylamino)methyl)-4H-1,2,4-triazole-3-thiol | CHEK2 |
| 4-(3-chlorophenyl)-5-((4-methoxyphenylamino)methyl)-4H-1,2,4-triazole-3- | PDGFRR-α |
| thiol | AURORA-A |
| | FYN |
| 4-ethyl-5-((3-(trifluoromethyl)phenylamino)methyl)-4H-1,2,4-triazole-3-thiol | GSK3-α |
| N-(4-(5-mercapto-4-(4-methoxyphenyl)-4H-1,2,4-triazol-3-yl)phenyl)-4- | GSK3-β |
| methylbenzenesulfonamide | GSK3-α |
| | AURORA-A |
| 5-((3-chlorophenylamino)methyl)-4-p-tolyl-4H-1,2,4-triazole-3-thiol | GSK3-β |
| | GSK3-α |
| 5-((p-toluidino)methyl)-4-(3-chlorophenyl)-4H-1,2,4-triazole-3-thiol | CDK2-cyclinA |
| | GSK3-α |
| 4-(4-methoxyphenyl)-5-((naphthalen-2-yloxy)methyl)-4H-1,2,4-triazole-3- | GSK3-α |
| thiol | CDK1 |
| 2-(5-mercapto-4-phenethyl-4H-1,2,4-triazol-3-yl)phenol | CDK2-cyclinE |
| | DAPK1 |
| 5-((5-methyl-4-p-tolyl-4H-1,2,4-triazol-3-ylthio)methyl)-4-phenyl-4H-1,2,4- | PDGFRR-α |
| triazole-3-thiol | |
| N-(4-(5-mercapto-4-methyl-4H-1,2,4-triazol-3-yl)phenyl)-4- | AURORA-A |
| methylbenzenesulfonamide | CDK2-cyclinA |
| | CHEK2 |
| 5-((2-methoxyphenoxy)methyl)-4-methyl-4H-1,2,4-triazole-3-thiol | GSK3-α |
| 4-(4-(3-chlorophenyl)-5-mercapto-4H-1,2,4-triazol-3-yl)phenol | CDK5 |
| | CDK2-cyclinA |
| | CDK2-cyclinE |
| 4-ethyl-5-((4-methoxyphenoxy)methyl)-4H-1,2,4-triazole-3-thiol | GSK3-β |
| | GSK3-α |
| | CDK2-cyclinE |
| 5-((4-methoxyphenylamino)methyl)-4-p-tolyl-4H-1,2,4-triazole-3-thiol | AURORA-A |
| | PDGFRR-α |
| | MSK2 |
| 5-((3-chlorophenylamino)methyl)-4-phenethyl-4H-1,2,4-triazole-3-thiol | GSK3-α |
| | GSK3-β |
| | AURORA-A |
| 5-(2-(1H-benzo[d]imidazol-2-yl)ethyl)-4-(3-chlorophenyl)-4H-1,2,4-triazole- | GSK3-α |
| 3-thiol | |
| 5-methyl-4-((tetrahydrofuran-2-yl)methyl)-4H-1,2,4-triazole-3-thiol | MAPKAPK-2 |
| 4-(5-mercapto-4-(4-methoxyphenyl)-4H-1,2,4-triazol-3-yl)phenol | CDK5 |
| | GSK3-β |
| | GSK3-α |
| 5-((4-fluorophenylamino)methyl)-4-phenyl-4H-1,2,4-triazole-3-thiol | AURORA-A |
| 4-benzyl-5-(2-methoxyphenyl)-4H-1,2,4-triazole-3-thiol | AURORA-A |
| 5-(2-(1H-benzo[d]imidazol-2-yl)ethyl)-4-methyl-4H-1,2,4-triazole-3-thiol | GSK3-α |
| | GSK3-β |
| 4-phenyl-5-((3-(trifluoromethyl)phenylamino)methyl)-4H-1,2,4-triazole-3- | GSK3-α |
| thiol | |
| 4-(4-methoxyphenyl)-5-((3-(trifluoromethyl)phenylamino)methyl)-4H-1,2,4- | GSK3-α |
| triazole-3-thiol | GSK3-β |
| 4-(3-chlorophenyl)-5-cyclohexyl-4H-1,2,4-triazole-3-thiol | AURORA-A |
| 5-((naphthalen-2-yloxy)methyl)-4-phenyl-4H-1,2,4-triazole-3-thiol | AURORA-A |
| 5-(2-(1H-benzo[d]imidazol-2-yl)ethyl)-4-ethyl-4H-1,2,4-triazole-3-thiol | GSK3-β |
| | GSK3-α |
| 4-(4-methoxyphenyl)-5-methyl-4H-1,2,4-triazole-3-thiol | AURORA-A |
| 4-benzyl-5-((4-chlorophenylamino)methyl)-4H-1,2,4-triazole-3-thiol | AURORA-A |
| 5-((4-ethoxyphenylamino)methyl)-4-p-tolyl-4H-1,2,4-triazole-3-thiol | AURORA-A |
| 4-ethyl-5-((2-methoxyphenoxy)methyl)-4H-1,2,4-triazole-3-thiol | GSK3-α |
| | GSK3-β |
| | AURORA-A |
| 4-benzyl-5-((2-methoxyphenoxy)methyl)-4H-1,2,4-triazole-3-thiol | AURORA-A |
| 4-(furan-2-ylmethyl)-5-((2-methoxyphenoxy)methyl)-4H-1,2,4-triazole-3- | AURORA-A |
| thiol | GSK3-β |
| | GSK3-α |
| 5-((4-chlorophenoxy)methyl)-4-(2-methoxyethyl)-4H-1,2,4-triazole-3-thiol | GSK3-α |
| 3-(5-mercapto-4-(2-methoxyethyl)-4H-1,2,4-triazol-3-yl)naphthalen-2-ol | AURORA-A |
| 4-methyl-5-((naphthalen-1-yloxy)methyl)-4H-1,2,4-triazole-3-thiol | GSK3-β |
| | GSK3-α |
| 5-((4-chloro-3-methylphenoxy)methyl)-4-ethyl-4H-1,2,4-triazole-3-thiol | GSK3-α |
| 5-((p-toluidino)methyl)-4-(4-chlorophenyl)-4H-1,2,4-triazole-3-thiol | PAK2 |
| | GSK3-α |
| 4-(5-mercapto-4-methyl-4H-1,2,4-triazol-3-yl)phenol | CDK5 |
| | CDK2-cyclinA |
| | CDK2-cyclinE |
| 5-((3-chloro4-methylphenylamino)methyl)-4-phenyl-4H-1,2,4-triazole-3- | GSK3-β |
| thiol | GSK3-α |
| | AURORA-A |
| 5-(2-methoxyphenyl)-4-(2-methylallyl)-4H-1,2,4-triazole-3-thiol | CDK2-cyclinE |
| 4-phenyl-5-(pyridin-4-yl)-4H-1,2,4-triazole-3-thiol | LCK |
| 4-cyclohexyl-5-(m-tolyloxymethyl)-4H-1,2,4-triazole-3-thiol | GSK3-α |
| | CHEK1 |
| | AURORA-A |
| 5-(2-methoxyphenyl)-4-methyl-4H-1,2,4-triazole-3-thiol | CDK2-cyclinE |
| 5-(2-bromophenyl)-4-(2-methylallyl)-4H-1,2,4-triazole-3-thiol | PDGFRR-α |
| N-(4-(5-mercapto-4-methyl-4H-1,2,4-triazol-3-yl)phenyl)benzamide | GSK3-α |
| | CHEK2 |
| 5-((3-(dimethylamino)phenoxy)methyl)-4-phenyl-4H-1,2,4-triazole-3-thiol | GSK3-α |
| 3-(4-allyl-5-mercapto-4H-1,2,4-triazol-3-yl)naphthalen-2-ol | MSK1 |
| 4-(4-amino-3,5-dichlorophenyl)-5-phenyl-4H-1,2,4-triazole-3-thiol | GSK3-β |
| | GSK3-α |
| | CDK2-cyclinE |
| 5-(4-methoxyphenyl)-4-p-tolyl-4H-1,2,4-triazole-3-thiol | GSK3-β |
| | GSK3-α |
| 4-phenyl-5-(4-propoxyphenethyl)-4H-1,2,4-triazole-3-thiol | GSK3-β |
| | GSK3-α |
| 4-(3-(3-cyclopropyl-1H-pyrazol-5-yl)-5-mercapto-4H-1,2,4-triazol-4- | GSK3-α |
| yl)benzoic acid | |
| 2-(5-(2-methoxyphenyl)-4-p-tolyl-4H-1,2,4-triazol-3-ylthio)acetonitrile | p38-β |
| | AURORA-A |
| 2-(5-((2,4-dimethylphenylamino)methyl)-4-phenyl-4H-1,2,4-triazol-3- | AKT1 |
| ylthio)acetonitrile | |
| 2-(5-((naphthalen-1-yloxy)methyl)-4-phenyl-4H-1,2,4-triazol-3- | MAPKAPK-2 |
| ylthio)acetonitrile | CDK1 |
| | CDK5 |
| 2-(4-(3-chlorophenyl)-5-((4,6-dimethylpyrimidin-2-ylthio)methyl)-4H-1,2,4- | LYNA |
| triazol-3-ylthio)acetonitrile | |
| 2-(4-(4-methoxyphenyl)-5-(pyridin-3-yl)-4H-1,2,4-triazol-3- | AURORA-A |
| ylthio)acetonitrile | |
| 2-(5-((3-chlorophenylamino)methyl)-4-ethyl-4H-1,2,4-triazol-3- | CDK2-cyclinA |
| ylthio)acetonitrile | |
| 2-(5-((3-chlorophenylamino)methyl)-4-p-tolyl-4H-1,2,4-triazol-3- | p38-α |
| ylthio)acetonitrile | |
| 2-(5-((4-ethoxyphenylamino)methyl)-4-p-tolyl-4H-1,2,4-triazol-3- | FYN |
| ylthio)acetonitrile | |
| 2-(5-(2-bromophenyl)-4-p-tolyl-4H-1,2,4-triazol-3-ylthio)acetonitrile | p38-β |
| 2-(4-phenyl-5-(pyridin-3-yl)-4H-1,2,4-triazol-3-ylthio)acetonitrile | AURORA-A |
| 2-(4-benzyl-5-((4-chlorophenylamino)methyl)-4H-1,2,4-triazol-3- | MSK1 |
| ylthio)acetonitrile | |
| 2-(4-(4-methoxyphenyl)-5-((naphthalen-1-yloxy)methyl)-4H-1,2,4-triazol-3- | MAPKAPK-2 |
| ylthio)acetonitrile | CDK1 |
| 2-(5-((4-chloro-3-methylphenoxy)methyl)-4-phenyl-4H-1,2,4-triazol-3- | p38-α |
| ylthio)acetonitrile | |
| 2-(5-((2-chloro-5-(trifluoromethyl)phenylamino)methyl)-4-(4- | CDK1 |
| methoxyphenyl)-4H-1,2,4-triazol-3-ylthio)acetonitrile | |
| 2-(5-((3-chlorophenylamino)methyl)-4-phenethyl-4H-1,2,4-triazol-3- | p38-α |
| ylthio)acetonitrile | CDK2-cyclinA |
| | AURORA-A |
| 2-(5-((p-toluidino)methyl)-4-ethyl-4H-1,2,4-triazol-3-ylthio)acetonitrile | PDGFRR-α |
| | CDK1 |
| 2-(5-(4-hydroxyphenyl)-4-(4-methoxyphenyl)-4H-1,2,4-triazol-3- | GSK3-β |
| ylthio)acetonitrile | GSK3-α |
| | p38-β |
| 2-(5-((p-toluidino)methyl)-4-(4-chlorophenyl)-4H-1,2,4-triazol-3- | PDGFRR-α |
| ylthio)acetonitrile | CDK1 |
| 2-(4-benzyl-5-((naphthalen-1-yloxy)methyl)-4H-1,2,4-triazol-3- | CDK1 |
| ylthio)acetonitrile | CHEK2 |
| 2-(5-(2-bromophenyl)-4-(4-methoxyphenyl)-4H-1,2,4-triazol-3- | p38-β |
| ylthio)acetonitrile | |
| N-(4-(5-(cyanomethylthio)-4-(4-methoxyphenyl)-4H-1,2,4-triazol-3- | CDK2-cyclinA |
| yl)phenyl)-4-methylbenzenesulfonamide | CDK1 |
| | CDK2-cyclinE |
| 2-(4-(4-methoxyphenyl)-5-m-tolyl-4H-1,2,4-triazol-3-ylthio)acetonitrile | p38-α |
| | p38-β |
| 2-(4-benzyl-5-(4-hydroxyphenyl)-4H-1,2,4-triazol-3-ylthio)acetonitrile | GSK3-β |
| | GSK3-α |
| 2-(4-(3-chlorophenyl)-5-((4-ethoxyphenylamino)methyl)-4H-1,2,4-triazol-3- | PDGFRR-α |
| ylthio)acetonitrile | FYN |
| 2-(5-((3-chloro-4-methylphenylamino)methyl)-4-p-tolyl-4H-1,2,4-triazol-3- | p38-α |
| ylthio)acetonitrile | PRAK |
| 2-(5-(furan-2-yl)-4-phenyl-4H-1,2,4-triazol-3-ylthio)acetonitrile | CHEK2 |
| 2-(5-cyclohexyl-4-phenyl-4H-1,2,4-triazol-3-ylthio)acetonitrile | CHEK2 |
| 2-(5-((3-chloro-4-methylphenylamino)methyl)-4-(4-methoxyphenyl)-4H- | p38-α |
| 1,2,4-triazol-3-ylthio)acetonitrile | |
| 2-(4-(2-methoxyphenyl)-5-((4-methoxyphenylamino)methyl)-4H-1,2,4- | FYN |
| triazol-3-ylthio)acetonitrile | |
| N-(4-(5-(cyanomethylthio)-4-(4-methoxyphenyl)-4H-1,2,4-triazol-3- | CHEK2 |
| yl)phenyl)benzamide | AKT1 |
| 2-(4-ethyl-5-((3-(trifluoromethyl)phenylamino)methyl)-4H-1,2,4-triazol-3- | CDK2 |
| ylthio)acetonitrile | CDK1 |
| | CHEK2 |
| 2-(4-phenyl-5-m-tolyl-4H-1,2,4-triazol-3-ylthio)acetonitrile | p38-β |
| 2-(5-((3-chloro-4-methylphenylamino)methyl)-4-phenyl-4H-1,2,4-triazol-3- | AURORA-A |
| ylthio)acetonitrile | |
| 2-(4-(2-methoxyphenyl)-5-phenyl-4H-1,2,4-triazol-3-ylthio)acetonitrile | p38-β |
| | p38-α |
| | c-TAK1 |
| 2-(4-ethyl-5-(4-(morpholinosulfonyl)phenyl)-4H-1,2,4-triazol-3- | AURORA-A |
| ylthio)acetonitrile | |
| 2-(4-ethyl-5-((4-ethylphenoxy)methyl)-4H-1,2,4-triazol-3-ylthio)acetonitrile | AURORA-A |
| 2-(4-ethyl-5-((naphthalen-1-yloxy)methyl)-4H-1,2,4-triazol-3- | AURORA-A |
| ylthio)acetonitrile | |
| 2-(5-((4-chloro-3-methylphenoxy)methyl)-4-ethyl-4H-1,2,4-triazol-3- | AURORA-A |
| ylthio)acetonitrile | |
| 2-(5-benzyl-4-methyl-4H-1,2,4-triazol-3-ylthio)acetonitrile | AURORA-A |
| 2-(4-benzyl-5-(pyridin-3-yl)-4H-1,2,4-triazol-3-ylthio)acetonitrile | AURORA-A |
| 2-(5-((3-chlorophenylamino)methyl)-4-methyl-4H-1,2,4-triazol-3- | AURORA-A |
| ylthio)acetonitrile | |
| 2-(5-((4-methoxyphenylamino)methyl)-4-p-tolyl-4H-1,2,4-triazol-3- | FYN |
| ylthio)acetonitrile | PDGFRR-α |
| | AURORA-A |
| 2-(4-(furan-2-ylmethyl)-5-(4-hydroxyphenyl)-4H-1,2,4-triazol-3- | GSK3-β |
| ylthio)acetonitrile | GSK3-α |
| | AURORA-A |
| 2-(5-(pyridin-4-yl)-4-p-tolyl-4H-1,2,4-triazol-3-ylthio)acetonitrile | AURORA-A |
| | GSK3-β |
| | GSK3-α |
| 2-(5-cyclohexyl-4-ethyl-4H-1,2,4-triazol-3-ylthio)acetonitrile | AURORA-A |
| | MSK1 |
| 2-(4-methyl-5-(p-tolyloxymethyl)-4H-1,2,4-triazol-3-ylthio)acetonitrile | CSK |
| 2-(5-(4-hydroxyphenyl)-4-methyl-4H-1,2,4-triazol-3-ylthio)acetonitrile | GSK3-β |
| | GSK3-α |
| | DAPK1 |
| 2-(5-(4-hydroxyphenyl)-4-phenyl-4H-1,2,4-triazol-3-ylthio)acetonitrile | GSK3-β |
| | GSK3-α |
| | p38-α |
| 2-(5-((4-chlorophenylamino)methyl)-4-ethyl-4H-1,2,4-triazol-3- | LYNA |
| ylthio)acetonitrile | DYRK2 |
| 2-(5-(2-hydroxyphenyl)-4-(2-methoxyphenyl)-4H-1,2,4-triazol-3- | AURORA-A |
| ylthio)acetonitrile | |
| 2-(5-tert-butyl-4H-1,2,4-triazol-3-ylthio)acetonitrile | DYRK2 |
| | GSK3-α |
| 2-(5-phenyl-4H-1,2,4-triazol-3-ylthio)acetonitrile | DYRK2 |
| | FLT-3 |
| | AURORA-A |
| 2-(5-(4-(trifluoromethoxy)phenyl)-4H-1,2,4-triazol-3-ylthio)acetonitrile | DYRK2 |
| | FLT-3 |
| | AURORA-A |
| 2-(5-(4-methoxyphenyl)-4H-1,2,4-triazol-3-ylthio)acetonitrile | FLT-3 |
| | DYRK2 |
| | AURORA-A |
| 2-(5-(4-fluorophenyl)-4H-1,2,4-triazol-3-ylthio)acetonitrile | FLT-3 |
| | DYRK2 |
| | GSK3-β |
| 2-(5-(thiophen-2-yl)-4H-1,2,4-triazol-3-ylthio)acetonitrile | DYRK2 |
| | FLT-3 |
| | AURORA-A |
| 2-(5-(2-(methylthio)ethyl)-4H-1,2,4-triazol-3-ylthio)acetonitrile | GSK3-α |
| 2-(5-cyclohexyl-4H-1,2,4-triazol-3-ylthio)acetonitrile | GSK3-α |
| 2-(5-(4-methoxyphenyl)-4-phenyl-4H-1,2,4-triazol-3-ylthio)acetonitrile | p38-α |
| 2-(4-allyl-5-(4-nitrophenyl)-4H-1,2,4-triazol-3-ylthio)acetic acid | GSK3-β |
| 2-(5-(3,4-dimethoxyphenyl)-4-phenyl-4H-1,2,4-triazol-3-ylthio)acetic acid | FYN |
| ethyl 2-(5-((benzo[d]thiazol-2-ylthio)methyl)-4-methyl-4H-1,2,4-triazol-3- | CSK |
| ylthio)acetate | |
| 2-(5-(furan-2-yl)-4-(2-methylallyl)-4H-1,2,4-triazol-3-ylthio)acetic acid | HCK |
| | CHEK2 |
| 2-(5-(4-methoxyphenyl)-4-(2-methylallyl)-4H-1,2,4-triazol-3-ylthio)acetic | MSK1 |
| acid | |
| methyl 2-(5-(pyridin-4-yl)-4H-1,2,4-triazol-3-ylthio)acetate | AURORA-A |
| | DYRK2 |
| | GSK3-β |
| ethyl 2-(5-(pyridin-4-yl)-4H-1,2,4-triazol-3-ylthio)acetate | CDK2-cyclinA |
| | AURORA-A |
| | CDK5 |
| 2-(5-(furan-2-yl)-4-((tetrahydrofuran-2-yl)methyl)-4H-1,2,4-triazol-3- | AURORA-A |
| ylthio)acetic acid | |
| 2-(5-(4-nitrophenyl)-4-p-tolyl-4H-1,2,4-triazol-3-ylthio)acetic acid | DAPK1 |
| 2-(4-ethyl-5-(4-(morpholinosulfonyl)phenyl)-4H-1,2,4-triazol-3-ylthio)acetic | CDK1 |
| acid | |
| 2-(5-(4-bromophenyl)-4-(4-methoxyphenyl)-4H-1,2,4-triazol-3-ylthio)acetic | p38-β |
| acid | |
| 2-(4,5-dip-tolyl-4H-1,2,4-triazol-3-ylthio)acetic acid | DAPK1 |
| 2-(4-(4-methoxyphenyl)-5-(pyridin-3-yl)-4H-1,2,4-triazol-3-ylthio)acetic | AURORA-A |
| acid | |
| 2-(5-((4-methoxyphenylamino)methyl)-4-p-tolyl-4H-1,2,4-triazol-3- | FYN |
| ylthio)acetic acid | |
| cyclohexyl 2-(5-(4-hydroxyphenyl)-4-methyl-4H-1,2,4-triazol-3- | GSK3-α |
| ylthio)acetate | |
| 2-(4-(furan-2-ylmethyl)-5-(pyridin-3-yl)-4H-1,2,4-triazol-3-ylthio)acetic acid | DAPK1 |
| | GSK3-β |
| | AURORA-A |
| 2-(4-phenyl-5-((3-(trifluoromethyl)phenylamino)methyl)-4H-1,2,4-triazol-3- | P38-α |
| ylthio)acetic acid | |
| 2-(5-((naphthalen-1-yloxy)methyl)-4-p-tolyl-4H-1,2,4-triazol-3- | AKT1 |
| ylthio)propanoic acid | |
| 2-(4-benzyl-5-((4-fluorophenylamino)methyl)-4H-1,2,4-triazol-3- | DAPK1 |
| ylthio)acetic acid | |
| 2-(5-(4-chlorophenyl)-4-p-tolyl-4H-1,2,4-triazol-3-ylthio)acetic acid | DAPK1 |
| | AKT1 |
| 2-(4,5-dip-tolyl-4H-1,2,4-triazol-3-ylthio)propanoic acid | DAPK1 |
| 2-(4-phenyl-5-(o-tolyloxymethyl)-4H-1,2,4-triazol-3-ylthio)acetic acid | p38-α |
| 2-(4-ethyl-5-((4-methoxyphenoxy)methyl)-4H-1,2,4-triazol-3-ylthio)acetic | AKT1 |
| acid | |
| 2-(5-(phenoxymethyl)-4-phenyl-4H-1,2,4-triazol-3-ylthio)propanoic acid | FYN |
| 2-(5-((3-chlorophenylamino)methyl)-4-phenyl-4H-1,2,4-triazol-3- | p38-α |
| ylthio)acetic acid | |
| 2-(4-(2-methoxyphenyl)-5-o-tolyl-4H-1,2,4-triazol-3-ylthio)acetic acid | CHEK2 |
| benzyl 2-(5-(2-hydroxyphenyl)-4-(2-methoxyphenyl)-4H-1,2,4-triazol-3- | KIT |
| ylthio)acetate | |
| 2-(5-((3-chlorophenylamino)methyl)-4-(4-methoxyphenyl)-4H-1,2,4-triazol- | p38-α |
| 3-ylthio)acetic acid | |
| 2-(5-((2-methoxyphenoxy)methyl)-4-phenethyl-4H-1,2,4-triazol-3- | p38-β |
| ylthio)propanoic acid | |
| cyclohexyl 2-(4-benzyl-5-(4-hydroxyphenyl)-4H-1,2,4-triazol-3- | GSK3-α |
| ylthio)acetate | GSK3-β |
| 2-(5-((4-ethoxyphenylamino)methyl)-4-p-tolyl-4H-1,2,4-triazol-3- | FYN |
| ylthio)acetic acid | |
| 2-(4-benzyl-5-((4-ethylphenoxy)methyl)-4H-1,2,4-triazol-3-ylthio)acetic acid | AKT1 |
| 2-(5-((p-toluidino)methyl)-4-(4-methoxyphenyl)-4H-1,2,4-triazol-3- | PDGFRR-α |
| ylthio)acetic acid | |
| 2-(4-(2-methoxyethyl)-5-(3-methyl-1-phenyl-1H-thieno[2,3-c]pyrazol-5-yl)- | AKT1 |
| 4H-1,2,4-triazol-3-ylthio)acetic acid | |
| 2-(4-benzyl-5-((4-chlorophenylamino)methyl)-4H-1,2,4-triazol-3- | DAPK1 |
| ylthio)propanoic acid | |
| 2-(4-(2-methoxyphenyl)-5-(pyridin-3-yl)-4H-1,2,4-triazol-3-ylthio)acetic | AURORA-A |
| acid | |
| 2-(5-((3-chlorophenylamino)methyl)-4-ethyl-4H-1,2,4-triazol-3-ylthio)acetic | CDK2-cyclinA |
| acid | |
| ethyl 2-(5-((3-chloro-4-methylphenylamino)methyl)-4-((tetrahydrofuran-2- | CHEK2 |
| yl)methyl)-4H-1,2,4-triazol-3-ylthio)acetate | |
| 2-(5-((4-methoxyphenylamino)methyl)-4-phenyl-4H-1,2,4-triazol-3- | FYN( |
| ylthio)propanoic acid | PDGFRR-α |
| 2-(5-((3-chloro-4-methylphenylamino)methyl)-4-ethyl-4H-1,2,4-triazol-3- | PDGFRR-α |
| ylthio)acetic acid | |
| 2-(5-(4-tert-butylphenyl)-4-(3-methoxypropyl)-4H-1,2,4-triazol-3- | CHEK2 |
| ylthio)acetic acid | |
| 2-(4-(4-methoxyphenyl)-5-(4-(4-methylphenylsulfonamido)phenyl)-4H- | CDK2-cyclinA |
| 1,2,4-triazol-3-ylthio)acetic acid | CDK2-cyclinE |
| | CDK1 |
| 2-(5-(4-benzamidophenyl)-4-(4-methoxyphenyl)-4H-1,2,4-triazol-3- | CHEK2 |
| ylthio)propanoic acid | AURORA-A |
| 2-(5-(4-(morpholinosulfonyl)phenyl)-4-phenyl-4H-1,2,4-triazol-3- | CHEK2 |
| ylthio)acetic acid | |
| 2-(4-(4-methoxyphenyl)-5-((4-methoxyphenylamino)methyl)-4H-1,2,4- | PDGFRR-α |
| triazol-3-ylthio)acetic acid | FYN |
| 2-(5-(pyridin-3-yl)-4-p-tolyl-4H-1,2,4-triazol-3-ylthio)acetic acid | AURORA-A |
| p-tolyl 2-(4-ethyl-5-((3-(trifluoromethyl)phenylamino)methyl)-4H-1,2,4- | CDK2-cyclinA |
| triazol-3-ylthio)acetate | CDK1 |
| | CHEK2 |
| 3-methoxyphenyl 2-(5-((2,6-dimethylphenylamino)methyl)-4-phenyl-4H- | C-TAK1 |
| 1,2,4-triazol-3-ylthio)acetate | |
| 2-(5-((3-chlorophenylamino)methyl)-4-phenethyl-4H-1,2,4-triazol-3- | AURORA-A |
| ylthio)acetic acid | CDK2-cyclinA |
| | p38-α |
| 2-(5-cyclohexyl-4-ethyl-4H-1,2,4-triazol-3-ylthio)propanoic acid | p38-α |
| 2-(5-(4-nitrophenyl)-4-((tetrahydrofuran-2-yl)methyl)-4H-1,2,4-triazol-3- | PDGFRR-α |
| ylthio)acetic acid | |
| 2-(4-(4-chlorophenyl)-5-((3-chlorophenylamino)methyl)-4H-1,2,4-triazol-3- | p38-α |
| ylthio)acetic acd | |
| 2-(5-(4-benzamidophenyl)-4-(4-methoxyphenyl)-4H-1,2,4-triazol-3- | CHEK2 |
| ylthio)acetic acid | |
| 2-(4-ethyl-5-(pyridin-3-yl)-4H-1,2,4-triazol-3-ylthio)propanoic acid | LYNA |
| 2-(5-((3-chlorophenylamino)methyl)-4-p-tolyl-4H-1,2,4-triazol-3- | p38-α |
| ylthio)acetic acid | AURORA-A |
| cyclohexyl 2-(5-(3-hydroxynaphthalen-2-yl)-4-(2-methoxyethyl)-4H-1,2,4- | DAPK1 |
| triazol-3-ylthio)acetate | |
| 2-(4-(4-methoxyphenyl)-5-phenyl-4H-1,2,4-triazol-3-ylthio)propanoic acid | AURORA-A |
| 2-(4-methyl-5-(phenoxymethyl)-4H-1,2,4-triazol-3-ylthio)propanoic acid | AURORA-A |
| 2-(4-(4-fluorophenyl)-5-(pyridin-3-yl)-4H-1,2,4-triazol-3-ylthio)acetic acid | AURORA-A |
| 2-(4-methyl-5-((naphthalen-1-yloxy)methyl)-4H-1,2,4-triazol-3-ylthio)acetic | CDK2-cyclinA |
| acid | AURORA-A |
| 2-(5-((4-chloro-3-methylphenoxy)methyl)-4-phenyl-4H-1,2,4-triazol-3- | AURORA-A |
| ylthio)propanoic acid | |
| 2-(4-(4-methoxyphenyl)-5-(m-tolyloxymethyl)-4H-1,2,4-triazol-3- | AURORA-A |
| ylthio)propanoic acid | |
| 2-(5-(pyridin-3-yl)-4-p-tolyl-4H-1,2,4-triazol-3-ylthio)propanoic acid | AURORA-A |
| 2-(4-phenyl-5-(p-tolyloxymethyl)-4H-1,2,4-triazol-3-ylthio)propanoic acid | AURORA-A |
| 2-(5-((4-methoxyphenoxy)methyl)-4-p-tolyl-4H-1,2,4-triazol-3-ylthio)acetic | AURORA-A |
| acid | |
| 2-(4-(4-methoxyphenyl)-5-((naphthalen-2-yloxy)methyl)-4H-1,2,4-triazol-3- | AURORA-A |
| ylthio)acetic acid | |
| 2-(4-phenyl-5-(o-tolyloxymethyl)-4H-1,2,4-triazol-3-ylthio)propanoic acid | p38-α |
| | AURORA-A |
| 2-(5-(phenoxymethyl)-4-p-tolyl-4H-1,2,4-triazol-3-ylthio)propanoic acid | p38-α |
| | DAPK1 |
| cyclohexyl 2-(5-(3-hydroxynaphthalen-2-yl)-4-methyl-4H-1,2,4-triazol-3- | AURORA-A |
| ylthio)acetate | DAPK1 |
| | c-TAK1 |
| 2-(5-((4-chloro-3-methylphenoxy)methyl)-4-ethyl-4H-1,2,4-triazol-3- | MSK1 |
| ylthio)acetic acid | |
| 2-(4-phenyl-5-(pyridin-3-yl)-4H-1,2,4-triazol-3-ylthio)propanoic acid | AURORA-A |
| 2-(5-((4-bromophenoxy)methyl)-4-methyl-4H-1,2,4-triazol-3-ylthio)acetic | AURORA-A |
| acid | |
| 2-(4-methyl-5-(m-tolyloxymethyl)-4H-1,2,4-triazol-3-ylthio)propanoic acid | AURORA-A |
| 2-(5-(phenoxymethyl)-4-p-tolyl-4H-1,2,4-triazol-3-ylthio)acetic acid | p38-α |
| | AURORA-A |
| | DAPK1 |
| 2-(5-(2-methoxyphenyl)-4-(4-methoxyphenyl)-4H-1,2,4-triazol-3- | AURORA-A |
| ylthio)acetic acid | |
| 2-(5-((4-fluorophenylamino)methyl)-4-p-tolyl-4H-1,2,4-triazol-3- | DAPK1 |
| ylthio)acetic acid | AURORA-A |
| 2-(4-(furan-2-ylmethyl)-5-((3-(trifluoromethyl)phenylamino)methyl)-4H- | SRC |
| 1,2,4-triazol-3-ylthio)acetic acid | AURORA-A |
| 2-(4-(furan-2-ylmethyl)-5-((3-(trifluoromethyl)phenylamino)methyl)-4H- | AURORA-A |
| 1,2,4-triazol-3-ylthio)acetic acid | |
| 2-(5-((2-chlorophenylamino)methyl)-4-phenyl-4H-1,2,4-triazol-3- | p38-α |
| ylthio)acetic acid | |
| 2-(4-benzyl-5-((3-methoxyphenoxy)methyl)-4H-1,2,4-triazol-3-ylthio)acetic | MSK1 |
| acid | |
| 2-(4-benzyl-5-((4-chloro-3-methylphenoxy)methyl)-4H-1,2,4-triazol-3- | HCK |
| ylthio)acetic acid | |
| cyclohexyl 2-(5-((3-chloro-4-methylphenylamino)methyl)-4-phenyl-4H- | PAK2 |
| 1,2,4-triazol-3-ylthio)acetate | CHEK1 |
| ethyl 2-(5-(3,4-dimethoxyphenyl)-4-phenyl-4H-1,2,4-triazol-3-ylthio)acetate | MAPKAPK-2 |
| | CDK2-cyclinE |
| methyl 2-(5-((4-methoxyphenylamino)methyl)-4-phenyl-4H-1,2,4-triazol-3- | PDGFRR-α |
| ylthio)acetate | |
| ethyl 2-(5-(3-hydroxynaphthalen-2-yl)-4-methyl-4H-1,2,4-triazol-3- | FYN |
| ylthio)acetate | |
| isopropyl 2-(5-(3-hydroxynaphthalen-2-yl)-4-(2-methoxyethyl)-4H-1,2,4- | FYN |
| triazol-3-ylthio)acetate | |
| methyl 2-(4-(furan-2-ylmethyl)-5-(3-hydroxynaphthalen-2-yl)-4H-1,2,4- | p38-α |
| triazol-3-ylthio)acetate | FYN |
| methyl 2-(5-((4-methoxyphenylamino)methyl)-4-methyl-4H-1,2,4-triazol-3- | PDGFRR-α |
| ylthio)acetate | FYN |
| methyl 2-(5-(2-hydroxyphenyl)-4-(2-methoxyphenyl)-4H-1,2,4-triazol-3- | p38-α |
| ylthio)acetate | AURORA-A |
| | p38-β |
| methyl 2-(4-(2-methoxyphenyl)-5-(4-(methylsulfonamido)phenyl)-4H-1,2,4- | p38-α |
| triazol-3-ylthio)acetate | |
| ethyl 2-(5-((2,5-dichlorophenylamino)methyl)-4-ethyl-4H-1,2,4-triazol-3- | DYRK2 |
| ylthio)acetate | |
| isopropyl 2-(4-(4-methoxyphenyl)-5-((4-methoxyphenylamino)methyl)-4H- | FYN |
| 1,2,4-triazol-3-ylthio)acetate | CDK2-cyclinE |
| ethyl 2-(5-(furan-2-yl)-4-p-tolyl-4H-1,2,4-triazol-3-ylthio)acetate | PAK2 |
| 2-(5-(4-hydroxyphenyl)-4-phenyl-4H-1,2,4-triazol-3-ylthio)acetic acid | GSK3-β |
| | GSK3-α |
| 2-(5-(4-hydroxyphenyl)-4-(4-methoxyphenyl)-4H-1,2,4-triazol-3- | GSK3-β |
| ylthio)propanoic acid | MAPKAPK-2 |
| ethyl 2-(4-methyl-5-((naphthalen-2-yloxy)methyl)-4H-1,2,4-triazol-3- | MAPKAPK-2 |
| ylthio)acetate | |
| methyl 2-(4-ethyl-5-(4-hydroxyphenyl)-4H-1,2,4-triazol-3-ylthio)acetate | GSK3-β |
| | GSK3-α |
| | CHEK1 |
| 2-(5-(4-hydroxyphenyl)-4-methyl-4H-1,2,4-triazol-3-ylthio)acetic acid | GSK3-β |
| | MAPKAPK-2 |
| methyl 2-(4-ethyl-5-phenyl-4H-1,2,4-triazol-3-ylthio)acetate | MAPKAPK-2 |
| methyl 2-(4-ethyl-5-(4-nitrophenyl)-4H-1,2,4-triazol-3-ylthio)acetate | PDGFRR-α |
| | FYN |
| ethyl 2-(4-(furan-2-ylmethyl)-5-(4-hydroxyphenyl)-4H-1,2,4-triazol-3- | GSK3-β |
| ylthio)acetate | GSK3-α |
| cyclohexyl 2-(4-methyl-5-(4-nitrophenyl)-4H-1,2,4-triazol-3-ylthio)acetate | PDGFRR-α |
| | FYN |
| | FLT-3 |
| isopropyl 2-(4-(furan-2-ylmethyl)-5-(pyridin-3-yl)-4H-1,2,4-triazol-3- | AKT1 |
| ylthio)acetate | |
| 2-(4-ethyl-5-(3-methyl-1-phenyl-1H-thieno[2,3-c]pyrazol-5-yl)-4H-1,2,4- | FYN |
| triazol-3-ylthio)acetic acid | |
| 2-(4-(furan-2-ylmethyl)-5-(4-hydroxyphenyl)-4H-1,2,4-triazol-3-ylthio)acetic | GSK3-β |
| acid | GSK3-α |
| ethyl 2-(5-(2-methoxyphenyl)-4-methyl-4H-1,2,4-triazol-3-ylthio)acetate | AURORA-A |
| | FYN |
| benzyl 2-(5-(3-hydroxyphenyl)-4-phenyl-4H-1,2,4-triazol-3-ylthio)acetate | p38-α |
| | P70S6K1 |
| benzyl 2-(5-(2-methoxyphenyl)-4-phenyl-4H-1,2,4-triazol-3-ylthio)acetate | p38-α |
| | P70S6K1 |
| benzyl 2-(5-(4-chlorophenyl)-4-(2-methoxyphenyl)-4H-1,2,4-triazol-3- | p38-α |
| ylthio)acetate | |
| benzyl 2-(4-(2-methoxyphenyl)-5-(thiophen-3-yl)-4H-1,2,4-triazol-3- | p38-α |
| ylthio)acetate | |
| benzyl 2-(4-(2-methoxyphenyl)-5-o-tolyl-4H-1,2,4-triazol-3-ylthio)acetate | p38-α |
| | P70S6K1 |
| benzyl 2-(4-(2,4-difluorophenyl)-5-(4-methoxyphenyl)-4H-1,2,4-triazol-3- | p38-α |
| ylthio)acetate | GSK3-α |
| | p38-β |
| benzyl 2-(4-(2,4-difluorophenyl)-5-(2-hydroxyphenyl)-4H-1,2,4-triazol-3- | p38-α |
| ylthio)acetate | |
| benzyl 2-(4-cyclohexyl-5-(4-methoxyphenyl)-4H-1,2,4-triazol-3- | p38-α |
| ylthio)acetate | P70S6K1 |
| benzyl 2-(4-cyclohexyl-5-(2-hydroxyphenyl)-4H-1,2,4-triazol-3- | P70S6K1 |
| ylthio)acetate | |
| benzyl 2-(4-(furan-2-ylmethyl)-5-(4-methoxyphenyl)-4H-1,2,4-triazol-3- | p38-α |
| ylthio)acetate | P70S6K1 |
| benzyl 2-(4-(furan-2-ylmethyl)-5-(2-hydroxyphenyl)-4H-1,2,4-triazol-3- | GSK3-α |
| ylthio)acetate | |
| benzyl 2-(5-(4-methoxyphenyl)-4-(pyridin-3-yl)-4H-1,2,4-triazol-3- | GSK3-β |
| ylthio)acetate | p38-α |
| benzyl 2-(5-(2-hydroxyphenyl)-4-(pyridin-3-yl)-4H-1,2,4-triazol-3- | p38-α |
| ylthio)acetate | GSK3-β |
| benzyl 2-(4-(2-chlorophenyl)-5-(4-methoxyphenyl)-4H-1,2,4-triazol-3- | p38-α |
| ylthio)acetate | MAPKAPK-2 |
| | P70S6K1 |
| benzyl 2-(4-(2-chlorophenyl)-5-(2-hydroxyphenyl)-4H-1,2,4-triazol-3- | KIT |
| ylthio)acetate | p38-β |
| benzyl 2-(4-ethyl-5-(4-methoxyphenyl)-4H-1,2,4-triazol-3-ylthio)acetate | p38-α |
| | P70S6K1 |
| benzyl 2-(4-ethyl-5-(2-hydroxyphenyl)-4H-1,2,4-triazol-3-ylthio)acetate | P70S6K1 |
| | MAPKAPK-2 |
| | GSK3-β |
| benzyl 2-(4-(4-chlorophenyl)-5-(4-methoxyphenyl)-4H-1,2,4-triazol-3- | P70S6K1 |
| ylthio)acetate | |
| benzyl 2-(4-(4-chlorophenyl)-5-(2-hydroxyphenyl)-4H-1,2,4-triazol-3- | P70S6K1 |
| ylthio)acetate | CDK1 |
| benzyl 2-(4-isobutyl-5-(4-methoxyphenyl)-4H-1,2,4-triazol-3-ylthio)acetate | p38-α |
| benzyl 2-(5-(2-hydroxyphenyl)-4-isobutyl-4H-1,2,4-triazol-3-ylthio)acetate | CDK2-cyclinE |
| benzyl 2-(4-(2-cyanophenyl)-5-(4-methoxyphenyl)-4H-1,2,4-triazol-3- | P70S6K1 |
| ylthio)acetate | CDK2-cyclinE |
| benzyl 2-(5-(4-methoxyphenyl)-4-(3-methoxypropyl)-4H-1,2,4-triazol-3- | CDK2-cyclinE |
| ylthio)acetate | MSK1 |
| benzyl 2-(5-(2-hydroxyphenyl)-4-(3-methoxypropyl)-4H-1,2,4-triazol-3- | P70S6K1 |
| ylthio)acetate | |
| benzyl 2-(4,5-diphenyl-4H-1,2,4-triazol-3-ylthio)acetate | p38-α |
| benzyl 2-(5-(4-fluorophenyl)-4-phenyl-4H-1,2,4-triazol-3-ylthio)acetate | p38-α |
| benzyl 2-(5-(2-chlorophenyl)-4-phenyl-4H-1,2,4-triazol-3-ylthio)acetate | P70S6K1 |
| | PDK1 |
| benzyl 2-(4-phenyl-5-(pyridin-4-yl)-4H-1,2,4-triazol-3-ylthio)acetate | P70S6K1 |
| benzyl 2-(5-(cyclohexylmethyl)-4-phenyl-4H-1,2,4-triazol-3-ylthio)acetate | PDK1 |
| | HCK |
| benzyl 2-(4-phenyl-5-m-tolyl-4H-1,2,4-triazol-3-ylthio)acetate | p38-α |
| | CDK2-cyclinE |
| | GSK3-α |
| benzyl 2-(5-(3-chlorophenyl)-4-phenyl-4H-1,2,4-triazol-3-ylthio)acetate | p38-α |
| | CDK2-cyclinE |
| benzyl 2-(4-phenyl-5-p-tolyl-4H-1,2,4-triazol-3-ylthio)acetate | P70S6K1 |
| benzyl 2-(4-phenyl-5-o-tolyl-4H-1,2,4-triazol-3-ylthio)acetate | P70S6K1 |
| benzyl 2-(5-(3-methoxyphenyl)-4-phenyl-4H-1,2,4-triazol-3-ylthio)acetate | p38-α |
| | P70S6K1 |
| benzyl 2-(5-(4-hydroxyphenyl)-4-phenyl-4H-1,2,4-triazol-3-ylthio)acetate | p38-β |
| benzyl 2-(5-(1H-indol-7-yl)-4-phenyl-4H-1,2,4-triazol-3-ylthio)acetate | p38-α |
| benzyl 2-(5-(2,4-difluorophenyl)-4-phenyl-4H-1,2,4-triazol-3-ylthio)acetate | p38-α |
| | P70S6K1 |
| benzyl 2-(4-phenyl-5-(thiophen-2-yl)-4H-1,2,4-triazol-3-ylthio)acetate | p38-α |
| | P70S6K1 |
| benzyl 2-(5-cyclohexyl-4-phenyl-4H-1,2,4-triazol-3-ylthio)acetate | p38-α |
| | P70S6K1 |
| benzyl 2-(5-(furan-2-yl)-4-phenyl-4H-1,2,4-triazol-3-ylthio)acetate | P70S6K1 |
| benzyl 2-(4-phenyl-5-(pyridin-2-yl)-4H-1,2,4-triazol-3-ylthio)acetate | p38-α |
| | P70S6K1 |
| benzyl 2-(4-(2-methoxyphenyl)-5-phenyl-4H-1,2,4-triazol-3-ylthio)acetate | p38-β |
| | P70S6K1 |
| benzyl 2-(5-benzyl-4-(2-methoxyphenyl)-4H-1,2,4-triazol-3-ylthio)acetate | P70S6K1 |
| benzyl 2-(4-(2-methoxyphenyl)-5-(4-methoxyphenyl)-4H-1,2,4-triazol-3- | p38-α |
| ylthio)acetate | p70S6K1 |
| benzyl 2-(5-(4-fluorophenyl)-4-(2-methoxyphenyl)-4H-1,2,4-triazol-3- | p38-α |
| ylthio)acetate | |
| benzyl 2-(5-(2-chlorophenyl)-4-(2-methoxyphenyl)-4H-1,2,4-triazol-3- | p38-α |
| ylthio)acetate | P70S6K1 |
| | AKT1 |
| benzyl 2-(4-(2-methoxyphenyl)-5-(pyridin-4-yl)-4H-1,2,4-triazol-3- | p38-α |
| ylthio)acetate | |
| benzyl 2-(5-(cyclohexylmethyl)-4-(2-methoxyphenyl)-4H-1,2,4-triazol-3- | p38-α |
| ylthio)acetate | |
| benzyl 2-(4-(2-methoxyphenyl)-5-m-tolyl-4H-1,2,4-triazol-3-ylthio)acetate | p38-α |
| | p38-β |
| benzyl 2-(5-(3-chlorophenyl)-4-(2-methoxyphenyl)-4H-1,2,4-triazol-3- | p38-α |
| ylthio)acetate | p38-β |
| | P70S6K1 |
| benzyl 2-(4-(2-methoxyphenyl)-5-p-tolyl-4H-1,2,4-triazol-3-ylthio)acetate | p38-α |
| benzyl 2-(5-(3-hydroxyphenyl)-4-(2-methoxyphenyl)-4H-1,2,4-triazol-3- | p38-β |
| ylthio)acetate | P70S6K1 |
| benzyl 2-(4,5-bis(2-methoxyphenyl)-4H-1,2,4-triazol-3-ylthio)acetate | p38-α |
| benzyl 2-(4-(2-methoxyphenyl)-5-(3-methoxyphenyl)-4H-1,2,4-triazol-3- | P70S6K1 |
| ylthio)acetate | |
| benzyl 2-(5-(4-hydroxyphenyl)-4-(2-methoxyphenyl)-4H-1,2,4-triazol-3- | p38-α |
| ylthio)acetate | GSK3-β |
| benzyl 2-(5-(2,4-difluorophenyl)-4-(2-methoxyphenyl)-4H-1,2,4-triazol-3- | p38-α |
| ylthio)acetate | P70S6K1 |
| | AURORA-A |
| benzyl 2-(4-(2-methoxyphenyl)-5-(thiophen-2-yl)-4H-1,2,4-triazol-3- | p38-α |
| ylthio)acetate | |
| benzyl 2-(5-cyclohexyl-4-(2-methoxyphenyl)-4H-1,2,4-triazol-3- | p38-α |
| ylthio)acetate | KIT |
| benzyl 2-(5-(furan-2-yl)-4-(2-methoxyphenyl)-4H-1,2,4-triazol-3- | GSK3-α |
| ylthio)acetate | AURORA-A |
| benzyl 2-(4-(2-methoxyphenyl)-5-(pyridin-2-yl)-4H-1,2,4-triazol-3- | p38-α |
| ylthio)acetate | SRC |
| benzyl 2-(4-(3-chlorophenyl)-5-(4-methoxyphenyl)-4H-1,2,4-triazol-3- | p38-α |
| ylthio)acetate | |
| benzyl 2-(4-(3-chlorophenyl)-5-(2-hydroxyphenyl)-4H-1,2,4-triazol-3- | p38-α |
| ylthio)acetate | P70S6K1 |
| benzyl 2-(4-(3-methoxyphenyl)-5-(4-methoxyphenyl)-4H-1,2,4-triazol-3- | p38-α |
| ylthio)acetate | p38-β |
| | GSK3-β |
| benzyl 2-(5-(2-hydroxyphenyl)-4-(3-methoxyphenyl)-4H-1,2,4-triazol-3- | p38-α |
| ylthio)acetate | P70S6K1 |
| | SRC |
| benzyl 2-(4-benzyl-5-(4-methoxyphenyl)-4H-1,2,4-triazol-3-ylthio)acetate | p38-α |
| | CHEK2 |
| benzyl 2-(4-benzyl-5-(2-hydroxyphenyl)-4H-1,2,4-triazol-3-ylthio)acetate | p38-α |
| benzyl 2-(4-(4-fluorophenyl)-5-(2-hydroxyphenyl)-4H-1,2,4-triazol-3- | P70S6K1 |
| ylthio)acetate | |
| benzyl 2-(5-(4-methoxyphenyl)-4-o-tolyl-4H-1,2,4-triazol-3-ylthio)acetate | p38-α |
| benzyl 2-(5-(2-hydroxyphenyl)-4-o-tolyl-4H-1,2,4-triazol-3-ylthio)acetate | p38-α |
| benzyl 2-(5-(4-methoxyphenyl)-4-p-tolyl-4H-1,2,4-triazol-3-ylthio)acetate | p38-α |
| benzyl 2-(5-(4-methoxyphenyl)-4-m-tolyl-4H-1,2,4-triazol-3-ylthio)acetate | p38-α |
| | c-TAK1 |
| benzyl 2-(5-(2-hydroxyphenyl)-4-m-tolyl-4H-1,2,4-triazol-3-ylthio)acetate | p38-α |
| | LCK |
| | P70S6K1 |
| benzyl 2-(5-(4-methoxyphenyl)-4-(3-(trifluoromethyl)phenyl)-4H-1,2,4- | p38-α |
| triazol-3-ylthio)acetate | |
| benzyl 2-(5-(2-hydroxyphenyl)-4-(3-(trifluoromethyl)phenyl)-4H-1,2,4- | p38-α |
| triazol-3-ylthio)acetate | MAPKAPK-2 |
| benzyl 2-(4,5-bis(4-methoxyphenyl)-4H-1,2,4-triazol-3-ylthio)acetate | AKT1 |
| benzyl 2-(5-(2-hydroxyphenyl)-4-(4-methoxyphenyl)-4H-1,2,4-triazol-3- | p38-α |
| ylthio)acetate | |
| ethyl 2-(5-(4-methoxyphenyl)-4-phenyl-4H-1,2,4-triazol-3-ylthio)acetate | p38-γ |
| | p38-δ |
| ethyl 2-(5-(2-hydroxyphenyl)-4-(2-methoxyphenyl)-4H-1,2,4-triazol-3- | AURORA-A |
| ylthio)acetate | |
| 2-(5-m-tolyl-4H-1,2,4-triazol-3-ylthio)acetic acid | GSK3-β |
| | DYRK2 |
| | AURORA-A |
| 2-(5-((4-chlorophenoxy)methyl)-4H-1,2,4-triazol-3-ylthio)acetic acid | GSK3-B β |
| | GSK3-α |
| 2-(5-(4-nitrophenyl)-4H-1,2,4-triazol-3-ylthio)acetic acid | GSK3-α |
| | GSK3-β |
| | DYRK2 |
| 2-(5-(3,4-dichlorophenyl)-4H-1,2,4-triazol-3-ylthio)acetic acid | GSK3-α |
| | GSK3-B β |
| | AURORA-A |
| 2-(5-(4-(trifluoromethoxy)phenyl)-4H-1,2,4-triazol-3-ylthio)acetic acid | GSK3-β |
| | GSK3-α |
| | DYRK2 |
| 2-(5-methyl-4H-1,2,4-triazol-3-ylthio)acetic acid | GSK3-β |
| | GSK3-α |
| 2-(5-(thiophen-2-yl)-4H-1,2,4-triazol-3-ylthio)acetic acid | DYRK2 |
| | GSK3-β |
| | GSK3-α |
| 2-(5-(2-(methylthio)ethyl)-4H-1,2,4-triazol-3-ylthio)acetic acid | GSK3-α |
| | GSK3-β |
| 2-(5-cyclohexyl-4-(2-methoxyphenyl)-4H-1,2,4-triazol-3-ylthio)acetic acid | GSK3-β |
| benzyl 2-(5-(3-chlorophenyl)-4-(3-(trifluoromethyl)phenyl)-4H-1,2,4-triazol- | p38-α |
| 3-ylthio)acetate | |
| 1-phenylethyl 2-(5-(3-chlorophenyl)-4-(3-(trifluoromethyl)phenyl)-4H-1,2,4- | p38-α |
| triazol-3-ylthio)acetate | |
| 2-(pyridin-2-yl)ethyl 2-(5-(4-methoxyphenyl)-4-(3-(trifluoromethyl)phenyl)- | p38-α |
| 4H-1,2,4-triazol-3-ylthio)acetate | |
| thiophen-2-ylmethyl 2-(5-(4-methoxyphenyl)-4-(3-(trifluoromethyl)phenyl)- | MAPKAPK-3 |
| 4H-1,2,4-triazol-3-ylthio)acetate | p38-α |
| 3-fluorobenzyl 2-(5-(4-methoxyphenyl)-4-(3-(trifluoromethyl)phenyl)-4H- | p38-α |
| 1,2,4-triazol-3-ylthio)acetate | |
| 2-chloro-4-fluorobenzyl 2-(5-(4-methoxyphenyl)-4-(3- | p38-α |
| (trifluoromethyl)phenyl)-4H-1,2,4-triazol-3-ylthio)acetate | |
| furan-2-ylmethyl 2-(5-(4-methoxyphenyl)-4-(3-(trifluoromethyl)phenyl)-4H- | MAPKAPK-3 |
| 1,2,4-triazol-3-ylthio)acetate | p38-α |
| furan-3-ylmethyl 2-(5-(4-methoxyphenyl)-4-(3-(trifluoromethyl)phenyl)-4H- | p38-α |
| 1,2,4-triazol-3-ylthio)acetate | |
| chroman-4-yl 2-(5-(4-methoxyphenyl)-4-(3-(trifluoromethyl)phenyl)-4H- | p38-α |
| 1,2,4-triazol-3-ylthio)acetate | |
| 3-methylphenethyl 2-(5-(4-methoxyphenyl)-4-(3-(trifluoromethyl)phenyl)- | p38-α |
| 4H-1,2,4-triazol-3-ylthio)acetate | |
| 4-fluorobenzyl 2-(5-(4-methoxyphenyl)-4-(3-(trifluoromethyl)phenyl)-4H- | p38-α |
| 1,2,4-triazol-3-ylthio)acetate | |
| 2-(thiophen-3-yl)ethyl 2-(5-(4-methoxyphenyl)-4-(3- | MAPKAPK-3 |
| (trifluoromethyl)phenyl)-4H-1,2,4-triazol-3-ylthio)acetate | p38-α |
| 4-chlorophenethyl 2-(5-(4-methoxyphenyl)-4-(3-(trifluoromethyl)phenyl)- | p38-α |
| 4H-1,2,4-triazol-3-ylthio)acetate | |
| 2-methoxyphenethyl 2-(5-(4-methoxyphenyl)-4-(3-(trifluoromethyl)phenyl)- | p38-α |
| 4H-1,2,4-triazol-3-ylthio)acetate | |
| 3-chlorobenzyl 2-(5-(4-methoxyphenyl)-4-(3-(trifluoromethyl)phenyl)-4H- | p38-α |
| 1,2,4-triazol-3-ylthio)acetate | |
| 2-(2-chlorophenoxy)ethyl 2-(5-(4-methoxyphenyl)-4-(3- | p38-α |
| (trifluoromethyl)phenyl)-4H-1,2,4-triazol-3-ylthio)acetate | |
| 3-methylbenzyl 2-(5-(4-methoxyphenyl)-4-(3-(trifluoromethyl)phenyl)-4H- | p38-α |
| 1,2,4-triazol-3-ylthio)acetate | |
| (2,3-dihydrobenzo[b][1,4]dioxin-2-yl)methyl 2-(5-(4-methoxyphenyl)-4-(3- | p38-α |
| (trifluoromethyl)phenyl)-4H-1,2,4-triazol-3-ylthio)acetate | |
| cycloheptyl 2-(5-(4-methoxyphenyl)-4-(3-(trifluoromethyl)phenyl)-4H-1,2,4- | FLT-3 |
| triazol-3-ylthio)acetate | |
| (4H-benzo[d][1,3]dioxin-2-yl)methyl 2-(5-(4-methoxyphenyl)-4-(3- | p38-α |
| (trifluoromethyl)phenyl)-4H-1,2,4-triazol-3-ylthio)acetate | |
| 2-methylphenethyl 2-(5-(4-methoxyphenyl)-4-(3-(trifluoromethyl)phenyl)- | p38-α |
| 4H-1,2,4-triazol-3-ylthio)acetate | |
| 2-chlorophenethyl 2-(5-(4-methoxyphenyl)-4-(3-(trifluoromethyl)phenyl)- | p38-α |
| 4H-1,2,4-triazol-3-ylthio)acetate | |
| 3-chlorophenethyl 2-(5-(4-methoxyphenyl)-4-(3-(trifluoromethyl)phenyl)- | p38-α |
| 4H-1,2,4-triazol-3-ylthio)acetate | |
| 2-chlorobenzyl 2-(5-(4-methoxyphenyl)-4-(3-(trifluoromethyl)phenyl)-4H- | MAPKAPK-3 |
| 1,2,4-triazol-3-ylthio)acetate | p38-α |
| 2-methylbenzyl 2-(5-(4-methoxyphenyl)-4-(3-(trifluoromethyl)phenyl)-4H- | p38-α |
| 1,2,4-triazol-3-ylthio)acetate | |
| phenethyl 2-(5-(4-methoxyphenyl)-4-(3-(trifluoromethyl)phenyl)-4H-1,2,4- | MAPKAP-3 |
| triazol-3-ylthio)acetate | p38-α |
| phenyl 2-(5-(3-chlorophenyl)-4-(2-methoxyphenyl)-4H-1,2,4-triazol-3- | MAPKAPK-3 |
| ylthio)propanoate | p38-α |
| benzyl 2-(4-(2-methoxyphenyl)-5-phenyl-4H-1,2,4-triazol-3- | p38-α |
| ylthio)propanoate | |
| phenethyl 2-(4-(2-methoxyphenyl)-5-phenyl-4H-1,2,4-triazol-3- | p38-α |
| ylthio)propanoate | |
| phenethyl 2-(5-cyclohexyl-4-(2-methoxyphenyl)-4H-1,2,4-triazol-3- | p38-α |
| ylthio)propanoate | |
| phenethyl 2-(5-(3-chlorophenyl)-4-(2-methoxyphenyl)-4H-1,2,4-triazol-3- | p38-α |
| ylthio)propanoate | |
| 1-(1H-indol-3-yl)-2-(5-methyl-4H-1,2,4-triazol-3-ylthio)ethanone | p38-α |
| | PDGFRR-α |
| | p38-β |
| 1-(1H-indol-3-yl)-2-(5-(pyridin-4-yl)-4H-1,2,4-triazol-3-ylthio)ethanone | CHEK2 |
| | AURORA-A |
| | PKA |
| 4-(5-(4-hydroxyphenyl)-4-methyl-4H-1,2,4-triazol-3-ylthio)-3-oxo-N- | AURORA-A |
| phenylbutanamide | |
| 2-(5-(4-hydroxyphenyl)-4-phenyl-4H-1,2,4-triazol-3-ylthio)-1- | GSK3-α |
| phenylethanone | |
| 2-(5-cyclohexyl-4-methyl-4H-1,2,4-triazol-3-ylthio)-1-(4- | SYK |
| methoxyphenyl)ethanone | |
| 2-(5-((p-toluidino)methyl)-4-phenyl-4H-1,2,4-triazol-3-ylthio)-1-(4- | CHEK2 |
| methoxyphenyl)ethanone | SYK |
| 4-(4-benzyl-5-(pyridin-3-yl)-4H-1,2,4-triazol-3-ylthio)-3-oxo-N- | AURORA-A |
| phenylbutanamide | |
| 1-phenyl-2-(5-(pyridin-4-yl)-4-p-tolyl-4H-1,2,4-triazol-3-ylthio)ethanone | AURORA-A |
| | GSK3-α |
| | p38-α |
| 3-oxo-N-phenyl-4-(4-phenyl-5-(pyridin-3-yl)-4H-1,2,4-triazol-3- | AURORA-A |
| ylthio)butanamide | |
| 2-(4-(2-methoxyethyl)-5-(4-methoxyphenyl)-4H-1,2,4-triazol-3-ylthio)-1- | AKT1 |
| phenylethanone | |
| 2-(4-ethyl-5-(pyridin-3-yl)-4H-1,2,4-triazol-3-ylthio)-1-phenylethanone | SYK |
| 2-(5-cyclohexyl-4-ethyl-4H-1,2,4-triazol-3-ylthio)-1-phenylethanone | SRC |
| 3-oxo-N-phenyl-4-(5-(pyridin-3-yl)-4-p-tolyl-4H-1,2,4-triazol-3- | AURORA-A |
| ylthio)butanamide | |
| 2-(4-ethyl-5-(4-hydroxyphenyl)-4H-1,2,4-triazol-3-ylthio)-1-(thiophen-2- | MSK1 |
| yl)ethanone | MSK2 |
| | GSK3-β |
| 4-(5-(4-hydroxyphenyl)-4-phenyl-4H-1,2,4-triazol-3-ylthio)-3-oxo-N- | AURORA-A |
| phenylbutanamide | GSK3-α |
| 2-(4-(furan-2-ylmethyl)-5-(4-hydroxyphenyl)-4H-1,2,4-triazol-3-ylthio)-1- | GSK3-β |
| phenylethanone | GSK3-α |
| | MSK2 |
| 1-(2,3-dihydrobenzo[b][1,4]dioxin-6-yl)-2-(5-(4-hydroxyphenyl)-4-methyl- | GSK3-β |
| 4H-1,2,4-triazol-3-ylthio)ethanone | GSK3-α |
| | MSK1 |
| 2-(4-benzyl-5-(4-hydroxyphenyl)-4H-1,2,4-triazol-3-ylthio)-1- | GSK3-α |
| phenylethanone | |
| 2-(5-(4-hydroxyphenyl)-4-methyl-4H-1,2,4-triazol-3-ylthio)-1- | GSK3-β |
| phenylethanone | GSK3-α |
| | MSK1 |
| 2-(5-(3-hydroxynaphthalen-2-yl)-4-(2-methoxyethyl)-4H-1,2,4-triazol-3- | AURORA-A |
| ylthio)-1-(4-methoxyphenyl)ethanone | DAPK1 |
| | p38-δ |
| 2-(5-(4-(azepan-1-ylsulfonyl)phenyl)-4-ethyl-4H-1,2,4-triazol-3-ylthio)-1-p- | AURORA-A |
| tolylethanone | |
| 1-(2,5-dimethoxyphenyl)-2-(5-(3-hydroxynaphthalen-2-yl)-4-(2- | p38-δ |
| methoxyethyl)-4H-1,2,4-triazol-3-ylthio)ethanone | AURORA-A |
| | p38-α |
| 2-(4-(furan-2-ylmethyl)-5-(3-hydroxynaphthalen-2-yl)-4H-1,2,4-triazol-3- | AURORA-A |
| ylthio)-1-(4-methoxyphenyl)ethanone | |
| 1-(4-fluorophenyl)-2-(4-(furan-2-ylmethyl)-5-(3-hydroxynaphthalen-2-yl)- | AURORA-A |
| 4H-1,2,4-triazol-3-ylthio)ethanone | |
| N-(4-(2-(4-(furan-2-ylmethyl)-5-(4-hydroxyphenyl)-4H-1,2,4-triazol-3- | GSK3-β |
| ylthio)acetyl)-2-methoxyphenyl)propionamide | GSK3-α |
| N-(4-(2-(4-(furan-2-ylmethyl)-5-(4-hydroxyphenyl)-4H-1,2,4-triazol-3- | GSK3-α |
| ylthio)acetyl)-2-methoxyphenyl)acetamide | |
| 2-(4H-1,2,4-triazol-3-ylthio)-1-(2,4-dihydroxyphenyl)ethanone | GSK3-α |
| 1-(3,4-dihydroxyphenyl)-2-(4-methyl-5-phenyl-4H-1,2,4-triazol-3- | MSK2 |
| ylthio)ethanone | FYN |
| | HCK |
| 1-(4-chlorophenyl)-2-(5-((3-chlorophenylamino)methyl)-4-p-tolyl-4H-1,2,4- | LYNA |
| triazol-3-ylthio)ethanone | |
| 1-(3,4-dihydroxyphenyl)-2-(5-(furan-2-yl)-4H-1,2,4-triazol-3-ylthio)ethanone | FYN |
| | PDGFRR-α |
| | FLT-3 |
| 1-(3,5-di-tert-butyl-4-hydroxyphenyl)-2-(4-(4-ethoxyphenyl)-5-(4- | FYN |
| ethoxyphenylamino)-4H-1,2,4-triazol-3-ylthio)ethanone | |
| 1-(2,3-dihydrobenzo[b][1,4]dioxin-6-yl)-2-(5-(4-hydroxyphenyl)-4- | GSK3-β |
| ((tetrahydrofuran-2-yl)methyl)-4H-1,2,4-triazol-3-ylthio)ethanone | SGK1 |
| 2-(4-methyl-5-(pyridin-4-yl)-4H-1,2,4-triazol-3-ylthio)-1-phenylethanone | PKA |
| | MSK1 |
| | AURORA-A |
| 1-(2,3-dihydrobenzo[b][1,4]dioxin-6-yl)-2-(4-methyl-5-(4- | AURORA-A |
| (morpholinosulfonyl)phenyl)-4H-1,2,4-triazol-3-ylthio)ethanone | |
| 2-(4-ethyl-5-(pyridin-4-yl)-4H-1,2,4-triazol-3-ylthio)-1-(4- | PKA |
| methoxyphenyl)ethanone | MAPKAPK-2 |
| | MSK1 |
| 2-(4-(4-fluorophenyl)-5-(2-morpholinoethyl)-4H-1,2,4-triazol-3-ylthio)-1- | FYN |
| phenylethanone | |
| 1-(2,3-dihydrobenzo[b][1,4]dioxin-6-yl)-2-(5-(furan-2-yl)-4-propyl-4H-1,2,4- | DYRK2 |
| triazol-3-ylthio)ethanone | |
| 2-(5-(2-aminophenyl)-4-methyl-4H-1,2,4-triazol-3-ylthio)-1-(2,5- | PDGFRR-α |
| dimethoxyphenyl)ethanone | |
| 2-(5-cyclopropyl-4-methyl-4H-1,2,4-triazol-3-ylthio)-1-(2,3- | AURORA-A |
| dihydrobenzo[b][1,4]dioxin-6-yl)ethanone | FYN |
| 2-(5-((4-methoxyphenoxy)methyl)-4-methyl-4H-1,2,4-triazol-3-ylthio)-1- | PAK2 |
| phenylethanone | |
| 1-(2,5-dimethoxyphenyl)-2-(4-ethyl-5-isopropyl-4H-1,2,4-triazol-3- | PDGFRR-α |
| ylthio)ethanone | FLT-3 |
| | FYN |
| 1-(5-(4-methoxyphenyl)-4-phenyl-4H-1,2,4-triazol-3-ylthio)propan-2-one | INSR |
| 1-(5-(2-hydroxyphenyl)-4-(2-methoxyphenyl)-4H-1,2,4-triazol-3- | p38-α |
| ylthio)propan-2-one | |
| 1-phenyl-2-(5-(pyridin-3-yl)-4H-1,2,4-triazol-3-ylthio)ethanone | AURORA-A |
| 1-phenyl-2-(5-m-tolyl-4H-1,2,4-triazol-3-ylthio)ethanone | FLT-3 |
| | AURORA-A |
| 1-(2,3-dihydrobenzo[b][1,4]dioxin-6-yl)-2-(5-m-tolyl-4H-1,2,4-triazol-3- | FLT-3 |
| ylthio)ethanone | GSK3-α |
| | AURORA-A |
| 1-(2,3-dihydrobenzo[b][1,4]dioxin-6-yl)-2-(5-(3-(trifluoromethyl)phenyl)- | AURORA-A |
| 4H-1,2,4-triazol-3-ylthio)ethanone | GSK3-α |
| | FLT-3 |
| 2-(5-((4-chlorophenoxy)methyl)-4H-1,2,4-triazol-3-ylthio)-1-phenylethanone | GSK3-α |
| | FLT-3 |
| | GSK3-β |
| 2-(5-(4-nitrophenyl)-4H-1,2,4-triazol-3-ylthio)-1-phenylethanone | FLT-3 |
| | GSK3-β |
| | AURORA-A |
| 2-(5-benzyl-4H-1,2,4-triazol-3-ylthio)-1-(2,3-dihydrobenzo[b][1,4]dioxin-6- | GSK3-α |
| yl)ethanone | |
| 2-(5-tert-butyl-4H-1,2,4-triazol-3-ylthio)-1-(2,3-dihydrobenzo[b][1,4]dioxin- | DYRK2 |
| 6-yl)ethanone | GSK3-α |
| 2-(5-methyl-4H-1,2,4-triazol-3-ylthio)-1-phenylethanone | GSK3-α |
| | GSK3-β |
| | DYRK2 |
| 1-(2,3-dihydrobenzo[b][1,4]dioxin-6-yl)-2-(5-methyl-4H-1,2,4-triazol-3- | GSK3-α |
| ylthio)ethanone | GSK3-β |
| | DYRK2 |
| 1-(4-(2-methoxyphenyl)-5-phenyl-4H-1,2,4-triazol-3-ylthio)-3-phenylpropan- | p38-α |
| 2-one | |
| 1-(4-(2-methoxyphenyl)-5-phenyl-4H-1,2,4-triazol-3-ylthio)-4-phenylbutan- | p38-α |
| 2-one | |
| 1-(5-cyclohexyl-4-(2-methoxyphenyl)-4H-1,2,4-triazol-3-ylthio)-3- | p38-α |
| phenylpropan-2-one | |
| 1-(5-cyclohexyl-4-(2-methoxyphenyl)-4H-1,2,4-triazol-3-ylthio)-4- | p38-α |
| phenylbutan-2-one | PDK1 |
| 2-(4-allyl-5-(3-chlorobenzo[b]thiophen-2-yl)-4H-1,2,4-triazol-3-ylthio)-N-(3- | LYNA |
| morpholinopropyl)acetamide | |
| (E)-1-morpholino-2-(4-(3-morpholinoprop-1-enyl)-5-(4-nitrophenyl)-4H- | FYN |
| 1,2,4-triazol-3-ylthio)ethanone | |
| 2-(5-((benzo[d]thiazol-2-ylthio)methyl)-4-phenyl-4H-1,2,4-triazol-3-ylthio)- | CSK |
| N-(2-morpholinoethyl)acetamide | |
| 2-(4-allyl-5-((benzo[d]thiazol-2-ylthio)methyl)-4H-1,2,4-triazol-3-ylthio)-N- | HCK |
| (2-hydroxyethyl)acetamide | |
| 2-(5-((benzo[d]thiazol-2-ylthio)methyl)-4-phenyl-4H-1,2,4-triazol-3-ylthio)- | FYN |
| N-(2-hydroxyethyl)acetamide | |
| 2-(5-((benzo[d]thiazol-2-ylthio)methyl)-4-phenyl-4H-1,2,4-triazol-3-ylthio)- | HCK |
| 1-(4-(2-hydroxyethyl)piperazin-1-yl)ethanone | |
| 2-(2-(4-ethyl-5-(4-hydroxyphenyl)-4H-1,2,4-triazol-3-ylthio)acetamido)-4,5- | AURORA-A |
| dimethoxybenzoic acid | |
| N-(5-ethyl-1,3,4-thiadiazol-2-yl)-2-(4-ethyl-5-(4-(4-methylpiperazin-1- | CHEK2 |
| ylsulfonyl)phenyl)-4H-1,2,4-triazol-3-ylthio)acetamide | |
| N-benzyl-2-(4-(3-chlorophenyl)-5-(4-hydroxyphenyl)-4H-1,2,4-triazol-3- | GSK3-α |
| ylthio)acetamide | |
| methyl 2-(2-(5-(4-hydroxyphenyl)-4-methyl-4H-1,2,4-triazol-3- | GSK3-β |
| ylthio)acetamido)benzoate | GSK3-α |
| 2-(5-(2-hydroxyphenyl)-4-(2-methoxyphenyl)-4H-1,2,4-triazol-3-ylthio)-N- | CDK1 |
| (2-methylbenzo[d]thiazol-5-yl)acetamide | |
| N-(3-acetylphenyl)-2-(4-ethyl-5-(4-hydroxyphenyl)-4H-1,2,4-triazol-3- | GSK3-α |
| ylthio)acetamide | |
| 2-(4-ethyl-5-(4-hydroxyphenyl)-4H-1,2,4-triazol-3-ylthio)-N-(furan-2- | p38-α |
| ylmethyl)acetamide | GSK3-α |
| 2-(5-((4-ethoxyphenylamino)methyl)-4-(2-methoxyphenyl)-4H-1,2,4-triazol- | FYN |
| 3-ylthio)-N-((tetrahydrofuran-2-yl)methyl)acetamide | |
| methyl 2-(2-(4-(furan-2-ylmethyl)-5-(4-hydroxyphenyl)-4H-1,2,4-triazol-3- | GSK3-β |
| ylthio)acetamido)benzoate | MSK2 |
| | GSK3-α |
| 2-(5-(4-hydroxyphenyl)-4-methyl-4H-1,2,4-triazol-3-ylthio)-N-(naphthalen- | p38-δ |
| 1-yl)acetamide | |
| 2-(5-(4-hydroxyphenyl)-4-(4-methoxyphenyl)-4H-1,2,4-triazol-3-ylthio)-N- | GSK3-α |
| phenylpropanamide | AKT1 |
| N-(3-acetylphenyl)-2-(5-(4-hydroxyphenyl)-4-(4-methoxyphenyl)-4H-1,2,4- | GSK3-β |
| triazol-3-ylthio)acetamide | GSK3-α |
| N-(2,6-dimethylphenyl)-2-(5-(4-hydroxyphenyl)-4-(4-methoxyphenyl)-4H- | AKT1 |
| 1,2,4-triazol-3-ylthio)propanamide | |
| 2-(5-((2,6-dimethylphenylamino)methyl)-4-(2-methoxyphenyl)-4H-1,2,4- | CHEK2 |
| triazol-3-ylthio)-N-(3-hydroxyphenyl)acetamide | |
| ethyl 3-(2-(4-benzyl-5-(4-hydroxyphenyl)-4H-1,2,4-triazol-3- | p38-β |
| ylthio)acetamido)benzoate | GSK3-β |
| | LYNA |
| 2-(4-ethyl-5-(4-hydroxyphenyl)-4H-1,2,4-triazol-3-ylthio)-N-(naphthalen-1- | p38-δ |
| yl)acetamide | |
| 2-(4-ethyl-5-(4-hydroxyphenyl)-4H-1,2,4-triazol-3-ylthio)-N,N- | SRC |
| diphenylacetamide | |
| N-(3-acetylphenyl)-2-(5-(4-hydroxyphenyl)-4-methyl-4H-1,2,4-triazol-3- | GSK3-β |
| ylthio)acetamide | GSK3-α |
| | CHEK2 |
| 2-(5-(4-hydroxyphenyl)-4-methyl-4H-1,2,4-triazol-3-ylthio)-N-(naphthalen- | p38-δ |
| 1-yl)propanamide | |
| 2-(4-ethyl-5-(4-hydroxyphenyl)-4H-1,2,4-triazol-3-ylthio)-N-(naphthalen-1- | p38-δ |
| yl)propanamide | |
| methyl 2-(2-(4-benzyl-5-(4-hydroxyphenyl)-4H-1,2,4-triazol-3- | AKT3 |
| ylthio)acetamido)benzoate | GSK3-β |
| | GSK3-α |
| 2-(4-ethyl-5-(4-hydroxyphenyl)-4H-1,2,4-triazol-3-ylthio)-N- | CHEK2 |
| phenylacetamide | |
| 2-(5-(pyridin-4-yl)-4-((tetrahydrofuran-2-yl)methyl)-4H-1,2,4-triazol-3- | GSK3-β |
| ylthio)-N-(thiazol-2-yl)acetamide | GSK3-α |
| | ABL1 |
| 2-(5-(3-hydroxynaphthalen-2-yl)-4-(2-methoxyethyl)-4H-1,2,4-triazol-3- | DAPK1 |
| ylthio)-1-(10H-phenothiazin-10-yl)ethanone | |
| N-(4-acetylphenyl)-2-(5-(4-hydroxyphenyl)-4-methyl-4H-1,2,4-triazol-3- | AKT1 |
| ylthio)acetamide | |
| N-benzyl-2-(4-ethyl-5-(4-hydroxyphenyl)-4H-1,2,4-triazol-3- | p38-α |
| ylthio)acetamide | |
| N-(6-ethoxybenzo[d]thiazol-2-yl)-2-(5-(pyridin-4-yl)-4-((tetrahydrofuran-2- | PDGFRR-α |
| yl)methyl)-4H-1,2,4-triazol-3-ylthio)acetamide | GSK3-α |
| | ABL1 |
| 1-(azepan-1-yl)-2-(4-methyl-5-(4-(pyrrolidin-1-ylsulfonyl)phenyl)-4H-1,2,4- | c-TAK1 |
| triazol-3-ylthio)ethanone | |
| N-(4-acetylphenyl)-2-(4-benzyl-5-(4-hydroxyphenyl)-4H-1,2,4-triazol-3- | p38-α |
| ylthio)propanamide | GSK3-α |
| 1-(3,4-dihydroquinolin-1(2H)-yl)-2-(5-((4-fluorophenylamino)methyl)-4- | AKT1 |
| ((tetrahydrofuran-2-yl)methyl)-4H-1,2,4-triazol-3-ylthio)ethanone | |
| N-benzyl-2-(5-(2-hydroxyphenyl)-4-(2-methoxyphenyl)-4H-1,2,4-triazol-3- | p38-β |
| ylthio)acetamide | |
| 2-(4-benzyl-5-(4-hydroxyphenyl)-4H-1,2,4-triazol-3-ylthio)-N-(furan-2- | p38-α |
| ylmethyl)acetamide | GSK3-α |
| | GSK3-β |
| N-(2,4-dimethoxyphenyl)-2-(5-(2-hydroxyphenyl)-4-p-tolyl-4H-1,2,4-triazol- | MSK2 |
| 3-ylthio)acetamide | CDK1 |
| 2-(4-(3-chlorophenyl)-5-(4-hydroxyphenyl)-4H-1,2,4-triazol-3-ylthio)-N- | p38-α |
| (furan-2-ylmethyl)acetamide | GSK3-β |
| | GSK3-α |
| N-(5-ethyl-1,3,4-thiadiazol-2-yl)-2-(5-(4-hydroxyphenyl)-4-(4- | GSK3-β |
| methoxyphenyl)-4H-1,2,4-triazol-3-ylthio)acetamide | GSK3-α |
| | AKT1 |
| 2-(5-(3-hydroxynaphthalen-2-yl)-4-(2-methoxyethyl)-4H-1,2,4-triazol-3- | p38-δ |
| ylthio)-N-(5-methylisoxazol-3-yl)acetamide | CHEK2 |
| | HCK |
| 2-(4-(furan-2-ylmethyl)-5-(4-hydroxyphenyl)-4H-1,2,4-triazol-3-ylthio)-N- | GSK3-β |
| (4,5,6,7-tetrahydrobenzo[d]thiazol-2-yl)acetamide | GSK3-α |
| 2-(5-(4-hydroxyphenyl)-4-methyl-4H-1,2,4-triazol-3-ylthio)-N-(5- | GSK3-β |
| methylisoxazol-3-yl)acetamide | GSK3-α |
| 2-(4-(3-chlorophenyl)-5-(4-hydroxyphenyl)-4H-1,2,4-triazol-3-ylthio)-N- | GSK3-α |
| phenylacetamide | |
| 2-(4-(furan-2-ylmethyl)-5-(4-hydroxyphenyl)-4H-1,2,4-triazol-3- | GSK3-β |
| ylthio)acetamide | GSK3-α |
| 2-(5-(4-hydroxyphenyl)-4-(4-methoxyphenyl)-4H-1,2,4-triazol-3-ylthio)-N- | GSK3-α |
| p-tolylpropanamide | |
| 2-(4-benzyl-5-phenyl-4H-1,2,4-triazol-3-ylthio)-N-(2- | AKT1 |
| methylcyclohexyl)acetamide | |
| N-(6-ethoxybenzo[d]thiazol-2-yl)-2-(4-(furan-2-ylmethyl)-5-(pyridin-4-yl)- | GSK3-α |
| 4H-1,2,4-triazol-3-ylthio)acetamide | AKT1 |
| 2-(4-(furan-2-ylmethyl)-5-(pyridin-3-yl)-4H-1,2,4-triazol-3-ylthio)-N- | GSK3-α |
| (thiazol-2-yl)acetamide | GSK3-β |
| | CDK2-cyclinE |
| 2-(2-(4-(4-methoxyphenyl)-5-((4-methoxyphenylamino)methyl)-4H-1,2,4- | FYN |
| triazol-3-ylthio)acetamido)benzoic acid | |
| N-(3-hydroxyphenyl)-2-(4-(2-methoxyphenyl)-5-((4- | CHEK2 |
| methoxyphenylamino)methyl)-4H-1,2,4-triazol-3-ylthio)acetamide | PDGFRR-α |
| | AURORA-A |
| 2-(4-(3-chlorophenyl)-5-(4-hydroxyphenyl)-4H-1,2,4-triazol-3-ylthio)-N-(5- | GSK3-β |
| ethyl-1,3,4-thiadiazol-2-yl)acetamide | GSK3-α |
| 2-(5-((1H-benzo[d][1,2,3]triazol-1-yl)methyl)-4-methyl-4H-1,2,4-triazol-3- | GSK3-α |
| ylthio)-N-(3-hydroxyphenyl)acetamide | |
| N-benzyl-2-(4-benzyl-5-(4-hydroxyphenyl)-4H-1,2,4-triazol-3- | p38-α |
| ylthio)acetamide | |
| 2-(4-(furan-2-ylmethyl)-5-(3-hydroxynaphthalen-2-yl)-4H-1,2,4-triazol-3- | SRC |
| ylthio)acetamide | |
| 2-(4-(3-chlorophenyl)-5-(4-hydroxyphenyl)-4H-1,2,4-triazol-3- | GSK3-β |
| ylthio)acetamide | GSK3-α |
| N-allyl-2-(5-(2-hydroxyphenyl)-4-phenethyl-4H-1,2,4-triazol-3- | p38-α |
| ylthio)acetamide | |
| 2-(5-(3-hydroxynaphthalen-2-yl)-4-methyl-4H-1,2,4-triazol-3-ylthio)-1-(4-(4- | DAPK1 |
| methoxyphenyl)piperazin-1-yl)ethanone | |
| N-(4-(2-(4-ethyl-5-(4-hydroxyphenyl)-4H-1,2,4-triazol-3-ylthio)acetamido)- | GSK3-α |
| 2-methylphenyl)propionamide | |
| 2-(5-(4-hydroxyphenyl)-4-phenyl-4H-1,2,4-triazol-3-ylthio)acetamide | GSK3-β |
| | GSK3-α |
| | INSR |
| 1-(3,4-dihydroisoquinolin-2(1H)-yl)-2-(4-(furan-2-ylmethyl)-5-(4- | GSK3-β |
| hydroxyphenyl)-4H-1,2,4-triazol-3-ylthio)ethanone | GSK3-α |
| | SRC |
| N-(3-chloro-4-methylphenyl)-2-(4-(4-methoxyphenyl)-5-methyl-4H-1,2,4- | p38-α |
| triazol-3-ylthio)acetamide | |
| 2-(4-(2-methoxyphenyl)-5-phenyl-4H-1,2,4-triazol-3-ylthio)-N-o- | p38-α |
| tolylacetamide | |
| 1-(3,4-dihydroquinolin-1(2H)-yl)-2-(5-methyl-4-phenyl-4H-1,2,4-triazol-3- | c-TAK1 |
| ylthio)ethanone | |
| N-(4-acetylphenyl)-2-(5-(phenoxymethyl)-4-p-tolyl-4H-1,2,4-triazol-3- | CHEK2 |
| ylthio)acetamide | |
| 2-(5-((4-ethoxyphenylamino)methyl)-4-(2-methoxyphenyl)-4H-1,2,4-triazol- | PDGFRR-α |
| 3-ylthio)-N-(5-methylisoxazol-3-yl)acetamide | FYN |
| 1-(4-benzylpiperidin-1-yl)-2-(5-((2,3-dimethylphenoxy)methyl)-4-methyl- | p38-α |
| 4H-1,2,4-triazol-3-ylthio)ethanone | DAPK1 |
| 2-(4-methyl-5-(3-methyl-1-phenyl-1H-thieno[2,3-c]pyrazol-5-yl)-4H-1,2,4- | p38-β |
| triazol-3-ylthio)acetamide | AKT1 |
| 2-(5-((4,6-dimethylpyrimidin-2-ylthio)methyl)-4-methyl-4H-1,2,4-triazol-3- | CDK1 |
| ylthio)-N-(3-(trifluoromethyl)phenyl)acetamide | |
| 2-(4-ethyl-5-phenyl-4H-1,2,4-triazol-3-ylthio)-N-(thiazol-2-yl)acetamide | GSK3-β |
| | GSK3-α |
| | CHEK2 |
| 2-(5-((4-ethoxyphenylamino)methyl)-4-(4-methoxyphenyl)-4H-1,2,4-triazol- | FYN |
| 3-ylthio)-N,N-diphenylpropanamide | |
| N-(3-(2-(4-(3-methoxypropyl)-5-(pyridin-4-yl)-4H-1,2,4-triazol-3- | p38-α |
| ylthio)acetamido)-4-methylphenyl)furan-2-carboxamide | p38-β |
| ethyl 2-(2-(4-allyl-5-(pyridin-4-yl)-4H-1,2,4-triazol-3-ylthio)acetamido)-5,6- | CDK1 |
| dihydro-4H-cyclopenta[b]thiophene-3-carboxylate | AURORA-A |
| 2-(4-ethyl-5-(4-(methylsulfonamido)phenyl)-4H-1,2,4-triazol-3-ylthio)-N- | p38-α |
| (thiophen-2-ylmethyl)acetamide | p38-β |
| 2-(4-(furan-2-ylmethyl)-5-(pyridin-4-yl)-4H-1,2,4-triazol-3-ylthio)-N-(3- | GSK3-β |
| iodophenyl)acetamide | AKT3 |
| | SRC |
| ethyl 4-(2-(4-(2-methoxyethyl)-5-methyl-4H-1,2,4-triazol-3- | AKT1 |
| ylthio)acetamido)benzoate | |
| ethyl 2-(2-(5-((4-fluorophenylamino)methyl)-4-phenyl-4H-1,2,4-triazol-3- | LCK |
| ylthio)-N-phenylacetamido)acetate | |
| N-(5-ethyl-1,3,4-thiadiazol-2-yl)-2-(4-(4-methoxyphenyl)-5-(pyridin-4-yl)- | p38-α |
| 4H-1,2,4-triazol-3-ylthio)acetamide | GSK3-α |
| N-phenyl-2-(4-phenyl-5-((5-phenyl-2H-tetrazol-2-yl)methyl)-4H-1,2,4- | GSK3-α |
| triazol-3-ylthio)acetamide | |
| 2-(5-((4-chlorophenoxy)methyl)-4-methyl-4H-1,2,4-triazol-3-ylthio)-N-(2- | PDGFRR-α |
| ethylphenyl)acetamide | |
| N-(3-chlorophenyl)-2-(5-((4-methoxyphenylamino)methyl)-4-methyl-4H- | PDGFRR-α |
| 1,2,4-triazol-3-ylthio)acetamide | AURORA-A |
| | FYN |
| N-(2,5-dimethoxy-4-(2-(4-methyl-5-(thiophen-2-yl)-4H-1,2,4-triazol-3- | PDGFRR-α |
| ylthio)acetamido)phenyl)furan-2-carboxamide | |
| N-(benzo[d][1,3]dioxol-5-yl)-2-(5-(4-methoxyphenyl)-4-methyl-4H-1,2,4- | AKT1 |
| triazol-3-ylthio)acetamide | |
| 2-(5-((4,6-dimethylpyrimidin-2-ylthio)methyl)-4-ethyl-4H-1,2,4-triazol-3- | GSK3-α |
| ylthio)-N-(thiazol-2-yl)acetamide | |
| N-(2-ethylphenyl)-2-(4-methyl-5-(p-tolyloxymethyl)-4H-1,2,4-triazol-3- | PDGFRR-α |
| ylthio)acetamide | |
| N-(3-cyano-4,5-dimethylthiophen-2-yl)-2-(4-ethyl-5-(4- | DAPK1 |
| (morpholinosulfonyl)phenyl)-4H-1,2,4-triazol-3-ylthio)acetamide | |
| 2-(5-((2-chlorophenylamino)methyl)-4-ethyl-4H-1,2,4-triazol-3-ylthio)-N-(4- | PDGFRR-α |
| nitrophenyl)propanamide | |
| 2-(5-(2,3-dihydrobenzo[b][1,4]dioxin-2-yl)-4-ethyl-4H-1,2,4-triazol-3- | GSK3-α |
| ylthio)-N-(thiazol-2-yl)acetamide | GSK3-β |
| 2-(4-methyl-5-(3,4,5-trimethoxyphenyl)-4H-1,2,4-triazol-3-ylthio)-N-(2- | MAPKAPK-2 |
| methyl-5-nitrophenyl)acetamide | |
| 2-(4-ethyl-5-(4-hydroxyphenyl)-4H-1,2,4-triazol-3-ylthio)-1-(4-(4- | SYK |
| methoxyphenyl)piperazin-1-yl)ethanone | |
| ethyl 2-(N-phenyl-2-(4-phenyl-5-(pyridin-4-yl)-4H-1,2,4-triazol-3- | PDGFRR-α |
| ylthio)acetamido)acetate | |
| 2-(5-((4-methoxyphenylamino)methyl)-4-phenyl-4H-1,2,4-triazol-3-ylthio)- | CHEK2 |
| N,N-diphenylacetamide | PDGFRR-α |
| | FYN α |
| N-(3-methoxyphenyl)-2-(4-methyl-5-(m-tolyloxymethyl)-4H-1,2,4-triazol-3- | GSK3-α |
| ylthio)acetamide | CDK2-cyclinE |
| 2-(5-((4-fluorophenylamino)methyl)-4-methyl-4H-1,2,4-triazol-3-ylthio)-N- | CHEK2 |
| (4-nitrophenyl)acetamide | |
| 2-(4-methyl-5-m-tolyl-4H-1,2,4-triazol-3-ylthio)-N-(3- | CDK2-cyclinE |
| (trifluoromethyl)phenyl)acetamide | |
| 2-(4-benzyl-5-cyclohexyl-4H-1,2,4-triazol-3-ylthio)-N-phenylacetamide | p38-β |
| | p38-α |
| N-(3-chlorophenyl)-2-(5-((4-methoxyphenylamino)methyl)-4-p-tolyl-4H- | PDGFRR-α |
| 1,2,4-triazol-3-ylthio)acetamide | FYN |
| | AURORA-A |
| 2-(5-benzyl-4-methyl-4H-1,2,4-triazol-3-ylthio)acetamide | HCK |
| 2-(5-((4-methoxyphenoxy)methyl)-4-methyl-4H-1,2,4-triazol-3-ylthio)-N-(2- | PDGFRR-α |
| (trifluoromethyl)phenyl)acetamide | p38-δ |
| 2-(5-((4-methoxyphenylamino)methyl)-4-phenyl-4H-1,2,4-triazol-3-ylthio)- | PDGFRR-α |
| 1-(10H-phenothiazin-10-yl)ethanone | |
| N-tert-butyl-2-(4-fluorophenyl)-2-(N-(4-methoxyphenyl)-2-(4-methyl-5- | p38-α |
| phenyl-4H-1,2,4-triazol-3-ylthio)acetamido)acetamide | p38-β |
| 2-(5-benzyl-4-p-tolyl-4H-1,2,4-triazol-3-ylthio)-1-(10H-phenothiazin-10- | CDK2-cyclinE |
| yl)ethanone | |
| N-cyclohexyl-2-(4-fluorophenyl)-2-(2-(4-(furan-2-ylmethyl)-5-methyl-4H- | p38-α |
| 1,2,4-triazol-3-ylthio)-N-phenylacetamido)acetamide | |
| 2-(5-((3-chlorophenylamino)methyl)-4-phenethyl-4H-1,2,4-triazol-3-ylthio)- | CDK2-cyclinE |
| N-cyclohexylacetamide | |
| 2-(5-((3-chlorophenylamino)methyl)-4-phenyl-4H-1,2,4-triazol-3-ylthio)-N- | MSK1 |
| (2-methoxyphenyl)acetamide | SGK1 |
| methyl 2-(2-(5-((3-chlorophenylamino)methyl)-4-methyl-4H-1,2,4-triazol-3- | AURORA-A |
| ylthio)acetamido)benzoate | |
| 2-(5-((p-toluidino)methyl)-4-methyl-4H-1,2,4-triazol-3-ylthio)-N-(3- | CHEK2 |
| methoxyphenyl)acetamide | |
| N-(biphenyl-2-yl)-2-(5-((2,4-dimethylphenylamino)methyl)-4-ethyl-4H- | GSK3-α |
| 1,2,4-triazol-3-ylthio)acetamide | |
| 2-(5-((p-toluidino)methyl)-4-ethyl-4H-1,2,4-triazol-3-ylthio)-N-(2- | AURORA-A |
| methoxyphenyl)acetamide | |
| N-(3-acetylphenyl)-2-(5-((4-ethoxyphenylamino)methyl)-4-phenyl-4H-1,2,4- | FYN |
| triazol-3-ylthio)acetamide | PDGFRR-α |
| | FLT-3 |
| 2-(5-((4-methoxyphenylamino)methyl)-4-methyl-4H-1,2,4-triazol-3- | PDGFRR-α |
| ylthio)acetamide | FYN |
| 2-(5-(2-methoxyphenyl)-4-methyl-4H-1,2,4-triazol-3-ylthio)-N- | AURORA-A |
| phenylacetamide | |
| N-(3-methyl-4-(2-(4-methyl-5-(thiophen-2-yl)-4H-1,2,4-triazol-3- | CHEK2 |
| ylthio)acetamido)phenyl)thiophene-2-carboxamide | p38-α |
| 2-(5-((4-chlorophenylamino)methyl)-4-ethyl-4H-1,2,4-triazol-3-ylthio)-N- | c-TAK1 |
| phenylacetamide | |
| N-(biphenyl-2-yl)-2-(5-((2,6-dimethylphenylamino)methyl)-4-methyl-4H- | LCK |
| 1,2,4-triazol-3-ylthio)acetamide | |
| 2-(5-((4-methoxyphenylamino)methyl)-4-phenyl-4H-1,2,4-triazol-3-ylthio)- | PDGFRR-α |
| N-(5-methylthiazol-2-yl)acetamide | FYN |
| N-(3-hydroxyphenyl)-2-(4-(2-methoxyethyl)-5-(pyridin-4-yl)-4H-1,2,4- | AKT1 |
| triazol-3-ylthio)acetamide | |
| ethyl 2-(2-(4-methyl-5-(4-nitrophenyl)-4H-1,2,4-triazol-3-ylthio)-N- | PDGFRR-α |
| phenylacetamido)acetate | FYN |
| methyl 2-(2-(5-(furan-2-yl)-4-methyl-4H-1,2,4-triazol-3-ylthio)acetamido)-4- | GSK3-β |
| methylthiazole-5-carboxylate | GSK3-α |
| 2-(5-((p-toluidino)methyl)-4-ethyl-4H-1,2,4-triazol-3-ylthio)-N-(3- | CHEK2 |
| chlorophenyl)acetamide | |
| ethyl 2-(2-(4-(2-methoxyphenyl)-5-phenyl-4H-1,2,4-triazol-3-ylthio)-N-(3- | GSK3-α |
| nitrophenyl)acetamido)acetate | |
| N-(biphenyl-3-yl)-2-(5-((4-methoxyphenylamino)methyl)-4-methyl-4H- | PDGFRR-α |
| 1,2,4-triazol-3-ylthio)acetamide | FYN |
| | CHEK2 |
| ethyl 4-(2-(5-((p-toluidino)methyl)-4-ethyl-4H-1,2,4-triazol-3- | CHEK2 |
| ylthio)acetamido)benzoate | |
| N-phenyl-2-(4-phenyl-5-((quinolin-8-yloxy)methyl)-4H-1,2,4-triazol-3- | SGK1 |
| ylthio)acetamide | CDK1 |
| N-(6-ethoxybenzo[d]thiazol-2-yl)-2-(4-(3-methoxypropyl)-5-(pyridin-4-yl)- | GSK3-β |
| 4H-1,2,4-triazol-3-ylthio)acetamide | PDGFRR-α |
| | GSK3-α |
| ethyl 2-(2-(5-((4-methoxyphenylamino)methyl)-4-methyl-4H-1,2,4-triazol-3- | PDGFRR-α |
| ylthio)acetamido)-4,5-dimethylthiophene-3-carboxylate | FLT-3 |
| | AURORA-A |
| 2-(5-((4-fluorophenylamino)methyl)-4-p-tolyl-4H-1,2,4-triazol-3-ylthio)-N- | AKT1 |
| (2-(trifluoromethyl)phenyl)acetamide | |
| 2-(5-((3-chlorophenylamino)methyl)-4-phenethyl-4H-1,2,4-triazol-3- | CDK2-cyclinA |
| ylthio)acetamide | CDK1 |
| | AURORA-A |
| N-(3-(2-(5-(4-methoxyphenyl)-4-phenyl-4H-1,2,4-triazol-3- | p38-α |
| ylthio)acetamido)-4-methylphenyl)furan-2-carboxamide | KIT |
| 2-(5-((4-methoxyphenylamino)methyl)-4-phenyl-4H-1,2,4-triazol-3-ylthio)- | PDGFRR-α |
| N-(2-(trifluoromethyl)phenyl)acetamide | |
| 2-(5-(4-tert-butylphenyl)-4-methyl-4H-1,2,4-triazol-3-ylthio)-N-(2- | DAPK1 |
| (cyclohexylamino)-2-oxoethyl)-N-cyclopentylacetamide | |
| 2-(4-(furan-2-ylmethyl)-5-(pyridin-4-yl)-4H-1,2,4-triazol-3-ylthio)-N- | SRC |
| phenylacetamide | GSK3-α |
| | AKT3 |
| 2-(5-((p-toluidino)methyl)-4-methyl-4H-1,2,4-triazol-3-ylthio)-1-(10H- | DAPK1 |
| phenothiazin-10-yl)ethanone | |
| 2-(5-((4,6-dimethylpyrimidin-2-ylthio)methyl)-4-phenyl-4H-1,2,4-triazol-3- | MAPKAPK-2 |
| ylthio)-N-phenylacetamide | |
| 3-methyl-N-(4-methyl-3-(2-(4-phenyl-5-(pyridin-4-yl)-4H-1,2,4-triazol-3- | PDGFRR-α |
| ylthio)acetamido)phenyl)benzamide | p38-β |
| | p38-α |
| N-(4-acetylphenyl)-2-(4-(4-methoxyphenyl)-5-((4- | PDGFRR-α |
| methoxyphenylamino)methyl)-4H-1,2,4-triazol-3-ylthio)acetamide | FYN |
| | c-TAK1 |
| ethyl 3-(2-(4-(4-methoxyphenyl)-5-((4-methoxyphenylamino)methyl)-4H- | AURORA-A |
| 1,2,4-triazol-3-ylthio)acetamido)benzoate | PDGFRR-α |
| | CHEK2 |
| 2-(5-((p-toluidino)methyl)-4-ethyl-4H-1,2,4-triazol-3-ylthio)-N-(5- | p38-α |
| methylisoxazol-3-yl)acetamide | p38-δ |
| N-benzyl-2-(5-(4-methoxyphenyl)-4-phenyl-4H-1,2,4-triazol-3- | PDK1 |
| ylthio)acetamide | p38-γ |
| N-(2-chlorophenyl)-2-(4-(2,4-dimethylphenyl)-5-(pyridin-3-yl)-4H-1,2,4- | p38-α |
| triazol-3-ylthio)acetamide | |
| 2-(4-ethyl-5-(4-nitrophenyl)-4H-1,2,4-triazol-3-ylthio)-N-o-tolylpropanamide | DAPK1 |
| 2-(4-ethyl-5-(pyridin-4-yl)-4H-1,2,4-triazol-3-ylthio)-1-(10H-phenothiazin- | p38-δ |
| 10-yl)ethanone | p38-γ |
| methyl 2-(2-(5-((3-chlorophenylamino)methyl)-4-ethyl-4H-1,2,4-triazol-3- | AURORA-A |
| ylthio)acetamido)benzoate | |
| 2-(4-(4-methoxyphenyl)-5-phenyl-4H-1,2,4-triazol-3-ylthio)-1-(4- | DAPK1 |
| methylpiperidin-1-yl)ethanone | |
| N-benzyl-2-(4-methyl-5-m-tolyl-4H-1,2,4-triazol-3-ylthio)acetamide | MAPKAPK-2 |
| | FYN |
| N-(3-acetylphenyl)-2-(4-(4-chlorophenyl)-5-((4- | PDGFRR-α |
| methoxyphenylamino)methyl)-4H-1,2,4-triazol-3-ylthio)acetamide | FYN |
| | FLT-3 |
| 2-(5-(4-(N,N-diethylsulfamoyl)phenyl)-4-ethyl-4H-1,2,4-triazol-3-ylthio)-N- | GSK3-α |
| (thiazol-2-yl)acetamide | GSK3-β |
| 2-(5-(4-tert-butylphenyl)-4-(3-methoxypropyl)-4H-1,2,4-triazol-3-ylthio)-1- | CDK5 |
| piperidin-1-yl)ethanone | |
| 2-(5-(4-bromophenyl)-4-phenyl-4H-1,2,4-triazol-3-ylthio)-N-(2-methoxy-5- | SYK |
| methylphenyl)acetamide | |
| 2-(4-(pyridin-2-yl)-4H-1,2,4-triazol-3-ylthio)-N-(thiazol-2-yl)acetamide | GSK3-α |
| N-(4-acetylphenyl)-2-(5-((4-methoxyphenylamino)methyl)-4-methyl-4H- | PDGFRR-α |
| 1,2,4-triazol-3-ylthio)acetamide | FLT-3 |
| | FYN |
| 2-(5-((2-chlorophenylamino)methyl)-4-(4-methoxyphenyl)-4H-1,2,4-triazol- | CHEK2 |
| 3-ylthio)-N-(4-nitrophenyl)acetamide | |
| methyl 2-(2-(5-((4-methoxyphenylamino)methyl)-4-p-tolyl-4H-1,2,4-triazol- | PDGFRR-α |
| 3-ylthio)acetamido)benzoate | AURORA-A |
| | FYN |
| 2-(5-((4-fluorophenylamino)methyl)-4-phenyl-4H-1,2,4-triazol-3- | FLT-3 |
| ylthio)acetamide | |
| N-(benzo[d]thiazol-2-yl)-2-(5-(furan-2-yl)-4-methyl-4H-1,2,4-triazol-3- | GSK3-α |
| ylthio)acetamide | |
| 2-(5-((4-methoxyphenylamino)methyl)-4-methyl-4H-1,2,4-triazol-3-ylthio)- | INSR |
| N-o-tolylacetamide | CHEK1 |
| | FYN |
| N-(biphenyl-2-yl)-2-(4-ethyl-5-((4-methoxyphenylamino)methyl)-4H-1,2,4- | PDGFRR-α |
| triazol-3-ylthio)acetamide | FYN |
| | GSK3-β |
| 2-(5-benzyl-4-(4-methoxyphenyl)-4H-1,2,4-triazol-3-ylthio)-N- | CHEK2 |
| phenylacetamide | |
| N-(3-cyano-4,5,6,7-tetrahydrobenzo[b]thiophen-2-yl)-2-(4-ethyl-5- | AKT1 |
| (phenoxymethyl)-4H-1,2,4-triazol-3-ylthio)acetamide | |
| N-(4-bromophenyl)-2-(5-((4-methoxyphenylamino)methyl)-4-methyl-4H- | PDGFRR-α |
| 1,2,4-triazol-3-ylthio)acetamide | FYN |
| | AURORA-A |
| N-(2-bromo-4-methylphenyl)-2-(5-((4-methoxyphenylamino)methyl)-4- | FYN |
| methyl-4H-1,2,4-triazol-3-ylthio)acetamide | PDGFRR-α |
| | FLT-3 |
| N-(3-acetylphenyl)-2-(5-((2-chlorophenylamino)methyl)-4-(4- | CHEK2 |
| methoxyphenyl)-4H-1,2,4-triazol-3-ylthio)acetamide | |
| 2-(5-((p-toluidino)methyl)-4-phenyl-4H-1,2,4-triazol-3-ylthio)-N-(2-bromo- | AURORA-A |
| 4-methylphenyl)acetamide | |
| N-(2-bromo-4-methylphenyl)-2-(5-((4-methoxyphenoxy)methyl)-4-phenyl- | CDK1 |
| 4H-1,2,4-triazol-3-ylthio)acetamide | |
| N-(2-bromophenyl)-2-(5-((4-ethoxyphenylamino)methyl)-4-phenyl-4H-1,2,4- | PDGFRR-α |
| triazol-3-yithio)acetamide | FYN |
| | FLT-3 |
| N-(biphenyl-2-yl)-2-(4,5-dibenzyl-4H-1,2,4-triazol-3-ylthio)acetamide | CDK2-cyclinE |
| N-(4-acetylphenyl)-2-(5-((3-chlorophenylamino)methyl)-4-ethyl-4H-1,2,4- | CDK1 |
| triazol-3-ylthio)acetamide | |
| 2-(4-methyl-5-(pyridin-3-yl)-4H-1,2,4-triazol-3-ylthio)-N-(4-methylthiazol- | PAK2( |
| 2-yl)acetamide | |
| N-(benzo[d]thiazol-2-yl)-2-(4-ethyl-5-(pyridin-3-yl)-4H-1,2,4-triazol-3- | GSK3-β |
| ylthio)acetamide | GSK3-α |
| | CDK2-cyclinE |
| 2-(5-((4-chlorophenylamino)methyl)-4-phenyl-4H-1,2,4-triazol-3-ylthio)-N- | CDK2-cyclinE |
| (2,6-dimethylphenyl)propanamide | |
| 2-(5-((4-chlorophenylamino)methyl)-4-ethyl-4H-1,2,4-triazol-3-ylthio)-N-(2- | LCK |
| ethyl-6-methylphenyl)acetamide | |
| N-(2-chlorophenyl)-2-(5-(2-methoxyphenyl)-4-p-tolyl-4H-1,2,4-triazol-3- | p38-α |
| ylthio)acetamide | |
| 2-(4-ethyl-5-(4-(piperidin-1-ylsulfonyl)phenyl)-4H-1,2,4-triazol-3-ylthio)-1- | PAK2 |
| morpholinoethanone | |
| 2-(5-((4-ethoxyphenylamino)methyl)-4-phenyl-4H-1,2,4-triazol-3-ylthio)-N- | FYN |
| (2-methyl-5-nitrophenyl)propanamide | PDGFRR-α |
| | p38-α |
| 2-(4-(4-fluorophenyl)-5-(pyridin-3-yl)-4H-1,2,4-triazol-3-ylthio)-N-(5- | FYN |
| methylisoxazol-3-yl)acetamide | |
| 2-(4-benzyl-5-(phenoxymethyl)-4H-1,2,4-triazol-3-ylthio)-N- | FYN |
| methylacetamide | |
| N-(4-(5-(2-(4-benzylpiperidin-1-yl)-2-oxoethylthio)-4-methyl-4H-1,2,4- | p38-α |
| triazol-3-yl)phenyl)methanesulfonamide | |
| 2-(5-((4-methoxyphenylamino)methyl)-4-p-tolyl-4H-1,2,4-triazol-3-ylthio)- | FYN |
| N-phenylacetamide | PDGFRR-α |
| | FLT-3 |
| 2-(5-((3-chlorophenylamino)methyl)-4-ethyl-4H-1,2,4-triazol-3-ylthio)-N- | PAK2( |
| (furan-2-ylmethyl)acetamide | |
| N-(2,6-dimethylphenyl)-2-(4-(3-methoxypropyl)-5-(pyridin-4-yl)-4H-1,2,4- | FYN |
| triazol-3-ylthio)acetamide | |
| 4-methyl-N-(4-(4-methyl-5-(2-oxo-2-(piperidin-1-yl)ethylthio)-4H-1,2,4- | AKT1 |
| triazol-3-yl)phenyl)benzenesulfonamide | |
| 2-(4-methyl-5-(pyridin-4-yl)-4H-1,2,4-triazol-3-ylthio)acetamide | AKT1 |
| 2-(4-benzyl-5-(pyridin-3-yl)-4H-1,2,4-triazol-3-ylthio)-N-phenylacetamide | p38-β |
| 2-(4-ethyl-5-((4-methoxyphenylamino)methyl)-4H-1,2,4-triazol-3-ylthio)-N- | FYN |
| (2-(trifluoromethyl)phenyl)acetamide | PDGFRR-α |
| | FLT-3 |
| 2-(5-((4-methoxyphenylamino)methyl)-4-methyl-4H-1,2,4-triazol-3-ylthio)- | PDGFRR-α |
| N-(3-(trifluoromethyl)phenyl)acetamide | FYN |
| | FLT-3 |
| 2-(5-((4-methoxyphenoxy)methyl)-4-methyl-4H-1,2,4-triazol-3-ylthio)-N-(2- | PDGFRR-α |
| methoxyphenyl)acetamide | PKA |
| 1-(3,4-dihydroquinolin-1(2H)-yl)-2-(5-((2,3-dimethylphenoxy)methyl)-4- | AKT1 |
| (furan-2-ylmethyl)-4H-1,2,4-triazol-3-ylthio)ethanone | |
| N-(3-(2-(4-ethyl-5-(4-hydroxyphenyl)-4H-1,2,4-triazol-3- | p38-α |
| ylthio)acetamido)phenyl)propionamide | GSK3-α |
| N-(3-hydroxyphenyl)-2-(4-(4-methoxyphenyl)-5-(pyridin-4-yl)-4H-1,2,4- | AURORA-A |
| triazol-3-ylthio)acetamide | GSK3-β |
| | p38-α |
| methyl 4-(2-(5-(2-hydroxyphenyl)-4-(2-methoxyphenyl)-4H-1,2,4-triazol-3- | AURORA-A |
| ylthio)acetamido)benzoate | |
| ethyl 2-(2-(5-(2-hydroxyhenyl)-4-phenethyl-4H-1,2,4-triazol-3-ylthio)-N-(3- | AURORA-A |
| nitrophenyl)acetamido)acetate | |
| 3-(2-(4-(pyridin-3-yl)-4H-1,2,4-triazol-3-ylthio)acetamido)benzoic acid | AURORA-A |
| ethyl 2-(2-(4-(3-(dimethylamino)propyl)-5-(pyridin-4-yl)-4H-1,2,4-triazol-3- | CDK1 |
| ylthio)-N-(3-nitrophenyl)acetamido)acetate | |
| N-(3-hydroxyphenyl)-2-(5-(pyridin-4-yl)-4-((tetrahydrofuran-2-yl)methyl)- | p38-β |
| 4H-1,2,4-triazol-3-ylthio)acetamide | |
| 2-(4-(furan-2-ylmethyl)-5-(4-hydroxyphenyl)-4H-1,2,4-triazol-3-ylthio)-1- | GSK3-β |
| (10H-phenothiazin-10-yl)ethanone | GSK3-α |
| | MSK2 |
| N-(2,6-dimethylphenyl)-2-(4-ethyl-5-(4-hydroxyphenyl)-4H-1,2,4-triazol-3- | p38-α |
| ylthio)acetamide | |
| 2-(5-(2-hydroxyphenyl)-4-(2-methoxyphenyl)-4H-1,2,4-triazol-3- | AURORA-A |
| ylthio)acetamide | |
| N-(5-(2-(4-ethyl-5-(4-hydroxyphenyl)-4H-1,2,4-triazol-3-ylthio)acetamido) | SGK1 |
| 2-methylphenyl)propionamide | |
| ethyl 2-(2-(5-(2-hydroxyphenyl)-4-phenethyl-4H-1,2,4-triazol-3- | CDK2-cyclinA |
| ylthio)acetamido)-4,5-dimethylthiophene-3-carboxylate | |
| 2-(5-(4-hydroxyphenyl)-4-methyl-4H-1,2,4-triazol-3-ylthio)-N-p- | GSK3-β |
| tolylacetamide | GSK3-α |
| ethyl 4-(2-(5-(4-hydroxyphenyl)-4-methyl-4H-1,2,4-triazol-3- | GSK3-β |
| ylthio)acetamido)benzoate | AURORA-A |
| | GSK3-α |
| 2-(4-benzyl-5-(4-hydroxyphenyl)-4H-1,2,4-triazol-3-ylthio)-N-(2,4- | p38-α |
| dimethylphenyl)acetamide | |
| 2-(4-benzyl-5-(2-hydroxyphenyl)-4H-1,2,4-triazol-3-ylthio)-N-(3- | SYK |
| iodophenyl)acetamide | |
| N-benzyl-2-(5-(2-hydroxyphenyl)-4-phenethyl-4H-1,2,4-triazol-3- | p38-α |
| ylthio)acetamide | INSR |
| | CHEK1 |
| 2-(4-benzyl-5-(2-hydroxyphenyl)-4H-1,2,4-triazol-3-ylthio)-N-(2- | p38-α |
| (trifluoromethyl)phenyl)acetamide | SYK |
| N-(2,3-dimethylphenyl)-2-(5-(4-hydroxyphenyl)-4-phenyl-4H-1,2,4-triazol- | MSK2 |
| 3-ylthio)acetamide | |
| 2-(5-(4-hydroxyphenyl)-4-methyl-4H-1,2,4-triazol-3-ylthio)-N-(3- | GSK3-β |
| (trifluoromethyl)phenyl)acetamide | SRC |
| | GSK3-α |
| N-(4-(2-(5-(4-hydroxyphenyl)-4-methyl-4H-1,2,4-triazol-3- | GSK3-β |
| ylthio)acetamido)-2-methylphenyl)-2-methylbenzamide | GSK3-α |
| 2-(5-((2,4-dimethylphenylamino)methyl)-4-(furan-2-ylmethyl)-4H-1,2,4- | CHEK2 |
| triazol-3-ylthio)-N-(3-hydroxyphenyl)acetamide | |
| ethyl 2-(2-(5-(4-hydroxyphenyl)-4-((tetrahydrofuran-2-yl)methyl)-4H-1,2,4- | GSK3-β |
| triazol-3-ylthio)acetamido)-4-methylthiazole-5-carboxylate | GSK3-α |
| N-benzyl-2-(4-(furan-2-ylmethyl)-5-(4-hydroxyphenyl)-4H-1,2,4-triazol-3- | p38-α |
| ylthio)acetamide | p38-β |
| N-(2,4-dimethoxyphenyl)-2-(5-(2-hydroxyphenyl)-4-phenyl-4H-1,2,4-triazol- | CDK1 |
| 3-ylthio)acetamide | |
| 3-(2-(5-(2-hydroxyphenyl)-4-(2-methoxyphenyl)-4H-1,2,4-triazol-3- | AURORA-A |
| ylthio)acetamido)benzoic acid | |
| 2-(4-(furan-2-ylmethyl)-5-(4-(methylsulfonamido)phenyl)-4H-1,2,4-triazol- | p38-α |
| 3-ylthio)-N-(3-hydroxyphenyl)acetamide | p38-β |
| N-(4-acetylphenyl)-2-(5-(4-hydroxyphenyl)-4-(4-methoxyphenyl)-4H-1,2,4- | GSK3-β |
| triazol-3-ylthio)propanamide | GSK3-α |
| 2-(5-(4-(azepan-1-ylsulfonyl)phenyl)-4-ethyl-4H-1,2,4-triazol-3-ylthio)-N-(2- | DAPK1 |
| methoxy-5-methylphenyl)acetamide | |
| N-(3-cyano-4,5,6,7-tetrahydrobenzo[b]thiophen-2-yl)-2-(4-ethyl-5-(4- | AURORA-A |
| hydroxyphenyl)-4H-1,2,4-triazol-3-ylthio)acetamide | |
| N-(2,5-diethoxy-4-(2-(5-(4-hydroxyphenyl)-4-methyl-4H-1,2,4-triazol-3- | PDGFRR-α |
| ylthio)acetamido)phenyl)thiophene-2-carboxamide | |
| 2-(5-(4-hydroxyphenyl)-4-methyl-4H-1,2,4-triazol-3-ylthio)-N-(2-methyl-5- | GSK3-β |
| nitrophenyl)acetamide | GSK3-α |
| 2-(5-(4-hydroxyphenyl)-4-(4-methoxyphenyl)-4H-1,2,4-triazol-3- | GSK3-β |
| ylthio)acetamide | GSK3-α |
| N-(3-acetylphenyl)-2-(4-benzyl-5-(4-hydroxyphenyl)-4H-1,2,4-triazol-3- | GSK3-β |
| ylthio)acetamide | p38-β |
| | GSK3-α |
| 2-(5-(4-(azepan-1-ylsulfonyl)phenyl)-4-ethyl-4H-1,2,4-triazol-3-ylthio)-N- | p38-α |
| (furan-2-ylmethyl)acetamide | |
| 2-(5-(4-(azepan-1-ylsulfonyl)phenyl)-4-ethyl-4H-1,2,4-triazol-3-ylthio)-N,N- | AURORA-A |
| diethylacetamide | |
| 2-(5-(4-(azepan-1-ylsulfonyl)phenyl)-4-ethyl-4H-1,2,4-triazol-3-ylthio)-1- | AURORA-A |
| (pyrrolidin-1-yl)ethanone | |
| 2-(5-(2-hydroxyphenyl)-4-phenethyl-4H-1,2,4-triazol-3-ylthio)-N-(5- | AURORA-A |
| (trifluoromethyl)-1,3,4-thiadiazol-2-yl)acetamide | |
| 2-(5-((4-fluorophenylamino)methyl)-4-((tetrahydrofuran-2-yl)methyl)-4H- | AURORA-A |
| 1,2,4-triazol-3-ylthio)-N-p-tolylacetamide | |
| 2-(4-(furan-2-ylmethyl)-5-(3-hydroxynaphthalen-2-yl)-4H-1,2,4-triazol-3- | AURORA-A |
| ylthio)-N-(5-methylisoxazol-3-yl)acetamide | MSK2 |
| | p38-δ |
| 1-(3,4-dihydroquinolin-1(2H)-yl)-2-(5-(3-hydroxynaphthalen-2-yl)-4-methyl- | FYN |
| 4H-1,2,4-triazol-3-ylthio)ethanone | AURORA-A |
| | DAPK1 |
| 2-(5-(3-hydroxynaphthalen-2-yl)-4-methyl-4H-1,2,4-triazol-3-ylthio)-N-(5- | AURORA-A |
| methylisoxazol-3-yl)acetamide | FYN |
| 2-(4-ethyl-5-(3-hydroxynaphthalen-2-yl)-4H-1,2,4-triazol-3-ylthio)-N- | AURORA-A |
| (4,5,6,7-tetrahydrobenzo[d]thiazol-2-yl)acetamide | CDK2-cyclinE |
| 2-(4-ethyl-5-(3-hydroxynaphthalen-2-yl)-4H-1,2,4-triazol-3-ylthio)-N- | AURORA-A |
| (4,5,6,7-tetrahydrobenzo[d]thiazol-2-yl)acetamide | |
| 2-(5-(3-hydroxynaphthalen-2-yl)-4-methyl-4H-1,2,4-triazol-3-ylthio)-1- | AURORA-A |
| (10H-phenothiazin-10-yl)ethanone | |
| N-(furan-2-ylmethyl)-2-(5-(3-hydroxynaphthalen-2-yl)-4-(2-methoxyethyl)- | p38-α |
| 4H-1,2,4-triazol-3-ylthio)acetamide | AURORA-A |
| | FYN |
| N-cyclohexyl-2-(5-(3-hydroxynaphthalen-2-yl)-4-(2-methoxyethyl)-4H- | DAPK 1 |
| 1,2,4-triazol-3-ylthio)acetamide | AURORA-A |
| | p38-δ |
| N-(benzo[d][1,3]dioxol-5-yl)-2-(5-(3-hydroxynaphthalen-2-yl)-4-(2- | AURORA-A |
| methoxyethyl)-4H-1,2,4-triazol-3-ylthio)acetamide | |
| N-(4,5-dimethylthiazol-2-yl)-2-(4-(furan-2-ylmethyl)-5-(4-hydroxyphenyl)- | GSK3-β |
| 4H-1,2,4-triazol-3-ylthio)acetamide | GSK3-α |
| | p38-α |
| N-(benzo[d][1,3]dioxol-5-ylmethyl)-2-(4-(furan-2-ylmethyl)-5-(4- | CDK2-cyclinA |
| hydroxyphenyl)-4H-1,2,4-triazol-3-ylthio)acetamide | CDK5 |
| | CDK2-cyclinE |
| N-allyl-2-(5-(2-hydroxyphenyl)-4-p-tolyl-4H-1,2,4-triazol-3-ylthio)acetamide | AURORA-A |
| N-(benzo[d][1,3]dioxol-5-ylmethyl)-2-(5-(3-hydroxynaphthalen-2-yl)-4- | CSK |
| methyl-4H-1,2,4-triazol-3-ylthio)acetamide | |
| 2-(4-benzyl-5-((4-fluorophenylamino)methyl)-4H-1,2,4-triazol-3-ylthio)-N- | CHEK2 |
| (3-hydroxyphenyl)acetamide | |
| 2-(4-ethyl-5-(4-hydroxyphenyl)-4H-1,2,4-triazol-3-ylthio)-N-p- | AURORA-A |
| tolylacetamide | |
| 2-(4-ethyl-5-(4-hydroxyphenyl)-4H-1,2,4-triazol-3-ylthio)-N-(thiophen-2- | p38-α |
| ylmethyl)acetamide | |
| 2-(5-benzyl-4-cyclohexyl-4H-1,2,4-triazol-3-ylthio)-N-(3- | AURORA-A |
| hydroxyphenyl)acetamide | |
| methyl 2-(2-(5-((4-fluorophenylamino)methyl)-4-((tetrahydrofuran-2- | AURORA-A |
| yl)methyl)-4H-1,2,4-triazol-3-ylthio)acetamido)benzoate | |
| 2-(5-(4-hydroxyphenyl)-4-((tetrahydrofuran-2-yl)methyl)-4H-1,2,4-triazol-3- | GSK3-β |
| ylthio)-N-(6-methoxybenzo[d]thiazol-2-yl)acetamide | PDGFRR-α |
| | GSK3-α |
| 2-(5-((4-ethoxyphenylamino)methyl)-4-p-tolyl-4H-1,2,4-triazol-3-ylthio)-1- | FYN |
| (4-methylpiperazin-1-yl)ethanone | AURORA-A |
| methyl 2-(2-(5-(2-hydroxyphenyl)-4-phenethyl-4H-1,2,4-triazol-3- | AURORA-A |
| ylthio)acetamido)benzoate | |
| 2-(5-(2-hydroxyphenyl)-4-p-tolyl-4H-1,2,4-triazol-3-ylthio)-N-p- | AURORA-A |
| tolylacetamide | |
| 2-(5-(3,4-dimethoxyphenyl)-4-phenyl-4H-1,2,4-triazol-3-ylthio)-N-(2- | AURORA-A |
| hydroxyphenyl)acetamide | |
| 2-(4-benzyl-5-(2-hydroxyphenyl)-4H-1,2,4-triazol-3-ylthio)-N,N- | CDK2-cyclinA |
| diphenylacetamide | AURORA-A |
| 1-(3,4-dihydroisoquinolin-2(1H)-yl)-2-(4-ethyl-5-(3-hydroxynaphthalen-2- | DAPK1 |
| yl)-4H-1,2,4-triazol-3-ylthio)ethanone | AURORA-A |
| 1-(3,4-dihydroquinolin-1(2H)-yl)-2-(4-ethyl-5-(3-hydroxynaphthalen-2-yl)- | CDK2-cyclinA |
| 4H-1,2,4-triazol-3-ylthio)ethanone | AURORA-A |
| | DAPK1 |
| N-(3,4-dimethoxyphenethyl)-2-(5-(3-hydroxynaphthalen-2-yl)-4-methyl-4H- | FYN |
| 1,2,4-triazol-3-ylthio)acetamide | |
| methyl 2-(2-(5-(3-hydroxynaphthalen-2-yl)-4-(2-methoxyethyl)-4H-1,2,4- | AURORA-A |
| triazol-3-ylthio)acetamido)benzoate | DAPK1 |
| N-(2-fluorophenyl)-2-(5-(3-hydroxynaphthalen-2-yl)-4-(2-methoxyethyl)- | AURORA-A |
| 4H-1,2,4-triazol-3-ylthio)acetamide | |
| 1-(3,4-dihydroquinolin-1(2H)-yl)-2-(5-(3-hydroxynaphthalen-2-yl)-4-(2- | AURORA-A |
| methoxyethyl)-4H-1,2,4-triazol-3-ylthio)ethanone | p38-δ |
| | DAPK1 |
| N-(benzo[d][1,3]dioxol-5-ylmethyl)-2-(5-(3-hydroxynaphthalen-2-yl)-4-(2- | AURORA-A |
| methoxyethyl)-4H-1,2,4-triazol-3-ylthio)acetamide | |
| 2-(5-(4-hydroxyphenyl)-4-((tetrahydrofuran-2-yl)methyl)-4H-1,2,4-triazol-3- | MSK1 |
| ylthio)-N-phenethylacetamide | |
| 2-(4-(furan-2-ylmethyl)-5-(4-hydroxyphenyl)-4H-1,2,4-triazol-3-ylthio)-1- | AURORA-A |
| (indolin-1-yl)ethanone | |
| 1-(3,4-dihydroquinolin-1(2H)-yl)-2-(4-(furan-2-ylmethyl)-5-(4- | GSK3-β |
| hydroxyphenyl)-4H-1,2,4-triazol-3-ylthio)ethanone | GSK3-α |
| 2-(4-(furan-2-ylmethyl)-5-(4-hydroxyphenyl)-4H-1,2,4-triazol-3-ylthio)-N- | GSK3-β |
| (thiazol-2-yl)acetamide | GSK3-α |
| | MSK2 |
| N-(3-hydroxyphenyl)-2-(4-methyl-5-(4-(methylsulfonamido)phenyl)-4H- | AURORA-A |
| 1,2,4-triazol-3-ylthio)acetamide | |
| 2-(5-((3-chloro-4-methylphenylamino)methyl)-4-((tetrahydrofuran-2- | AURORA-A |
| yl)methyl)-4H-1,2,4-triazol-3-ylthio)acetamide | |
| 2-(5-(4-hydroxyphenyl)-4-methyl-4H-1,2,4-triazol-3-ylthio)-1- | AURORA-A |
| morpholinoethanone | |
| 2-(5-(4-hydroxyphenyl)-4-methyl-4H-1,2,4-triazol-3-ylthio)-N- | p38-δ |
| phenylpropanamide | MSK1 |
| 2-(4-(4-fluorophenyl)-5-(pyridin-4-yl)-4H-1,2,4-triazol-3-ylthio)-N-(3- | AURORA-A |
| hydroxyphenyl)acetamide | |
| 2-(5-(4-hydroxyphenyl)-4-methyl-4H-1,2,4-triazol-3-ylthio)-N-(4,5,6,7- | GSK3-β |
| tetrahydrobenzo[d]thiazol-2-yl)acetamide | GSK3-α |
| | CDK2-cyclinE |
| N-(furan-2-ylmethyl)-2-(5-(2-hydroxyphenyl)-4-(2-methoxyphenyl)-4H- | p38-β |
| 1,2,4-triazol-3-ylthio)acetamide | |
| 2-(4-ethyl-5-(3-hydroxynaphthalen-2-yl)-4H-1,2,4-triazol-3-ylthio)-N-(furan- | p38-α |
| 2-ylmethyl)acetamide | |
| 2-(4-benzyl-5-(4-hydroxyphenyl)-4H-1,2,4-triazol-3-ylthio)-1-(piperidin-1- | GSK3-α |
| yl)ethanone | GSK3-β |
| 2-(4-(furan-2-ylmethyl)-5-(4-hydroxyphenyl)-4H-1,2,4-triazol-3-ylthio)-N- | p38-α |
| (thiophen-2-ylmethyl)acetamide | CDK2-cyclinA |
| | p38-β |
| 2-(5-(2-hydroxyphenyl)-4-(2-methoxyphenyl)-4H-1,2,4-triazol-3-ylthio)-1- | P70S6K1 |
| morpholinoethanone | |
| 2-(5-(4-hydroxyphenyl)-4-phenyl-4H-1,2,4-triazol-3-ylthio)-N-p- | HCK |
| tolylpropanamide | |
| 2-(4-ethyl-5-(3-hydroxynaphthalen-2-yl)-4H-1,2,4-triazol-3-ylthio)-N,N- | c-TAK1 |
| diisopropylacetamide | |
| 2-(4-(furan-2-ylmethyl)-5-(4-hydroxyphenyl)-4H-1,2,4-triazol-3-ylthio)-N- | LCK |
| (4-nitrophenyl)propanamide | |
| 1-(3,4-dihydroquinolin-1(2H)-yl)-2-(5-((4-methoxyphenoxy)methyl)-4-p- | LCK |
| tolyl-4H-1,2,4-triazol-3-ylthio)ethanone | |
| 2-(4-allyl-5-(pyridin-4-yl)-4H-1,2,4-triazol-3-ylthio)-1-(10H-phenothiazin- | p38-γ |
| 10-yl)ethanone | MSK2 |
| | AKT3 |
| 2-(4H-1,2,4-triazol-3-ylthio)-1-(10H-phenothiazin-10-yl)ethanone | LCK |
| N-(2-chloro-5-(trifluoromethyl)phenyl)-2-(5-((4- | PAK2 |
| ethoxyphenylamino)methyl)-4-(4-methoxyphenyl)-4H-1,2,4-triazol-3- | AURORA-A |
| ylthio)acetamide | FYN |
| ethyl 5-acetyl-2-(2-(5-(2-hydroxyphenyl)-4-p-tolyl-4H-1,2,4-triazol-3- | LCK |
| ylthio)acetamido)-4-methylthiophene-3-carboxylate | |
| 2-(5-benzyl-4-(4-methoxyphenyl)-4H-1,2,4-triazol-3-ylthio)-1-(10H- | SRC |
| phenothiazin-10-yl)ethanone | LCK |
| 2-(5-methyl-4H-1,2,4-triazol-3-ylthio)-1-(10H-phenothiazin-10-yl)ethanone | MAPKAPK-2 |
| | CDK2-cyclinE |
| | LCK |
| 2-(4-phenyl-5-((3-(trifluoromethyl)phenylamino)methyl)-4H-1,2,4-triazol-3- | MAPKAPK-2 |
| ylthio)-N-p-tolylacetamide | |
| 2-(5-(4-aminophenyl)-4-methyl-4H-1,2,4-triazol-3-ylthio)-1-(indolin-1- | MAPKAPK-2 |
| yl)ethanone | |
| N-(4-ethoxyphenyl)-2-(5-(4-hydroxyphenyl)-4-phenyl-4H-1,2,4-triazol-3- | HCK |
| ylthio)acetamide | |
| N-(2,3-dimethylphenyl)-2-(5-(4-hydroxyphenyl)-4-methyl-4H-1,2,4-triazol- | p38-δ |
| 3-ylthio)acetamide | MAPKAPK-2 |
| | DYRK2 |
| 2-(4-methyl-4H-1,2,4-triazol-3-ylthio)-1-(10H-phenothiazin-10-yl)ethanone | LCK |
| 2-(5-(furan-2-yl)-4-methyl-4H-1,2,4-triazol-3-ylthio)-N-(3- | CSK |
| hydroxyphenyl)acetamide | |
| N-(4-acetylphenyl)-2-(5-((3-chloro-4-methylphenylamino)methyl)-4-phenyl- | p38-α |
| 4H-1,2,4-triazol-3-ylthio)propanamide | CSK |
| 2-(5-benzyl-4-(3-chlorophenyl)-4H-1,2,4-triazol-3-ylthio)-1-(4- | LCK |
| methylpiperidin-1-yl)ethanone | |
| N-(furan-2-ylmethyl)-2-(5-(4-hydroxyphenyl)-4-phenyl-4H-1,2,4-triazol-3- | GSK3-β |
| ylthio)acetamide | GSK3-α |
| 2-(5-(3,4-dimethoxyphenyl)-4-methyl-4H-1,2,4-triazol-3-ylthio)-N-(4- | p38-δ |
| ethylphenyl)acetamide | |
| N-(3-cyano-4,5,6,7-tetrahydrobenzo[b]thiophen-2-yl)-2-(5-(4- | PRAK |
| hydroxyphenyl)-4-methyl-4H-1,2,4-triazol-3-ylthio)acetamide | CDK2-cyclinE |
| | DYRK2 |
| 2-(5-(3-hydroxynaphthalen-2-yl)-4-methyl-4H-1,2,4-triazol-3-ylthio)-N- | GSK3-β |
| (thiazol-2-yl)acetamide | GSK3-α |
| N-(5-ethyl-1,3,4-thiadiazol-2-yl)-2-(5-(3-hydroxynaphthalen-2-yl)-4-(2- | p38-δ |
| methoxyethyl)-4H-1,2,4-triazol-3-ylthio)acetamide | FYN |
| methyl 4-methyl-2-(2-(4-methyl-5-(4-(methylsulfonamido)phenyl)-4H-1,2,4- | GSK3-β |
| triazol-3-ylthio)acetamido)thiazole-5-carboxylate | GSK3-α |
| N-(4-acetylphenyl)-2-(5-methyl-4-phenyl-4H-1,2,4-triazol-3- | AKT1 |
| ylthio)acetamide | |
| 2-(4-(4-fluorophenyl)-5-(pyridin-4-yl)-4H-1,2,4-triazol-3-ylthio)-N-(thiazol- | GSK3-α |
| 2-yl)acetamide | GSK3-β |
| | CDK2-cyclinE |
| 2-(5-methyl-4-phenyl-4H-1,2,4-triazol-3-ylthio)acetamide | DYRK2 |
| 2-(5-(3-hydroxynaphthalen-2-yl)-4-methyl-4H-1,2,4-triazol-3-ylthio)-N- | FYN |
| methylacetamide | |
| N-(furan-2-ylmethyl)-2-(4-(2-methoxyphenyl)-5-((4- | PDGFRR-α |
| methoxyphenylamino)methyl)-4H-1,2,4-triazol-3-ylthio)acetamide | FYN |
| 2-(4-methyl-5-(4-(morpholinosulfonyl)phenyl)-4H-1,2,4-triazol-3-ylthio)-N- | GSK3-α |
| (thiazol-2-yl)acetamide | GSK3-β |
| 2-(4-methyl-5-(4-nitrophenyl)-4H-1,2,4-triazol-3-ylthio)acetamide | PDGFRR-α |
| | FYN |
| ethyl 5-acetyl-2-(2-(5-((4-methoxyphenylamino)methyl)-4-methyl-4H-1,2,4- | AURORA-A |
| triazol-3-ylthio)acetamido)-4-methylthiophene-3-carboxylate | FYN |
| | PDGFRR-α |
| N-(5-ethyl-1,3,4-thiadiazol-2-yl)-2-(4-ethyl-5-m-tolyl-4H-1,2,4-triazol-3- | LCK |
| ylthio)acetamide | |
| ethyl 2-(2-(4-(2-methoxyethyl)-5-(4-methoxyphenyl)-4H-1,2,4-triazol-3- | GSK3-β |
| ylthio)acetamido)-4-methylthiazole-5-carboxylate | GSK3-α |
| 2-(5-(3,4-dimethoxyphenyl)-4-phenyl-4H-1,2,4-triazol-3-ylthio)-1- | AKT1 |
| morpholinoethanone | |
| 2-(4-(furan-2-ylmethyl)-5-(pyridin-4-yl)-4H-1,2,4-triazol-3-ylthio)-N-(3- | GSK3-β |
| hydroxyphenyl)acetamide | GSK3-α |
| | AKT3 |
| 2-(4-methyl-5-(4-nitrophenyl)-4H-1,2,4-triazol-3-ylthio)-N-(2-methyl-5- | PDGFRR-α |
| nitrophenyl)acetamide | |
| ethyl 2-(2-(5-((1H-benzo[d][1,2,3]triazol-1-yl)methyl)-4-methyl-4H-1,2,4- | GSK3-β |
| triazol-3-ylthio)acetamido)-4-methylthiazole-5-carboxylate | |
| 2-(5-(3,4-dimethoxyphenyl)-4-methyl-4H-1,2,4-triazol-3-ylthio)-N-(4- | AURORA-A |
| propoxybenzyl)acetamide | DYRK2 |
| 2-(4-ethyl-5-(4-(pyrrolidin-1-ylsulfonyl)phenyl)-4H-1,2,4-triazol-3-ylthio)- | GSK3-β |
| N-(thiazol-2-yl)acetamide | GSK3-α |
| 2-(4-(3-methoxypropyl)-5-methyl-4H-1,2,4-triazol-3-ylthio)-N-(2- | FYN |
| nitrophenyl)acetamide | |
| N-(benzo[d][1,3]dioxol-5-yl)-2-(4-methyl-5-((3- | AURORA-A |
| (trifluoromethyl)phenylamino)methyl)-4H-1,2,4-triazol-3-ylthio)acetamide | |
| 2-(4-(furan-2-ylmethyl)-5-(pyridin-4-yl)-4H-1,2,4-triazol-3-ylthio)-N-(2- | FYN |
| methoxy-4-nitrophenyl)acetamide | PDGFRR-α |
| | FLT-3 |
| 2-(5-benzyl-4-ethyl-4H-1,2,4-triazol-3-ylthio)-1-(3,4-dihydroquinolin-1(2H)- | FYN |
| yl)ethanone | |
| 2-(4-ethyl-5-(4-(4-methylpiperazin-1-ylsulfonyl)phenyl)-4H-1,2,4-triazol-3- | FYN |
| ylthio)-1-(3-methylpiperidin-1-yl)ethanone | |
| N-(furan-2-ylmethyl)-2-(4-(furan-2-ylmethyl)-5-(4- | p38-α |
| (methylsulfonamido)phenyl)-4H-1,2,4-triazol-3-ylthio)acetamide | p38-β |
| N-(3-aminophenyl)-2-(5-methyl-4-phenyl-4H-1,2,4-triazol-3- | FYN |
| ylthio)acetamide | |
| 2-(5-((2,4-dimethylphenylamino)methyl)-4-((tetrahydrofuran-2-yl)methyl)- | PDGFRR-α |
| 4H-1,2,4-triazol-3-ylthio)-N-(furan-2-ylmethyl)acetamide | FYN |
| 1-morpholino-2-(5-(4-nitrophenyl)-4-((tetrahydrofuran-2-yl)methyl)-4H- | PDGFRR-α |
| 1,2,4-triazol-3-ylthio)ethanone | |
| 2-(4-ethyl-5-((naphthalen-1-yloxy)methyl)-4H-1,2,4-triazol-3-ylthio)-N- | FYN |
| ((tetrahydrofuran-2-yl)methyl)acetamide | |
| 2-(2-(5-(pyridin-4-yl)-4-((tetrahydrofuran-2-yl)methyl)-4H-1,2,4-triazol-3- | GSK3-β |
| ylthio)acetamido)-4,5,6,7-tetrahydrobenzo[b]thiophene-3-carboxamide | GSK3-α |
| | AURORA-A |
| 2-(4-(4-methoxyphenyl)-5-(4-(pyrrolidin-1-ylsulfonyl)phenyl)-4H-1,2,4- | MAPKAPK-2 |
| triazol-3-ylthio)acetamide | |
| 2-(4-(2,4-dimethylphenyl)-5-(4-(piperidin-1-ylsulfonyl)phenyl)-4H-1,2,4- | p38-β |
| triazol-3-ylthio)acetamide | |
| 2-(5-(furan-2-yl)-4-phenyl-4H-1,2,4-triazol-3-ylthio)-N-phenethylacetamide | AKT1 |
| 2-(2-(4-methyl-5-((4-nitrophenoxy)methyl)-4H-1,2,4-triazol-3- | GSK3-β |
| ylthio)acetamido)-4,5,6,7-tetrahydrobenzo[b]thiophene-3-carboxamide | GSK3-α |
| | AURORA-A |
| 2-(4-methyl-5-m-tolyl-4H-1,2,4-triazol-3-ylthio)-N-m-tolylacetamide | p38-δ |
| 2-(4-phenyl-5-((quinolin-8-yloxy)methyl)-4H-1,2,4-triazol-3- | MAPKAPK-2 |
| ylthio)acetamide | |
| 2-(5-((2,4-dimethylphenylamino)methyl)-4-(furan-2-ylmethyl)-4H-1,2,4- | PDGFRR-α |
| triazol-3-ylthio)acetamide | |
| 1-(4-benzylpiperidin-1-yl)-2-(5-(furan-2-yl)-4-methyl-4H-1,2,4-triazol-3- | p38-α |
| ylthio)ethanone | |
| 1-(3,4-dihydroquinolin-1(2H)-yl)-2-(4-ethyl-5-((4- | PDGFRR-α |
| methoxyphenylamino)methyl)-4H-1,2,4-triazol-3-ylthio)ethanone | FYN |
| N-(3-chloro-2-methylphenyl)-2-(4-methyl-5-(pyridin-3-yl)-4H-1,2,4-triazol- | p38-δ |
| 3-ylthio)acetamide | |
| N-(3-acetamidophenyl)-2-(4-methyl-5-(4-(morpholinosulfonyl)phenyl)-4H- | p38-δ |
| 1,2,4-triazol-3-ylthio)acetamide | |
| N-tert-butyl-2-(4-(3-methoxyphenyl)-5-(pyridin-3-yl)-4H-1,2,4-triazol-3- | AURORA-A |
| ylthio)acetamide | |
| N-(3-(2-(4-methyl-5-(pyridin-4-yl)-4H-1,2,4-triazol-3- | GSK3-β |
| ylthio)acetamido)phenyl)furan-2-carboxamide | AURORA-A |
| | GSK3-α |
| ethyl 2-(2-(4-(furan-2-ylmethyl)-5-(pyridin-4-yl)-4H-1,2,4-triazol-3-ylthio)- | FYN |
| N-(3-nitrophenyl)acetamido)acetate | GSK3-α |
| methyl 2-(2-(4-(2-methoxyethyl)-5-(pyridin-4-yl)-4H-1,2,4-triazol-3- | GSK3-β |
| ylthio)acetamido)benzoate | GSK3-α |
| 2-(4-(4-chlorophenyl)-5-((3-chlorophenylamino)methyl)-4H-1,2,4-triazol-3- | p38-α |
| ylthio)acetamide | |
| 2-(5-((1H-benzo[d][1,2,3]triazol-1-yl)methyl)-4-methyl-4H-1,2,4-triazol-3- | GSK3-β |
| ylthio)-N-(thiazol-2-yl)acetamide | GSK3-α |
| | CDK2-cyclinE |
| 2-(4-amino-4H-1,2,4-triazol-3-ylthio)-N-(3,5-dimethoxyphenyl)acetamide | DYRK2 |
| 2-(5-((4-chloro-3-methylphenoxy)methyl)-4-ethyl-4H-1,2,4-triazol-3-ylthio)- | FYN |
| 1-(piperidin-1-yl)ethanone | |
| N-(furan-2-ylmethyl)-2-(4-(3-methoxypropyl)-5-methyl-4H-1,2,4-triazol-3- | FYN |
| ylthio)acetamide | |
| 2-(4-benzyl-5-methyl-4H-1,2,4-triazol-3-ylthio)-N-phenylpropanamide | PKA |
| | DAPK1 |
| 2-(4-ethyl-5-(m-tolyloxymethyl)-4H-1,2,4-triazol-3-ylthio)-1-(piperidin-1- | FYN |
| yl)ethanone | |
| N-(3-chlorophenyl)-2-(4,5-dimethyl-4H-1,2,4-triazol-3-ylthio)acetamide | DAPK1 |
| N-(benzo[d][1,3]dioxol-5-yl)-2-(4-methyl-5-(pyridin-3-yl)-4H-1,2,4-triazol- | c-TAK1 |
| 3-ylthio)acetamide | p38-δ |
| 2-(5-(4-(piperidin-1-ylsulfonyl)phenyl)-4H-1,2,4-triazol-3-ylthio)acetamide | GSK3-β |
| | AURORA-A |
| | GSK3-α |
| 2-(4-allyl-5-(pyridin-4-yl)-4H-1,2,4-triazol-3-ylthio)-N-(3- | FYN |
| nitrophenyl)acetamide | |
| N-(5-ethyl-1,3,4-thiadiazol-2-yl)-2-(4-methyl-5-(4-(4- | PDGFRR-α |
| methylphenylsulfonamido)phenyl)-4H-1,2,4-triazol-3-ylthio)acetamide | |
| N-cyclopentyl-2-(4-ethyl-5-(3-hydroxynaphthalen-2-yl)-4H-1,2,4-triazol-3- | p38-α |
| ylthio)acetamide | FYN |
| N-(5-ethyl-1,3,4-thiadiazol-2-yl)-2-(5-(4-hydroxyphenyl)-4-methyl-4H-1,2,4- | GSK3-β |
| triazol-3-ylthio)acetamide | GSK3-α |
| 2-(4-methyl-5-(4-(morpholinosulfonyl)phenyl)-4H-1,2,4-triazol-3-ylthio)-1- | AURORA-A |
| (pyrrolidin-1-yl)ethanone | |
| 2-(5-((1H-benzo[d]imidazol-1-yl)methyl)-4-methyl-4H-1,2,4-triazol-3- | FYN |
| ylthio)-N-(2-chlorophenyl)acetamide | |
| 2-(5-(furan-2-yl)-4-propyl-4H-1,2,4-triazol-3-ylthio)-N-(furan-2- | p38-α |
| ylmethyl)acetamide | |
| N-(4,5-dimethylthiazol-2-yl)-2-(4-(furan-2-ylmethyl)-5-(pyridin-3-yl)-4H- | GSK3-β |
| 1,2,4-triazol-3-ylthio)acetamide | AURORA-A |
| 2-(5-(4-hydroxyphenyl)-4-((tetrahydrofuran-2-yl)methyl)-4H-1,2,4-triazol-3- | GSK3-β |
| ylthio)-N-(4,5,6,7-tetrahydrobenzo[d]thiazol-2-yl)acetamide | AURORA-A |
| 2-(2-(4-(furan-2-ylmethyl)-5-phenyl-4H-1,2,4-triazol-3-ylthio)acetamido)- | GSK3-α |
| 5,6-dihydro-4H-cyclopenta[b]thiophene-3-carboxamide | GSK3-β |
| | PAK2 |
| 2-(4-phenyl-5-(4-(phenylsulfonamido)phenyl)-4H-1,2,4-triazol-3- | AURORA-A |
| ylthio)acetamide | |
| 2-(4-methyl-5-((4-phenylthiazol-2-ylamino)methyl)-4H-1,2,4-triazol-3- | FYN |
| ylthio)acetamide | |
| N-benzyl-2-(4-methyl-5-((4-phenylthiazol-2-ylamino)methyl)-4H-1,2,4- | PDGFRR-α |
| triazol-3-ylthio)acetamide | PKA |
| N-benzyl-2-(4-ethyl-5-m-tolyl-4H-1,2,4-triazol-3-ylthio)acetamide | p38-α |
| | FYN |
| 1-(piperidin-1-yl)-2-(5-(pyridin-4-yl)-4-p-tolyl-4H-1,2,4-triazol-3- | FYN |
| ylthio)ethanone | |
| 2-(5-(2-aminophenyl)-4-methyl-4H-1,2,4-triazol-3-ylthio)-N,N- | FYN |
| diisopropylacetamide | PRAK |
| 2-(5-cyclopropyl-4-methyl-4H-1,2,4-triazol-3-ylthio)-N-(4,5- | GSK3-β |
| dimethylthiazol-2-yl)acetamide | GSK3-α |
| 2-(5-cyclopropyl-4-methyl-4H-1,2,4-triazol-3-ylthio)-N-(4,5,6,7- | CDK2-cyclinE |
| tetrahydrobenzo[d]thiazol-2-yl)acetamide | |
| 2-(4-(2,5-dimethylphenyl)-5-(o-tolyloxymethyl)-4H-1,2,4-triazol-3- | p38-β |
| ylthio)acetamide | |
| N-cyclohexyl-2-(5-(3,4-dimethoxyphenyl)-4-methyl-4H-1,2,4-triazol-3- | p38-δ |
| ylthio)acetamide | |
| 4-(5-(2-(2,6-dimethylpiperidin-1-yl)-2-oxoethylthio)-4-ethyl-4H-1,2,4- | PDGFRR-α |
| triazol-3-yl)-N,N-diethylbenzenesulfonamide | FYN |
| ethyl 3-(2-(5-(furan-2-yl)-4-methyl-4H-1,2,4-triazol-3- | p38-δ |
| ylthio)acetamido)benzoate | |
| 2-(5-(furan-2-yl)-4-methyl-4H-1,2,4-triazol-3-ylthio)-N-(3- | p38-δ |
| (trifluoromethyl)phenyl)acetamide | |
| 2-(4-(2,5-dimethylphenyl)-5-(4-hydroxyphenyl)-4H-1,2,4-triazol-3- | GSK3-β |
| ylthio)acetamide | GSK3-α |
| | CDK2-cyclinE |
| ethyl 4-(2-(4-ethyl-5-((4-methoxyphenoxy)methyl)-4H-1,2,4-triazol-3- | FYN |
| ylthio)acetamido)benzoate | |
| 2-(5-(2-hydroxyphenyl)-4-(3-methoxyphenyl)-4H-1,2,4-triazol-3- | p38-α |
| ylthio)acetamide | |
| ethyl 2-(2-(5-(4-hydroxyphenyl)-4-methyl-4H-1,2,4-triazol-3- | GSK3-β |
| ylthio)acetamido)-4-methylthiazole-5-carboxylate | GSK3-α |
| | p38-γ |
| N-(5-ethyl-1,3,4-thiadiazol-2-yl)-2-(5-(pyridin-4-yl)-4-p-tolyl-4H-1,2,4- | GSK3-β |
| triazol-3-ylthio)acetamide | GSK3-α |
| | p38-α |
| N-(furan-2-ylmethyl)-2-(4-(furan-2-ylmethyl)-5-(pyridin-4-yl)-4H-1,2,4- | FYN |
| triazol-3-ylthio)acetamide | |
| N-(4-fluorophenyl)-2-(4-(2-methoxyethyl)-5-(pyridin-4-yl)-4H-1,2,4-triazol- | FYN |
| 3-ylthio)acetamide | |
| N-(2-nitrophenyl)-2-(4-(pyridin-3-yl)-4H-1,2,4-triazol-3-ylthio)acetamide | FYN |
| 2-(5-((4-ethoxyphenylamino)methyl)-4-phenyl-4H-1,2,4-triazol-3- | FYN |
| ythio)acetamide | PDGFRR-α |
| | FLT-3 |
| N-(3-cyano-4,5,6,7-tetrahydrobenzo[b]thiophen-2-yl)-2-(5-((4- | DYRK2 |
| methoxyphenylamino)methyl)-4-methyl-4H-1,2,4-triazol-3-ylthio)acetamide | CDK2-cyclinE |
| 2-(4-ethyl-5-phenyl-4H-1,2,4-triazol-3-ylthio)-N-(furan-2- | FYN |
| ylmethyl)acetamide | |
| 2-(4-benzyl-5-((4-fluorophenylamino)methyl)-4H-1,2,4-triazol-3- | GSK3-β |
| ylthio)acetamide | AKT1 |
| | GSK3-α |
| 2-(4-ethyl-5-isopropyl-4H-1,2,4-triazol-3-ylthio)-N-o-tolylacetamide | PDGFRR-α |
| 2-(5-(furan-2-yl)-4-methyl-4H-1,2,4-triazol-3-ylthio)-1-morpholinoethanone | FYN |
| 2-(4-(3-(dimethylamino)propyl)-5-(pyridin-4-yl)-4H-1,2,4-triazol-3-ylthio)- | PDGFRR-α |
| N-(furan-2-ylmethyl)acetamide | PAK2 |
| 2-(4-ethyl-5-(4-(4-methylpiperazin-1-ylsulfonyl)phenyl)-4H-1,2,4-triazol-3- | FYN |
| ylthio)-N-(4-methylthiazol-2-yl)acetamide | |
| 2-(5-((4-methoxyphenoxy)methyl)-4-methyl-4H-1,2,4-triazol-3-ylthio)-N-o- | PDGFRR-α |
| tolylpropanamide | |
| N-(4-(5-(2-(furan-2-ylmethylamino)-2-oxoethylthio)-4-methyl-4H-1,2,4- | CHEK2 |
| triazol-3-yl)phenyl)benzamide | |
| ethyl 2-(2-(2-(4-(4-methoxyphenyl)-5-(pyridin-4-yl)-4H-1,2,4-triazol-3- | p38-α |
| ylthio)acetamido)thiazol-4-yl)acetate | |
| 2-(4-methyl-5-((naphthalen-2-yloxy)methyl)-4H-1,2,4-triazol-3- | FYN |
| ylthio)acetamide | |
| N-(3-(2-(5-(4-methoxyphenyl)-4-phenyl-4H-1,2,4-triazol-3- | PRAK |
| ylthio)acetamido)phenyl)-tetrahydrofuran-2-carboxamide | CHEK2 |
| | PKA |
| N-(3-(2-(5-(4-methoxyphenyl)-4-phenyl-4H-1,2,4-triazol-3- | PRAK |
| ylthio)acetamido)phenyl)furan-2-carboxamide | CHEK2 |
| | PKA |
| N-(2-methoxyethyl)-2-(5-(4-methoxyphenyl)-4-phenyl-4H-1,2,4-triazol-3- | P70S6K1 |
| ylthio)acetamide | |
| N-(cyclohexylmethyl)-2-(5-(4-methoxyphenyl)-4-phenyl-4H-1,2,4-triazol-3- | AKT3 |
| ylthio)acetamide | |
| N-(furan-2-ylmethyl)-2-(5-(4-methoxyphenyl)-4-phenyl-4H-1,2,4-triazol-3- | p38-α |
| ylthio)acetamide | P70S6K1 |
| 2-(5-(4-methoxyphenyl)-4-phenyl-4H-1,2,4-triazol-3-ylthio)-N- | PRAK |
| phenylacetamide | CHEK2 |
| N-(2-chlorophenyl)-2-(5-(4-methoxyphenyl)-4-phenyl-4H-1,2,4-triazol-3- | INSR |
| ylthio)acetamide | SYK |
| N-(3-chlorophenyl)-2-(5-(4-methoxyphenyl)-4-phenyl-4H-1,2,4-triazol-3- | PRAK |
| ylthio)acetamide | CHEK2 |
| | AURORA-A |
| N-(4-chlorophenyl)-2-(5-(4-methoxyphenyl)-4-phenyl-4H-1,2,4-triazol-3- | P70S6K1 |
| ylthio)acetamide | GSK3-β |
| 2-(5-(4-methoxyphenyl)-4-phenyl-4H-1,2,4-triazol-3-ylthio)-N-(thiophen-2- | P70S6K1 |
| ylmethyl)acetamide | p38-γ |
| N-cyclohexyl-2-(5-(4-methoxyphenyl)-4-phenyl-4H-1,2,4-triazol-3- | P70S6K1 |
| ylthio)acetamide | p38-γ |
| | GSK3-β |
| N-butyl-2-(5-(4-methoxyphenyl)-4-phenyl-4H-1,2,4-triazol-3- | P70S6K1 |
| ylthio)acetamide | p38-γ |
| N-(2-hydroxyethyl)-2-(5-(4-methoxyphenyl)-4-phenyl-4H-1,2,4-triazol-3- | PDK1 |
| ylthio)acetamide | P70S6K1 |
| 2-(5-(4-methoxyphenyl)-4-phenyl-4H-1,2,4-triazol-3-ylthio)-1-(pyrrolidin-1- | P70S6K1 |
| yl)ethanone | |
| N-benzyl-2-(5-(4-methoxyphenyl)-4-phenyl-4H-1,2,4-triazol-3-ylthio)-N- | p38-γ |
| methylacetamide | AKT1 |
| 2-(5-(4-methoxyphenyl)-4-phenyl-4H-1,2,4-triazol-3-ylthio)-1- | INSR |
| morpholinoethanone | PRAK |
| 2-(5-(4-methoxyphenyl)-4-phenyl-4H-1,2,4-triazol-3-ylthio)-N,N- | p38-γ |
| dipropylacetamide | GSK3-α |
| 2-(5-(4-methoxyphenyl)-4-phenyl-4H-1,2,4-triazol-3-ylthio)-N- | SYK |
| methylacetamide | PRAK |
| 2-(5-(4-methoxyphenyl)-4-phenyl-4H-1,2,4-triazol-3-ylthio)acetamide | PDK1 |
| 2-(5-(4-methoxyphenyl)-4-phenyl-4H-1,2,4-triazol-3-ylthio)-N,N- | PDK1 |
| dimethylacetamide | SYK |
| | INSR |

| | |
|---|---|
| N-(3-(2-(5-(2-hydroxyphenyl)-4-(2-methoxyphenyl)-4H-1,2,4-triazol-3- | AURORA-A |
| ylthio)acetamido)phenyl)-tetrahydrofuran-2-carboxamide | |
| N-(3-(2-(5-(2-hydroxyphenyl)-4-(2-methoxyphenyl)-4H-1,2,4-triazol-3- | AURORA-A |
| ylthio)acetamido)phenyl)furan-2-carboxamide | |
| N-(3-(2-(5-(2-hydroxyphenyl)-4-(2-methoxyphenyl)-4H-1,2,4-triazol-3- | AURORA-A |
| ylthio)acetamido)-4-methylphenyl)furan-2-carboxamide | |
| N-(2-(dimethylamino)ethyl)-2-(5 N-(2-hydroxyphenyl)-4-(2-methoxyphenyl)- | P70S6K1 |
| H-1,2,4-triazol-3-ylthio)acetamide | |
| 2-(5-(2-hydroxyphenyl)-4-(2-methoxyphenyl)-4H-1,2,4-triazol-3-ylthio)-N- | KIT |
| 2-methoxyethyl)acetamide | |
| N-(cyclohexylmethyl)-2-(5-(2-hydroxyphenyl)-4-(2-methoxyphenyl)-4H- | SRC |
| 1,2,4-triazol-3-ylthio)acetamide | PDGFRR-α |
| | P70S6K1 |
| 2-(5-(2-hydroxyphenyl)-4-(2-methoxyphenyl)-4H-1,2,4-triazol-3-ylthio)-N- | AURORA-A |
| phenylacetamide | |
| N-(2-chlorophenyl)-2-(5-(2-hydroxyphenyl)-4-(2-methoxyphenyl)-4H-1,2,4- | p38-α |
| triazol-3-ylthio)acetamide | KIT |
| N-(3-chlorophenyl)-2-(5-(2-hydroxyphenyl)-4-(2-methoxyphenyl)-4H-1,2,4- | KIT |
| triazol-3-ylthio)acetamide | P70S6K1 |
| N-(4-chlorophenyl)-2-(5-(2-hydroxyphenyl)-4-(2-methoxyphenyl)-4H-1,2,4- | KIT |
| triazol-3-ylthio)acetamide | AURORA-A |
| 2-(5-(2-hydroxyphenyl)-4-(2-methoxyphenyl)-4H-1,2,4-triazol-3-ylthio)-N- | p38-β |
| (thiophen-2-ylmethyl)acetamide | SYK |
| | PAK2 |
| N-cyclohexyl-2-(5-(2-hydroxyphenyl)-4-(2-methoxyphenyl)-4H-1,2,4- | SYK |
| triazol-3-ylthio)acetamide | PAK2 |
| | p38-δ |
| N-butyl-2-(5-(2-hydroxyphenyl)-4-(2-methoxyphenyl)-4H-1,2,4-triazol-3- | INSR |
| ylthio)acetamide | KIT |
| | SYK |
| N-(2-hydroxyethyl)-2-(5-(2-hydroxyphenyl)-4-(2-methoxyphenyl)-4H-1,2,4- | P70S6K1 |
| triazol-3-ylthio)acetamide | |
| 2-(5-(2-hydroxyphenyl)-4-(2-methoxyphenyl)-4H-1,2,4-triazol-3-ylthio)-1- | PRAK |
| (pyrrolidin-1-yl)ethanone | |
| 2-(5-(2-hydroxyphenyl)-4-(2-rnethoxyphenyl)-4H-1,2,4-triazol-3 N-ylthio)-N- | p38-α |
| ethyl-N-phenylacetamide | |
| N-benzyl-2-(5-(2-hydroxyphenyl)-4-(2-methoxyphenyl)-4H-1,2,4-triazol-3- | p38-α |
| ylthio)-N-methylacetamide | PAK2 |
| 2-(5-(2-hydroxyphenyl)-4-(2-methoxyphenyl)-4H-1,2,4-triazol-3-ylthio)- | KIT |
| N,N-dipropylacetamide | AURORA-A |
| | PKA |
| 2-(5-(2-hydroxyphenyl)-4-(2-methoxyphenyl)-4H-1,2,4-triazol-3-ylthio)-N- | AURORA-A |
| methylacetamide | |
| N-(5-(4-methoxyphenyl)-4-phenyl-4H-1,2,4-triazol-3-ylthio)-1-(4- | P70S6K1 |
| methylpiperazin-1-yl)ethanone | |
| 2-(5-(2-hydroxyphenyl)-4-(2-methoxyphenyl)-4H-1,2,4-triazol-3 N-ylthio)-1- | p38-α |
| (4-methylpiperazin-1-yl)ethanone | P70S6K1 |
| 2-(5-m-tolyl-4H-1,2,4-triazol-3-ylthio)acetamide | CHEK2 |
| | AURORA-A |
| | FLT-3 |
| 2-(5-(4-(trifluoromethoxy)phenyl)-4H-1,2,4-triazol-3 N-ylthio)acetamide | AURORA-A |
| | GSK3-β |
| | DYRK2 |
| N-benzyl-2-(4-(2-methoxyphenyl)-5-phenyl-4H-1,2,4-triazol-3- | p38-α |
| ylthio)acetamide | |
| N-(furan-2-ylmethyl)-3-(4-(2-methoxyphenyl)-5-phenyl-4H-1,2,4-triazol-3- | p38-α |
| ylthio)propanamide | |
| N-(5-(3-chlorophenyl)-4-(2-methoxyphenyl)-4H-1,2,4-triazol-3-ylthio)-N- | p38-α |
| furan-2-ylmethyl)propanamide | |
| N-benzyl-3-(4-(2-methoxyphenyl)-5-phenyl-4H-1,2,4-triazol-3- | p38-α |
| ylthio)propanamide | |
| N-benzyl-3-(5-(3-chlorophenyl)-4-(2-methoxyphenyl)-4H-1,2,4-triazol-3- | p38-α |
| ylthio)propanamide | |
| 3-(4-(2-methoxyphenyl)-5-phenyl-4H-1,2,4-triazol-3-ylthio)-N-(thiophen-2- | p38-α |
| ylmethyl)propanamide | |
| 3-(5-(3-chlorophenyl)-4-(2-methoxyphenyl)-4H-1,2,4-triazol-3-ylthio)-N- | p38-a |
| (thiophen-2-ylmethyl)propanamide | |
| M-(3-(3-(4-(2-methoxyphenyl)-5-phenyl-4H-1,2,4-triazol-3- | MAPKAPK-3 |
| ylthio)propanamido)-4-methylphenyl)furan-2-carboxamide | p38-α |
| | p38-β |
| N-(3-(3-(5-(3-chlorophenyl)-4-(2-methoxyphenyl)-4H-1,2,4-triazol-3- | MAPKAPK-3 |
| ylthio)propanamido)-4-methylphenyl)furan-2-carboxamide | p38-α |
| | p38-β |
| N-(3-(3-(4-(2-methoxyphenyl)-5-phenyl-4H-1,2,4-triazol-3- | p38-α |
| ylthio)propanamido)phenyl)furan-2-carboxamide | |
| N-(furan-2-ylmethyl)-2-(4-(2-methoxyphenyl)-5-phenyl-4H-1,2,4-triazol-3- | AKT1 |
| ylthio)propanamide | |
| 2-(5-cyclohexyl-4-(2-methoxyphenyl)-4H-1,2,4-triazol-3-ylthio)-N- | p38-α |
| phenylpropanamide | |
| N-(4-chlorophenyl)-2-(4-(2-methoxyphenyl)-5-phenyl-4H-1,2,4-triazol-3- | p38-α |
| ylthio)propanamide | |
| N-(2-chlorophenyl)-2-(5-cyclohexyl-4-(2-methoxyphenyl)-4H-1,2,4-triazol- | P70S6K1 |
| 3-ylthio)propanamide | |
| 2-(5-cyclohexyl-4-(2-methoxyphenyl)-4H-1,2,4-triazol-3-ylthio)-N-o- | p38-α |
| tolylpropanamide | |
| 2-(5-cyclohexyl-4-(2-methoxyphenyl)-4H-1,2,4-triazol-3-ylthio)-N-methyl- | p38-α |
| N-phenylpropanamide | |
| N-(3-(2-(4-(2-methoxyphenyl)-5-phenyl-4H-1,2,4-triazol-3- | p38-α |
| ylthio)propanamido)-4-methylphenyl)furan-2-carboxamide | p38-β |
| N-(3-(2-(5-cyclohexyl-4-(2-methoxyphenyl)-4H-1,2,4-triazol-3- | MAPKAPK-3 |
| ylthio)propanamido)-4-methylphenyl)furan-2-carboxamide | p38-α |
| N-(3-(2-(5-(3-chlorophenyl)-4-(2-methoxyphenyl)-4H-1,2,4-triazol-3- | MAPKAP-3 |
| ylthio)propanamido)-4-methylphenyl)furan-2-carboxamide | p38-α |
| | p38-β |
| N-(3-(2-(4-(2-methoxyphenyl)-5-phenyl-4H-1,2,4-triazol-3- | p38-α |
| ylthio)propanamido)phenyl)furan-2-carboxamide | |
| N-(3-(2-(5-(3-chlorophenyl)-4-(2-methoxyphenyl)-4H-1,2,4-triazol-3- | ROCK2 |
| ylthio)propanamido)phenyl)furan-2-carboxamide | |
| N′-(2-(5-((4,6-dimethylpyrimidin-2-ylthio)methyl)-4-methyl-4H-1,2,4- | SRC |
| triazol-3-ylthio)acetyl)-2-hydroxybenzohydrazide | FLT-3 |
| | FYN |
| N′-(2-(4-(furan-2-ylmethyl)-5-(4-hydroxyphenyl)-4H-1,2,4-triazol-3- | GSK3-β |
| ylthio)acetyl)-4-methoxybenzohydrazide | AURORA-A |
| | PDGFRR-α |
| N-bromo-N′-(2-(5-(4-hydroxyphenyl)-4-phenyl-4H-1,2,4-triazol-3- | AURORA-A |
| ylthio)acetyl)benzohydrazide | |
| N-hydroxy-N′-(2-(5-(2-hydroxyphenyl)-4-(2-methoxyphenyl)-4H-1,2,4- | FYN |
| triazol-3-ylthio)acetyl)benzohydrazide | FLT-3 |
| | SYK |
| N′-(2-(4-ethyl-5-(4-hydroxyphenyl)-4H-1,2,4-triazol-3-ylthio)acetyl)-4- | GSK3-a |
| methoxybenzohydrazide | AURORA-A |
| N′-(2-(5-benzyl-4-cyclohexyl-4H-1,2,4-triazol-3-ylthio)acetyl)-2- | CDK2-cyclinE |
| hydroxybenzohydrazide | p38-β |
| N′-(2-(5-((4,6-dimethylpyrimidin-2-ylthio)methyl)-4-phenyl-4H-1,2,4- | FYN |
| triazol-3-ylthio)acetyl)-2-hydroxybenzohydrazide | SRC |
| | HCK |
| N′-(2-(4-benzyl-5-(2-hydroxyphenyl)-4H-1,2,4-triazol-3-ylthio)acetyl)-2- | FYN |
| hydroxybenzohydrazide | HCK |
| 4-methoxy-N′-(2-(5-methyl-4-phenethyl-4H-1,2,4-triazol-3- | DGFRR-α |
| ylthio)acetyl)benzohydrazide | |
| N′-(2-(5-((3-chlorophenylamino)methyl)-4-phenethyl-4H-1,2,4-triazol-3- | PDGFRR-α |
| ylthio)acetyl)-4-methoxybenzohydrazide | GSK3-β |
| | CDK1 |
| 4-chloro-N′-(2-(5-(4-hydroxyphenyl)-4-phenyl-4H-1,2,4-triazol-3- | AURORA-A |
| ylthio)acetyl)benzohydrazide | |
| 4-methoxy-N′-(2-(5-methyl-4-((tetrahydrofuran-2-yl)methyl)-4H-1,2,4- | SYK |
| triazol-3-ylthio)acetyl)benzohydrazide | |
| 2-hydroxy-N′-(2-(5-(2-hydroxyphenyl)-4-p-tolyl-4H-1,2,4-triazol-3- | AURORA-A |
| ylthio)acetyl)benzohydrazide | |
| 2-(5-(4-bromophenyl)-4-(2-methylallyl)-4H-1,2,4-triazol-3- | LCK |
| ylthio)acetohydrazide | |
| N′-(2-(5-((4-cyanophenoxy)methyl)-4-phenyl-4H-1,2,4-triazol-3- | LCK |
| ylthio)acetyl)-4-methylbenzohydrazide | |
| N-bromo-N′-(2-(5-cyclohexyl-4-methyl-4H-1,2,4-triazol-3- | SGK1 |
| ylthio)acetyl)benzohydrazide | |
| N′-(2-(4-(4-fluorophenyl)-5-(pyridin-4-yl)-4H-1,2,4-triazol-3-ylthio)acetyl)- | FYN |
| N-hydroxybenzohydrazide | |
| 2-hydroxy-N′-(2-(4-methyl-5-phenyl-4H-1,2,4-triazol-3- | FYN |
| ylthio)acetyl)benzohydrazide | FLT-3 |
| | CSK |
| N′-(2-(5-((2-chlorophenylamino)methyl)-4-ethyl-4H-1,2,4-triazol-3- | AURORA-A |
| ylthio)acetyl)-4-methoxybenzohydrazide | DYRK2 |
| N′-(2-(4-ethyl-5-(pyridin-4-yl)-4H-1,2,4-triazol-3- | AURORA-A |
| ylthio)acetyl)cyclohexanecarbohydrazide | |
| N′-(2-(4,5-dibenzyl-4H-1,2,4-triazol-3-ylthio)acetyl)-4- | DAPK-1 |
| methylbenzohydrazide | |
| N′-(2-(4-benzyl-5-phenyl-4H-1,2,4-triazol-3-ylthio)acetyl)-2- | FYN |
| hydroxybenzohydrazide | CSK |
| N-hydroxy-N′-(2-(4-(3-methoxypropyl)-5-(pyridin-4-yl)-4H-1,2,4-triazol-3- | AURORA-A |
| ylthio)acetyl)benzohydrazide | |
| 2-(5-benzyl-4-p-tolyl-4H-1,2,4-triazol-3-yltbio)-N′-(2- | p38-β |
| phenylacetyl)acetohydrazide | |
| N′-(2-(4-benzyl-5-(pyridin-3-yl)-4H-1,2,4-triazol-3-ylthio)acetyl)-4- | PDGFRR-α |
| bromobenzohydrazide | AURORA-A |
| N′-(2-(5-((4-methoxyphenylamino)methyl)-4-methyl-4H-1,2,4-triazol-3- | PDGFRR-α |
| ylthio)acetyl)-4-methylbenzohydrazide | FYN |
| | FLT-3 |
| N′-acetyl-2-(4-(4-methoxyphenyl)-5-(pyridin-4-yl)-4H-1,2,4-triazol-3- | FYN |
| ylthio)acetohydrazide | |
| 2-(5-(4 N-chlorophenyl)-4-ethyl-4H-1,2,4-triazol-3-ylthio)-N′-(2- | FYN |
| phenylacetyl)acetohydrazide | |
| 4-bromo-N′-(2-(5-(4-methoxyphenyl)-4-methyl-4H-1,2,4-triazol-3- | CHEK2 |
| ylthio)acetyl)benzohydrazide | GSK3-β |
| | GSK3-α |
| N′-(2-(5-(3,4-dimethoxyphenyl)-4-phenyl-4H-1,2,4-triazol-3-ylthio)acetyl)-2- | FYN |
| hydroxybenzohydrazide | FLT-3 |
| 3-(4-(4-methoxyphenyl)-5-((naphthalen-1-yloxy)methyl)-4H-1,2,4-triazol-3- | p38-β |
| ylthio)propanoic acid | |
| 2-(2-(5-(allylthio)-4-methyl-4H-1,2,4-triazol-3-yl)ethyl)-1H- | p38-δ |
| benzo[d]imidazole | |
| 4-(5-(ethylthio)-4-(3-methoxypropyl)-4H-1,2,4-triazol-3-yl)pyridine | AURORA-A |
| 3-(5-((naphthalen-1-yloxy)methyl)-4-phenyl-4H-1,2,4-triazol-3- | p38-α |
| ylthio)propanoic acid | |
| 3-(4-phenyl-5-((3-(trifluoromethyl)phenylamino)methyl)-4H-1,2,4-triazol-3- | p38-α |
| ylthio)propanoic acid | |
| 1,2-bis(5-(2-methoxyphenyl)-4-p-tolyl-4H-1,2,4-triazol-3-ylthio)ethane | CDK1 |
| 3-benzyl-4-(4-methoxyphenyl)-5-(methylthio)-4H-1,2,4-triazole | AKT1 |
| 3-(4-(3-chlorophenyl)-5-(2-methoxyphenyl)-4H-1,2,4-triazol-3- | CHEK2 |
| ylthio)propanoic acid | |
| 3-(5-(4-benzamidophenyl)-4-(4-methoxyphenyl)-4H-1,2,4-triazol-3- | CHEK2 |
| ylthio)propanoic acid | AURORA-A |
| 3-(5-((naphthalen-1-yloxy)methyl)4-p-tolyl-4H-1,2,4-triazol-3- | p38-α |
| ylthio)propanoic acid | |
| 3-(4-phenyl-5-(o-tolyloxymethyl)-4H-1,2,4-triazol-3-ylthio)propanoic acid | p38-α |
| 4-fluoro-N-((4-(4-methoxyphenyl)-5-(methylthio)-4H-1,2,4-triazol-3- | AKT1 |
| yl)methyl)benzenamine | |
| 3-(methylthio)-4-phenethyl-5-(phenoxymethyl)-4H-1,2,4-triazole | p38-α |
| | p38-β |
| 4-(4-benzyl-5-(methylthio)-4H-1,2,4-triazol-3-yl)phenol | GSK3-α |
| | GSK3-β |
| 3-(ethylthio)-4-methyl-5-(4-nitrophenyl)-4H-1,2,4-triazole | PDGFRR-α |
| | FYN |
| 3-(4-phenyl-5-(m-tolyloxymethyl)-4H-1,2,4-triazol-3-ylthio)propanoic acid | p38-α |
| 2-(5-(pyridin-4-yl)-4-p-tolyl-4H-1,2,4-triazol-3-ylthio)ethanol | GSK3-β |
| | GSK3-α |
| 3-(isopropylthio)-5-(phenoxymethyl)-4-p-tolyl-4H-1,2,4-triazole | p38-β |
| 2-(2-(5-(butylthio)-4-methyl-4H-1,2,4-triazol-3-yl)ethyl)-1H- | p38-γ |
| 3-(5-((2-methoxyphenoxy)methyl)-4-(4-methoxyphenyl)-4H-1,2,4-triazol-3- | CDK1 |
| ylthio)propanoic acid | |
| 4-(5-(ethylthio)-4-p-tolyl-4H-1,2,4-triazol-3-yl)pyridine | GSK3-β |
| | GSK3-α |
| | p38-α |
| 4-(5-(methylthio)-4-phenyl-4H-1,2,4-triazol-3-yl)phenol | GSK3-β |
| | GSK3-α |
| | CDK2-cyclinA |
| 1,2-bis(5-cyclohexyl-4-ethyl-4H-1,2,4-triazol-3-ylthio)ethane | CHEK2 |
| 3-(4-(3-chlorophenyl)-5-(4-hydroxyphenyl)-4H-1,2,4-triazol-3- | GSK3-β |
| ylthio)propanoic acid | GSK3-α |
| 4-ethyl-3-(phenethylthio)-5-(phenoxymethyl)-4H-1,2,4-triazole | p38-α |
| | CDK1 |
| 3-(5-((4,6-dimethylpyrimidin-2-ylthio)methyl)-4-phenyl-4H-1,2,4-triazol-3- | CHEK2 |
| ylthio)propanoic acid | |
| 4-(4-methoxyphenyl)-3-(methylthio)-5-(m-tolyloxymethyl)-4H-1,2,4-triazole | p38-β |
| 3-((4-chloro-3-methylphenoxy)methyl)-4-ethyl-5-(isopropylthio)-4H-1,2,4- | AKT1 |
| triazole | |
| 3-(4-(2-methoxyphenyl)-5-(pyridin-3-yl)-4H-1,2,4-triazol-3-ylthio)propanoic | AURORA-A |
| acid | |
| 4-ethyl-3-(isobutylthio)-5-(o-tolyloxymethyl)-4H-1,2,4-triazole | p38-α |
| | CHEK2 |
| | SYK |
| 3-(2-bromophenyl)-4-(4-methoxyphenyl)-5-(methylthio)-4H-1,2,4-triazole | p38-β |
| N-(4-(5-(isopropylthio)-4-methyl-4H-1,2,4-triazol-3- | CDK2-cyclinA |
| yl)phenyl)methanesulfonamide | |
| 1-((5-(allylthio)-4-methyl-4H-1,2,4-triazol-3-yl)methyl)-1H- | HCK |
| benzo[d]imidazole | |
| 3-(ethylthio)-4-(2-methoxyphenyl)-5-o-tolyl-4H-1,2,4-triazole | p38-α |
| N-fluoro-N-((5-(methylthio)-4-p-tolyl-4H-1,2,4-triazol-3- | p38-β |
| yl)methyl)benzenamine | |
| 4-benzyl-3-(methylthio)-5-(o-tolyloxymethyl)-4H-1,2,4-triazole | MSK2 |
| 4-(4-benzyl-5-(ethylthio)-4H-1,2,4-triazol-3-yl)phenol | GSK3-β |
| | GSK3-α |
| 3-(5-((3-chloro-4-methylphenylamino)methyl)-4-phenyl-4H-1,2,4-triazol-3- | p38-α |
| ylthio)propanoic acid | |
| 2-(5-(ethylthio)-4-(2-methoxyphenyl)-4H-1,2,4-triazol-3-yl)phenol | p38-β |
| 4-(4-methoxyphenyl)-3-(methylthio)-5-((naphthalen-1-yloxy)methyl)-4H- | p38-α |
| 1,2,4-triazole | MSK2 |
| 3-(5-(phenoxymethyl)-4-p-tolyl-4H-1,2,4-triazol-3-ylthio)propanoic acid | p38-α |
| 3-((4-ethylphenoxy)methyl)-5-(ethylthio)-4-methyl-4H-1,2,4-triazole | AURORA-A |
| 3-((4-bromophenoxy)methyl)-5-(butylthio)-4-ethyl-4H-1,2,4-triazole | CDK2-cyclinA |
| | AURORA-A |
| 3-(4-ethyl-5-((4-ethylphenoxy)rnethyl)-4H-1,2,4-triazol-3-ylthio)propanoic | MSK1 |
| acid | |
| 3-(4,5-dip-tolyl-4H-1,2,4-triazol-3-ylthio)propanoic acid | DAPK1 |
| 3-(5-((4-bromophenoxy)methyl)-4-ethyl-4H-1,2,4-triazol-3-ylthio)propanoic | AURORA-A |
| acid | |
| 3-(4-ethyl-5-((naphthalen-1-yloxy)methyl)-4H-1,2,4-triazol-3- | AURORA-A |
| ylthio)propanoic acid | |
| 3-((3-methoxyphenoxy)methyl)-4-methyl-5-(methylthio)-4H-1,2,4-triazole | AURORA-A |
| 2-(5-(methylthio)-4-p-tolyl-4H-1,2,4-triazol-3-yl)phenol | AURORA-A |
| | p38-γ |
| | p38-α |
| 3-(5-((4-chloro-3-methylphenoxy)methyl)-4-methyl-4H-1,2,4-triazol-3- | AURORA-A |
| ylthio)propanoic acid | |
| 3-(4-phenyl-5-(pyridin-3-yl)-4H-1,2,4-triazol-3-ylthio)propanoic acid | AURORA-A |
| 4-ethyl-3-(isobutylthio)-5-((naphthalen-1-yloxy)methyl)-4H-1,2,4-triazole | c-TAK1 |
| | CDK1 |
| 3-(4-benzyl-5-(4-hydroxyphenyl)-4H-1,2,4-triazol-3-ylthio)propanoic acid | GSK3-β |
| | GSK3-α |
| (E)-4-(5-(cinnamylthio)-4-phenyl-4H-1,2,4-triazol-3-yl)pyridine | GSK3-β |
| | GSK3-α |
| | MSK1 |
| 1-(9H-carbazol-9-yl)-3-(4-cyclohexyl-5-phenyl-4H-1,2,4-triazol-3- | p38-α |
| ylthio)propan-2-ol | |
| ethyl 4-(5-(2,3-dimethyl-1H-indol-5-yl)-4-ethyl-4H-1,2,4-triazol-3- | FLT-3 |
| ylthio)butanoate | |
| 2-(5-benzyl-4-phenyl-4H-1,2,4-triazol-3-ylthio)-N-phenylethanesulfonamide | AKT2 |
| 3-(2-methoxyethylthio)-5-(4-methoxyphenyl)-4-phenyl-4H-1,2,4-triazole | SYK |
| | INSR |
| | PRAK |
| 2-(5-(2-methoxyethylthio)-4-(2-methoxyphenyl)-4H-1,2,4-triazol-3-yl)phenol | p38-α |
| | P70S6K1 |
| | p38-β |
| N-(2-(5-(3-(trifluoromethyl)phenyl)-4H-1,2,4-triazol-3- | GSK3-β |
| ylthio)ethyl)benzamide | GSK3-α |
| | CHEK2 |
| 3-(butylthio)-5-(4-methoxyphenyl)-4H-1,2,4-triazole | AURORA-A |
| | FLT-3 |
| | GSK3-α |
| 4-(furan-2-ylmethyl)-3-methyl-5-(3-methylbut-2-enylthio)-4H-1,2,4-triazole | GSK3-β |
| | GSK3-α |
| 3-ethyl-5-(3-methylbut-2-enylthio)-4-phenethyl-4H-1,2,4-triazole | GSK3-α |
| 4-(benzo[d][1,3]dioxol-5-ylmethyl)-3-ethyl-5-(3-methylbut-2-enylthio)-4H- | GSK3-β |
| 1,2,4-triazole | GSK3-α |
| 3-benzyl-4-(3,4-dichlorobenzyl)-5-(3-methylbut-2-enylthio)-4H-1,2,4- | c-TAK1 |
| triazole | |
| 4-allyl-3-(2-cyclopentylethyl)-5-(3-methylbut-2-enylthio)-4H-1,2,4-triazole | DYRK2 |
| 4-(3-(3-(2-cyclopentylethyl)-5-(3-methylbut-2-enylthio)-4H-1,2,4-triazol-4- | GSK3-α |
| yl)propyl)morpholine | |
| 3-(2-cyclopentylethyl)-4-(3,4-dimethoxyphenethyl)-5-(3-methylbut-2- | GSK3-β |
| enylthio)-4H-1,2,4-triazole | GSK3-α |
| 3-(butylthio)-5-phenyl-4H-1,2,4-triazole | AURORA-A |
| | FLT-3 |
| | c-TAK1 |
| 2-(5-phenyl-4H-1,2,4-triazol-3-ylthio)ethanol | FLT-3 |
| | AURORA-A |
| | DYRK2 |
| N-(2-(5-phenyl-4H-1,2,4-triazol-3-ylthio)ethyl)benzamide | GSK3-β |
| | DYRK2 |
| | AURORA-A |
| 2-(2-(5-phenyl-4H-1,2,4-triazol-3-ylthio)ethyl)isoindoline-1,3-dione | AURORA-A |
| | GSK3-β |
| | GSK3-α |
| 3-(2-ethoxyethylthio)-5-phenyl-4H-1,2,4-triazole AURORA-A | |
| | FLT-3 |
| | DYRK2 |
| 3-(5-(butylthio)-4H-1,2,4-triazol-3-yl)pyridme | AURORA-A |
| | FLT-3 |
| | c-TAK1 |
| 3-(5-(pyridin-3-yl)-4H-1,2,4-triazol-3-ylthio)propane-1,2-diol | AURORA-A |
| 3-(5-(2-ethoxyethylthio)-4H-1,2,4-triazol-3-yl)pyridine | AURORA-A |
| 1,1,1-trifluoro-3-(5-(pyridin-3-yl)-4H-1,2,4-triazol-3-ylthio)propan-2-ol | AURORA-A |
| ethyl 3-(5-(pyridin-3-yl)-4H-1,2,4-triazol-3-ylthio)propanoate | AURORA-A |
| | DYRK2 |
| 3-(2-ethoxyethylthio)-5-m-tolyl-4H-1,2,4-tnazole | AURORA-A |
| | FLT-3 |
| | GSK3-β |
| 3-(butylthio)-5-(3-(trifluoromethyl)phenyl)-4H-1,2,4-triazole | AURORA-A |
| | FLT-3 |
| | GSK3-α |
| 3-(5-(3-(trifluoromethyl)phenyl)-4H-1,2,4-triazol-3-ylthio)propane-1,2-diol | AURORA-A |
| | FLT-3 |
| | CHEK2 |
| 3-(phenethylthio)-5-(3-(trifluoromethyl)phenyl)-4H-1,2,4-triazole | AURORA-A |
| | GSK3-α |
| 2-(2-(5-(3-(trifluoromethyl)phenyl)-4H-1,2,4-triazol-3- | GSK3-β |
| ylthio)ethyl)isoindoline-1,3-dione | PKA |
| | GSK3-α |
| 1,1,1-trifluoro-3-(5-(3-(trifluoromethyl)phenyl)-4H-1,2,4-triazol-3- | AURORA-A |
| ylthio)propan-2-ol | FLT-3 |
| | GSK3-β |
| N,N-dimethyl-3-(5-(3-(trifluoromethyl)phenyl)-4H-1,2,4-triazol N-3- | CHEK2 |
| ylthio)propan-1-amine | c-TAK1 |
| | GSK3-β |
| ethyl 3-(5-(3-(trifluoromethyl)phenyl)-4H-1,2,4-triazol-3-ylthio)propanoate | AURORA-A |
| | FLT-3 |
| | GSK3-β |
| 3-(butylthio)-5-((4-chlorophenoxy)methyl)-4H-1,2,4-triazole | FLT-3 |
| 3-(butylthio)-5-(4-nitrophenyl)-4H-1,2,4-triazole | AURORA-A |
| | GSK3-α |
| 3-(5-(4-nitrophenyl)-4H-1,2,4-triazol-3-ylthio)propane-1,2-diol | AURORA-A |
| | FLT-3 |
| | DYRK2 |
| 2-(2-(5-(4-nitrophenyl)-4H-1,2,4-triazol-3-ylthio)ethyl)isoindoline-1,3-dione | GSK3-α |
| 3-(2-ethoxyethylthio)-5-(4-nitrophenyl)-4H-1,2,4-tnazole | FLT-3 |
| | AURORA-A |
| | GSK3-α |
| N,N-dimethyl-3-(5-(4-nitrophenyl)-4H-1,2,4-triazol-3-ylthio)propan-1-amine | c-TAK1 |
| 3-(butylthio)-5-(3 ,4-dichlorophenyl)-4H-1,2,4-triazole | AURORA-A |
| | FLT-3 |
| | GSK3-α |
| 2-(5-(3,4-dichlorophenyl)-4H-1,2,4-triazol-3-ylthio)ethanol | AURORA-A |
| | FLT-3 |
| | CHEK2 |
| 3-(3,4-dichlorophenyl)-5-(2-ethoxyethylthio)-4H-1,2,4-triazole | AURORA-A |
| | GSK3-β |
| | GSK3-α |
| 3-(5-(3,4-dichlorophenyl)-4H-1,2,4-triazol-3-ylthio)-1,1,1-trifluoropropan-2- | AURORA-A |
| ol | FLT-3 |
| | GSK3-β |
| 3-(5-(4-(trifluoromethoxy)phenyl)-4H-1,2,4-triazol-3-ylthio)propane- | AURORA-A |
| 1,2-diol | FLT-3 |
| | DYRK2 |
| 3-(2-ethoxyethylthio)-5-(4-(trifluoromethoxy)phenyl)-4H-1,2,4-triazole | AURORA-A |
| | GSK3-β |
| N,N-dimethyl-3-(5-(4-(trifluoromethoxy)phenyl)-4H-1,2,4-triazol-3- | c-TAK1 |
| ylthio)propan-1-amine | AURORA-A |
| | CHEK2 |
| 3-(5-tert-butyl-4H-1,2,4-triazol-3-ylthio)-N,N-dimethylpropan-1-amine | c-TAK1 |
| 3-methyl-5-(phenethylthio)-4H-1,2,4-triazole | AURORA-A |
| | GSK3-β |
| 3-(5-(3-chlorophenyl)-4H-1,2,4-triazol-3-ylthio)propane-1,2-diol | AURORA-A |
| | FLT-3 |
| | CHEK2 |
| 3-(5-(4-methoxyphenyl)-4H-1,2,4-triazol-3-ylthio)propane-1,2-diol | AURORA-A |
| | FLT-3 |
| | DYRK2 |
| 3-(butylthio)-5-(4-fluorophenyl)-4H-1,2,4-triazole | AURORA-A |
| | FLT-3 |
| | DYRK2 |
| 3-(biphenyl-4-yl)-5-(phenethylthio)-4H-1,2,4-triazole | AURORA-A |
| 2-(5-(thiophen-2-yl)-4H-1,2,4-triazol-3-ylthio)ethanol | DYRK2 |
| | FLT-3 |
| | AURORA-A |
| 3-(phenethylthio)-5-(thiophen-2-yl)-4H-1,2,4-triazole | AURORA-A |
| | FLT-3 |
| 2-(2-(5-(thiophen-2-yl)-4H-1,2,4-triazol-3-ylthio)ethyl)isoindoline-1,3-dione | AURORA-A |
| | GSK3-α |
| | DYRK2 |
| 3-(2-ethoxyethylthio)-5-(thiophen-2-yl)-4H-1,2,4-triazole | AURORA-A |
| | DYRK2 |
| | GSK3-β |
| 1,1,1-trifluoro-3-(5-(thiophen-2-yl)-4H-1,2,4-triazol-3-ylthio)propan-2-ol | AURORA-A |
| | DYRK2 |
| N,N-dimethyl-3-(5-(thiophen-2-yl)-4H-1,2,4-triazol-3-ylthio)propan-1-amine | c-TAK1 |
| | AURORA-A |
| | GSK3-α |
| 1,1,1-trifluoro-3-(5-(2-(methylthio)ethyl)-4H-1,2,4-triazol-3-ylthio)propan-2- | GSK3-α |
| ol | |
| N-(5-cyclopentyl-4H-1,2,4-triazol-3-ylthio)-N,N-dimethylpropan-1-amine | c-TAK1 |
| N-(5-phenethyl-4H-1,2,4-triazol-3-ylthio)propane-1,2-diol | GSK3-α |
| N-(2-(5-(thiophen-2-yl)-4H-1,2,4-triazol-3-ylthio)ethyl)benzamide | DYRK2 |
| | GSK3-α |
| | GSK3-β |
| 3-(5-cyclohexyl-4-(2-methoxyphenyl)-4H-1,2,4-triazol-3-ylthio)propanoic | GSK3-α |
| acid | |
| benzyl 4-(5-cyclohexyl-4-(2-methoxyphenyl)-4H-1,2,4-triazol-3- | AURORA-A |
| ylthio)butanoate | P70S6K1 |
| | CDK1 |
| benzyl 3-(4-(2-methoxyphenyl)-5-phenyl-4H-1,2,4-triazol-3- | p38-α |
| ylthio)propanoate | AURORA-A |
| 4-(4-(2-methoxyphenyl)-5-phenyl-4H-1,2,4-triazol-3-ylthio)butanoic acid | c-TAK1 |
| benzyl 4-(4-(2-methoxyphenyl)-5-phenyl-4H-1,2,4-triazol-3-ylthio)butanoate | AURORA-A |
| 4-(4-(2-methoxyphenyl)-5-phenyl-4H-1,2,4-triazol-3-ylthio)butanenitrile | p38-α |
| | p38-γ |
| 5-(3-(4-(2-methoxyphenyl)-5-phenyl-4H-1,2,4-triazol-3-ylthio)propyl)-2H- | P70S6K1 |
| tetrazole | AURORA-A |
| benzyl 3-(5-cyclohexyl-4-(2-methoxyphenyl)-4H-1,2,4-triazol-3- | AURORA-A |
| ylthio)propanoate | |
| 3-(3-chlorophenyl)-4-(2-methoxyphenyl)-5-(3-phenylpropylthio)-4H-1,2,4- | p38-α |
| triazole | p38-β |
| | MAPKAPK-3 |
| 4-(2-methoxyphenyl)-3-phenyl-5-(4-phenylbutylthio)-4H-1,2,4-triazole | MAPKAPK-3 |
| | p38-α |
| | p38-β |
| 3-cyclohexyl-4-(2-methoxyphenyl)-5-(4-phenylbutylthio)-4H-1,2,4-triazole | MAPKAPK-3 |
| | p38-α |
| 3-(3-chlorophenyl)-4-(2-methoxyphenyl)-5-(4-phenylbutylthio)-4H-1,2,4- | MAPKAPK-3 |
| triazole | p38-α |
| 3-cyclohexyl-4-(2-methoxyphenyl)-5-(3-phenylpropylthio)-4H-1,2,4-triazole | p38-α |
| 4-(2-methoxyphenyl)-3-phenyl-5-(3-phenylpropylthio)-4H-1,2,4-triazole | p38-α |
| | p38-β |
| 3-(3-chlorophenyl)-4-(2-methoxyphenyl)-5-(5-phenylpentylthio)-4H-1,2,4- | MAPKAPK-3 |
| triazole | p38-α |
| 3-(4-nitrobenzylthio)-4H-1,2,4-triazole | MSK2 |
| | SRC |
| 3-(4-benzyl-5-(2-chlorobenzylthio)-4H-1,2,4-triazol-3-yl)pyridine | p38-α |
| | p38-β |
| N-((5-(benzylthio)-4-ethyl-4H-1,2,4-triazol-3-yl)methyl)-4- | p38-α |
| fluorobenzenamine | |
| N-((5-(benzylthio)-4-(4-methoxyphenyl)-4H-1,2,4-triazol-3-yl)methyl)-4- | FLT-3 |
| methylbenzenamine | PDGFRR-α |
| 3-benzyl-5-(benzylthio)-4-ethyl-4H-1,2,4-triazole | p38-α |
| M-((5-(benzylthio)-4-ethyl-4H-1,2,4-triazol-3-yl)methyl)-4- | p38-α |
| chlorobenzenamine | AKT1 |
| 3-((4-(4-fluorophenyl)-5-(pyridin-4-yl)-4H-1,2,4-triazol-3- | p38-α |
| ylthio)methyl)pyridine | |
| 3-((4-(4-fluorophenyl)-5-(pyridin-4-yl)-4H-1,2,4-triazol-3- | MSK2 |
| ylthio)methyl)pyridine | |
| 3-(2-chiorobenzylthio)-4-phenyl-5-(3,4,5-trimethoxyphenyl)-4H-1,2,4- | CDK1 |
| triazole | p38-α |
| | MSK2 |
| 3-benzyl-5-(2-chlorobenzylthio)-4-methyl-4H-1,2,4-triazole | MAPKAPK-2 |
| | p38-α |
| 4-(5-(4-tert-butylbenzylthio)-4-ethyl-4H-1,2,4-triazol-3-yl)pyridine | MAPKAPK-2 |
| 2-chloro-N-((5-(4-chlorobenzylthio)-4-(4-methoxyphenyl)-4H-1,2,4-triazol- | MAPKALPK-2 |
| 3-yl)methyl)benzenamine | AKT1 |
| 4-(5-(2,4-dichlorobenzylthio)-4-(furan-2-ylmethyl)-4H-1,2,4-triazol-3- | MSK2 |
| yl)pyridine | MSK1 |
| | AKT3 |
| 3-(benzylthio)-5-(2-bromophenyl)-4-phenyl-4H-1,2,4-triazole | p38-α |
| | MSK2 |
| | CDK1 |
| 3-(5-(4-tert-butylbenzylthio)-4-methyl-4H-1,2,4-triazol-3-yl)pyridine | PDGFRR-α |
| 3-(benzylthio)-4-phenyl-5-(3,4,5-trimethoxyphenyl)-4H-1,2,4-triazole | p38-δ |
| 4-methyl-3-(4-methylbenzylthio)-5-o-tolyl-4H-1,2,4-triazole | AKT1 |
| 4-(5-(benzylthio)-4-p-tolyl-4H-1,2,4-triazol-3-yl)pyridine | AURORA-A |
| | p38-α |
| 4-(5-(4-methylbenzylthio)-4-phenyl-4H-1,2,4-triazol-3-yl)pyridine | AURORA-A |
| 2-((5-(2-hydroxyphenyl)-4-phenethyl-4H-1,2,4-triazol-3- | AKT1 |
| ylthio)methyl)quinazolin-4(3H)-one | |
| 3-(5-(4-chlorobenzylthio)-4-methyl-4H-1,2,4-triazol-3-yl)pyridine | CHEK2 |
| 4-(4-benzyl-5-(benzylthio)-4H-1,2,4-triazol-3-yl)phenol | GSK3-β |
| | GSK3-α |
| | LYNA |
| 3-(5-(benzylthio)-4-p-tolyl-4H-1,2,4-triazol-3-yl)pyridine | AURORA-A |
| 3-(4-chlorobenzylthio)-4-ethyl-5-((4-methoxyphenoxy)methyl)-4H-1,2,4- | CDK2-cyclinE |
| triazole | |
| 2-((4-(4-methoxyphenyl)-5-(pyridin-4-yl)-4H-1,2,4-triazol-3-ylthio)methyl)- | GSK3-α |
| 5-phenyl-1,3,4-oxadiazole | |
| 3-(2-chlorobenzylthio)-4-ethyl-5-(2-methoxyphenyl)-4H-1,2,4-triazole | p38-α |
| | MSK2 |
| 2,6-dimethyl-N-((4-methyl-5-(4-methylbenzylthio)-4H-1,2,4-triazol-3- | p38-γ |
| yl)methyl)benzenamine | |
| 3-(5-(4-tert-butylbenzylthio)-4-ethyl-4H-1,2,4-triazol-3-yl)pyridine | MAPKAPK-2 |
| 4-(5-(2-chlorobenzylthio)-4-ethyl-4H-1,2,4-triazol-3-yl)phenol | p38-α |
| | p38 -γ |
| 3-benzyl-5-(benzylthio)-4-(3 ,4-dimethoxyphenethyl)-4H-1,2,4-triazole | p38-α |
| | AKT1 |
| 4-(5-(2-chlorobenzylthio)-4-rnethyl-4H-1,2,4-triazol-3-yl)phenol | GSK3-β |
| | GSK3-α |
| | p38-α |
| 3-(2-chlorobenzylthio)-4-methyl-5-m-tolyl-4H-1,2,4-triazole | p38-γ |
| 3-(5-(benzylthio)-4-phenethyl-4H-1,2,4-triazol-3-yl)pyridine | MSK1 |
| 3-(4-tert-butylbenzylthi o)-4-(3-methoxypropyl)-5-methyl-4H-1,2,4-triazole | MAPKALPK-2 |
| | AKT1 |
| N-((5-(benzylthio)-4-ethyl-4H-1,2,4-triazol-3-yl)methyl)-2,4- | p38-α |
| dimethylbenzenamine | |
| 3-(5-(4-chiorobenzylthio)-4-ethyl-4H-1,2,4-triazol-3-yl)pyridine | p38-α |
| 3-(4-chlorophenyl)-5-(3,4-dichlorobenzylthio)-4-methyl-4H-1,2,4-triazole | PRAK |
| | FYN |
| 3-(4-chlorobenzylthio)-4-ethyl-5-((2-methoxyphenoxy)methyl)-4H- | CDK2-cyclinE |
| 1,2,4-triazole | |
| 4-(5-(4-chlorobenzylthio)-4-(furan-2-ylmethyl)-4H-1,2,4-triazol-3- | MSK1 |
| yl)pyridine | MSK2 |
| | AKT3 |
| 3-(4-tert-butylphenyl)-5-(2-chlorobenzylthio)-4-(3-methoxypropyl)-4H-1,2,4- | CDK1 |
| triazole | |
| N,N-diethyl-4-(4-ethyl-5-((5-phenyl-1,3 ,4-oxadiazol-2-yl)methylthio)-4H- | GSK3-α |
| 1,2,4-triazol-3-yl)benzenesulfonamide | |
| N-((5-(benzylthio)-4-(4-methoxyphenyl)-4H-1,2,4-triazol-3-yl)methyl)-4- | PDGFRR-α |
| methoxybenzenamine | FLT-3 |
| | FYN |
| M-((5-(2,6-dichlorobenzylthio)-4-phenyl-4H-1,2,4-triazol-3-yl)methyl)-4- | PDGFRR-α |
| methoxybenzenamine | FLT-3 |
| | FYN |
| 4-(5-(benzylthio)-4-(furan-2-ylmethyl)-4H-1,2,4-triazol-3-yl)phenol | GSK3-β |
| | GSK3-α |
| | MSK2 |
| 2-((5-(3,4-dimethoxyphenyl)-4-methyl-4H-1,2,4-triazol-3-ylthio)methyl)-5- | GSK3-α |
| phenyl-1,3,4-oxadiazole | CHEK2 |
| N-(4-ethoxyphenyl)-4-((4-ethyl-5-(pyridin-3-ylmethylthio)-4H-1,2,4-triazol- | CHEK2 |
| 3-yl)methyl)thiazol-2-amine | AURORA-A |
| N-((5-(3,4-dichlorobenzylthio)-4-ethyl-4H-1,2,4-triazol-3-yl)methyl)-4- | AURORA-A |
| methylbenzenamine | CDK2-cyclinE |
| 3-(4-methyl-5-((5-phenyl-1,3,4-oxadiazol-2-yl)methylthio)-4H-1,2,4-triazol- | GSK3-β |
| N-yl)naphthalen-2-ol | GSK3-α |
| N-((5-(4-chlorobenzylthio)-4-ethyl-4H-1,2,4-triazol-3-yl)methyl)-4- | PDGFRR-α |
| methoxybenzenamine | FYN( |
| 3-(benzylthio)-4-ethyl-5-(phenoxymethyl)-4H-1,2,4-triazole | p38-α |
| | CDK1 |
| | CDK2-cyclinE |
| 2-((5-(phenoxymethyl)-4-p-tolyl-4H-1,2,4-triazol-3-ylthio)methyl)-5-p-tolyl- | GSK3-α |
| 1,3,4-oxadiazole | |
| 4-(5-(benzylthio)-4-(3-chlorophenyl)-4H-1,2,4-triazol-3-yl)pyridine | GSK3-β |
| | AURORA-A |
| | CDK1 |
| 3-(3-(2-chlorobenzylthio)-4H-1,2,4-triazol-4-yl)pyridine | PDGFRR-α |
| 4-(5-(4-chlorobenzylthio)-4-phenyl-4H-1,2,4-triazol-3-yl)pyridine | AURORA-A |
| | PKA |
| | MSK1 |
| 3-(4-tert-butylphenyl)-5-(4-chlorobenzylthio)-4-(3-methoxypropyl)-4H-1,2,4- | DAPK1 |
| triazole | |
| 4-(5-(4-tert-butylbenzylthio)-4-(furan-2-ylmethyl)-4H-1,2,4-triazol-3- | MAPKAPK-2 |
| yl)pyridine | |
| 4-((5-(4-chlorophenyl)-4-p-tolyl-4H-1,2,4-triazol-3-ylthio)methyl)benzoic | SYK |
| acid | |
| 3-(5-(2-chlorobenzylthio)-4-phenyl-4H-1,2,4-triazol-3-yl)pyridine | p38-α |
| 4-(5-(benzylthio)-4-phenyl-4H-1,2,4-triazol-3-yl)pyridine | AURORA-A |
| 4-(4-(4-methyl-5-((5-p-tolyl-1,3,4-oxadiazol-2-yl)methylthio)-4H-1,2,4- | GSK3-β |
| triazol-3-yl)phenylsulfonyl)morpholine | GSK3-α |
| 3-(2-chlorobenzylthio)-4-ethyl-5-(phenoxymethyl)-4H-1,2,4-triazole | MAPKAPK-2 |
| 4-((4-methyl-5-(phenoxymethyl)-4H-1,2,4-triazol-3-ylthio)methyl)quinolm- | GSK3a |
| 2(1H)-one | |
| 2-((5-(benzylthio)-4-(furan-2-ylmethyl)-4H-1,2,4-triazol-3-yl)rnethylthio)- | CDK1 |
| 4,6-dimethylpyrimidine | |
| 3-(2-chlorobenzylthio)-4-ethyl-5-(phenoxymethyl)-4H-1,2,4-triazole | p38-α |
| | GSK3-α |
| | MSK2 |
| 4-((4-(furan-2-ylmethyl)-5-(pyridin-3-yl)-4H-1,2,4-triazol-3- | p38-β |
| ylthio)methyl)quinolin-2(1H)-one | |
| N-((5-(4-chlorobenzylthio)-4-phenyl-4H-1,2,4-triazol-3-yl)methyl)-4- | PDGFRR-α |
| methoxybenzenamine | FYN |
| 4-(5-(4-chlorobenzylthio)-4-ethyl-4H-1,2,4-triazol-3-yl)phenol | MSK2 |
| | GSK3-β |
| | MSK1 |
| 2-phenyl-5-((5-(pyridin-4-yl)-4-p-tolyl-4H-1,2,4-triazol-3-ylthio)methyl)- | GSK3-β |
| 1,3,4-oxadiazole | GSK3-α |
| 4-(5-(2,6-dichlorobenzylthio)-4-phenyl-4H-1,2,4-triazol-3-yl)pyridine | p38-α |
| | AKT3 |
| | HCK |
| 4-((4-(furan-2-ylmethyl)-5-(pyridin-4-yl)-4H-1,2,4-triazol-3- | GSK3-α |
| ylthio)methyl)benzoic acid | GSK3-β |
| 4-methoxy-N-((4-methyl-5-(4-nitrobenzylthio)-4H-1,2,4-triazol-3- | FYN |
| yl)methyl)benzenamine | |
| N-((5-(2-chloro-6-fluorobenzylthio)-4-ethyl-4H-1,2,4-triazol-3-yl)methyl)-4- | PDGFRR-α |
| methoxybenzenamine | FYN |
| | FLT-3 |
| 4-(4-(5-(2-chlorobenzylthio)-4-ethyl-4H-1,2,4-triazol-3- | p38-α |
| yl)phenylsulfonyl)morpholine | MSK2 |
| N-((5-(4-chlorobenzylthio)-4-phenyl-4H-1,2,4-triazol-3-yl)methyl)-3- | PDGFRR-α |
| methylbenzenamine | MSK2 |
| 3-(benzylthio)-5-(4-methoxyphenyl)-4-phenyl-4H-1,2,4-triazole | INSR |
| | SYK |
| | PAK2 |
| N-(4-(5-(benzylthio)-4-(4-methoxyphenyl)-4H-1,2,4-triazol-3-yl)phenyl)-4- | INSR |
| methylbenzenesulfonamide | p38-α |
| N-((5-(benzylthio)-4-ethyl-4H-1,2,4-triazol-3-yl)methyl)-2- | p38-α |
| chlorobenzenamine | |
| 3-(4-tert-butylbenzylthio)-5-((4-chlorophenoxy)methyl)-4-phenyl-4H-1,2,4- | SYK |
| triazole | |
| 3-benzyl-5-(4-chlorobenzylthio)-4-ethyl-4H-1,2,4-triazole | p38-α |
| | CDK2-cyclinE |
| 4-benzyl-3-(2-chlorobenzylthio)-5-cyclohexyl-4H-1,2,4-triazole | MSK2 |
| | p38-α |
| | CDK1 |
| 4-(5-(4-tert-butylbenzylthio)-4-(3-methoxypropyl)-4H-1,2,4-triazol-3- | DYRK2 |
| yl)pyridine | |
| 3-(benzylthio)-4-ethyl-5-(4-methoxyphenyl)-4H-1,2,4-triazole | p38-α |
| 4-ethyl-3-(4-methylbenzylthio)-5-(p-tolyloxymethyl)-4H-1,2,4-triazole | AURORA-A |
| N-((5-(2-chloro-6-fluorobenzylthio)-4-ethyl-4H-1,2,4-triazol-3-yl)methyl)- | p38-α |
| 2,4-dimethylbenzenamine | |
| 2,4-dimethyl-N-((5-((5-phenyl-1,3,4-oxadiazol-2-yl)methylthio)-4- | SYK |
| ((tetrahydrofuran-2-yl)methyl)-4H-1,2,4-triazol-3-yl)methyl)benzenamine | |
| N-((4-(furan-2-ylmethyl)-5-((5-phenyl-1,3,4-oxadiazol-2-yl)methylthio)-4H- | AURORA-A |
| 1,2,4-triazol-3-yl)methyl)-2,4-dimethylbenzenamine | |
| N-((4-(furan-2-ylmethyl)-5-(4-nitrobenzylthio)-4H-1,2,4-triazol-3- | LYNA |
| yl)methylthio)-4,6-dimethylpyrimidine | |
| 2-((4-(furan-2-ylmethyl)-5-(4-methylbenzylthio)-4H-1,2,4-triazol-3- | CDK2-cyclinA |
| yl)methylthio)-4,6-dimethylpyrimidine | |
| 3-(benzylthio)-5-((4-chlorophenoxy)methyl)-4-phenyl-4H-1,2,4-triazole | p38-α |
| 3-(4-chlorobenzylthio)-5-((naphthalen-1-yloxy)methyl)-4-phenyl-4H-1,2,4- | p38-α |
| triazole | |
| 4-(5-(4-chlorobenzylthio)-4-ethyl-4H-1,2,4-triazol-3-yl)pyridine | PKA |
| | AURORA-A |
| | MSK2 |
| 4-(4-(5-(benzylthio)-4-ethyl-4H-1,2,4-triazol-3-yl)phenylsulfonyl) | p38-α |
| morpholine | SYK |
| N-((5-(4-tert-butylbenzylthio)-4-p-tolyl-4H-1,2,4-triazol-3-yl)methyl)-4- | AURORA-A |
| methylbenzenamine | SYK |
| 3-(2-chlorobenzylthio)-4-phenyl-5-(p-tolyloxymethyl)-4H-1,2,4-triazole | p38-α |
| 3-(benzylthio)-5-((naphthalen-1-yloxy)methyl)-4-phenyl-4H-1,2,4-triazole | p38-α |
| N-((5-(2-chlorobenzylthio)-4-ethyl-4H-1,2,4-triazol-3-yl)methyl)-4- | p38-α |
| methylbenzenamine | |
| 3-(benzylthio)-5-((4-bromophenoxy)methyl)-4-ethyl-4H-1,2,4-triazole | p38-α |
| 3-(benzylthio)-5-((2-methoxyphenoxy)methyl)-4-phenyl-4H-1,2,4-triazole | p38-α |
| | AURORA-A |
| 2-(4-benzyl-5-(4-fluorobenzylthio)-4H-1,2,4-triazol-3-yl)phenol | AURORA-A |
| 4-((4-ethyl-5-(3-hydroxynaphthalen-2-yl)-4H-1,2,4-triazol-3- | AURORA-A |
| ylthio)methyl)quinolin-2(1H)-one | |
| 3-(4-chlorobenzylthio)-5-(3,4-dimethoxyphenyl)-4-phenyl-4H-1,2,4-triazole | CDK1 |
| N-((5-(benzylthio)-4-methyl-4H-1,2,4-triazol-3-yl)methyl)-4- | AURORA-A |
| methylbenzenamine | |
| 4-(5-(4-chlorobenzylthio)-4-methyl-4H-1,2,4-triazol-3-yl)pyridine | AURORA-A |
| | MSK1 |
| 4-(5-(benzylthio)-4-methyl-4H-1,2,4-triazol-3-yl)pyridine | AURORA-A |
| 2-(4-nitrophenyl)-5-((4-(pyridin-2-yl)-4H-1,2,4-triazol-3-ylthio)methyl)- | PDGFRR-α |
| 1,3,4-oxadiazole | |
| 4-(5-(benzylthio)-4-(4-methoxyphenyl)-4H-1,2,4-triazol-3-yl)pyridine | AURORA-A |
| | AKT3 |
| | p38-α |
| 4-(5-(benzylthio)-4-methyl-4H-1,2,4-triazol-3-yl)phenol | GSK3-β |
| | GSK3-α |
| | MSK1 |
| 4-(5-(benzylthio)-4-(4-fluorophenyl)-4H-1,2,4-triazol-3-yl)pyridine | AURORA-A |
| | p38-α |
| | AKT3 |
| 4-(4-methyl-5-(pyridin-2-ylmethylthio)-4H-1,2,4-triazol-3-yl)phenol | GSK3-β |
| | GSK3-α |
| 4-((5-(2-chloro-6-fluorobenzylthio)-4-phenyl-4H-1,2,4-triazol-3- | AKT3 |
| yl)methoxy)benzonitrile | p38-α |
| 3-(2-chlorobenzylthio)-5-(phenoxymethyl)-4-phenyl-4H-1,2,4-triazole | p38-α |
| N-((5-(2,6-dichlorobenzylthio)-4-methyl-4H-1,2,4-triazol-3-yl)methyl)-4- | GSK3-β |
| fluorobenzenamine | GSK3-α |
| 2-chloro-N-((5-(2-chlorobenzylthio)-4-(4-methoxyphenyl)-4H-1,2,4-triazol- | CDK1 |
| 3-yl)methyl)-5-(trifluoromethyl)benzenamine | |
| 4-benzyl-3-(benzylthio)-5-(phenoxymethyl)-4H-1,2,4-triazole | p38-α |
| N-((5-(2-chlorobenzylthio)-4-phenyl-4H-1,2,4-triazol-3-yl)methyl)-2-methyl- | p38-α |
| 5-nitrobenzenamine | |
| 3-(benzylthio)-4-ethyl-5-(o-tolyloxymethyl)-4H-1,2,4-triazole | p38-α |
| N-(3-chlorophenyl)-N-((5-(2,4-dichlorobenzylthio)-4-phenyl-4H-1,2,4- | LCK |
| triazol-3-yl)methyl)-4-methylbenzenesulfonamide | |
| 3-chloro-N-((5-(2,4-dichlorobenzylthio)-4-phenyl-4H-1,2,4-triazol-3- | LCK |
| yl)methyl)benzenamine | |
| 3-chloro-N-((5-(2-chlorobenzylthio)-4-phenyl-4H-1,2,4-triazol-3- | LCK |
| yl)methyl)benzenamine | |
| 4-(4-ethyl-S N-(4-methylbenzylthio)-4H-1,2,4-triazol-3-yl)pyridine | PKA |
| | p38-γ |
| 3,3′-(2,5-dimethoxy-1,4-phenylene)bis(methylene)bis(sulfanediyl)bis(4H- | CSK |
| 1,2,4-triazole) | LCK |
| 4-(5-(2-chloro-6-fluorobenzylthio)-4-methyl-4H-1,2,4-triazol-3-yl)pyridine | GSK3-β |
| | AURORA-A |
| 2-(3,4-dichlorobenzyl)-4-((4-methyl-4H-1,2,4-triazol-3- | GSK3-α |
| ylthio)methyl)thiazole | |
| 3-(2-chlorobenzylthio)-5-(4-methoxyphenyl)-4-phenyl-4H-1,2,4-triazole | p38-α |
| | P70S6K1 |
| 3-(3-chlorobenzylthio)-5-(4-methoxyphenyl)4-phenyl-4H-1,2,4-triazole | p38-α |
| 2-(5-(benzylthio)-4-(2-methoxyphenyl)-4H-1,2,4-triazol-3-yl)phenol | |
| 2-(5-(4-chlorobenzylthio)-4-(2-methoxyphenyl)-4H-1,2,4-triazol-3-yl)phenol | p38-α |
| | P70S6K1 |
| 2-(5-(2-chlorobenzylthio)-4-(2-methoxyphenyl)-4H-1,2,4-triazol-3-yl)phenol | CDK2-cyclinA |
| | PRAK |
| 2-(5-(3-chlorobenzylthio)-4-(2-methoxyphenyl)-4H-1,2,4-triazol-3-yl)phenol | p38-β |
| ethyl 5-((5-(4-methoxybenzyl)-4H-1,2,4-triazol-3-ylthio)methyl)fiiran-2- | FLT-3 |
| carboxylate | |
| 3-((5-(4-methoxyphenyl)-4H-1,2,4-triazol-3-ylthio)methyl)pyridine | AURORA-A |
| | FLT-3 |
| | DYRK2 |
| 3-(4-fluorobenzylthio)-5-(thiophen-2-yl)-4H-1,2,4-triazole | AURORA-A |
| | FLT-3 |
| | DYRK2 |
| 3-(benzylthio)-4-ethyl-5-methyl-4H-1,2,4-triazole | GSK3-α |
| 3-(benzylthio)-4-(furan-2-ylmethyl)-5-methyl-4H-1,2,4-triazole | GSK3-~ |
| | GSK3-α |
| 4-benzyl-3-(benzylthio)-5-methyl-4H-1,2,4-triazole | GSK3-β |
| | GSK3-α |
| 3-(benzylthio)-5-methyl-4-phenethyl-4H-1,2,4-triazole | GSK3-β |
| | GSK3-α |
| | c-TAK1 |
| 3-(benzylthio)-4-(3,4-dichlorobenzyl)-5-methyl-4H-1,2,4-triazole | GSK3-α |
| | GSK3-β |
| | c-TAK1 |
| 4-(benzo[d][1,3]dioxol-5-ylmethyl)-3-(benzylthio)-5-ethyl-4H-1,2,4-triazole | GSK3-β |
| | GSK3-α |
| 4-(benzylthio)-4-(3,4-dichlorobenzyl)-5-ethyl-4H-1,2,4-triazole | GSK3-α |
| | GSK3-β |
| | AURORA-A |
| 3-benzyl-5-(benzylthio)-4-phenethyl-4H-1,2,4-triazole | AURORA-A |
| 3-benzyl-5-(benzylthio)-4-(4-methylphenethyl)-4H-1,2,4-triazole | FLT-3 |
| | PRAK |
| | AURORA-A |
| 3-benzyl-5-(benzylthio)-4-(3,4-dichlorobenzyl)-4H-1,2,4-triazole | AURORA-A |
| 3-((3-(benzylthio)-5-(2-cyclopentylethyl)-4H-1,2,4-triazol-4- | GSK3-α |
| yl)methyl)pyridine | |
| 3-(benzylthio)-5-(2-cyclopentylethyl)-4-(4-methylphenethyl)-4H-1,2,4- | AURORA-A |
| triazole | c-TAK1 |
| 3-(benzylthio)-5-(2-cyclopentylethyl)-4-(3,4-dichlorobenzyl)-4H-1,2,4- | GSK3-β |
| triazole | AURORA-A |
| | c-TAK1 |
| 3-(benzylthio)-5-cyclohexyl-4-(3,4-dichlorobenzyl)-4H-1,2,4-triazole | c-TAK1 |
| 3-(benzylthio)-4-(4-methylphenethyl)-5-(1-phenylpropyl)-4H-1,2,4-triazole | c-TAK1 |
| 3-(benzylthio)-4-(3,4-dichlorobenzyl)-5-isobutyl-4H-1,2,4-triazole | AURORA-A |
| 3-(benzylthio)-5-(but-3-enyl)-4-(3,4-dichlorobenzyl)-4H-1,2,4-triazole | AURORA-A |
| | c-TAK1 |
| 2-((5-phenyl-4H-1,2,4-triazol-3-ylthio)methyl)benzonitrile | AURORA-A |
| | CHEK2 |
| | GSK3-β |
| ethyl 5-((5-phenyl-4H-1,2,4-triazol-3-ylthio)methyl)furan-2-carboxylate | AURORA-A |
| | FLT-3 |
| | GSK3-β |
| 3-(3-iodobenzylthio)-5-phenyl-4H-1,2,4-triazole | AURORA-A |
| | c-TAK1 |
| 4-((5-(pyridin-3-yl)-4H-1,2,4-triazol-3-ylthio)methyl)benzoic acid | AURORA-A |
| | DYRK2 |
| 2-((5-(pyridin-3-yl)-4H-1,2,4-triazol-3-ylthio)methyl)benzonitrile | AURORA-A |
| | GSK3-α |
| | CHEK2 |
| 4-((5-m-tolyl-4H-1,2,4-triazol-3-ylthio)methyl)benzoic acid | FLT-3 |
| | DYRK2 |
| | AURORA-A |
| methyl 4-((5-m-tolyl-4H-1,2,4-triazol-3-ylthio)methyl)benzoate | AURORA-A |
| | FLT-3 |
| | c-TAK1 |
| 3-(naphthalen-1-ylmethylthio)-5-m-tolyl-4H-1,2,4-triazole | AURORA-A |
| ethyl 5-((5-m-tolyl-4H-1,2,4-triazol-3-ylthio)methyl)furan-2-carboxylate | AURORA-A |
| | FLT-3 |
| | CHEK2 |
| 3-(3-iodobenzylthio)-5-(3-(trifluoromethyl)phenyl)-4H-1,2,4-triazole | CHEK2 |
| 3-((5-((4-chlorophenoxy)methyl)-4H-1,2,4-triazol-3-ylthio)methyl)pyridine | FLT-3 |
| 4-((5-(4-nitrophenyl)-4H-1,2,4-triazol-3-ylthio)methyl)pyridine | FLT-3 |
| | GSK3-β |
| | GSK3-α |
| ethyl 5-((5-(4-nitrophenyl)-4H-1,2,4-triazol-3-ylthio)methyl)furan-2- | AURORA-A |
| carboxylate | |
| 3-(4-methoxybenzyl)-5-(naphthalen-1-ylmethylthio)-4H-1,2,4-triazole | AURORA-A |
| 3-benzyl-5-(4-methylbenzylthio)-4H-1,2,4-triazole | AURORA-A |
| 4-((5-(3,4-dichlorophenyl)-4H-1,2,4-triazol-3-ylthio)methyl)pyridine | FLT-3 |
| | GSK3-β |
| 4-((5-(4-(trifluoromethoxy)phenyl)-4H-1,2,4-triazol-3-ylthio)methyl)benzoic | FLT-3 |
| acid | AURORA-A |
| | DYRK2 |
| 2-((5-(4-(trifluoromethoxy)phenyl)-4H-1,2,4-triazol-3- | AURORA-A |
| ylthio)methyl)benzonitrile | |
| 3-(4-fluorobenzylthio)-5-(4-(trifluoromethoxy)phenyl)-4H-1,2,4-triazole | FLT-3 |
| | AURORA-A |
| 4-((5-(4-(trifluoromethoxy)phenyl)-4H-1,2,4-triazol-3-ylthio)methyl)pyridine | FLT-3 |
| | c-TAK1 |
| | GSK3-β |
| ethyl 5-((5-(4-(trifluoromethoxy)phenyl)-4H-1,2,4-triazol-3- | FLT-3 |
| ylthio)methyl)furan-2-carboxylate | GSK3-β |
| 4-((5-tert-butyl-4H-1,2,4-triazol-3-ylthio)methyl)pyridine | GSK3-α |
| 2-((5-methyl-4H-1,2,4-triazol-3-ylthio)methyl)benzonitrile | AURORA-A |
| | GSK3-β |
| | GSK3-α |
| methyl 4-((5-methyl-4H-1,2,4-triazol-3-ylthio)methyl)benzoate | DYRK2 |
| | AURORA-A |
| 3-methyl-5-(naphthalen-1-ylmethylthio)-4H-1,2,4-triazole | AURORA-A |
| | DYRK2 |
| | GSK3-α |
| 3-(benzylthio)-5-methyl-4H-1,2,4-triazole | AURORA-A |
| | DYRK2 |
| 2-((5-methyl-4H-1,2,4-triazol-3-ylthio)methyl)-1H-benzo[d]imidazole | DYRK2 |
| | FLT-3 |
| | AURORA-A |
| 3-(3-iodobenzylthio)-5-methyl-4H-1,2,4-triazole | AURORA-A |
| | GSK3-α |
| | DYRK2 |
| 4-((5-(3-chlorophenyl)-4H-1,2,4-triazol-3-ylthio)methyl)benzoic acid | FLT-3 |
| | DYRK2 |
| | AURORA-A |
| 3-(benzylthio)-5-(3-chlorophenyl)-4H-1,2,4-triazole | AURORA-A |
| | c-TAK1 |
| | FLT-3 |
| 2-((5-(4-methoxyphenyl)-4H-1,2,4-triazol-3-ylthio)methyl)benzonitrile | AURORA-A |
| | CHEK2 |
| | GSK3-α |
| 4-((5-(4-methoxyphenyl)-4H-1,2,4-triazol-3-ylthio)methyl)pyridine | FLT-3 |
| | DYRK2 |
| | GSK3-α |
| 3-(4-methoxyphenyl)-5-(4-(methylsulfonyl)benzylthio)-4H-1,2,4-triazole | FLT-3 |
| 3-(3-iodobenzylthio)-5-(4-methoxyphenyl)-4H-1,2,4-triazole | AURORA-A |
| 4-((5-(1-phenylpropyl)-4H-1,2,4-triazol-3-ylthio)methyl)benzoic acid | AURORA-A |
| methyl 4-((5-(1-phenylpropyl)-4H-1,2,4-triazol-3-ylthio)methyl)benzoate | FLT-3 |
| 3-(naphthalen-1-ylmethylthio)-5-(1-phenylpropyl)-4H-1,2,4-triazole | AURORA-A |
| 4-((5-(thiophen-2-yl)-4H-1,2,4-triazol-3-ylthio)methyl)benzoic acid | DYRK2 |
| | AURORA-A |
| | FLT-3 |
| 4-((5-(thiophen-2-yl)-4H-1,2,4-triazol-3-ylthio)methyl)pyridine | DYRK2 |
| | AURORA-A |
| | FLT-3 |
| 3-((5-(thiophen-2-yl)-4H-1,2,4-trazol-3-ylthio)methyl)benzonitrile | AURORA-A |
| | GSK3-β |
| | GSK3-α |
| 2-((5-(thiophen-2-yl)-4H-1,2,4-triazol-3-ylthio)methyl)benzonitrile | AURORA-A |
| | CHEK2 |
| | GSK3-α |
| methyl 4-((5-(thiophen-2-yl)-4H-1,2,4-triazol-3-ylthio)methyl)benzoate | AURORA-A |
| | FLT-3 |
| | c-TAK1 |
| 3-(3,4-dichlorophenyl)-5-(4-(methylsulfonyl)benzylthio)-4H-1,2,4-triazole | AURORA-A |
| | FLT-3 |
| 2-((5-cyclopentyl-4H-1,2,4-triazol-3-ylthio)methyl)-1H-benzo[d]imidazole | GSK3-α |
| | AURORA-A |
| | DYRK2 |
| 4-(5-(2-bromobenzylthio)-4-ethyl-4H-1,2,4-triazol-3-yl)-2-phenylquinoline | CHEK2 |
| 4-(5-(2-chlorobenzylthio)-4-ethyl-4H-1,2,4-triazol-3-yl)-2-phenylquinoline | CHEK2 |
| 4-(5-(2,4-difluorobenzylthio)-4-methyl-4H-1,2,4-triazol-3-yl)-6-ethoxy-2-(4- | CHEK2 |
| methoxyphenyl)quinoline | |
| 4-(4-((4-ethyl-5-(2-phenylquinolin-4-yl)-4H-1,2,4-triazol-3- | c-TAK1 |
| ylthio)methyl)phenyl)-1,2,3-thiadiazole | |
| 6-ethoxy-4-(5-(4-fluoro-2-(trifluoromethyl)benzylthio)-4-methyl-4H-1,2,4- | AURORA-A |
| triazol-3-yl)-2-(4-methoxyphenyl)quinoline | |
| 4-(5-(2-(difluoromethoxy)benzylthio)-4-(furan-2-ylmethyl)-4H-1,2,4-triazol- | c-TAK1 |
| 3-yl)-2-phenylquinoline | |
| 5-((5-cyclohexyl-4-(2-methoxyphenyl)-4H-1,2,4-triazol-3-ylthio)methyl)-2H- | AURORA-A |
| tetrazole | P70S6K1 |
| 5-benzyl-2-((5-(3-chlorophenyl)-4-(2-methoxyphenyl)-4H-1,2,4-triazol-3- | p38-α |
| ylthio)methyl)oxazole | |
| 5-benzyl-2-((5-(4-methoxyphenyl)-4-(3-(trifluoromethyl)phenyl)-4H-1,2,4- | p38-α |
| triazol-3-ylthio)methyl)oxazole | |
| 2-((5-(3-chlorophenyl)-4-(2-methoxyphenyl)-4H-1,2,4-triazol-3- | p38-α |
| ylthio)methyl)-5-phenethyloxazole | |
| N-((4-(3-chlorophenyl)-5-(2-piperidin-1-yl)ethylthio)-4H-1,2,4-triazol-3- | FYN |
| yl)methyl)-4-methoxybenzenamine | PDGFRR-α |
| | FLT-3 |
| 4-ethoxy-N-((5-(2-morpholinoethylthio)-4-p-tolyl-4H-1,2,4-triazol-3- | FYN |
| yl)methyl)benzenamine | |
| 4-fluoro-N-((4-methyl-5-(2-morpholinoethylthio)-4H-1,2,4-triazol-3- | PDGFRR-α |
| yl)methyl)benzenamine | |
| 4-ethoxy-N-((5-(2-morpholinoethylthio)-4-phenyl-4H-1,2,4-triazol-3- | FYN |
| yl)methyl)benzenamine | |
| N-((4-(3-chlorophenyl)-5-(2-morpholinoethylthio)-4H-1,2,4-triazol-3- | FYN |
| yl)methyl)-4-ethoxybenzenamine | FLT-3 |
| | PDGFRR-α |
| N-((4-(4-methoxyphenyl)-5-(2-(piperidin-1-yl)ethylthio)-4H-1,2,4-triazol-3- | p38-γ |
| yl)methyl)-4-methylbenzenamine | |
| N-((5-(3-cyclohexylpropylthio)-4-methyl-4H-1,2,4-triazol-3-yl)methyl)-4- | PDGFRR-α |
| methoxybenzenamine | FYN |
| | FLT-3 |
| 4-ethoxy-N-((4-(4-methoxyphenyl)-5-(2-morpholinoethylthio)-4H-1,2,4- | FYN |
| triazol-3-yl)methyl)benzenamine | |
| 4-methoxy-N-((4-(4-methoxyphenyl)-5-(2-(piperidin-1-yl)ethylthio)-4H- | FYN |
| 1,2,4-triazol-3-yl)methyl)benzenamine | |
| 4-methoxy-N-((4-(2-methoxyphenyl)-5-(2-(piperidin-1-yl)ethylthio)-4H- | FYN |
| 1,2,4-triazol-3-yl)methyl)benzenamine | PDGFRR-α |
| | FLT-3 |
| 2-chloro-N-((4-ethyl-5-(2-(piperidin-1-yl)ethylthio)-4H-1,2,4-triazol-3- | MAPKAPK-2 |
| yl)methyl)benzenamine | |
| 4-(2-(4-methyl-5-m-tolyl-4H-1,2,4-triazol-3-ylthio)ethyl)morpholine | AKT1 |
| 4-(5-(2-morpholinoethylthio)-4-phenyl-4H-1,2,4-triazol-3-yl)phenol | GSK3-α |
| 3-methyl-5-(4-methyl-5-(2-(piperidin-1-yl)ethylthio)-4H-1,2,4-triazol-3-yl)- | GSK3-α |
| 1-phenyl-1H-thieno[2,3-c]pyrazole | CHEK2 |
| 1-(2-(4-ethyl-5-(4-nitrophenyl)-4H-1,2,4-triazol-3-ylthio)ethyl)piperidine | PDGFRR-α |
| 4-ethoxy-N-((5-(2-(piperidin-1-yl)ethylthio)-4-p-tolyl-4H-1,2,4-triazol-3- | FYN |
| yl)methyl)benzenamine | PDGFRR-α |
| 4-(4-(4-methoxyphenyl)-5-(2-(piperidin-1-yl)ethylthio)-4H-1,2,4-triazol-3- | p38-γ |
| yl)phenol | |
| 4-methoxy-N-((4-methyl-5-(2-(piperidin-1-yl)ethylthio)-4H-1,2,4-triazol-3- | FYN |
| yl)methyl)benzenamine | PDGFRR-α |
| 3-(2-bromophenyl)-5-(cyclohexylmethylthio)-4-phenyl-4H-1,2,4-triazole | p38-α |
| 4-ethoxy-N-((4-phenyl-5-(2-(piperidin-1-yl)ethylthio)-4H-1,2,4-triazol-3- | PDGFRR-α |
| yl)methyl)benzenantine | FYN |
| 3-(4-(2-methoxyethyl)-5-(2 N-morpholinoethylthio)-4H-1,2,4-triazol-3- | CHEK2 |
| yl)naphthalen-2-ol | |
| 4-fluoro-N-((5-(2-morpholinoethylthio)-4-phenyl-4H-1,2,4-triazol-3- | CDK2-cyclinA |
| yl)methyl)benzenamine | |
| 4-(4-(4-methoxyphenyl)-5-(2-morpholinoethylthio)-4H-1,2,4-triazol-3- | GSK3-α |
| yl)phenol | GSK3-β |
| 4-(2-(4-methyl-5-(3-methyl-1-phenyl-1H-thieno[2,3-c]pyrazol-5-yl)-4H- | GSK3-α |
| 1,2,4-triazol-3-ylthio)ethyl)morpholine | GSK3-β |
| | DAPK1 |
| 3-chloro-N-((5-(2-morpholinoethylthio)-4-phenethyl-4H-1,2,4-triazol-3- | p38-α |
| yl)methyl)benzenamine | |
| 2-((4-(4-methoxyphenyl)-5-(2-(piperidin-1-yl)ethylthio)-4H-1,2,4-triazol-3- | p38-β |
| yl)methylthio)-4,6-dimethylpyrimidine | |
| 4-(2-(4-(4-methoxyphenyl)-5-(pyridin-3-yl)-4H-1,2,4-triazol-3- | p38-γ |
| ylthio)ethyl)morpholine | |
| 1-(2-(5-((4-chlorophenoxy)methyl)-4-ethyl-4H-1,2,4-triazol-3- | INSR |
| ylthio)ethyl)piperidine | PDGFRR-α |
| | CHEK1 |
| 4-(2-(4-(4-methoxyphenyl)-5-((naphthalen-1-yloxy)methyl)-4H-1,2,4-triazol- | p38-α |
| 3-ylthio)ethyl)morpholine | |
| 4-(2-(4-(4-methoxyphenyl)-5-((naphthalen-2-yloxy)methyl)-4H-1,2,4-triazol- | CDK2-cyclinE |
| 3-ylthio)ethyl)morpholine | |
| 4-(2-(4-benzyl-5-((4-chloro-3-methylphenoxy)methyl)-4H-1,2,4-triazol-3- | AURORA-A |
| ylthio)ethyl)morpholine | |
| 4-(2-(5-((4-chloro-3-methylphenoxy)methyl)-4-phenyl-4H-1,2,4-triazol-3- | p38-α |
| ylthio)ethyl)morpholine | AKT3 |
| 3-chloro-4-methyl-N-((5-(2-morpholinoethylthio)-4-((tetrahydrofuran-2- | p38-α |
| yl)methyl)-4H-1,2,4-triazol-3-yl)methyl)benzenamine | |
| 2-(2-(4-methyl-5-(2-(piperidin-1-yl)ethylthio)-4H-1,2,4-triazol-3-yl)ethyl)- | AURORA-A |
| 1H-benzo[d]imidazole | |
| 3-(4-(2-methoxyphenyl)-5-(2-(piperidin-1-yl)ethylthio)-4H-1,2,4-triazol-3- | AURORA-A |
| yl)pyridine | |
| 3-(4-(furan-2-ylmethyl)-5-(2-(piperidin-1-yl)ethylthio)-4H-1,2,4-triazol-3- | p38-α |
| yl)naphthalen-2-ol | FYN |
| 4-(2-(4-(4-methoxyphenyl)-5-(m-tolyloxymethyl)-4H-1,2,4-triazol-3- | p38-β |
| ylthio)ethyl)morpholine | |
| 4-(2-(5-(4-nitrophenyl)-4-phenyl-4H-1,2,4-triazol-3-ylthio)ethyl)morpholine | PDGFRR-α |
| 3-(cyclohexylmethylthio)-5-(3,4-dimethoxyphenyl)-4-phenyl-4H-1,2,4- | p38-α |
| triazole | CDK1 |
| 4-(4-(furan-2-ylmethyl)-5-(2-(piperidin-1-yl)ethylthio)-4H-1,2,4-triazol-3- | GSK3-α |
| yl)phenol | |
| 3-(cyclohexylmethylthio)-5-phenyl-4H-1,2,4-triazole | AURORA-A |
| | c-TAK1 |
| | CHEK2 |
| 3-phenyl-5-(2-(pyrrolidin-1-yl)ethylthio)-4H-1,2,4-triazole | GSK3-α |
| | GSK3-β |
| | CHEK2 |
| 3-(5-(2-(piperidm-1-yl)ethylthio)-4H-1,2,4-triazol-3-yl)pyridine | GSK3-α |
| 3-(5-(cyclopropylmethylthio)-4H-1,2,4-triazol-3-yl)pyridine | FLT-3 |
| | AURORA-A |
| 3-(5-((tetrahydrofuran-2-yl)methylthio)-4H-1,2,4-triazol-3-yl)pyridine | AURORA-A |
| 3-(cyclohexylmethylthio)-5-m-tolyl-4H-1,2,4-triazole | AURORA-A |
| | c-TAK1 |
| 4-(2-(5-m-tolyl-4H-1,2,4-triazol-3-ylthio)ethyl)morpholine | AURORA-A |
| | GSK3-β |
| | GSK3-α |
| 3-((tetrahydrofuran-2-yl)methylthio)-5-(3-(trifluoromethyl)phenyl)-4H-1,2,4- | AURORA-A |
| triazole | |
| 4-(2-(5-(4-nitrophenyl)-4H-1,2,4-triazol-3-ylthio)ethyl)morpholine | GSK3-β |
| | GSK3-α |
| | AURORA-A |
| 4-(2-(5-(3,4-dichlorophenyl)-4H-1,2,4-triazol-3-ylthio)ethyl)morpholine | AURORA-A |
| | GSK3-α |
| | GSK3-β |
| 3-(3,4-dichlorophenyl)-5-((tetrahydrofuran-2-yl)methylthio)-4H-1,2,4- | GSK3-β |
| | AURORA-A |
| 3-(cyclopropylmethylthio)-5-(4-(trifluoromethoxy)phenyl)-4H-1,2,4-triazole | FLT-3 |
| | AURORA-A |
| 3-(cyclohexylmethylthio)-5-methyl-4H-1,2,4-triazole | AURORA-A |
| | GSK3-α |
| 3-(cyclohexylmethylthio)-5-(4-methoxyphenyl)-4H-1,2,4-triazole | AURORA-A |
| | c-TAK1 |
| 4-(2-(5-(4-methoxyphenyl)-4H-1,2,4-triazol-3-ylthio)ethyl)morpholine | GSK3-α |
| | AURORA-A |
| | GSK3-β |
| 1-(2-(5-(4-methoxyphenyl)-4H-1,2,4-triazol-3-ylthio)ethyl)piperidine | GSK3-α |
| | DYRK2 |
| | GSK3-β |
| 3-(cyclopropylmethylthio)-5-(phenoxymethyl)-4H-1,2,4-triazole | GSK3-α |
| | GSK3-β |
| | AURORA-A |
| 3-(cyclohexylmethylthio)-5-(thiophen-2-yl)-4H-1,2,4-triazole | AURORA-A |
| | DYRK2 |
| 4-(2-(5-(thiophen-2-yl)-4H-1,2,4-triazol-3-ylthio)ethyl)morpholine | AURORA-A |
| | GSK3-β |
| | GSK3-α |
| 3-(2-(pyrrolidin-1-yl)ethylthio)-5-(thiophen-2-yl)-4H-1,2,4-triazole | GSK3-α |
| | GSK3-β |
| | DYRK2 |
| 2-(5-(cyclohexylmethylthio)-4-(2-methoxyphenyl)-4H-1,2,4-triazol-3- | p38-α |
| yl)phenol | P70S6K1 |
| 3-benzyl-4-(2-methoxyphenyl)-5-(4-phenylbutylthio)-4H-1,2,4-triazole | AURORA-A |
| | p38-α |
| 4-(2-methoxyphenyl)-3-(4-methoxyphenyl)-5-(4-phenylbutylthio)-4H-1,2,4- | p38-α |
| triazole | p38-β |
| | MAPKAPK-3 |
| 3-(4-fluorophenyl)-4-(2-methoxyphenyl)-5-(4-phenylbutylthio)-4H-1,2,4- | p38-α |
| triazole | p38-β |
| | MAPKAPK-3 |
| 2-(4-(2-methoxyphenyl)-5-(4-phenylbutylthio)-4H-1,2,4-triazol-3-yl)pyrazin | p38-α |
| | p38-β |
| 3-(4-(2-methoxyphenyl)-5-(4-phenylbutylthio)-4H-1,2,4-triazol-3-yl)pyridine | p38-α |
| | p38-β |
| | MAPKAPK-3 |
| 4-(4-(2-methoxyphenyl)-5-(4-phenylbutylthio)-4H-1,2,4-triazol-3- | p38-α |
| yl)benzenesulfonamide | p38-β |
| | MAPKALPK-3 |
| 4-(2-methoxyphenyl)-3-(4-phenylbutylthio)-5-(thiophen-2-yl)-4H-1,2,4- | p38-α |
| triazole | p38-β |
| | MAPKAPK-3 |
| 3-(2-chlorophenyl)-4-(2-methoxyphenyl)-5-(4-phenylbutylthio)-4H-1,2,4- | p38-α |
| triazole | p38-β |
| | MAPKAPK-3 |
| 3-(benzo[d][1,3]dioxol-5-yl)-4-(2-methoxyphenyl)-5-(4-phenylbutylthio)- | p38-α |
| 4H-1,2,4-triazole | p38-β |
| | MAPKAPK-3 |
| 3-(4-(2-methoxyphenyl)-5-(4-phenylbutylthio)-4H-1,2,4-triazol-3-yl)-N,N-38-β | p38-α |
| dimethylbenzenamine | p38-β |
| | MAPKAPK-3 |
| 4-(4-(2-methoxyphenyl)-5-(4-phenylbutylthio)-4H-1,2,4-triazol-3-yl)pyridine | p38-α |
| | ROCK2 |
| | p38-β |
| | PKA |
| | MAPKAPK-3 |
| 4-(2-methoxyphenyl)-3-(4-phenylbutylthio)-5-(thiophen-2-ylmethyl)-4H- | p38-α |
| 1,2,4-tnazole | |
| 3-(furan-2-yl)-4-(2-methoxyphenyl)-5-(4-phenylbutylthio)-4H-1,2,4-triazole | p38-α |
| | MAPKAPK-3 |
| | p38-β |
| 3-(2-fluorobenzyl)-4-(2-methoxyphenyl)-5-(4-phenylbutylthio)-4H-1,2,4- | p38-α |
| triazole | |
| 3-((4-(2-methoxyphenyl)-5-(4-phenylbutylthio)-4H-1,2,4-triazol-3- | p38-α |
| yl)methyl)-1H-indole | |
| 3-(4-chlorophenyl)-4-(2-methoxyphenyl)-5-(4-phenylbutylthio)-4H-1,2,4- | p38-α |
| triazole | AURORA-A |
| 4-(2-methoxyphenyl)-3-(4-phenylbutylthio)-5-(3-(trifluoromethyl)phenyl)- | p38-α |
| 4H-1,2,4-triazole | |
| 4-(2-methoxyphenyl)-3-(4-phenylbutylthio)-5-m-tolyl-4H-1,2,4-triazole | p38-α |
| | p38-β |
| | MAPKAPK-3 |
| 4-(2-methoxyphenyl)-3-(4-phenylbutylthio)-5-p-tolyl-4H-1,2,4-triazole | p38-α |
| | p38-β |
| | MAPKAPK-3 |
| 4-(2-methoxyphenyl)-3-(4-phenylbutylthio)-5-(thiophen-3-yl)-4H-1,2,4- | p38-α |
| triazole | p38-β |
| | MAPKAPK-3 |
| 4-(2-methoxyphenyl)-3-(4-phenylbutylthio)-5-o-tolyl-4H-1,2,4-triazole | p38-α |
| | p38-β |
| 3,4-bis(2-methoxyphenyl)-5-(4-phenylbutylthio)-4H-1,2,4-triazole | p38-α |
| | p38-β |
| 4-(2-methoxyphenyl)-3-(3-methoxyphenyl)-5-(4-phenylbutylthio)-4H-1,2,4- | p38-α |
| triazole | p38-β |
| | MAPKALPK-3 |
| 2-(4-(2-methoxyphenyl)-5-(4-phenylbutylthio)-4H-1,2,4-triazol-3-yl)pyridine | p38-α |
| | p38-β |
| | MAPKAPK-3 |
| 3-(2-methoxyphenyl)-3-(4-phenylbutylthio)-5-(2-(trifluoromethoxy)phenyl)- | p38-α |
| 4H-1,2,4-triazole | |
| 4-(methoxymethyl)-4-(2-methoxyphenyl)-5-(4-phenylbutylthio)-4H-1,2,4- | p38-α |
| triazole | |
| 4-(4-(2-methoxyphenyl)-5-(4-phenylbutylthio)-4H-1,2,4-triazol-3-yl)-N,N- | p38-α |
| dimethylbenzenamine | p38-β |
| | MAPKAPK-3 |
| 3-(2-fluorophenyl)-4-(2-methoxyphenyl)-5-(4-phenylbutylthio)-4H-1,2,4- | p38-α |
| triazole | p38-β |
| | LYNA |
| | MAPKAPK-3 |
| 3-((4-fluorophenoxy)methyl)-4-(2-methoxyphenyl)-5-(4-phenylbutylthio)- | p38-α |
| 4H-1,2,4-triazole | |
| 3-(2,4-difluorophenyl)-4-(2-methoxyphenyl)-5-(4-phenylbutylthio)-4H-1,2,4- | p38-α |
| triazole | p38-β |
| 4-(2-methoxyphenyl)-3-(4-phenylbutylthio)-5-(4-(trifluoromethoxy)phenyl)- | p38-α |
| 4H-1,2,4-triazole | |
| 3-(3,4-difluorophenyl)-4-(2-methoxyphenyl)-5-(4-phenylbutylthio)-4H-1,2,4- | p38-α |
| triazole | AURORA-A |
| 1-(4-(2-methoxyphenyl)-5-(4-phenylbutylthio)-4H-1,2,4-triazol-3-yl)ethanol | p38-α |
| 3-(3-chloro-4-methylthiophen-2-yl)-4-(2-methoxyphenyl)-5-(4- | p38-α |
| phenylbutylthio)-4H-1,2,4-triazole | p38-β |
| | MAPKAPK-3 |
| 3-(3-methoxybenzyl)-4-(2-methoxyphenyl)-5-(4-phenylbutylthio)-4H-1,2,4- | p38-α |
| triazole | |
| 2-chloro-5-(4-(2-methoxyphenyl)-5-(4-phenylbutylthio)-4H-1,2,4-triazol-3- | p38-α |
| yl)pyridine | |
| 4-(2-methoxyphenyl)-3-(4-phenylbutylthio)-5-(thiophen-3-ylmethyl)-4H- | p38-α |
| 1,2,4-triazole | |
| 4-(2-methoxyphenyl)-3-(3-methyl-1H-pyrazol-5-yI)-5-(4-phenylbutylthio)- | p38-α |
| 4H-1,2,4-triazole | p38-β |
| | MAPKAPK-3 |
| 4-(2-methoxyphenyl)-3-(1-methyl-1H-imidazol-5-yl)-5-(4-phenylbutylthio)- | p38-α |
| 4H-1,2,4-triazole | p38-β |
| 2-((4-(2-methoxyphenyl)-5-(4-phenylbutylthio)-4H-1,2,4-triazol-3- | p38-α |
| yl)methylthio)-1H-benzo[d]imidazole | |
| 3-(4-(2-methoxyphenyl)-5-(4-phenylbutylthio)-4H-1,2,4-triazol-3- | p38-α |
| yl)propan-1-ol | |
| 4-(2-methoxyphenyl)-3-(4-phenylbutylthio)-5-(4-(trifluoromethyl)phenyl)- | p38-α |
| 4H-1,2,4-triazole | |
| (4-(2-methoxyphenyl)-5-(4-phenylbutylthio)-4H-1,2,4-triazol-3-yl)-N,N- | p38-α |
| dimethylmethanamine | |
| 4-(4-(2-methoxyphenyl)-5-(4-phenylbutylthio)-4H-1,2,4-triazol-3-yl)pyridin- | p38-α |
| 2-amine | p38-β |
| | ROCK2 |
| | KIT |
| | MAPKAPK-3 |
| 3-(3-(benzyloxy)phenyl)-4-(2-methoxyphenyl)-5-(4-phenylbutylthio)-4H- | p38-α |
| 1,2,4-triazole | |
| 4-(2-methoxyphenyl)-3-(4-phenylbutylthio)-5-(3-(trifluoromethoxy)phenyl)- | p38-α |
| 4H-1,2,4-triazole | |
| 4-(2-methoxyphenyl)-3-(3-phenoxyphenyl)-5-(4-phenylbutylthio)-4H-1,2,4- | p38-α |
| triazole | |
| 4-(2-methoxyphenyl)-3-(2-phenoxyphenyl)-5-(4-phenylbutylthio)-4H-1,2,4- | p38-α |
| triazole | |
| 4-(2-methoxyphenyl)-3-(4-phenylbutylthio)-5-(3,4,5-trimethoxyphenyl)-4H- | p38-α |
| 1,2,4-triazole | p38-β |
| | MAPKAPK-3 |
| 4-(2-methoxyphenyl)-3-(4-phenylbutylthio)-5-(2-(trifluoromethyl)phenyl)- | p38-α |
| 4H-1,2,4-triazole | p38-β |
| | MAPKAPK-3 |
| 4-(2-methoxyphenyl)-3-(naphthalen-2-yl)-5-(4-phenylbutylthio)-4H-1,2,4- | p38-α |
| triazole | MAPKAPK-3 |
| 3-(3-chloro-4-methylphenyl)-4-(2-methoxyphenyl)-5-(4-phenylbutylthio)- | p38-α |
| 4H-1,2,4-triazole | MAPKAPK-3 |
| 3-(4-(2-methoxyphenyl)-5-(4-phenylbutylthio)-4H-1,2,4-triazol-3-yl)-2- | p38-α |
| (methylthio)pyridine | p38-β |
| 3-(5-chlorothiophen-2-yl)-4-(2-methoxyphenyl)-5-(4-phenylbutylthio)-4H- | p38-α |
| 1,2,4-triazole | MAPKAPK-3 |
| | p38-β |
| 3-(3,5-difluorophenyl)-4-(2-methoxyphenyl)-5-(4-phenylbutylthio)-4H-1,2,4- | p38-α |
| triazole | |
| 4-(2-methoxyphenyl)-3-(5-methylthiophen-2-yl)-5-(4-phenylbutylthio)-4H- | p38-α |
| 1,2,4-triazole | p38-β |
| | MAPKAPK-3 |
| 4-(2-methoxyphenyl)-3-(4-phenylbutylthio)-5-propyl-4H-1,2,4-triazole | p38-α |
| 7-(4-(2-methoxyphenyl)-5-(4-phenylbutylthio)-4H-1,2,4-triazol-3-yl)-1H- | p38-α |
| indole | MAPKAPK-3 |
| 3-((2-chlorophenoxy)methyl)-4-(2-methoxyphenyl)-5-(4-phenylbutylthio)- | p38-α |
| 4H-1,2,4-triazole | |
| 4-(2-methoxyphenyl)-3-(4-phenylbutylthio)-5-(1H-pyrrol-2-yl)-4H-1,2,4- | p38-α |
| triazole | MAPKAPK-3 |
| | PIM-1-KINASE |
| 4-(4-(2-methoxyphenyl)-5-(4-phenylbutylthio)-4H-1,2,4-triazol-3-yl)thiazole | p38-α |
| | p38-β |
| | MAPKAPK-3 |
| 2-(4-(2-methoxyphenyl)-5-(4-phenylbutylthio)-4H-1,2,4-triazol-3-yl)oxazole | p38-α |
| | MAPKALPK-3 |
| 4-(2-methoxyphenyl)-3-methyl-5-(4-phenylbutylthio)-4H-1,2,4-triazole | p38-α |
| 3-(3-fluorophenyl)-4-(2-methoxyphenyl)-5-(4-phenylbutylthio)-4H-1,2,4- | p38-α |
| triazole | p38-β |
| | MAPKAPK-3 |
| 3-(3-chlorophenyl)-4-phenyl-5-(4-phenylbutylthio)-4H-1,2,4-triazole | p38-α |
| 3,4-bis(3-chlorophenyl)-5-(4-phenylbutylthio)-4H-1,2,4-triazole | p38-α |
| 3-(3-chlorophenyl)-4-(3-methoxyphenyl)-5-(4-phenylbutylthio)-4H-1,2,4- | p38-α |
| triazole | |
| 3-(3-chlorophenyl)-4-(2,4-dimethoxyphenyl)-5-(4-phenylbutylthio)-4H- | p38-α |
| 1,2,4-triazole | |
| 4-benzyl-3-(3-chlorophenyl)-5-(4-phenylbutylthio)-4H-1,2,4-triazole | p38-α |
| 3-(3-chlorophenyl)-4-(2-fluorophenyl)-5-(4-phenylbutylthio)-4H-1,2,4- | p38-α |
| triazole | AURORA-A |
| | MAPKAPK-3 |
| 3-(3-chlorophenyl)-5-(4-phenylbutylthio)-4-o-tolyl-4H-1,2,4-triazole | p38-α |
| | MAPKAPK-3 |
| | p38-β |
| 3-(3-chlorophenyl)-5-(4-phenylbutylthio)-4-m-tolyl-4H-1,2,4-triazole | p38-α |
| 3-(3-chlorophenyl)-4-(2,4-difluorophenyl)-5-(4-phenylbutylthio)-4H-1,2,4- | p38-α |
| triazole | |
| 3-(3-chlorophenyl)-4-cyclohexyl-5-(4-phenylbutylthio)-4H-1,2,4-triazole | p38-α |
| 4-(3-(3-chlorophenyl)-5-(4-phenylbutylthio)-4H-1,2,4-triazol-4-yl)-N,N- | p38-α |
| dimethylnaphthalen-1-amine | |
| 3-(3-chlorophenyl)-4-(fixran-2-ylmethyl)-5-(4-phenylbutylthio)-4H-1,2,4- | AURORA-A |
| triazole | p38-α |
| 3-(3-chlorophenyl)-5-(4-phenylbutylthio)-4-(1-phenylethyl)-4H-1,2,4-triazol | p38-α |
| 4-(3-(3-(3-chlorophenyl)-5-(4-phenylbutylthio)-4H-1,2,4-triazol-4- | p38-α |
| yl)propyl)morpholine | |
| 3-(3-(3-chlorophenyl)-5-(4-phenylbutylthio)-4H-1,2,4-triazol-4-yl)benzoic | p38-α |
| acid | |
| 3-(3-(3-chlorophenyl)-5-(4-phenylbutylthio)-4H-1,2,4-triazol-4-yl)pyridine | p38-α |
| 3-(3-chlorophenyl)-4-(2-methoxybenzyl)-5-(4-phenylbutylthio)-4H-1,2,4- | p38-α |
| triazole | |
| 3-(3-chlorophenyl)-4-(2-methoxyethyl)-5-(4-phenylbutylthio)-4H-1,2,4- | p38-α |
| triazole | |
| 3-(3-chlorophenyl)-4-(4-fluorobenzyl)-5-(4-phenylbutylthio)-4H-1,2,4- | p38-α |
| triazole | |
| 4-(benzo[d][1,3]dioxol-5-yl)-3-(3-chlorophenyl)-5-(4-phenylbutylthio)-4H- | AURORA-A |
| 1,2,4-triazole | p38-α |
| 4-(2-chlorophenyl)-3-(3-chlorophenyl)-5-(4-phenylbutylthio)-4H-1,2,4- | p38-α |
| triazole | MAPKAPK-3 |
| 3-(3-chlorophenyl)-4-isobutyl-5-(4-phenylbutylthio)-4H-1,2,4-triazole | p38-α |
| 3-(3-chlorophenyl)-5-(4-phenylbutylthio)-4-((tetrahydrofuran-2-yl)methyl)- | p38-α |
| 4H-1,2,4-triazole | |
| 3-(3-chlorophenyl)-4-(2-(difluoromethoxy)phenyl)-5-(4-phenylbutylthio)- | p38-α |
| 4H-1,2,4-triazole | p38-β |
| | MAPKAPK-3 |
| 4-(3-(3-chlorophenyl)-5-(4-phenylbutylthio)-4H-1,2,4-triazol-4-yl)-3,5- | p38-α |
| dimethylisoxazole | |
| 4-(3-(3-chlorophenyl)-5-(4-phenylbutylthio)-4H-1,2,4-triazol-4- | p38-α |
| yl)benzo[c][1,2,5]thiadiazole | MAPKAPK-3 |
| 4-(3-(3-chlorophenyl)-5-(4-phenylbutylthio)-4H-1,2,4-triazol-4-yl)benzoic | p38-α |
| acid | |
| 4-butyl-3-(3-chloroplienyl)-5-(4-phenylbutylthio)-4H-1,2,4-triazole | p38-α |
| 3-(3-chlorophenyl)-4-(3-fluorophenyl)-5-(4-phenylbutylthio)-4H-1,2,4- | p38-α |
| triazole | |
| 3-(3-chlorophenyl)-5-(4-phenylbutylthio)-4-(2-(trifluoromethoxy)phenyl)- | p38-α |
| 4H-1,2,4-triazole | MAPKAPK-3 |
| 3-(3-chlorophenyl)-5-(4-phenylbutylthio)-4-(2-(trifluoromethyl)phenyl)-4H- | p38-α |
| 1,2,4-triazole | MAPKAPK-3 |
| | p38-β |
| 1-(3-(3-(3-chlorophenyl)-5-(4-phenylbutylihio)-4H-1,2,4-triazol-4- | p38-α |
| yl)phenyl)ethanone | |
| 1-(4-(3-(3-chlorophenyl)-5-(4-phenylbutylthio)-4H-1,2,4-triazol-4- | INSR |
| yl)phenyl)ethanone | |
| 3-(3-chlorophenyl)-5-(4-phenylbutylthio)-4-(thiophen-2-ylmethyl)-4H-1,2,4- | p38-α |
| triazole | |
| 3-(3-chlorophenyl)-4-(3-methoxypropyl)-5-(4-phenylbutylthio)-4H-1,2,4- | p38-α |
| triazole | |
| 2-(5-(3-chlorophenyl)-4-(2-methoxyphenyl)-4H-1,2,4-triazol-3-ylthio)-1- | ROCK2 |
| phenylethanone | p38-α |
| 2-(5-(3-chlorophenyl)-4-(2-methoxyphenyl)-4H-1,2,4-triazol-3-ylthio)ethanol | ROCK2 |
| 4-((5-(3-chlorophenyl)-4-(2-methoxyphenyl)-4H-1,2,4-triazol-3- | AURORA-A |
| ylthio)methyl)benzoic acid | |
| 3-((5-(3-chlorophenyl)-4-(2-methoxyphenyl)-4H-1,2,4-triazol-3- | |
| ylthio)methyl)pyridine | |
| 3-((5-(3-chlorophenyl)-4-(2-methoxyphenyl)-4H-1,2,4-triazol-3- | p38-α |
| ylthio)methyl)benzonitrile | |
| 3-(3-chlorophenyl)-4-(2-methoxyphenyl)-5-(phenethylthio)-4H-1,2,4-triazole | p38-α |
| 5-(2-(5-(3-chlorophenyl)-4-(2-methoxyphenyl)-4H-1,2,4-triazol-3- | ROCK2 |
| ylthio)ethyl)morpholine | |
| 3-(3-chlorophenyl)-4-(2-methoxyphenyl)-5-(propylthio)-4H-1,2,4-triazole | p38-α |
| | ROCK2 |
| 2-(5-(3-chlorophenyl)-4-(2-methoxyphenyl)-4H-1,2,4-triazol-3-ylthio)-1-(5- | ROCK2 |
| chlorothiophen-2-yl)ethanone | |
| 3-(3-chlorophenyl)-5-(cyclopropylmethylthio)-4-(2-methoxyphenyl)-4H- | p38-α |
| 1,2,4-triazole | ROCK2 |
| 3-(3-chlorophenyl)-4-(2-methoxyphenyl)-5-(3-(trifluoromethyl)benzylthio)- | p38-α |
| 4H-1,2,4-triazole | |
| 2-(5-(3-chlorophenyl)-4-(2-methoxyphenyl)-4H-1,2,4-triazol-3- | PIM-1-KINASE |
| ylthio)ethanamine | |
| 3-(3-chlorophenyl)-4-(2-methoxyphenyl)-5-(4-methylpent-3-enylthio)-4H- | p38-α |
| 1,2,4-triazole | |
| 3-((5-(3-chlorophenyl)-4-(2-methoxyphenyl)-4H-1,2,4-triazol-3- | ROCK2 |
| ylthio)methyl)benzoic acid | |
| 4-(5-(3-chlorophenyl)-4-(2-methoxyphenyl)-4H-1,2,4-triazol-3-ylthio)-1- | CHEK2 |
| phenylbutan-1-one | p38-α |
| 3-(3-chlorophenyl)-5-(cinnamylthio)-4-(2-methoxyphenyl)-4H-1,2,4-triazole | p38-α |
| 3-(3-chlorophenyl)-4-(2-methoxyphenyl)-5-(2-phenoxyethylthio)-4H-1,2,4- | p38-α |
| triazole | |
| 3-(3-chlorophenyl)-5-((2,3-dihydrobenzo[b][1,4]dioxin-2-yl)methylthio)-4- | p38-α |
| (2-methoxyphenyl) N-4H-1,2,4-triazole | MAPKAPK-3 |
| 3-(3-chlorophenyl)-4-(2-methoxyphenyl)-5-(4-phenoxybutylthio)-4H-1,2,4- | p38-α |
| triazole | MAPKAPK-3 |
| 2-chloro-5-((5-(3-chlorophenyl)-4-(2-methoxyphenyl)-4H-1,2,4-triazol-3- | ROCK2 |
| ylthio)methyl)pyridine | |
| 2-(5-(3-chlorophenyl)-4-(2-methoxyphenyl)-4H-1,2,4-triazol-3-ylthio)-1- | ROCK2 |
| (2,3-dihydrobenzo[b][1,4]dioxin-6-yl)ethanone | |
| 3-(2-(1H-pyrrol-1-yl)ethylthio)-5-(3-chlorophenyl)-4-(2-methoxyphenyl)-4H- | ROCK2 |
| 1,2,4-triazole | |
| 1-(5-(3-chlorophenyl)-4-(2-methoxyphenyl)-4H-1,2,4-triazol-3-ylthio)butan- | ROCK2 |
| 2-ol | |
| 4-(2-(5-(3-chlorophenyl)-4-(2-methoxyphenyl)-4H-1,2,4-triazol-3- | p38-α |
| ylthio)ethyl)benzoic acid | |
| 3-(3-chlorophenyl)-5-(3,4-difluorobenzyltbio)-4-(2-methoxyphenyl)-4H- | ROCK2 |
| 1,2,4-triazole | |
| 3-(3-chlorophenyl)-5-(3-fluorophenethylthio)-4-(2-methoxyphenyl)-4H- | p38-α |
| 1,2,4-triazole | |
| 4-((5-(3-chlorophenyl)-4-(2-methoxyphenyl)-4H-1,2,4-triazol-3- | p38-α |
| ylthio)methyl)pyridine | 38-β |
| 2-(5-(3-chlorophenyl)-4-(2-methoxyphenyl)-4H-1,2,4-triazol-3- | ROCK2 |
| ylthio)acetonitrile | p38-β |
| 2-(5-(3-chlorophenyl)-4-(2-methoxyphenyl)-4H-1,2,4-triazol-3-ylthio)-N- | ROCK2 |
| phenylacetamide | |
| 2-(5-(3-chlorophenyl)-4-(2-methoxyphenyl)-4H-1,2,4-triazol-3- | PIM-1-KINASE |
| ylthio)acetimidamide | ROCK2 |
| | ATK1 |
| | RSK2 |
| p38-α | |
| 4-((5-(3-chlorophenyl)-4-(2-methoxyphenyl)-4H-1,2,4-triazol-3- | ROCK2 |
| ylthio)methyl)-2-methylthiazole | |
| 4-(5-(3-chlorophenyl)-4-(2-methoxyphenyl)-4H-1,2,4-triazol-3-ylthio)-1- | p38-α |
| (thiophen-2-yl)butan-1-one | |
| 3-((5-(3-chlorophenyl)-4-(2-methoxyphenyl)-4H-1,2,4-triazol-3- | p38-α |
| ylthio)methyl)-5-(4-methoxyphenyl)-1,2,4-oxadiazole | |
| 2-(5-(3-chlorophenyl)-4-(2-methoxyphenyl)-4H-1,2,4-triazol-3-ylthio)-1- | ROCK2 |
| phenylethanol | |
| 3-(3-chlorophenyl)-4-(2-methoxyphenyl)-5-(2-(phenylthio)ethylthio)-4H- | p38-α |
| 1,2,4-triazole | p38-β |
| | MAPKAPK-3 |
| | ROCK2 |
| 1-(3-(5-(3-chlorophenyl)-4-(2-methoxyphenyl)-4H-1,2,4-triazol-3- | p38-α |
| ylthio)propyl)-1H-benzo[d]imidazol-2(3H)-one | |
| 1-(4-(2-(5-(3-chlorophenyl)-4-(2-methoxyphenyl)-4H-1,2,4-triazol-3- | ROCK2 |
| ylthio)ethyl)phenyl)ethanone | p38-α |
| N-allyl-2-(5-(3-chlorophenyl)-4-(2-methoxyphenyl)-4H-1,2,4-triazol-3- | p38-α |
| ylthio)acetamide | |
| 2-(5-(3-chlorophenyl)-4-(2-methoxyphenyl)-4H-1,2,4-triazol-3-ylthio)-1-(4- | ROCK2 |
| fluorophenyl)ethanone | |
| 3-(3-chlorophenyl)-5-(4-fluorophenethylthio)-4-(2-methoxyphenyl)- | p38-α |
| 1,2,4-triazole | |
| 3-(3-chlorophenyl)-5-(3-(4-fluorophenoxy)propylthio)-4-(2-methoxyphenyl)- | p38-α |
| 4H-1,2,4-triazole | |
| 3-(3-chlorophenyl)-4-(2-methoxyphenyl)-5-(4,4,4-trifluorobutylthio)-4H- | ROCK2 |
| 1,2,4-triazole | p38-α |
| 4-(5-(3-chlorophenyl)-4-(2-methoxyphenyl)-4H-1,2,4-triazol-3-ylthio)-1-(4- | p38-α |
| fluorophenyl)butan-1-one | |
| 2-(5-(3-chlorophenyl)-4-(2-methoxyphenyl)-4H-1,2,4-triazol-3-ylthio)-2,3- | ROCK2 |
| dihydroinden-1-one | |
| 1-(4-chlorophenyl)-4-(5-(3-chlorophenyl)-4-(2-methoxyphenyl)-4H-1,2,4- | p38-α |
| triazol-3-ylthio)butan-1-one | |
| 3-(4-(2-methoxyphenyl)-5-(4-phenylbutylthio)-4H-1,2,4-triazol-3-yl)pyrazin- | p38-α |
| 2-amine | GSK-3-β |
| | GSK-3-α |
| | AURORA-A |
| | p38-β |
| N-((4-(2-methoxyphenyl)-5-(4-phenylbutylthio)-4H-1,2,4-triazol-3- | p38-α |
| yl)methyl)furan-2-carboxamide | |
| 5-(4-(2-methoxyphenyl)-5-(4-phenylbutylthio)-4H-1,2,4-triazol-3-yl)-2- | p38-α |
| methylpyridine | p38-β |
| | MAPKAPK-3 |
| 2-((4-(2-methoxyphenyl)-5-(4-phenylbutylthio)-4H-1,2,4-triazol-3- | p38-α |
| yl)methyl)pyridine | |
| 3-(1H-imidazol-4-yl)-4-(2-methoxyphenyl)-5-(4-phenylbutylthio)-4H-1,2,4- | p38-α |
| triazole | p38-β |
| | MAPKAPK-3 |
| 2-((4-(2-methoxyphenyl)-5-(4-phenylbutylthio)-4H-1,2,4-triazol-3- | p38-α |
| yl)methyl)-2H-benzo[b][1,4]oxazin-3(4H)-one | |
| 2-chloro-4-(4-(2-methoxyphenyl)-5-(4-phenylbutylthio)-4H-1,2,4-triazol-3- | p38-α |
| yl)pyridine | MAPKAPK-3 |
| 5-(4-(2-methoxyphenyl)-5-(4-phenylbutylthio)-41-I-1,2,4-triazol-3-yl)pyridin- | p38-α |
| 2-amine | p38-β |
| | MAPKAPK-3 |
| 5-(4-(2-methoxyphenyl)-5-(4-phenylbutylthio)-4H-1,2,4-triazol-3-yl)pyridin- | p38-α |
| N-amine | |
| 5-(4-(2-methoxyphenyl)-5-(4-phenylbutylthio)-4H-1,2,4-triazol-3-yl)-1H- | p38-α |
| benzo[d]imidazole | p38-β |
| | MAPKAPK-3 |
| | KIT |
| 2-(4-(2-methoxyphenyl)-5-(4-phenylbutylthio)-4H-1,2,4-triazol-3-yl)-5- | p38-α |
| methylpyrazine | |
| 5-(4-(2-methoxyphenyl)-5-(4-phenylbutylthio)-4H-1,2,4-triazol-3-yl)-2- | p38-α |
| trifluoromethyl)pyridine | AMP-KINASE |
| 3-(4-(2-methoxyphenyl)-5-(4-phenylbutylthio)-4H-1,2,4-triazol-3-yl)-5- | p38-α |
| methylisoxazole | MAPKAPK-3 |
| 2-methoxy-5-(4-(2-methoxyphenyl)-5-(4-phenylbutylthio)-4H-1,2,4-triazol- | p38-α |
| 3-yl)pyridine | MAPKAPK-3 |
| | p38-β |
| 3-((furan-2-ylmethylthio)methyl)-4-(2-methoxyphenyl)-5-(4- | p38-α |
| phenylbutylthio)-4H-1,2,4-triazole | AURORA-A |
| 6-(4-(2-methoxyphenyl)-5-(4-phenylbutylthio)-4H-1,2,4-triazol-3- | p38-α |
| yl)quinoxaline | MAPKAPK-3 |
| 3-(4-(2-methoxyphenyl)-5-(4-phenylbutylthio)-4H-1,2,4-triazol-3- | p38-α |
| yl)isoquinoline | AURORA-A |
| 3-(2-(4-(2-methoxyphenyl)-5-(4-phenylbutylthio)-4H-1,2,4-triazol-3- | p38-α |
| yl)ethyl)pyridine | |
| N-(2-(4-(2-methoxyphenyl)-5-(4-phenylbutylthio)-4H-1,2,4-triazol-3- | p38-α |
| yl)thiophen-3-yl)acetamide | MAPKAPK-3 |
| | p38-β |
| 3-(4-(2-methoxyphenyl)-5-(4-phenylbutylthio)-4H-1,2,4-triazol-3-yl)pyridin- | p38-α |
| 2(1H)-one | 38-β |
| N-(3-(4-(2-methoxyphenyl)-5-(4-phenylbutylthio)-4H-1,2,4-triazol-3- | p38-α |
| yl)propyl)pyrimidin-2-amine | |
| 5-(4-(2-methoxyphenyl)-5-(4-phenylbutylthio)-4H-1,2,4-triazol-3-yl)pyridin- | p38-α |
| 2(1H)-one | |
| 2-(4-(2-mcthoxyphenyl)-5-(4-phenylbutylthio)-4H-1,2,4-triazol-3- | p38-α |
| yl)quinolin-4(1H)-one | |
| 3-(4-(2-methoxyphenyl)-5-(4-phenylbutylthio)-4H-1,2,4-triazol-3-yl)-N- | p38-α |
| methylpyrazin-2-amine | |
| 4-(2-methoxyphenyl)-5-(4-phenylbutylthio)-3,3′-bi(4H-1,2,4-triazole) | p38-α |
| 2-(4-(2-methoxyphenyl)-5-(4-phenylbutylthio)-4H-1,2,4-triazol-3-yl)indoline | p38-α |
| 2-(4-(2-methoxyphenyl)-5-(4-phenylbutylthio)-4H-1,2,4-triazol-3-yl)-1,2,3,4- | p38-α |
| tetrahydroquinoline | |
| 4-(2-methoxyphenyl)-3-(4-methoxythiophen-3-yl)-5-(4-phenylbutylthio)-4H- | p38-α |
| 1,2,4-triazole | p38-β |
| | MAPKAPK-3 |
| 2-(4-(2-methoxyphenyl)-5-(4-phenylbutylthio)-4H-1,2,4-triazol-3-yl)-1H- | p38-α |
| indole | |
| 2-(3-(3-chlorophenyl)-5-(4-phenylbutoxy)-4H-1,2,4-triazol-4-yl)phenol | p38-α |
| 3-(4-fluorophenyl)-4-(2-methoxyphenyl)-5-(5-phenylpentyloxy)-4H-1,2,4- | p38-α |
| triazole | |
| 4-(2-methoxyphenyl)-3-(5-phenylpentyloxy)-5-(thiophen-2-yl)-4H-1,2,4- | p38-α |
| triazole | |
| 2-(4-(2-methoxyphenyl)-5-(5-phenylpentyloxy)-4H-1,2,4-triazol-3- | p38-α |
| yl)pyridine | |
| 3-(4-fluorophenyl)-4-(2-methoxyphenyl)-5-(4-phenylbutoxy)-4H-1,2,4- | p38-α |
| triazole | |
| 4-(2-methoxyphenyl)-3-(4-phenylbutoxy)-5-(thiophen-2-yl)-4H-1,2,4-triazol | p38-α |
| 2-(4-(2-methoxyphenyl)-5-(4-phenylbutoxy)-4H-1,2,4-triazol-3-yl)pyridine | p38-α |
| 3-(4-fluorophenyl)-4-(2-methoxyphenyl)-5-(4-phenoxybutoxy)-4H-1,2,4- | p38-α |
| triazole | |
| 4-(2-methoxyphenyl)-3-(4-phenoxybutoxy)-5-(thiophen-2-yl)-4H-1,2,4- | p38-α |
| triazole | |
| 3-(3-chlorophenyl)-4-(2-methoxyphenyl)-5-(4-phenoxybutoxy)-4H-1,2,4- | p38-α |
| triazole | |
| 2-(4-(2-methoxyphenyl)-5-(4-phenoxybutoxy)-4H-1,2,4-triazol-3-yl)pyridine | p38-α |
| 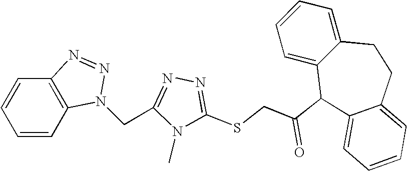 | MAPKAPK-2 |
| 2-(3-(3-chlorophenyl)-5-(4-phenylbutoxy)-4H-1,2,4-triazol-4-yl)phenol | p38-a |
| 3-(4-fluorophenyl)-4-(2-methoxyphenyl)-5-(5-phenylpentyloxy)-4H-1,2,4- | p38-a |
| triazole | |
| 4-(2-methoxyphenyl)-3-(5-phenylpentyloxy)-5-(thiophen-2-yl)-4H-1,2,4- | p38-a |
| triazole | |
| 2-(4-(2-methoxyphenyl)-5-(5-phenylpentyloxy)-4H-1,2,4-triazol-3- | p38-a |
| yl)pyridine | |
| 3-(4-fluorophenyl)-4-(2-methoxyphenyl)-5-(4-phenylbutoxy)-4H-1,2,4- | p38-a |
| triazole | |
| 4-(2-methoxyphenyl)-3-(4-phenylbutoxy)-5-(thiophen-2-yI)-4H-1,2,4-triazole | p38-a |
| 2-(4-(2-methoxyphenyl)-5-(4-phenylbutoxy)-4H-1,2,4-triazol-3-yl)pyridine | p38-a |
| 3-(4-fluorophenyl)-4-(2-methoxyphenyl)-5-(4-phenoxybutoxy)-4H-1,2,4- | p38-a |
| triazole | |
| 4-(2-methoxyphenyl)-3-(4-phenoxybutoxy)-5-(thiophen-2-yl)-4H-1,2,4- | p38-a |
| triazole | |
| 3-(3-chlorophenyl)-4-(2-methoxyphenyl)-5-(4-phenoxybutoxy)-4H-1,2,4- | p38-a |
| triazole | |
| 2-(4-(2-methoxyphenyl)-5-(4-phenoxybutoxy)-4H-1,2,4-triazol-3-yI)pyridine | p38-a |
| 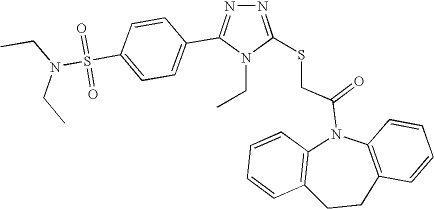 | DAPK1 |
| 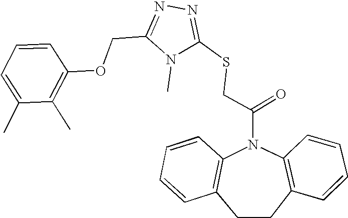 | MSK2 |
| 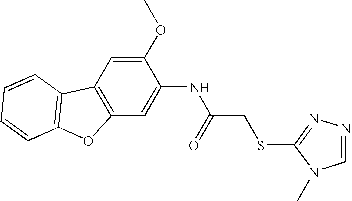 | CHEK2 INSR AURORA-A |
| 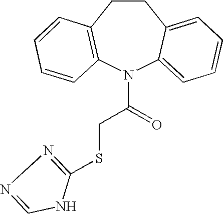 | LCK |

Other embodiments of the present disclosure will be apparent to those skilled in the art from consideration of the specification and practice of the present disclosure disclosed herein. It is intended that the specification and examples be considered as exemplary only, with a true scope and spirit of the present disclosure being indicated by the following claims.

1. At least one chemical entity chosen from compounds of Formula I, 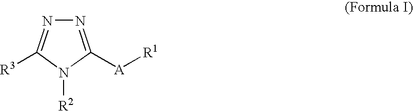
and pharmaceutically acceptable salts, solvates, crystal forms, chelates, non-covalent complexes, and prodrugs thereof, wherein:
A is chosen from S, O, and —NR¹⁷— wherein R¹⁷ is chosen from hydrogen, alkyl, substituted alkyl, cycloalkyl, and substituted cycloalkyl;
R¹ is chosen from —(CR⁴R⁵)ₙQ, wherein
n is an integer chosen from 0 to 8;
each R⁴ and R⁵ is independently chosen from hydrogen, hydroxy, alkyl, and substituted alkyl;
Q is chosen from hydrogen, sulfanyl, sulfonyl, alkoxy, substituted alkyl, optionally substituted amino, —CN, —SCN, —C(O)Z, alkenyl, substituted alkenyl, cycloalkyl, substituted cycloalkyl, aryl, substituted aryl, heterocycloalkyl, substituted heterocycloalkyl, heteroaryl, and substituted heteroaryl, wherein
Z is chosen from —OR¹⁰, —R¹¹, —NR¹²R¹³, and —NHNHY, wherein
R¹⁰ is chosen from hydrogen, alkyl, substituted alkyl, cycloalkyl, substituted cycloalkyl, aryl, substituted aryl, heterocycloalkyl, substituted heterocycloalkyl, heteroaryl, and substituted heteroaryl;
R¹¹ is chosen from alkyl, substituted alkyl, aryl, substituted aryl, heteroaryl, and substituted heteroaryl;
R¹² is chosen from hydrogen, alkyl, substituted alkyl, cycloalkyl, substituted cycloalkyl, aryl, and substituted aryl;
R¹³ is chosen from hydrogen, alkyl, substituted alkyl, alkenyl, substituted alkenyl, cycloalkyl, substituted cycloalkyl, aryl, substituted aryl, heteroaryl, and substituted heteroaryl;
or optionally R¹² and R¹³ together with the nitrogen atom to which R¹² and R¹³ are attached form a 5 to 7 member unsubstituted heterocyclic ring, or a 5 to 7 member substituted heterocyclic ring; and
Y is chosen from hydrogen and —C(O)R¹⁶, wherein
R¹⁶ is chosen from alkyl, substituted alkyl, cycloalkyl, substituted cycloalkyl, aryl, and substituted aryl;
R² is chosen from hydrogen, alkyl, substituted alkyl, alkenyl, substituted alkenyl, cycloalkyl, substituted cycloalkyl, aryl, substituted aryl, heterocycloalkyl, substituted heterocycloalkyl, heteroaryl, substituted heteroaryl, and —NH₂; and
R³ is chosen from hydrogen, alkyl, substituted alkyl, alkenyl, substituted alkenyl, cycloalkyl, substituted cycloalkyl, heterocycloalkyl, substituted heterocycloalkyl, aryl, substituted aryl, heteroaryl, and substituted heteroaryl,
provided that
when A is S, R¹ is not chosen from SCN, an aminopyridopyrimidine derivative, dopamine derivative, a dopa derivative, quinazoline derivative, a quinazolinone derivative, a benzoquinoxaline derivative, a phthalazine derivative, a pyrimidinyl derivative, a fused pyrimidine derivative, substituted pyridinyl and substituted aryl wherein the substituent on the substituted aryl is chosen from ether-, thio-, or amino-substituted groups, wherein the substituent is a 3-cyanoquinoline or aromatic tricyclic derivative;
when A is S, R² is not chosen from substituted alkenyl, wherein the substituent is chosen from an indolinone derivative,
when A is S, R³ is not chosen from substituted diarylamine and 1,2,3-triazole derivatives;
when A is S, R¹ is —(CR⁴R⁵)ₙQ, R² is H and R³ is hydrogen, then Q is not chosen from substituted alkyl, wherein the substituent is chosen from an amidothioxanthene, an alkylthioxanthene ether, a carbazole derivative, and a quinazolinone derivative;
when A is S, R¹ is —(CR⁴R⁵)ₙQ, Q is not chosen from substituted arylalkyl wherein the substituent on the arylalkyl group is chosen from an aminopyridopyrimidine derivative; substituted alkyl wherein the substituent on the alkyl group is a quinazolinone derivative; substituted heteroarylalkyl and substituted arylalkyl, wherein the substituent on the substituted heteroarylalkyl and on the substituted arylalkyl is chosen from ether, thio, and amino; 3-cyanoquinoline, an aromatic tricyclic derivative; a 3-substituted phenyl group wherein the 3-substituent is chosen from —C(O)NH and —NHCO; an indolocarbazole derivative; substituted pyridinyl, pyrimidinyl, and phenyl wherein the substituent is chosen from ether, thio, and amino, wherein the substituent is chosen from a 3-cyanoquinoline derivative and an aromatic tricyclic derivative; a phthalazine derivative; and substituted cycloheteroalkyl and substituted cycloheteroalkylalkyl, wherein the substituent is chosen from a phenylaminopyridopyrimidine derivative and an indolocarbazole derivative;
when A is S, Q is —C(O)Z, Z is —R¹¹, and R² and R³ are phenyl, then R¹¹ is not α-benzeneacetonitrile;
when A is S, Q is —C(O)Z, Z is —NR¹²R¹³, R² and R³ are phenyl, and R¹² is H; then R¹³ is not 2-benzoic acid methyl ester;
when A is S, Q is —C(O)Z, Z is —NR¹²R¹³, R² is 3-trifluoromethylphenyl, R³ is 4-methoxyphenyl, and R¹² is hydrogen, then R¹³ is not chosen from 4-cyclohexylphenyl and 4-benzoylphenyl;
when A is S, Q is —C(O)Z, Z is —NR¹²R¹³, R² is phenyl, R³ is chosen from 4-[[(phenylamino) thioxomethyl]amino]phenyl and 4-chloro-2-methoxyphenyl, and R¹² is hydrogen, then R¹³ is not chosen from 4-benzoyl L-aspartic acid and 4-benzoyl L-glutamic acid;
when A is S, Q is —C(O)Z, Z is —NR¹²R¹³, R² is chosen from phenyl and 4-chlorophenyl, R³ is 4-[(1H-indol-3-ylmethylene)amino]phenyl, and R¹² is hydrogen, then R¹³ is not chosen from phenyl, 2-methylphenyl, 4-methoxyphenyl, 2-methoxyphenyl, 4-chlorophenyl, 3-chlorophenyl, and 3-nitrophenyl;
when A is S, Q is —C(O)Z, Z is —NR¹²R¹³ and R², R³, and R¹² are hydrogen, then R¹³ is not chosen from a thioxanthene derivative;
when A is O, R² is chosen from alkyl and substituted alkyl, then R¹ is not chosen from alkyl-G¹, wherein G¹ is chosen from a phenyl-substituted oxadiazolyl and phenyl-substituted isoxazolyl;
when A is O, R³ is chosen from substituted imidazo[1,2-a]pyridyl, and R² is methyl; then R¹ is not methyl;
when A is O, R² is chosen from aryl, and R³ is biphenyl, then R¹ is not methyl;
when A is O, R³ is chosen from alkyl, alkenyl, and cycloalkyl, and R² is chosen from phenyl and pyridyl, then R¹ is not N-benzylpiperidin-4-yl-methyl;
when A is O, R³ is chosen from 4-heteroarylmethoxy-phenyl, and R² is methyl, then R¹ is not chosen from methyl and trifluoromethyl;
when A is O, R³ is chosen from aryl and heteroaryl, and R² is chosen from alkyl and cycloalkyl, then R¹ is not chosen from alkylene-B—Ar², wherein B is chosen from piperidinyl, piperazinyl, and tetrahydropyridinyl, and Ar² is chosen from phenyl, pyridyl, pyrimidinyl, and triazinyl;
when A is O, R³ is chosen from phenyl and pentafluoroethyl, and R² is methyl, then R¹ is not 4-(N-sulfonamido) phenyl;
when A is 0, R³ is trifluoromethyl, and R² is 2-biphenyl, then R¹ is not methoxymethyl;
when A is 0, R³ is N-sulfonamido-substituted phenyl, and R² is chosen from hydrogen, alkyl, and substituted alkyl, then R¹ is not chosen from alkyl, substituted alkyl, phenyl, and benzyl;
when A is O, R³ is n-butyl, and R² is 2′-tetrazolyl-4-biphenylmethyl, then R¹ is not chosen from benzyl and phenethyl;
when A is O, R³ is phenyl, and R² is chosen from n-propyl, tert-butyl, and phenyl, then R¹ is not chosen from —CH₂CO₂CH₂CH₃, —CH₂CONH₂NH₂, and CH₂-G², wherein G² is chosen from 1,2,4-triazole-3-thione, 1,3,4-oxadiazole-2-thione, and 1,24-triazolo[3,4b][1,3,4]thiadiazole;
when A is O, R³ is cyclohexyl, and R² is cyclohexyl, then R¹ is not methyl;
when A is O, R³ is phenyl, and R² is phenyl, then R¹ is not chosen from phenyl, substituted phenyl and methyl;
when A is O, R³ is 3-(4-biphenyloxycarbonyl)phenyl, and R² is n-butyl, then R¹ is not methyl;
when A is O, R³ is phenyl, and R² is methyl, then R¹ is not methyl;
when A is 0, R³ is methyl, and R² is methyl, then R¹ is not methyl;
when A is O, R³ is 2-furyl, and R² is methyl, then R¹ is not methyl;
when A is NR¹⁷, R³ is phenyl; and R² is chosen from phenyl and substituted phenyl, then R¹ and R¹⁷ are not both methyl;
when A is NR¹⁷, R³ is chosen from 2-hydroxyphenyl and 2-furyl and R² is phenyl, then R¹ and R¹⁷ are not both ethyl;
when A is NR¹⁷, R¹⁷ is hydrogen, R³ is 2-hydroxyphenyl, and R² is phenyl, then R¹ is not chosen from isopropyl, 4-(4-pyridinyl)butyl, and 3,4-dimethoxyphenethyl;
when A is NR¹⁷, R¹⁷ is H; R³ is chosen from 4-pyridyl and 4-pyrimidinyl, and R² is hydrogen, then R¹ is not chosen from —CH₂CONHG³, wherein G³ is chosen from aryl and heteroaryl;
when A is NR¹⁷, R¹⁷ is hydrogen, R³ is hydrogen, and R² is methyl, then R¹ is not 3-[2-(dimethylamino)ethyl]-1H-indol-5-ylmethyl; and
when A is NR¹⁷, then the compound is not chosen from 1-(3-Amino-[1,2,4]triazol-4-yl)-2-(4-chloro-phenyl)-ethanone and 5-(2-Methoxy-phenyl)-4H[1,2,4]triazol-3-ylamine.

2. At least one chemical entity of claim 1 wherein A is S.

3. At least one chemical entity of claim 1 wherein n is 0.

4. At least one chemical entity of claim 3 wherein Q is H.

5. At least one chemical entity of claim 3 wherein Q is substituted heteroaryl.

6. At least one chemical entity of claim 5 wherein Q is chosen from 5-bromo-2-phenyl-2H-pyridazin-3-one-4-yl, 2-hydroxy-4-phenyl-quinolin-3-yl, and 8-nitro-quinolin-5-yl.

7. At least one chemical entity of claim 1 wherein n is 1.

8. At least one chemical entity of claim 7 wherein Q is —SCN.

9. At least one chemical entity of claim 8 wherein Q is —CN.

10. At least one chemical entity of claim 7 wherein Q is —C(O)Z wherein Z is —NHNHY.

11. At least one chemical entity of claim 10 wherein Y is —C(O)R¹⁶ wherein R¹⁶ is chosen from cyclohexyl, aryl, substituted aryl, arylalkyl, and substituted arylalkyl.

12. At least one chemical entity of claim 11 wherein R¹⁶ is chosen from benzyl and substituted phenyl wherein the phenyl is substituted with one, two, or three groups chosen from hydroxy, lower alkoxy, halo, and lower alkyl.

13. At least one chemical entity of claim 7 wherein Q is chosen from aryl, substituted aryl, heteroaryl, and substituted heteroaryl.

14. At least one chemical entity of claim 13 wherein Q is chosen from phenyl and phenyl substituted with one or two groups chosen from nitro, halo, lower alkyl, carboxy, cyano, alkoxycarbonyl, sulfonyl, lower alkoxy, trifluoromethyl, trifluoromethoxy, and difluoromethoxy.

15. At least one chemical entity of claim 7 wherein Q is chosen from —C(O)Z wherein Z is —OR¹⁰.

16. At least one chemical entity of claim 15 wherein R¹⁰ is chosen from hydrogen, lower alkyl, benzyl, phenethyl, substituted benzyl, and substituted phenethyl, wherein the phenyl group of the substituted benzyl and substituted phenethyl is independently substituted with one or two groups chosen from halo, lower alkyl, lower alkoxy, and hydroxy.

17. At least one chemical entity of claim 7 wherein Q is chosen from —C(O)Z wherein Z is R¹¹ and R¹¹ is chosen from heteroaryl, substituted heteroaryl, phenyl, and substituted phenyl.

18. At least one chemical entity of claim 17 wherein R¹¹ is is chosen from phenyl, 2,3-dihydrobenzo[b][1,4]dioxin-6-yl, benzo[d][1,3]dioxol-5-yl, and phenyl substituted with one or two groups chosen from lower alkoxy, lower alkyl, halo, and hydroxy.

19. At least one chemical entity of claim 7 wherein Q is chosen from —C(O)Z wherein Z is —N¹²R¹³.

20. At least one chemical entity of claim 19 wherein R¹² is chosen from hydrogen and alkyl; and R¹³ is chosen from aryl, substituted aryl, arylalkyl, heteroarylalkyl, and substituted heteroarylalkyl.

21. At least one chemical entity of claim 20 wherein R¹² is hydrogen, and R¹³ is chosen from aryl, substituted aryl, heteroarylalkyl, and substituted heteroarylalkyl.

22. At least one chemical entity of claim 21, wherein R¹³ is chosen from hydrogen, methyl, ethyl, propyl, isopropyl, tert-butyl, butyl, methoxyethyl, 2-hydroxyethyl, 3-hydroxypropyl, propene-3-yl, phenyl, substituted phenyl, benzyl, substituted benzyl, substituted cyclohexyl, cyclopentyl, phenethyl, substituted phenethyl, cyclohexylmethyl, thiophen-2-ylmethyl, substituted [1,3,4]-thiadiazol-2-yl, 10,11-dihydro-5H-dibenzo[b,f]azepine-N-yl, morpholin-4-ylpropyl, morpholin-4-yl-ethyl, substituted benzothiazol-2-yl, substituted benzothiazol-5-yl, substituted propyl, furan-2-ylmethyl, tetrahydrofuran-2-yl-methyl, naphthalen-1-yl, thiazol-2-yl, substituted [1,3,4]thiadiazol-2-yl, 10H-phenothiazine-N-yl, 1,2,3,4-tetrahydroquinolin-1-yl, isoxazol-3-yl, substituted isoxazol-3-yl, 4,5,6,7-tetrahydrobenzothiazol-2-yl, substituted piperazin-1-yl, substituted piperidin-1-yl, substituted 5,6,-dihydro-4H-cyclopenta[b]thiophen-2-yl, 2-thiophen-2-ylmethyl, 3,4-methylenedioxyphenyl, substituted thiophen-2-yl, (3,4-methylenedioxyphenyl)methyl, substituted dibenzofuran-3-yl, 4,5,6,7-tetrahydro-benzo[b]thiophen-2-yl, —NHCOCH₂CH₃, 3-(furan-2-yl-carbonylamino)phenyl, and 3-(furan-2-yl-carbonylamino)-6-methylphenyl.

23. At least one chemical entity of claim 19 wherein R¹² and R¹³ together with the nitrogen atom to which R¹² and R¹³ are attached form a heterocyclic ring or substituted heterocyclic ring, wherein the heterocyclic ring is chosen from morpholine, quinoline, pyrrolidone, pyrrolidine, substituted piperazine, 2,3-dihydro-1H-indole, piperidine, substituted pyridine, pyridine, substituted pyrazine, 10H-phenthiazine, azepane, 1,2,3,4,-tetrahydroisoquinoline, and 1,2,3,4-tetrahydroquinoline.

24. At least one chemical entity of claim 23, wherein the substituents on the substituted heterocyclic ring are independently chosen from halo, —NH₂, —OH, —CF₃, —CN, —NO₂, —COOH, methyl, ethyl, methoxy, ethoxy, propoxy, phenyl, —COCH₃, —COOCH₃, —COOCH₂CH₃, —CONH₂, —CH₂COOCH₂CH₃, —NHCO-tetrahydrofuran-2-yl, 2-hydroxyethyl, —NHCO-furan-2-yl, —NHCO-thiophen-2-yl, —NHCO-furan-2-yl, and 4-methoxyphenyl.

25. At least one chemical entity of claim 2 wherein n is chosen from 1 and 2.

26. At least one chemical entity of claim 25 wherein Q is chosen from hydrogen, heterocycloalkyl and substituted heterocycloalkyl.

27. At least one chemical entity of claim 26 wherein Q is chosen from hydrogen, piperidin-1-yl, morpholin-4-yl, cyclohexyl, pyrrolidin-1-yl, cyclopropyl, and tetrahydrofuran-2-yl.

28. At least one chemical entity of claim 2 wherein n is 2 and Q is chosen from —C(O)OR¹⁰ wherein R¹⁰ is chosen from hydrogen and lower alkyl.

29. At least one chemical entity of claim 2 wherein A is O.

30. At least one chemical entity of claim 29 wherein n is 1.

31. At least one chemical entity of claim 30 wherein Q is —C(O)Z wherein Z is —OR¹⁰.

32. At least one chemical entity of claim 31 wherein R¹⁰ is chosen from C₁₋₄ alkyl-phenyl.

33. At least one chemical entity of claim 32 wherein R¹⁰ is chosen from benzyl and phenethyl.

34. At least one chemical entity of claim 30 wherein Q is —C(O)Z wherein Z is —NR¹²R¹³.

35. At least one chemical entity of claim 34 wherein R¹² is hydrogen and R¹³ is chosen from furan-2-ylmethyl and substituted phenyl.

36. At least one chemical entity of claim 35 wherein the substituents on the substituted phenyl are chosen from hydroxy, halo, lower alkyl, and lower alkoxy.

37. At least one chemical entity of claim 29 wherein n is chosen from 3, 4, and 5.

38. At least one chemical entity of claim 37 wherein Q is chosen from phenyl and substituted phenyl.

39. At least one chemical entity of claim 38 wherein Q is phenyl.

40. At least one chemical entity of claim 1 wherein A is NR¹⁷.

41. At least one chemical entity of claim 40 wherein R¹⁷ is hydrogen.

42. At least one chemical entity of claim 40 wherein n is 0 and Q is hydrogen.

43. At least one chemical entity of claim 40 wherien n is 1.

44. At least one chemical entity of claim 43 wherein Q is —C(O)Z wherein Z is chosen from —OR¹⁰ and —NR¹²R¹³.

45. At least one chemical entity of claim 1 wherein R² is chosen from hydrogen, lower alkyl, substituted lower alkyl, alkenyl, cycloalkyl, substituted cycloalkyl, aryl, substituted aryl, heteroaryl, and substituted heteroaryl.

46. At least one chemical entity of claim 44 wherein R² is chosen from hydrogen, lower alkyl, substituted lower alkyl, alkenyl, cyclohexyl, phenyl, and substituted phenyl.

47. At least one chemical entity of claim 46 wherein R² is chosen from phenyl and phenyl substituted with one or two groups chosen from —OH, halo, —CN, carboxy, trifluoromethyl, trifluoromethoxy, C₁₋₈ alkyl, and C₁₋₈ alkoxy.

48. At least one chemical entity of claim 47 wherein R² is chosen from phenyl and phenyl substituted with one or two groups chosen from lower alkyl, lower alkoxy, halo, trifluoromethyl, and trifluoromethoxy.

49. At least one chemical entity of claim 45 wherein R² is chosen from hydrogen, methyl, ethyl, propyl, propen-3-yl, propen-2-yl, isobutyl, isobutene-3-yl, phenyl, 4-chlorophenyl-acetyl, benzyl, cyclohexyl, phenethyl, 1-propen-3-yl, 1-isobuten-3-yl, 2-methoxyethyl, 2-methoxypropyl, propyloxymethyl, pyridin-2-yl, pyridin-3-yl, tetrahydrofuran-2-yl-methyl, furan-2-ylmethyl, N-propen-3-yl-morpholine, amino, N,N-dimethylaminopropyl, phenyl, and substituted phenyl wherein the substituents are independently chosen from halo, methyl, trifluoromethyl, ethyl, cyclohexyl, —NH₂, carboxy, cyano, methoxy, ethoxy, methoxypropyl, benzyl, phenethyl, methoxyethyl, furan-2-ylmethyl, tetrahydrofuran-2-yl-methyl, furan-2-yl-ethyl, 3-cyclohexylmethyl-furan-2-yl, 1H-benzimidazol-2-yl-methyl, 3,4-methylenedioxyphenyl, and morpholin-4-yl-propyl.

50. At least one chemical entity of any one of claim 1 wherein R³ is chosen from hydrogen, substituted lower alkyl, cycloalkyl, substituted cycloalkyl, aryl, and substituted aryl.

51. At least one chemical entity of claim 50 where R³ is —CH₂X wherein X is chosen from aryl, heteroaryl, —OR⁶, —SR⁷, and —NR⁸R⁹, wherein
R⁶ is chosen from aryl, and substituted aryl;
R⁷ is chosen from heteroaryl, and substituted heteroaryl;
R⁸ is H; and
R⁹ is substituted aryl.

52. At least one chemical entity of claim 50 wherein R³ is chosen from aryl and aryl substituted with a group chosen from —OH, halo, —CN, —CF₃, C₁₋₈ alkyl, and C₁₋₈ alkoxy.

53. At least one chemical entity of claim 52 wherein R³ is chosen from phenyl and phenyl substituted with a group chosen from —OH, halo, —CN, —CF₃, C₁₋₈ alkyl, and C₁₋₈ alkoxy.

54. At least one chemical entity of claim 53, wherein R³ is chosen from phenyl and phenyl substituted with a group chosen from halo, —OH and C₁₋₈ alkoxy.

55. At least one chemical entity of claim 50 wherein R³ is hydrogen.

56. At least one chemical entity of claim 1 wherein R⁴ and R⁵ are independently chosen from hydrogen and lower alkyl.

57. At least one chemical entity of claim 56 wherein R⁴ and R⁵ are independently chosen from hydrogen and methyl.

58. At least one chemical entity of claim 57 wherein R⁴ and R⁵ are hydrogen.

59. At least one chemical entity of claim 1 wherein the compound of Formula I is chosen from any one of the compounds set forth in Tables 1, 2, and 3.

60. At least one chemical entity of claim 1 wherein the at least one chemical entity exhibits selective activity for one of the following protein kinases or pair of protein kinases: ABL1, AKT1, AKT2, AKT3, AURORA-A, c-TAK1, CDK1, CDK2/cyclinA, CDK2/cyclinE, CDK5, CHEK1, CHEK2, CSK, DAPK1, DYRK2, FLT-3, FYN, GSK3-α, GSK3-β, HCK, INSR, KIT, LCK, LYNA, MAPKAPK2, MAPKAPK3, MSK1, MSK2, p38-α, p38-β, p38-δ, p38-γ, P70S6K, PAK2, PDGFR-α, PAK1, PKA, PRAK, ROCK2, SGK1, SRC, SYK, PIM-1-kinase, PDK1, and RSK2.

61. A pharmaceutical composition comprising at least one chemical entity chosen from claim 1, and at least one pharmaceutically acceptable vehicle chosen from carriers, adjuvants, and excipients.

62. The pharmaceutical composition of claim 61, wherein the at least one chemical entity is present in an amount effective for the treatment in a patient of at least one disease chosen from Alzheimer's disease, stroke, diabetes, obesity, inflammation, and cancer.

63. A method of treating a patient having at least one disease responsive to inhibition of at least one ATP-utilizing enzyme comprising administering to the patient a therapeutically effective amount of at least one chemical entity chosen from claim 1.

64. The method of claim 63, wherein the disease is chosen from Alzheimer's disease, stroke, diabetes, obesity, inflammation, and cancer.

65. A method of inhibiting at least one ATP-utilizing enzyme in a subject comprising administering to the subject at least one chemical entity chosen from claim 1.

66. The method of claim 66, where the ATP-utilizing enzyme is a protein kinase.

67. The method of claim 67, wherein the ATP-utilizing enzyme is chosen from p38-α, p38-β, p38-δ, and p38-γ.

68. A method of inhibiting at least one ATP-utilizing enzyme comprising contacting the ATP-utilizing enzyme with at least one chemical entity chosen from claim 1.

69. The method of claim 68, where the ATP-utilizing enzyme is chosen from a protein kinase.

70. The method of claim 69, wherein the ATP-utilizing enzyme is chosen from p38-α, p38-β, p38-δ, and p38-γ.

71. A method of treating at least one disease regulated by at least one ATP-utilizing enzyme in a subject in need of such treatment comprising administering to the subject a therapeutically effective amount of at least one chemical entity of claim 1.

72. The method of claim 71, wherein the ATP-utilizing enzyme is chosen from a protein kinase.

73. The method of claim 72, wherein the protein kinase is chosen from ABL1, AKT1, AKT2, AKT3, AURORA-A, c-TAK1, CDK1, CDK2/cyclinA, CDK2/cyclinE, CDK5, CHEK1, CHEK2, CSK, DAPK1, DYRK2, FLT-3, FYN, GSK3-α, GSK3-β, HCK, INSR, KIT, LCK, LYNA, MAPKAPK2, MAPKAPK3, MSK1, MSK2, p38-α, p38-β, p38-δ, p38-γ, P70S6K, PAK2, PDGFR-α, PAK1, PKA, PRAK, ROCK2, SGK1, SRC, SYK, PIM-1-kinase, PDK1, and RSK2.

74. The method of claim 73, wherein the protein kinase is chosen from a AGC kinase, a CMGC kinase, a CAMK kinase, a TK kinase, and a STE kinase.

75. The method of claim 74, wherein the protein kinase is a AGC protein kinase chosen from AKT1, AKT2, AKT3, AURORA-A, MSK1, MSK2, P70S6K, PAK1, PKA, ROCK2, SGK1, PDK1, and RSK2.

76. The method of claim 74, wherein the protein kinase is a CMGC protein kinase chosen from CDK1, CDK2/cyclinA, CDK2/cyclinE, CDK5, DYRK2, GSK3-α, GSK3-β, p38-α, p38-β, p38-δ, and p38-γ.

77. The method of claim 74, wherein the protein kinase is a CAMK protein kinase chosen from DAPK1, MAPKAPK2, MAPKAPK3, CHEK1, CHEK2, PRAK, c-TAK1, and PIM-1-kinase.

78. The method of claim 74, wherein the protein kinase is a TK protein kinase chosen from ABL1, CSK, FLT3, FYN, HCK, INSR, KIT, LCK, PDGFRR-α, LYNA, SYK, and SRC.

79. The method of claim 74, wherein the protein kinase is a STE protein kinase chosen from PAK2.

80. The method of claim 76, wherein the protein kinase is a CMGC protein kinase chosen from p38-α, p38-β, p38-δ, and p38-γ.

81. (canceled)

82. (canceled)

83. (canceled)

84. (canceled)

85. (canceled)

86. (canceled)
